SEQUENCE LISTING (79 sequences)

SEQ ID NO: 1 - protein, 9 aa, Human immunodeficiency virus
RPQVPLRPM

SEQ ID NO: 2 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 3 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 4 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 5 - protein, 173 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 6 - protein, 189 aa, Homo sapiens
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKDSYSKDANDVITMDPKDNWSKDANDTLLLQLTNTSAYYMYLLLLLKSVVYFAIITCCLLGRTAFCCNGEKS

SEQ ID NO: 7 - protein, 153 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 8 - protein, 15 aa, synthetic construct
  REGION 1..15: note = Synthetic peptide
GGGGSGGGGSGGGGS

SEQ ID NO: 9 - protein, 5 aa, synthetic construct
  REGION 1..5: note = Synthetic peptide
GGGGS

SEQ ID NO: 10 - protein, 14 aa, synthetic construct
  REGION 1..14: note = Synthetic peptide
GGGGSGGGGSGGGG

SEQ ID NO: 11 - protein, 13 aa, synthetic construct
  REGION 1..13: note = Synthetic peptide
EFGAGLVLGGQFM

SEQ ID NO: 12 - protein, 21 aa, synthetic construct
  REGION 1..21: note = Synthetic peptide
  REGION 1..3: note = MISC_FEATURE - This region may or may not be present
GSGEGRGSLLTCGDVEENPGP

SEQ ID NO: 13 - protein, 22 aa, synthetic construct
  REGION 1..22: note = Synthetic peptide
  REGION 1..3: note = MISC_FEATURE - This region may or may not be present
GSGATNFSLLKQAGDVEENPGP

SEQ ID NO: 14 - protein, 23 aa, synthetic construct
  REGION 1..23: note = Synthetic peptide
  REGION 1..3: note = MISC_FEATURE - This region may or may not be present
GSGQCTNYALLKLAGDVESNPGP

SEQ ID NO: 15 - protein, 24 aa, synthetic construct
  REGION 1..24: note = Synthetic peptide
  REGION 1..3: note = MISC_FEATURE - This region may or may not be present
GSGVKQTLNFDLLKLAGDVESNPG

SEQ ID NO: 16 - DNA, 355 bp, synthetic construct
  misc_feature 1..355: note = Synthetic polynucleotide
  source 1..355: mol_type = other DNA
tatgctagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctctagtttcactttccctagtttcactttccctagtttcactttccctagtttcactttccctagtttcactttcccctcgaggatatcaagatctggcctcggcggccag

SEQ ID NO: 17 - DNA, 80 bp, Woodchuck hepatitis virus
  source 1..80: mol_type = unassigned DNA
gccacggcggaactcatcgccgcctgccttgcccgctgctggacaggggctcggctgttgggcactgacaattccgtggt

SEQ ID NO: 18 - DNA, 592 bp, Woodchuck hepatitis virus
  source 1..592: mol_type = unassigned DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 19 - DNA, 8904 bp, synthetic construct
  misc_feature 1..8904: note = Synthetic polynucleotide
  source 1..8904: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagttctagagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 20 - DNA, 8904 bp, synthetic construct
  misc_feature 1..8904: note = Synthetic polynucleotide
  source 1..8904: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcaagcgtgaggctccggtgcccgtcagtgggcagagcgcacatcgcccacagtccccgagaagttggggggaggggtcggcaattgatccggagcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagttctagagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacccgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgcttgcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 21 - DNA, 12037 bp, synthetic construct
  misc_feature 1..12037: note = Synthetic polynucleotide
  source 1..12037: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattcgccaccatgagcctcgggctcctgtgctgtggggccttttctctcctgtgggcaggaccggtggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtcggacgcccaacattcagtacttcggcgccgggacccggctctcagtgctggaggacctgaaaaacgtgttcccacccgaactagtcgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcgggagcggagccacgaacttctctctgttaaagcaagcaggagacgtggaagaaaaccccggtcctatgacacgagttagcttgctgtgggcagtcgtggtctccacctgtctcgagtccggcatgggtcaacagctgaatcagagtcctcaatctatgtttatccaggaaggagaagatgtctccatgaactgcacttcttcaagcatatttaacacctggctatggtacaagcaggaacctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggcacataccagaaagttacctttggaactggaacaaagctccaagtcatcccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgcgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgactcgagggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtctggaacaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttatgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggagggggctgtccccgtgagctcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 22 - DNA, 12031 bp, synthetic construct
  misc_feature 1..12031: note = Synthetic polynucleotide
  source 1..12031: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattcgccaccatgggacccaggctcctcttctgggcactgctttgtctcctcggaaccggtccggttgaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtcggacgcccaacattcagtacttcggcgccgggacccggctctcagtgctggaggacctgaaaaacgtgttcccacccgaggcggccgcgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcgggagcggagccacgaacttctctctgttaaagcaagcaggagacgtggaagaaaaccccggtcctatgaactcctctctggactttctaattctgatcttaatgtttggaggaactagtggtcaacagctgaatcagagtcctcaatctatgtttatccaggaaggagaagatgtctccatgaactgcacttcttcaagcatatttaacacctggctatggtacaagcaggaacctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggcacataccagaaagttacctttggaactggaacaaagctccaagtcatcccaaatatccagaaccctgaccctgccgtgtaccagctgagagactctaaatccagtgacaagtctgcatgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgactcgagggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtctggaacaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttatgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggagggggctgtccccgtgagctcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 23 - DNA, 12037 bp, synthetic construct
  misc_feature 1..12037: note = Synthetic polynucleotide
  source 1..12037: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattcgccaccatgggcacaaggttgttcttctatgtggccctttgtctcctgtggaccggtcacatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtcggacgcccaacattcagtacttcggcgccgggacccggctctcagtgctggaggacctgaaaaacgtgttcccacccgaggtcgctgtgtttgagccatcagaagcagagatctcccacacccaaaaggccacactagtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcgggagcggagccacgaacttctctctgttaaagcaagcaggagacgtggaagaaaaccccggtcctatggcaggcattcgagctttatttatgtacttgtggctgcagctggactgggtctcgagaggtcaacagctgaatcagagtcctcaatctatgtttatccaggaaggagaagatgtctccatgaactgcacttcttcaagcatatttaacacctggctatggtacaagcaggaacctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggcacataccagaaagttacctttggaactggaacaaagctccaagtcatcccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgcggccgcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgactcgagggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtctggaacaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttatgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggagggggctgtccccgtgagctcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 24 - DNA, 12037 bp, synthetic construct
  misc_feature 1..12037: note = Synthetic polynucleotide
  source 1..12037: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattcgccaccatgggaatcaggctcctgtgtcgtgtggccttttgtttcctggctgtaggactagtagaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtcggacgcccaacattcagtacttcggcgccgggacccggctctcagtgctggaggacctgaaaaacgtgttcccacccgaggtcgctgtgtttgagccatcagaagcagagatctcccacacccaaaaggccacactggcatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcgggagcggagccacgaacttctctctgttaaagcaagcaggagacgtggaagaaaaccccggtcctatgacacgagttagcttgctgtgggcagtcgtggtctccacctgtctcgagtccggcatgggtcaacagctgaatcagagtcctcaatctatgtttatccaggaaggagaagatgtctccatgaactgcacttcttcaagcatatttaacacctggctatggtacaagcaggaacctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggcacataccagaaagttacctttggaactggaacaaagctccaagtcatcccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgcggccgcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgactcgagggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtctggaacaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttatgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggagggggctgtccccgtgagctcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 25 - DNA, 12031 bp, synthetic construct
  misc_feature 1..12031: note = Synthetic polynucleotide
  source 1..12031: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattcgccaccatggcctccctgctcttcttctgtggggccttttatctcctgggaaccggttccatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtcggacgcccaacattcagtacttcggcgccgggacccggctctcagtgctggaggacctgaaaaacgtgttcccacccgaggtcgctgtgtttgagccatcagaagcagagatctcccacacccaaaaggccacactagtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcgggagcggagccacgaacttctctctgttaaagcaagcaggagacgtggaagaaaaccccggtcctatgaactcctctctggactttctaattctgatcttaatgtttggaggaactagtggtcaacagctgaatcagagtcctcaatctatgtttatccaggaaggagaagatgtctccatgaactgcacttcttcaagcatatttaacacctggctatggtacaagcaggaacctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggcacataccagaaagttacctttggaactggaacaaagctccaagtcatcccaaatatccagaaccctgaccctgccgtgtaccagctgagagactctaaatccagtgacaagtctgcatgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgactcgagggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtctggaacaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttatgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggagggggctgtccccgtgagctcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 26 - DNA, 9961 bp, synthetic construct
  misc_feature 1..9961: note = Synthetic polynucleotide
  source 1..9961: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattcgccaccatgagcctcgggctcctgtgctgtggggccttttctctcctgtgggcaggaccggtggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtcggacgcccaacattcagtacttcggcgccgggacccggctctcagtgctggaggacctgaaaaacgtgttcccacccgaactagtcgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcgggagcggagccacgaacttctctctgttaaagcaagcaggagacgtggaagaaaaccccggtcctatgacacgagttagcttgctgtgggcagtcgtggtctccacctgtctcgagtccggcatgggtcaacagctgaatcagagtcctcaatctatgtttatccaggaaggagaagatgtctccatgaactgcacttcttcaagcatatttaacacctggctatggtacaagcaggaacctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggcacataccagaaagttacctttggaactggaacaaagctccaagtcatcccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgcgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgactcgagggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 27 - DNA, 9955 bp, synthetic construct
  misc_feature 1..9955: note = Synthetic polynucleotide
  source 1..9955: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattcgccaccatgggacccaggctcctcttctgggcactgctttgtctcctcggaaccggtccggttgaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtcggacgcccaacattcagtacttcggcgccgggacccggctctcagtgctggaggacctgaaaaacgtgttcccacccgaggcggccgcgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcgggagcggagccacgaacttctctctgttaaagcaagcaggagacgtggaagaaaaccccggtcctatgaactcctctctggactttctaattctgatcttaatgtttggaggaactagtggtcaacagctgaatcagagtcctcaatctatgtttatccaggaaggagaagatgtctccatgaactgcacttcttcaagcatatttaacacctggctatggtacaagcaggaacctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggcacataccagaaagttacctttggaactggaacaaagctccaagtcatcccaaatatccagaaccctgaccctgccgtgtaccagctgagagactctaaatccagtgacaagtctgcatgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgactcgagggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 28 - DNA, 9961 bp, synthetic construct
  misc_feature 1..9961: note = Synthetic polynucleotide
  source 1..9961: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattcgccaccatgggcacaaggttgttcttctatgtggccctttgtctcctgtggaccggtcacatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtcggacgcccaacattcagtacttcggcgccgggacccggctctcagtgctggaggacctgaaaaacgtgttcccacccgaggtcgctgtgtttgagccatcagaagcagagatctcccacacccaaaaggccacactagtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcgggagcggagccacgaacttctctctgttaaagcaagcaggagacgtggaagaaaaccccggtcctatggcaggcattcgagctttatttatgtacttgtggctgcagctggactgggtctcgagaggtcaacagctgaatcagagtcctcaatctatgtttatccaggaaggagaagatgtctccatgaactgcacttcttcaagcatatttaacacctggctatggtacaagcaggaacctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggcacataccagaaagttacctttggaactggaacaaagctccaagtcatcccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgcggccgcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgactcgagggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 29 - DNA, 9961 bp, synthetic construct
  misc_feature 1..9961: note = Synthetic polynucleotide
  source 1..9961: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattcgccaccatgggaatcaggctcctgtgtcgtgtggccttttgtttcctggctgtaggactagtagaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtcggacgcccaacattcagtacttcggcgccgggacccggctctcagtgctggaggacctgaaaaacgtgttcccacccgaggtcgctgtgtttgagccatcagaagcagagatctcccacacccaaaaggccacactggcatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcgggagcggagccacgaacttctctctgttaaagcaagcaggagacgtggaagaaaaccccggtcctatgacacgagttagcttgctgtgggcagtcgtggtctccacctgtctcgagtccggcatgggtcaacagctgaatcagagtcctcaatctatgtttatccaggaaggagaagatgtctccatgaactgcacttcttcaagcatatttaacacctggctatggtacaagcaggaacctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggcacataccagaaagttacctttggaactggaacaaagctccaagtcatcccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgcggccgcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgactcgagggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 30 - DNA, 9955 bp, synthetic construct
  misc_feature 1..9955: note = Synthetic polynucleotide
  source 1..9955: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattcgccaccatggcctccctgctcttcttctgtggggccttttatctcctgggaaccggttccatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtcggacgcccaacattcagtacttcggcgccgggacccggctctcagtgctggaggacctgaaaaacgtgttcccacccgaggtcgctgtgtttgagccatcagaagcagagatctcccacacccaaaaggccacactagtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcgggagcggagccacgaacttctctctgttaaagcaagcaggagacgtggaagaaaaccccggtcctatgaactcctctctggactttctaattctgatcttaatgtttggaggaactagtggtcaacagctgaatcagagtcctcaatctatgtttatccaggaaggagaagatgtctccatgaactgcacttcttcaagcatatttaacacctggctatggtacaagcaggaacctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggcacataccagaaagttacctttggaactggaacaaagctccaagtcatcccaaatatccagaaccctgaccctgccgtgtaccagctgagagactctaaatccagtgacaagtctgcatgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgactcgagggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 31 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 32 - protein, 825 aa, Homo sapiens
MLDICLEKRVGTTLAAPKCNSSTVRFQGLAEGTKGTMKMDMEDADMTLWTEAEFEEKCTYIVNDHPWDSGADGGTSVQAEASLPRNLLFKYATNSEEVIGVMSKEYIPKGTRFGPLIGEIYTNDTVPKNANRKYFWRIYSRGELHHFIDGFNEEKSNWMRYVNPAHSPREQNLAACQNGMNIYFYTIKPIPANQELLVWYCRDFAERLHYPYPGELTMMNLTQTQSSLKQPSTEKNELCPKNVPKREYSVKEILKLDSNPSKGKDLYRSNISPLTSEKDLDDFRRRGSPEMPFYPRVVYPIRAPLPEDFLKASLAYGIERPTYITRSPIPSSTTPSPSARSSPDQSLKSSSPHSSPGNTVSPVGPGSQEHRDSYAYLNASYGTEGLGSYPGYAPLPHLPPAFIPSYNAHYPKFLLPPYGMNCNGLSAVSSMNGINNFGLFPRLCPVYSNLLGGGSLPHPMLNPTSLPSSLPSDGARRLLQPEHPREVLVPAPHSAFSFTGAAASMKDKACSPTSGSPTAGTAATAEHVVQPKATSAAMAAPSSDEAMNLIKNKRNMTGYKTLPYPLKKQNGKIKYECNVCAKTFGQLSNLKVHLRVHSGERPFKCQTCNKGFTQLAHLQKHYLVHTGEKPHECQVCHKRFSSTSNLKTHLRLHSGEKPYQCKVCPAKFTQFVHLKLHKRLHTRERPHKCSQCHKNYIHLCSLKVHLKGNCAAAPAPGLPLEDLTRINEEIEKFDISDNADRLEDVEDDISVISVVEKEILAVVRKEKEETGLKVSLQRNMGNGLLSSGCSLYESSDLPLMKLPPSNPLPLVPVKVKQETVEPMDP

SEQ ID NO: 33 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 34 - DNA, 42 bp, synthetic construct
  misc_feature 1..42: note = Synthetic primer
  source 1..42: mol_type = other DNA
cgcagtagcggtaaacggcgatgctgaagtcrcmcagactcc

SEQ ID NO: 35 - DNA, 39 bp, synthetic construct
  misc_feature 1..39: note = Synthetic primer
  source 1..39: mol_type = other DNA
cgcagtagcggtaaacggcgatgcwgmtgttwcccagac

SEQ ID NO: 36 - DNA, 42 bp, synthetic construct
  misc_feature 1..42: note = Synthetic primer
  source 1..42: mol_type = other DNA
cgcagtagcggtaaacggcgacactgragtyacscagacacc

SEQ ID NO: 37 - DNA, 39 bp, synthetic construct
  misc_feature 1..39: note = Synthetic primer
  source 1..39: mol_type = other DNA
cgcagtagcggtaaacggcgaggctggagtcachcaaas

SEQ ID NO: 38 - DNA, 39 bp, synthetic construct
  misc_feature 1..39: note = Synthetic primer
  source 1..39: mol_type = other DNA
cgcagtagcggtaaacggcgagcctggwgtcasycagac

SEQ ID NO: 39 - DNA, 38 bp, synthetic construct
  misc_feature 1..38: note = Synthetic primer
  source 1..38: mol_type = other DNA
cgcagtagcggtaaacggcggtgctggagtykcccagw

SEQ ID NO: 40 - DNA, 38 bp, synthetic construct
  misc_feature 1..38: note = Synthetic primer
  source 1..38: mol_type = other DNA
cgcagtagcggtaaacggcgatgctgrratcacccagr

SEQ ID NO: 41 - DNA, 39 bp, synthetic construct
  misc_feature 1..39: note = Synthetic primer
  source 1..39: mol_type = other DNA
cgcagtagcggtaaacggcgaagctgaagttgcccagtc

SEQ ID NO: 42 - DNA, 39 bp, synthetic construct
  misc_feature 1..39: note = Synthetic primer
  source 1..39: mol_type = other DNA
cgcagtagcggtaaacggcgatgctggagtyatccagtc

SEQ ID NO: 43 - DNA, 38 bp, synthetic construct
  misc_feature 1..38: note = Synthetic primer
  source 1..38: mol_type = other DNA
cgcagtagcggtaaacggcgaagctggagtkrytcagt

SEQ ID NO: 44 - DNA, 39 bp, synthetic construct
  misc_feature 1..39: note = Synthetic primer
  source 1..39: mol_type = other DNA
cgcagtagcggtaaacggcgatgccatggtcatccagaa

SEQ ID NO: 45 - DNA, 39 bp, synthetic construct
  misc_feature 1..39: note = Synthetic primer
  source 1..39: mol_type = other DNA
cgcagtagcggtaaacggcaatgccggcgtcatgcagaa

SEQ ID NO: 46 - DNA, 39 bp, synthetic construct
  misc_feature 1..39: note = Synthetic primer
  source 1..39: mol_type = other DNA
cgcagtagcggtaaacggcgatggtggaatcactcagtc

SEQ ID NO: 47 - DNA, 39 bp, synthetic construct
  misc_feature 1..39: note = Synthetic primer
  source 1..39: mol_type = other DNA
cgcagtagcggtaaacggcagtgctgtcrtctctcaama

SEQ ID NO: 48 - DNA, 39 bp, synthetic construct
  misc_feature 1..39: note = Synthetic primer
  source 1..39: mol_type = other DNA
cgcagtagcggtaaacggcgaagctgacatctaccagac

SEQ ID NO: 49 - DNA, 42 bp, synthetic construct
  misc_feature 1..42: note = Synthetic primer
  source 1..42: mol_type = other DNA
cgcagtagcggtaaacggcgatgtgaaagtracccagarcyc

SEQ ID NO: 50 - DNA, 39 bp, synthetic construct
  misc_feature 1..39: note = Synthetic primer
  source 1..39: mol_type = other DNA
cgcagtagcggtaaacggcacactccaggcacagagata

SEQ ID NO: 51 - DNA, 55 bp, synthetic construct
  misc_feature 1..55: note = Synthetic primer
  source 1..55: mol_type = other DNA
tgctaaaacgctagctgctggttcgtcgaccaaggcagtatctggagtcattgag

SEQ ID NO: 52 - DNA, 19 bp, synthetic construct
  misc_feature 1..19: note = Synthetic primer
  source 1..19: mol_type = other DNA
cgcagtagcggtaaacggc

SEQ ID NO: 53 - DNA, 41 bp, synthetic construct
  misc_feature 1..41: note = Synthetic primer
  source 1..41: mol_type = other DNA
ccagcagctagcgttttagcaggacaaarcmttgascagcc

SEQ ID NO: 54 - DNA, 41 bp, synthetic construct
  misc_feature 1..41: note = Synthetic primer
  source 1..41: mol_type = other DNA
ccagcagctagcgttttagcaaaggaccaagtgtttcagcc

SEQ ID NO: 55 - DNA, 41 bp, synthetic construct
  misc_feature 1..41: note = Synthetic primer
  source 1..41: mol_type = other DNA
ccagcagctagcgttttagcagctcagtcagtgrcycagcc

SEQ ID NO: 56 - DNA, 41 bp, synthetic construct
  misc_feature 1..41: note = Synthetic primer
  source 1..41: mol_type = other DNA
ccagcagctagcgttttagcagatgctaagaccacmcagcc

SEQ ID NO: 57 - DNA, 43 bp, synthetic construct
  misc_feature 1..43: note = Synthetic primer
  source 1..43: mol_type = other DNA
ccagcagctagcgttttagcaagaaaasawstggagcagagtc

SEQ ID NO: 58 - DNA, 41 bp, synthetic construct
  misc_feature 1..41: note = Synthetic primer
  source 1..41: mol_type = other DNA
ccagcagctagcgttttagcaagccaaaagatagaacagaa

SEQ ID NO: 59 - DNA, 41 bp, synthetic construct
  misc_feature 1..41: note = Synthetic primer
  source 1..41: mol_type = other DNA
ccagcagctagcgttttagcagaaaaccaggtggagcacag

SEQ ID NO: 60 - DNA, 41 bp, synthetic construct
  misc_feature 1..41: note = Synthetic primer
  source 1..41: mol_type = other DNA
ccagcagctagcgttttagcagcccagtckgtgasccagcw

SEQ ID NO: 61 - DNA, 41 bp, synthetic construct
  misc_feature 1..41: note = Synthetic primer
  source 1..41: mol_type = other DNA
ccagcagctagcgttttagcaggaaattcagtgryccagay

SEQ ID NO: 62 - DNA, 44 bp, synthetic construct
  misc_feature 1..44: note = Synthetic primer
  source 1..44: mol_type = other DNA
ccagcagctagcgttttagcacagaaggaggtggagcagratyc

SEQ ID NO: 63 - DNA, 41 bp, synthetic construct
  misc_feature 1..41: note = Synthetic primer
  source 1..41: mol_type = other DNA
ccagcagctagcgttttagcaggagagartgtggrgcwgca

SEQ ID NO: 64 - DNA, 41 bp, synthetic construct
  misc_feature 1..41: note = Synthetic primer
  source 1..41: mol_type = other DNA
ccagcagctagcgttttagcagcccagaagrtwactcaarc

SEQ ID NO: 65 - DNA, 41 bp, synthetic construct
  misc_feature 1..41: note = Synthetic primer
  source 1..41: mol_type = other DNA
ccagcagctagcgttttagcagcccagasagtsactcagyc

SEQ ID NO: 66 - DNA, 41 bp, synthetic construct
  misc_feature 1..41: note = Synthetic primer
  source 1..41: mol_type = other DNA
ccagcagctagcgttttagcaagtcaacagggagaagagga

SEQ ID NO: 67 - DNA, 41 bp, synthetic construct
  misc_feature 1..41: note = Synthetic primer
  source 1..41: mol_type = other DNA
ccagcagctagcgttttagcaggagactcggttacccagac

SEQ ID NO: 68 - DNA, 44 bp, synthetic construct
  misc_feature 1..44: note = Synthetic primer
  source 1..44: mol_type = other DNA
ccagcagctagcgttttagcaaaacaggaggtgacgcagaktcc

SEQ ID NO: 69 - DNA, 41 bp, synthetic construct
  misc_feature 1..41: note = Synthetic primer
  source 1..41: mol_type = other DNA
ccagcagctagcgttttagcaggccaacagaaggagaaaag

SEQ ID NO: 70 - DNA, 41 bp, synthetic construct
  misc_feature 1..41: note = Synthetic primer
  source 1..41: mol_type = other DNA
ccagcagctagcgttttagcagagctgaamgtggaacaaar

SEQ ID NO: 71 - DNA, 41 bp, synthetic construct
  misc_feature 1..41: note = Synthetic primer
  source 1..41: mol_type = other DNA
ccagcagctagcgttttagcaggacaacaggtaatgcaaat

SEQ ID NO: 72 - DNA, 41 bp, synthetic construct
  misc_feature 1..41: note = Synthetic primer
  source 1..41: mol_type = other DNA
ccagcagctagcgttttagcaacccagctgctggagcagag

SEQ ID NO: 73 - DNA, 41 bp, synthetic construct
  misc_feature 1..41: note = Synthetic primer
  source 1..41: mol_type = other DNA
ccagcagctagcgttttagcaagtcaacagaagaatgatga

SEQ ID NO: 74 - DNA, 41 bp, synthetic construct
  misc_feature 1..41: note = Synthetic primer
  source 1..41: mol_type = other DNA
ccagcagctagcgttttagcacaacaaccagtgcagagtcc

SEQ ID NO: 75 - DNA, 41 bp, synthetic construct
  misc_feature 1..41: note = Synthetic primer
  source 1..41: mol_type = other DNA
ccagcagctagcgttttagcaggtcaacagctgaatcagag

SEQ ID NO: 76 - DNA, 41 bp, synthetic construct
  misc_feature 1..41: note = Synthetic primer
  source 1..41: mol_type = other DNA
ccagcagctagcgttttagcagaagacaaggtggtacaaag

SEQ ID NO: 77 - DNA, 41 bp, synthetic construct
  misc_feature 1..41: note = Synthetic primer
  source 1..41: mol_type = other DNA
ccagcagctagcgttttagcaagcaattcagtcaagcagac

SEQ ID NO: 78 - DNA, 20 bp, synthetic construct
  misc_feature 1..20: note = Synthetic primer
  source 1..20: mol_type = other DNA
cgaccagcttgacatcacag

SEQ ID NO: 79 - protein, 9 aa, Epstein-Barr virus
FLRGRAYGL

(19) United States
(12) Patent Application Publication
(10) Pub. No.: US 2025/0154222 A1
(43) Pub. Date: May 15, 2025

(54) TECHNIQUES FOR GENERATING CELL-BASED THERAPEUTICS USING RECOMBINANT T CELL RECEPTOR GENES

(71) Applicant: University of Kansas, Lawrence, KS (US)

(72) Inventors: Brandon DEKOSKY, Lawrence, KS (US); Cheng-Yu Chung, Lawrence, KS (US)

(73) Assignee: University of Kansas, Lawrence, KS (US)

(21) Appl. No.: 18/955,752

(22) Filed: Nov. 21, 2024

Related U.S. Application Data
(62) Division of application No. 16/976,983, filed on Aug. 31, 2020, now Pat. No. 12,178,831.
(60) Provisional application No. 62/637,240, filed on Mar. 1, 2018.

(51) Int. Cl. C07K 14/725; A61K 35/17; A61K 40/11; A61K 40/22; A61K 40/32; A61K 40/41; A61K 40/42; A61K 40/46; A61P 35/00; C12N 5/0783

(52) U.S. Cl. CPC C07K 14/7051; A61K 35/17; A61K 40/11; A61K 40/22; A61K 40/32; A61K 40/418; A61K 40/42; A61K 40/46; A61P 35/00; C12N 5/0636; A61K 2239/31; A61K 2239/50; A61K 2239/56; A61K 2239/59

(57) ABSTRACT

The present technology relates generally to compositions and methods for creating recombinant T cell receptor (TCR) libraries and methods of their therapeutic use. The compositions and methods of the present technology are useful for rapid isolation of antigen-specific TCR repertoires as personalized, targeted therapies for cancer and viral infection.

### CROSS-REFERENCE TO RELATED APPLICATIONS

This application is a divisional of U.S. patent application Ser. No. 16/976,983 filed Aug. 31, 2020, which is a National Stage of PCT/US2019/019754, filed Feb. 27, 2019, which claims the benefit of and priority to U.S. Provisional Patent Application No. 62/637,240, filed Mar. 1, 2018, the contents of each of which are incorporated herein by reference in their entireties.

### U.S. GOVERNMENT SUPPORT

This invention was made with government support under GM103418 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Feb. 12, 2024, is named 104434-0336_SL.xml and is 229,405 bytes in size.

### FIELD OF TECHNOLOGY

The present technology relates generally to methods of high-throughput isolation and manipulation of genes from single T cells that encode T cell receptors as cellular therapies, compositions of T cell libraries, and methods of their therapeutic use.

### BACKGROUND

The following description is provided to assist the understanding of the reader. None of the information provided or references cited is admitted to be prior art to the present methods.

Humans have many thousands of T cell receptors that provide a major component of adaptive immune systems, and T cell receptor responses have been demonstrated to provide important contributions to protection from diseases including viral infections, cancer, and autoimmunity. However, molecularly defined T cell receptor therapies have had narrow impact on clinical care because the methods for translating T cell receptor responses into treatments have not fully been elucidated at a scale that is practical for translational therapies. Current methods rely on low-throughput T cell receptor identification technologies, cumbersome practices for cloning of T cell receptors, and a limited ability to direct the cell state of T cell libraries.

There is a need to establish methods for the rapid translation of T cell receptor responses as drugs for cellular therapeutics, particularly for the treatment of cancer. The present technology addresses this need, in part by leveraging the contributions of TCR repertoires to adaptive immunity for clinical treatment or prevention of cancer.

### SUMMARY

The present technology relates generally to novel compositions and methods for creating recombinant TCR libraries, and methods of their therapeutic use. The compositions and methods of the present technology are useful for rapid isolation of antigen-specific TCR repertoires for development of personalized, targeted therapies for cancer and viral infections.

Accordingly, in one aspect, the present technology provides a recombinant T cell receptor (TCR) library vector comprising: (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA isolated from a single lysed T cell that is present in a compartment. In some embodiments, the mRNA of the single lysed T cell is isolated using an mRNA capture reagent or reverse transcription-PCR (RT-PCR). Additionally or alternatively, in some embodiments, the compartment containing the contents of the single lysed T cell is a microwell (e.g., a microwell within a 96-well plate) or a droplet.

In some embodiments of the vector, the first polynucleotide and the second polynucleotide are operably linked, optionally via a linker polynucleotide. In some embodiments of the vector, the first polynucleotide and the second polynucleotide have been operably linked by reverse transcription and PCR amplification of the captured T cell mRNA. In some embodiments of the vector, the first polynucleotide and the second polynucleotide have been cloned into the vector backbone by cleavage at a target restriction endonuclease site that is natively found in TCR genes. In certain embodiments, the target restriction endonuclease site occurs in TCR genes with low frequency. In some embodiments, the first polynucleotide and the second polynucleotide have been altered to incorporate at least one target restriction endonuclease site disclosed in Table 7 or 8. In certain embodiments, the target restriction endonuclease site comprises a silent mutation.

In certain embodiments of the vector, the mRNA capture reagent is selected from the group consisting of a poly(dT) coated bead, an oligonucleotide-coated bead, a hydrogel bead, and a printed oligo on the surface of a microarray well. In some embodiments, the compartment is an emulsion droplet or a well. In certain embodiments, the well is located in a printed polymer slide, a plastic plate, a microtiter plate, or a gel. In some embodiments, the volume of the compartment is 5 nL or less.

In certain embodiments of the vector, the vector further comprises at least one polynucleotide encoding an expression control element operably linked to the first polynucleotide and/or the second polynucleotide. In some embodiments, the expression control element is selected from the group consisting of: a promoter, a p2a sequence, and an IRES sequence. In particular embodiments, the promoter is an EF1α promoter or a CMV promoter. In certain embodiments, the polynucleotide encoding the expression control element is located between the first polynucleotide and the second polynucleotide.

In some embodiments of the vector, the vector is circularized. In some embodiments, the vector has been circularized prior to incorporation of the expression control element into the vector. In other embodiments, the vector has been circularized after incorporation of the expression control element into the vector. In some embodiments, the vector is linear (e.g., not circularized).

In certain embodiments of the vector, the expression control element has been incorporated near a protospacer adjacent motif (PAM). In other embodiments, the expression control element has been incorporated into the vector using a DNA-modifying enzyme selected from a restriction enzyme or a TALEN. In other embodiments, the vector further comprises one or more polynucleotides encoding a transposon operably linked to at least one of the first polynucleotide and the second polynucleotide.

In some embodiments of the vector, the vector further comprises one or more of: a polynucleotide encoding a detectable marker, a polynucleotide encoding a selectable marker, a polynucleotide encoding a switch mechanism for controlling expression and/or activation of the first polynucleotide and the second polynucleotide, and a polynucleotide encoding a Kozak consensus sequence or an enhancer.

In certain embodiments of the vector, the vector backbone is selected from a group consisting of a retroviral, a lentiviral, an adenoviral, and an adeno-associated viral vector backbone. In certain embodiments, the vector may comprise linear DNA for CRISPR/Cas9 integration. In certain embodiments, the vector may comprise DNA that can be incorporated into a host using a recombinase enzyme. In some embodiments, the vector may comprise DNA that can be incorporated into a host using a transposase enzyme.

In some embodiments of the vector, the encoded T cell receptor (e.g., TCRαβ or TCRγδ) is reactive against a disease antigen or target cell. In certain embodiments, the disease antigen is a viral antigen derived from a virus selected from the group consisting of adenovirus, CMV, coronavirus, coxsackievirus, Dengue virus, Epstein-Barr virus (EBV), enterovirus 71 (EV71), Ebola virus, hepatitis A (HAV), hepatitis B (HBV), cytomegalovirus (CMV), hepatitis C (HCV), hepatitis D (HDV), hepatitis E (HEV), human immunodeficiency virus (HIV), human papillomavirus (HPV), herpes simplex virus (HSV), human T-lymphotropic virus (HTLV), influenza A virus, influenza B virus, Japanese encephalitis, leukemia virus, measles virus, molluscum contagiosum, orf virus, parvovirus, rabies virus, respiratory syncytial virus, rift valley fever virus, rubella virus, rotavirus, tick-borne encephalitis (TBEV), simian immunodeficiency virus, tobacco etch virus (TEV), varicella zoster virus, variola, West Nile virus, Zika virus, and Chikungunya virus. In other embodiments, the disease antigen is a tumor antigen selected from the group consisting of CD45, glypican-3, IGF2B3, Kallikrein 4, KIF20A, Lengsin, Meloe, mucin 5AC (MUC5AC), survivin, cyclin-A1, MAGE-A1, MAGE-C1, MAGE-C2, SSX2, XAGElb/GAGED2A, CD19, CD20, CD22, CD52, epidermal growth factor receptor (EGFR), human epidermal growth factor receptor 2 (HER2), tumor necrosis factor receptor superfamily, member 10a (TRAILR1), receptor activator of nuclear factor kappa-B ligand (RANKL), insulin-like growth factor 1 receptor (IGF1R), epithelial cell adhesion molecule (EpCAM), and carcinoembryonic antigen (CEA).

In another aspect, the present technology provides a recombinant cell comprising a vector, wherein the vector comprises (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent in a compartment, optionally wherein the recombinant cell is a bacterial cell, mammalian cell, or a yeast cell. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet.

In one aspect, the present technology provides a recombinant TCR vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell that was captured by an mRNA capture reagent in a compartment. In some embodiments, the plurality of vectors comprises a TCR repertoire. In some embodiments, the individual vectors in the TCR vector library were selected for inclusion in the TCR library on the basis of one or more of the following characteristics: TCR clonal prevalence, TCR enrichment characteristics from in vitro assays, TCR binding specificity, TCR V segment sequence, TCR D segment sequence, TCR J segment sequence, TCR gene motifs, and/or CDR3 gene motifs. In some embodiments, the individual vectors in the library are mixed in a defined ratio to generate a synthetically-derived TCR library.

In another aspect, the present technology provides an isolated immune cell comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell that was captured by an mRNA capture reagent in a compartment. In some embodiments the immune cell is a hematopoietic stem cell, a hematopoietic progenitor cell, a T cell, or a natural killer (NK) cell.

In one aspect, the present technology provides a cell population comprising a recombinant TCR vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet. In some embodiments, the population comprises hematopoietic stem cells, hematopoietic progenitor cells, T cells, or NK cells.

In another aspect, also provided herein is a method for preparing a recombinant TCR library, the method comprising transforming a population of cells with a vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet. In some embodiments, the population comprises hematopoietic stem cells, hematopoietic progenitor cells, T cells, or NK cells. In some embodiments, the plurality of vectors is circularized at certain steps of the method, and linearized at other steps.

In some embodiments of the method, the library is screened for specific binding to a target cell. In certain embodiments, the cell is a cancer cell or a cell infected with a virus. In some embodiments, the target cell was isolated from a subject.

In other embodiments of the method, the library is screened for specific binding to an antigen:MHC complex. In some embodiments the antigen of the antigen:MHC complex is a viral antigen derived from a virus selected from the group consisting of adenovirus, CMV, coronavirus, coxsackievirus, Dengue virus, Epstein-Barr virus (EBV), enterovirus 71 (EV71), Ebola virus, hepatitis A (HAV), hepatitis B (HBV), cytomegalovirus (CMV), hepatitis C (HCV), hepatitis D (HDV), hepatitis E (HEV), human immunodeficiency virus (HIV), human papillomavirus (HPV), herpes simplex virus (HSV), human T-lymphotropic virus (HTLV), influenza A virus, influenza B virus, Japanese encephalitis, leukemia virus, measles virus, molluscum contagiosum, orf virus, parvovirus, rabies virus, respiratory syncytial virus, rift valley fever virus, rubella virus, rotavirus, tick-borne encephalitis (TBEV), simian immunodeficiency virus, tobacco etch virus (TEV), varicella zoster virus, variola, West Nile virus, Zika virus, and Chikungunya virus. In other embodiments, the antigen of the antigen:MHC complex is a tumor antigen selected from the group consisting of CD45, glypican-3, IGF2B3, Kallikrein 4, KIF20A, Lengsin, Meloe, mucin 5AC (MUC5AC), survivin, cyclin-A1, MAGE-A1, MAGE-C1, MAGE-C2, SSX2, XAGElb/GAGED2A, CD19, CD20, CD22, CD52, EGFR, HER2, TRAILR1, RANKL, IGF1R, EpCAM, and CEA.

In some embodiments of the method, the library is screened for T cell phenotypic markers. In other embodiments of the method, the library is screened for hematopoietic stem cell phenotypic markers. In other embodiments of the method, the library is screened for natural killer cell phenotypic markers.

In certain embodiments of the method, the library is screened for activity in a co-culture system, wherein the co-culture system comprises at least one of the following: (a) a cancer cell line; (b) a plurality of cells infected with a known virus; (c) a plurality of tumor cells isolated from a cancer patient; (d) an immortalized cell line; or (e) a plurality of cells derived from a patient tissue biopsy.

In some embodiments of the method, the transformed cells are activated in vitro. In particular embodiments, activation is performed using one or more of the following stimulants: anti-CD3 antibody, anti-CD8 antibody, anti-CD27 antibody, IL-2, IL-4, IL-21, anti-PD1 antibody, anti-CTLA4 antibody, tumor cell lysate, cellular co-culture with virus-infected cells, and tumor cell lines.

In certain embodiments of the method, the population of cells is transformed with a transcription factor. The transcription factor may influence the behavior or phenotype of the transformed cells. In some embodiments, the transcription factor is selected from the group consisting of FOXP3, BLIMP-1, Helios, Ikaros, and TGF-beta.

In another aspect, provided herein is a recombinant TCR library prepared by a method comprising transforming a population of cells with a vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet. In some embodiments, the population comprises hematopoietic stem cells, hematopoietic progenitor cells, T cells, or NK cells.

In one aspect, the present technology provides a composition comprising a recombinant TCR library prepared by a method comprising transforming a population of cells with a vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment, and a carrier. In some embodiments, the carrier is a pharmaceutically acceptable carrier. In some embodiments, the mRNA of the single lysed T cell is isolated using an mRNA capture reagent. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet.

In one aspect, provided herein is a method of treating a subject in need thereof, the method comprising administering to the subject an effective amount of a recombinant TCR library or a composition comprising a recombinant TCR library, wherein the recombinant TCR library was prepared by a method comprising transforming a population of cells with a vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet.

In another aspect, provided herein is a method of treating cancer in a subject in need thereof, the method comprising administering to the subject an effective amount of a recombinant TCR library or a composition comprising a recombinant TCR library, wherein the recombinant TCR library was prepared by a method comprising transforming a population of cells with a vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet.

In some embodiments, the cancer is acute lymphoblastic leukemia (ALL); acute myeloid leukemia (AML); adrenocortical carcinoma; AIDS-related cancers; anal cancer; appendix cancer; astrocytoma; atypical teratoid/rhabdoid tumor, brain cancer; basal cell carcinoma of the skin; bile duct cancer; bladder cancer; bone cancer; breast cancer; bronchial tumors; Burkitt lymphoma; carcinoid tumor (gastrointestinal); germ cell tumor; primary CNS lymphoma; cervical cancer; cholangiocarcinoma; chordoma; chronic lymphocytic leukemia (CLL); chronic myelogenous leukemia (CML); chronic myeloproliferative neoplasms; colorectal cancer; craniopharyngioma; cutaneous T-cell lymphoma; ductal carcinoma in situ (DCIS); endometrial cancer; ependymoma; esophageal cancer; esthesioneuroblastoma; extracranial germ cell tumor; extragonadal germ cell tumor; eye cancer; intraocular melanoma; retinoblastoma; fallopian tube cancer; fibrous histiocytoma of bone, malignant, and osteosarcoma; gallbladder cancer; gastric cancer; gastrointestinal carcinoid tumor; gastrointestinal stromal tumors (GIST); germ cell tumors; gestational trophoblastic disease; hairy cell leukemia; head and neck cancer; heart tumors; hepatocellular cancer; histiocytosis, Langerhans cell; Hodgkin lymphoma; hypopharyngeal cancer; intraocular melanoma; islet cell tumors, pancreatic neuroendocrine tumors; kidney cancer; laryngeal cancer; leukemia; lip and oral cavity cancer; liver cancer; lung cancer; lymphoma; male breast cancer; malignant fibrous histiocytoma of bone and osteosarcoma; melanoma; Merkel cell carcinoma; mesothelioma; metastatic cancer; mouth cancer; multiple endocrine neoplasia syndrome; multiple myeloma/plasma cell neoplasms; mycosis fungoides; myelodysplastic syndrome, myeloproliferative neoplasm, chronic; nasopharyngeal cancer; neuroblastoma; Non-Hodgkin lymphoma; non-small cell lung cancer; oral cancer, oropharyngeal cancer; osteosarcoma; ovarian cancer; pancreatic cancer; pancreatic neuroendocrine tumors; papillomatosis; paraganglioma; paranasal sinus cancer; parathyroid cancer; pharyngeal cancer; pheochromocytoma; pituitary tumor; pleuropulmonary blastoma; prostate cancer; rectal cancer; recurrent cancer; renal cell cancer; retinoblastoma; rhabdomyosarcoma; salivary gland cancer; sarcoma; Ewing sarcoma; Kaposi sarcoma; osteosarcoma; uterine sarcoma; Sézary syndrome; skin cancer; small cell lung cancer; small intestine cancer; soft tissue sarcoma; squamous cell carcinoma of the skin; squamous neck cancer; stomach cancer; T cell lymphoma; testicular cancer; throat cancer; nasopharyngeal cancer; hypopharyngeal cancer; thymic carcinoma; thyroid cancer; urethral cancer; uterine cancer; vaginal cancer; vascular tumors; vulvar cancer; or Wilms tumor.

In one aspect, provided herein is a method of inhibiting tumor growth in a subject in need thereof, the method comprising administering to the subject an effective amount of a recombinant TCR library or a composition comprising a recombinant TCR library, wherein the recombinant TCR library was prepared by a method comprising transforming a population of cells with a vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet. In some embodiments, the tumor is a solid tumor.

In another aspect, provided herein is a method of treating a viral infection in a subject in need thereof, the method comprising administering to the subject an effective amount of a recombinant TCR library or a composition comprising a recombinant TCR library, wherein the recombinant TCR library was prepared by a method comprising transforming a population of cells with a vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet. In some embodiments, the viral infection is caused by a virus selected from the group consisting of adenovirus, CMV, coronavirus, coxsackievirus, Dengue virus, Epstein-Barr virus (EBV), enterovirus 71 (EV71), Ebola virus, hepatitis A (HAV), hepatitis B (HBV), cytomegalovirus (CMV), hepatitis C (HCV), hepatitis D (HDV), hepatitis E (HEV), human immunodeficiency virus (HIV), human papillomavirus (HPV), herpes simplex virus (HSV), human T-lymphotropic virus (HTLV), influenza A virus, influenza B virus, Japanese encephalitis, leukemia virus, measles virus, molluscum contagiosum, orf virus, parvovirus, rabies virus, respiratory syncytial virus, rift valley fever virus, rubella virus, rotavirus, tick-borne encephalitis (TBEV), simian immunodeficiency virus, tobacco etch virus (TEV), varicella zoster virus, variola, West Nile virus, Zika virus, and Chikungunya virus.

In some embodiments, the methods of treatment provided herein further comprise administering one or more additional doses of the recombinant TCR library or the composition to the subject.

In some embodiments, the recombinant TCR library comprises cells that are autologous or allogenic to the subject being treated.

In some embodiments, the subject is a human, an animal, a non-human primate, a dog, cat, a sheep, a mouse, a horse, or a cow. In a particular embodiment, the subject is a human.

### BRIEF DESCRIPTION OF THE DRAWINGS

FIG. 1 shows a gel image of overlap extension (OE-PCR) to obtain the full-length TCR α:β variable region. After the OE-PCR, the PCR products were purified and analyzed by DNA gel electrophoresis. The TCR α:β amplicon (around 1000 b.p.) was visible, and was further enriched using nested PCR. After DNA gel electrophoresis, gel extraction was performed to obtain the TCR α:β amplicon. 5 ng of purified PCR product was used as a template for performing semi-nested PCR.

FIG. 2 shows gel electrophoresis of nested PCR products. After DNA electrophoresis, the TCR α:β amplicon was excised and gel purification was performed using Zymoclean™ Gel DNA Recovery Kits (Zymo Research). The purified amplicon was subjected to zero-blunt cloning (Thermo Fisher Scientific) to analyze TCR α:β amplicon sequences. The sequences were identified by the NCBI IGBLAST T cell receptor gene database.

FIG. 3 shows a vector map for the exemplary pLVX-CMV-TCR1-pTert-iCas9 vector. The vector comprises an ampicillin resistance gene, 5′ and 3′ LTRs, a CMV promoter, TCR beta sequences (TRBV6-2 leader peptide, TRBV25, TRBC) and TCR alpha sequences (TRAV38-1, TRAV35, and TRAC), a p2A sequence inserted between TCR beta and TCR alpha, an IRES sequence inserted following the TCR alpha sequence, an mCherry fluorescent marker, an i-Caspase9 suicide switch controlled by a tet inducible promoter and flanked by cHS4 insulator sequences, and a WPRE enhancer.

FIG. 4 shows a vector map for pLVX-CMV-TCR2-pTert-iCas9. The vector comprises an ampicillin resistance gene, 5′ and 3′ LTRs, a CMV promoter, TCR beta sequences (TRBV5-8 leader peptide, TRBV25, TRBC) and TCR alpha sequences (TRAV40, TRAV35, and TRAC), a p2A sequence inserted between TCR beta and TCR alpha, an IRES sequence inserted following the TCR alpha sequence, an mCherry fluorescent marker, an i-Caspase9 suicide switch controlled by a tet inducible promoter and flanked by cHS4 insulator sequences, and a WPRE enhancer.

FIG. 5 shows a vector map for pLVX-CMV-TCR3-pTert-iCas9. The vector comprises an ampicillin resistance gene, 5′ and 3′ LTRs, a CMV promoter, TCR beta sequences (TRBV10-3 leader peptide, TRBV25, TRBC) and TCR alpha sequences (TRAV13-2, TRAV35, and TRAC), a p2A sequence inserted between TCR beta and TCR alpha, an IRES sequence inserted following the TCR alpha sequence, an mCherry fluorescent marker, an i-Caspase9 suicide switch controlled by a tet inducible promoter and flanked by cHS4 insulator sequences, and a WPRE enhancer.

FIG. 6 shows a vector map for pLVX-CMV-TCR4-pTert-iCas9. The vector comprises an ampicillin resistance gene, 5′ and 3′ LTRs, a CMV promoter, TCR beta sequences (TRBV28-1 leader peptide, TRBV25, TRBC) and TCR alpha sequences (TRAV38-1, TRAV35, and TRAC), a p2A sequence inserted between TCR beta and TCR alpha, an IRES sequence inserted following the TCR alpha sequence, an mCherry fluorescent marker, an i-Caspase9 suicide switch controlled by a tet inducible promoter and flanked by cHS4 insulator sequences, and a WPRE enhancer.

FIG. 7 shows a vector map for pLVX-CMV-TCR5-pTert-iCas9. The vector comprises an ampicillin resistance gene, 5′ and 3′ LTRs, a CMV promoter, TCR beta sequences (TRBV24-1 leader peptide, TRBV25, TRBC) and TCR alpha sequences (TRAV38, TRAV35, and TRAC), a p2A sequence inserted between TCR beta and TCR alpha, an IRES sequence inserted following the TCR alpha sequence, an mCherry fluorescent marker, an i-Caspase9 suicide switch controlled by a tet inducible promoter and flanked by cHS4 insulator sequences, and a WPRE enhancer.

FIG. 8 shows a vector map for pLVX-TCR1-pTert-iCas9. The vector comprises an ampicillin resistance gene, 5′ and 3′ LTRs, a CMV promoter, TCR beta sequences (TRBV6-2 leader peptide, TRBV25, TRBC) and TCR alpha sequences (TRAV38-1, TRAV35, and TRAC), a p2A sequence inserted between TCR beta and TCR alpha, an IRES sequence inserted following the TCR alpha sequence, an mCherry fluorescent marker, and a WPRE enhancer.

FIG. 9 shows a vector map for pLVX-TCR2-pTert-iCas9. The vector comprises an ampicillin resistance gene, 5′ and 3′ LTRs, a CMV promoter, TCR beta sequences (TRBV5-8 leader peptide, TRBV25, TRBC) and TCR alpha sequences (TRAV40, TRAV35, and TRAC), a p2A sequence inserted between TCR beta and TCR alpha, an IRES sequence inserted following the TCR alpha sequence, an mCherry fluorescent marker, and a WPRE enhancer.

FIG. 10 shows a vector map for pLVX-TCR3-pTert-iCas9. The vector comprises an ampicillin resistance gene, 5′ and 3′ LTRs, a CMV promoter, TCR beta sequences (TRBV10-3 leader peptide, TRBV25, TRBC) and TCR alpha sequences (TRAV13-2, TRAV35, and TRAC), a p2A sequence inserted between TCR beta and TCR alpha, an IRES sequence inserted following the TCR alpha sequence, an mCherry fluorescent marker, and a WPRE enhancer.

FIG. 11 shows a vector map for pLVX-TCR4-pTert-iCas9. vector comprises an ampicillin resistance gene, 5′ and 3′ LTRs, a CMV promoter, TCR beta sequences (TRBV28-1 leader peptide, TRBV25, TRBC) and TCR alpha sequences (TRAV38-1, TRAV35, and TRAC), a p2A sequence inserted between TCR beta and TCR alpha, an IRES sequence inserted following the TCR alpha sequence, an mCherry fluorescent marker, and a WPRE enhancer.

FIG. 12 shows a vector map for pLVX-TCR5-pTert-iCas9. The vector comprises an ampicillin resistance gene, 5′ and 3′ LTRs, a CMV promoter, TCR beta sequences (TRBV24-1 leader peptide, TRBV25, TRBC) and TCR alpha sequences (TRAV38, TRAV35, and TRAC), a p2A sequence inserted between TCR beta and TCR alpha, an IRES sequence inserted following the TCR alpha sequence, an mCherry fluorescent marker, and a WPRE enhancer.

FIG. 13 shows a vector map for the original pLVX-EFla-IRES-mCherry vector.

FIG. 14 shows a vector map for the modified pLVX-EFla-IRES-mCherry vector, in which the excess AgeI, SphI, NheI and MluI restriction sites were eliminated by performing site-directed mutagenesis.

FIG. 15 shows DNA electrophoresis analysis of the T cell receptor (TCR) amplicon (alpha:beta chain) from single-cell emulsification overlap-extension RT-PCR. Left panel indicates PCR amplification scheme of the paired alpha:beta chain TCR amplicon. Right panel displays the DNA electrophoresis results of the 1^{st} semi-nested, 2^{nd} semi-nested and Mi-seq PCR. T cell receptor amplicons were highlighted in red square. The Miseq primers annealed at the FR3 region of the TRBV genes, yielding an ˜550 bp paired alpha:beta PCR amplicon for natively paired DNA sequence analysis.

FIG. 16 shows a summary of linked alpha:beta T cell receptor gene distribution. After high-throughput sequencing, raw DNA sequences were quality-filtered and annotated for TCR gene usage via NCBI IgBLAST and a CDR3-motif algorithm, paired by a and p chains, and compiled into a TCR repertoire.

FIG. 17 shows the evaluation of the expression of mutant anti-HIV-Nef-Rm9 TCRs and their binding affinity to pMHC, where these mutant TCRs include a leader peptide different from the original wild-type anti-HIV-Nef-Rm9 leader peptide. These non-native leader peptides all have silent DNA mutation sequences in order to include restriction enzyme cutting sites (The original and non-silent mutant leader peptide DNA sequences and the introduced restriction cutting sites are summarized in Tables 7-8).

FIG. 18 shows the evaluation of the expression of mutant anti-HIV-Nef-Rm9 TCRs and their binding affinity to pMHC. These mutant TCRs include one non-silent or one silent mutation at the TCR constant region for incorporation of a restriction cutting site for TCR cloning (See all the silent mutations in Table 7 and non-silent mutations in Table 8).

FIG. 19(a) shows isolation of human effector T cells for tumor-specific T cell analysis of humanized mouse models and cancer patients. FIG. 19(b) shows Vα:Vβ gene usage in 31,718 human CD8+ TCR clusters that were isolated, RT-PCR amplified, analyzed via NGS.

FIG. 20 shows a lentiviral TCR cell display platform of the present technology. TCRα:β genes separated by p2a were inserted into the multiple cloning site (MCS) of an IRES TCR expression vector. Co-transfection in HEK293T with packaging and envelope vectors generates lentiviral particles that transduce J.RT3/CD8 cell lines with TCR genes (which are expressed at the cell surface) and a mCherry reporter. Flow plot: J.RT3/CD8 with anti-B*0702 NEF TM9 TCR, stained with HIV Nef B*0702 RM9, RPQVPLRPM (SEQ ID NO: 1).

FIG. 21(a) shows RKO tumor size in humanized mice treated with immune checkpoint inhibitors. TCR screening will be performed to discover the antigen-specific TCRs targeting RKO cells. FIG. 21(b) shows the treatment groups in a mouse colon cancer model that will be used evaluate identified TCRs for antigen-specific immunotherapy.

FIG. 22 shows the workflow for testing and optimizing antigen-specific cancer immunotherapy in PDX mouse models. (Left) Patient tumors were isolated and cultured as mouse xenografts, cryopreserved in LN2, and propagated in culture. (Center) TCR genes were collected and screened against PDX cells. (Right) Anti-tumor TCR genes were used as gene therapies in PDX models to test and optimize treatment efficacy.

### DETAILED DESCRIPTION

Immune checkpoint inhibitors have catalyzed tremendous progress in cancer therapy, highlighting the critical role of immune cells, particularly CD8+ T cells, in controlling tumor growth. However, while recent progress in immune receptor sequencing has provided insights regarding clonal rank-based features of adaptive immunity, the inability to sequence and functionally screen paired TCR alpha and beta chains has precluded the collection of broader functional data regarding tumor immunosuppression and complicated the discovery and rapid therapeutic use of anti-cancer T cell receptor genes for gene therapies (Bonter, K. et al., Regen. Med. 12, 623-636 (2017); Malherbe, L., Ann. Allergy. Asthma. Immunol. 103, 76-79 (2009); Ahmad, T. A. et al., Vaccine Rep. 6, 13-22 (2016); Maus, M. V. et al., Annu. Rev. Immunol. 32, 189-225 (2014); Yee, C. J. Transl. Med. 3, 17 (2005)). To date, cloning and functional analysis of tumor-specific T cells is practically limited to just a few common peptide-MHC combinations per sample due to requirements for sorting primary, viable T cells (Malherbe, L., Ann. Allergy. Asthma. Immunol. 103, 76-79 (2009); Ahmad, T. A. et al., Vaccine Rep. 6, 13-22 (2016); Sanchez-Trincado, J. L. et al., Journal of Immunology Research (2017)). The low number of peptide-MHCs that can be effectively screened is further complicated by the fact that repetitive screening is necessary for neoantigen discovery because each tumor sample has its own unique landscape of somatic mutations (Martincorena, I. & Campbell, P. J. Science 349, 1483-1489 (2015); Tran, E. et al., Science aad1253 (2015); Choudhury, N. J. et al., Eur. Urol. Focus 2, 445-452 (2016)). Alternative methods such as T cell proliferation and ELISPOT rely on live T cells, which are highly limited in many patient samples, as well as having limitations in terms of cell growth rates, time required for application as cell-based therapies, specificity and maintenance of appropriate T cell phenotype (Bonter, K. et al., Regen. Med. 12, 623-636 (2017); Maus, M. V. et al., Annu. Rev. Immunol. 32, 189-225 (2014); Yee, C. J. Transl. Med. 3, 17 (2005); Redeker, A. & Arens, R., Front. Immunol. 7, (2016)).

The methods and compositions described herein directly address these bottlenecks and provide the first comprehensive sequence- and function-based annotation of epitope-specific T cell responses in patients, providing new molecular-scale technologies to guide the development of targeted cancer therapeutics. The compositions and methods of the present technology are generally useful for rapid isolation of antigen-specific TCR repertoires that can be screened, modified, and used as personalized, targeted therapies for, e.g., cancer and viral infections. The targeted therapies described herein can be developed and administered more rapidly (in as few as five days to two weeks) than presently available cell-based therapies. In addition, these therapies comprise multiple, distinct TCRs that have a greater likelihood of avoiding immune escape, a mechanism wherein target cells can evade immune detection by suppression of a targeted epitope or antigen.

Furthermore, specific embodiments of the methods and compositions described herein provide the following distinct advantages over previously described approaches to creating TCR libraries: (1) in some embodiments, described herein is a method wherein the TCR library can be grown as colonies in bacteria and numerous colonies can be selected, sequenced, and mixed together an re-delivered as a defined product; (2) in some embodiments, described herein is a separation and characterization step that minimizes the likelihood of PCR error variants which may be included in the final drug product, thereby reducing the risk that the final therapeutic cell composition contains uncharacterizable and potentially very dangerous variants which may induce side effects; (3) in some embodiments, described herein is the inclusion of a suicide switch to reduce the risk of harm to the patient in the event of complications and off-target effects; (4) in some embodiments, the cells are pre-stimulated prior to administration to achieve or enhance the desired TCR function in vivo; (5) in some embodiments, the methods include the ability co-express transcription factors in the cells to influence T cell development into a potent anti-cancer phenotype; (6) in some embodiments, the in vitro activation techniques may be modified as needed depending on the patient's specific response to therapy administration; and (7) in some embodiments, the disclosed methods can be used to provide repeated doses of cell therapy to the patient if the disease condition persists or recurs.

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art. All references cited herein are incorporated herein by reference in their entireties and for all purposes to the same extent as if each individual publication, patent, or patent application was specifically and individually incorporated by reference in its entirety for all purposes.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), rectally, or topically. Administration includes self-administration and the administration by another.

An “adjuvant” refers to one or more substances that cause stimulation of the immune system. In this context, an adjuvant is used to enhance an immune response to one or more vaccine antigens or antibodies. An adjuvant may be administered to a subject before, in combination with, or after administration of the vaccine. Examples of chemical compounds used as adjuvants include aluminum compounds, oils, block polymers, immune stimulating complexes, vitamins and minerals (e.g., vitamin E, vitamin A, selenium, and vitamin B12), Quil A (saponins), bacterial and fungal cell wall components (e.g., lipopolysaccarides, lipoproteins, and glycoproteins), hormones, cytokines, and co-stimulatory factors.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. The term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

More particularly, antibody refers to a polypeptide ligand comprising at least a light chain immunoglobulin variable region or heavy chain immunoglobulin variable region, which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes), which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a ρ-sheet conformation and the CDRs form loops which connect, and in some cases form part of, the β-sheet structure. Thus, framework regions act to form a scaffold that provides for positioning the CDRs in correct orientation by inter-chain, non-covalent interactions.

As used herein, “complementarity determining region” or “CDR” refers to a region of an antibody or TCR that is primarily responsible for binding to an epitope of an antigen or an antigen:MHC complex. CDRs are also referred to as hypervariable regions. The CDRs of each TCR or antibody chain are typically referred to as CDR1, CDR2, and CDR3, numbered sequentially starting from the N-terminus, and are also typically identified by the chain in which the particular CDR is located. Antibodies and TCRs with different specificities (i.e. different combining sites for different antigens) have different CDRs. Only a limited number of amino acid positions within the CDRs are directly involved in antigen binding. These positions within the CDRs are called specificity determining residues (SDRs).

As used herein, an “antigen” refers to a molecule to which an antibody can selectively bind. The target antigen may be a protein (e.g., an antigenic peptide), carbohydrate, nucleic acid, lipid, hapten, or other naturally occurring or synthetic compound. The target antigen may be a polypeptide or peptide mimic. An antigen may also be administered to an animal to generate an immune response in the animal. In some embodiments, the antigen comprises one or more epitopes. In some embodiments, the antigen or an epitope derived from the antigen, can be loaded into an MHC class I or MHC class II complex.

As used herein, the term “autologous,” in reference to cells refers to cells that are isolated and administered back into the same subject (e.g., recipient, donor, or host). “Allogeneic” refers to non-autologous cells.

As used herein, “binding affinity” refers to the strength of the total noncovalent interactions between a single binding site of a molecule (e.g., an antibody) and its binding partner (e.g., an antigen or antigenic peptide). The affinity of a molecule X for its partner Y can generally be represented by the dissociation constant (K_{d}). Affinity can be measured by standard methods known in the art, including those described herein. A low-affinity complex contains an antibody that generally tends to dissociate readily from the antigen, whereas a high-affinity complex contains an antibody that generally tends to remain bound to the antigen for a longer duration.

As used herein, the term “B cell,” refers to a type of lymphocyte in the humoral immunity of the adaptive immune system. B cells principally function to make antibodies, serve as antigen presenting cells, release cytokines, and develop memory B cells after activation by antigen interaction. B cells are distinguished from other lymphocytes, such as T cells, by the presence of a B-cell receptor on the cell surface. B cells may either be isolated or obtained from a commercially available source. Non-limiting examples of commercially available B cell lines include lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75](ATCC® CRL-2625™), DS-1 (ATCC® CRL-11102™) EB-3 [EB3](ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), AND SUP-B15 (ATCC CRL-1929). Further examples include but are not limited to cell lines derived from anaplastic and large cell lymphomas, e.g., DEL, DL-40, FE-PD, JB6, Karpas 299, Ki-JK, Mac-2A Plyl, SR-786, SU-DHL-1, -2, -4,-5,-6,-7,-8,-9,-10, and -16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, SU/RH-HD-1. Non-limiting exemplary sources for such commercially available cell lines include the American Type Culture Collection, or ATCC, (www.atcc.org/) and the German Collection of Microorganisms and Cell Cultures (https://www.dsmz.de/).

As used herein, the term “cognate” refers to a relationship signifying correspondence between two molecules (e.g., between a receptor and its ligand). In the context of a TCR, a “cognate pair” refers to the relationship of two distinct TCR polypeptides or polynucleotides encoding polypeptides derived from a single T cell (e.g., a TCR alpha chain and a TCR beta chain derived from a single T cell). “Cognate” may also refer the relationship between a TCR and the corresponding antigen:MHC complex to which it specifically binds.

As used herein, the term “CRISPR” refers to a technique of sequence specific genetic manipulation relying on the clustered regularly interspaced short palindromic repeats pathway (CRISPR). CRISPR can be used to perform gene editing and/or gene regulation, as well as to simply target proteins to a specific genomic location. Gene editing refers to a type of genetic engineering in which the nucleotide sequence of a target polynucleotide is changed through introduction of deletions, insertions, or base substitutions to the polynucleotide sequence. In some aspects, CRISPR-mediated gene editing utilizes the pathways of nonhomologous end-joining (NHEJ) or homologous recombination to perform the edits. Gene regulation refers to increasing or decreasing the production of specific gene products such as protein or RNA.

The term “guide RNA” or “gRNA” as used herein refers to the guide RNA sequences used to target the CRISPR complex to a specific nucleotide sequence such as a specific region of a cell's genome. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 (2016) 74-83).

As used herein, the term “detectable marker” refers to at least one marker capable of directly or indirectly, producing a detectable signal. A non-exhaustive list of exemplary markers includes enzymes which produce a detectable signal, for example by colorimetry, fluorescence, luminescence, such as horseradish peroxidase, alkaline phosphatase, 0-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S, or ¹²⁵I.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “effective amount” or “therapeutically effective amount” of a composition, is a quantity sufficient to achieve a desired therapeutic effect, e.g., an amount which results in the decrease in the symptoms associated with a disease that is being treated, e.g., the diseases or medical conditions associated with cancer or viral infection. The amount of a composition of the present technology administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions of the present technology can also be administered in combination with one or more additional therapeutic compounds. In some embodiments, effective amount refers to the quantity of cells of the present technology that is partially or fully effective in neutralizing the cancer or viral infection.

As used herein, the term “epitope” means a protein determinant capable of specific binding to an antibody or TCR. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, “elevated expression” refers to an increase in gene expression or protein expression, as compared to a control or a reference sample (e.g., an increase of at least 2-fold, from about 2-fold to about 150-fold, from 5-fold to 150-fold, from 5-fold to 100-fold, from 10-fold to 150-fold, from 10-fold to 100-fold, from 50-fold to 150-fold, from 50-fold to 100-fold, from 75-fold to 150-fold, or from 75-fold to 100-fold, as compared to a control or a normal reference sample). By “decreased expression” refers to an overall reduction in gene expression or protein expression, as compared to a control or a reference sample (e.g., 20% or greater, of 50% or greater, or of 75%, 80%, 85%, 90%, 95%, or greater.

An increase or decrease in gene expression or protein expression can be determined using any useful methods known in the art or described herein (e.g., ELISA).For therapeutic applications, to “decrease” can refer to the reduction in the level of polypeptides or proteins associated with the disorder (e.g., a tauopathy, TBI, or stroke). For diagnostic or monitoring applications, to “decrease” can refer to a decrease in the level of protein or nucleic acid detected by the diagnostic or monitoring assays.

As used herein, the term “gene” means a segment of DNA that contains all the information for the regulated biosynthesis of an RNA product, including promoters, exons, introns, and other untranslated regions that control expression.

As used herein, the term “humanized” forms of non-human proteins (e.g., murine TCRs) are chimeric proteins which contain minimal sequence derived from non-human homologs of the protein. For the most part, humanized proteins are human TCRs in which variable region residues of the recipient are replaced by variable region residues from a non-human species (donor TCR) such as mouse, rat, rabbit or nonhuman primate having the desired specificity, affinity, and capacity.

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein), when compared and aligned for maximum correspondence over a comparison window or designated region) as measured using a BLAST or BLAST 2.0 sequence comparison algorithms with default parameters described below, or by manual alignment and visual inspection, e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

A “Kozak consensus sequence” or “Kozak sequence” is an mRNA sequence that is recognized by a ribosome as a translation start site. A Kozak sequence comprises a start codon (also known as an initiation codon) for initiation of translation and additional flanking nucleotides. The start codon specifies a methionine amino acid at the N-terminus of a translated polypeptide. The Kozak consensus sequence for vertebrates is known in the art (e.g. Kozak, M. 1987 Nucleic Acids Res. 15(20): 8125-48). In some embodiments, Kozak sequences can be modified to be “strong”, meaning that the nucleotide sequence closely matches the consensus sequence, particularly at nucleotides +4 and −3 relative to the number one nucleotide. An “adequate” Kozak sequence has just one of these matching nucleotides while a “weak” Kozak sequence has neither matching nucleotide. The strength of a Kozak sequence directly correlates with the amount of polypeptides translated from an expressed mRNA. In general, strong Kozak sequences result in greater efficiency of translation of an expressed mRNA while fewer polypeptides are transcribed from mRNAs with weak Kozak sequences.

As used herein, “major histocompatibility complex” or “MHC” refers to a cell surface protein that presents antigens to T cells. Class I MHC molecules are recognized by CD8+ T cells. Class II MHC molecules are recognized by CD4+ T cells. An MHC molecule loaded with an antigen or epitope thereof is referred to as an antigen:MHC complex.

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells may either be isolated or obtained from a commercially available source. Non-limiting examples of commercial NK cell lines include lines NK-92 (ATCC® CRL-2407™), NK-92MI (ATCC® CRL-2408™). Further examples include but are not limited to NK lines HANK1, KHYG-1, NKL, NK-YS, NOI-90, and YT. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the term “operably linked” refers to two or more polynucleotides that are joined as part of the same nucleic acid molecule. In some embodiments, the joined polynucleotides are suitably positioned and oriented for transcription to be initiated from the same expression control element. In some embodiments, transcription of a polynucleotide operably linked to an expression control element (e.g., a promoter) is controlled, regulated, or influenced by the expression control element.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide”, “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

A polypeptide, peptide, polynucleotide, or cell may be said to be “isolated” or “substantially pure” when physical, mechanical, or chemical methods have been employed to remove the polypeptide, peptide, polynucleotide, or cell from other cells or cellular constituents. An isolated polypeptide, peptide, polynucleotide, or cell (e.g., an isolated cell), “substantially pure” or “substantially pure and isolated” polypeptide, peptide, polynucleotide, or cell is typically considered removed from cellular constituents and substantially pure when it is at least 60% by weight free from the proteins and naturally occurring organic molecules with which it is naturally associated. The polypeptide may be at least 75%, 80%, 85%, 90%, 95%, or 99% by weight pure. A substantially pure polypeptide, peptide, polynucleotide, or cell (e.g., a substantially pure antibody or fragment thereof) may be obtained by standard techniques, for example, by expression of a recombinant nucleic acid encoding the polypeptide, or by chemically synthesizing the polypeptide. Purity can be measured by any appropriate method, e.g., by column chromatography, polyacrylamide gel electrophoresis, or HPLC analysis.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, the term “separate” therapeutic use refers to an administration of at least two active ingredients at the same time or at substantially the same time by different routes.

As used herein, the term “repeat” therapeutic use refers to administration of active ingredients at different times, the administration route being identical or different. More particularly, sequential use refers to the whole administration of an active ingredient before a second administration of the same or different active ingredient commences. It is thus possible to administer one of the active ingredients over several minutes, hours, days, months, or years before a second administration.

As used herein, the terms “subject”, “individual” and “patient” are used interchangeably and refer to a human or non-human animal, e.g., domestic animals (e.g., dogs, cats and the like), farm animals (e.g., cows, sheep, pigs, horses and the like), wild animals, (bats, raccoons, foxes, skunks, squirrels, chipmunks, mice, rabbits, and the like), and laboratory animals (e.g., monkey, rats, mice, rabbits, guinea pigs and the like). In some embodiments, the subject is a mammal. In particular embodiments, the subject is a human.

As used herein, the term “switch” refers to a mechanism by which the expression, activation, or stability of a recombinant TCR or a component of a recombinant TCR is controlled (i.e. a mechanism to turn TCRs “on” or “off”). Switch mechanisms include but are not limited to TCR expression systems that require co-expression of more than one construct to be activated, suicide switches, safety switches, and TCRs that require multimerization for activation. In some embodiments, a switch is inducible.

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™) TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); and cutaneous T-cell lymphoma lines, e.g., HuT78 (ATCC CRM-TIB-161), MJ[G11](ATCC CRL-8294), HuT102 (ATCC TIB-162). Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the term “T cell receptor” or “TCR” refers to a heterodimeric cell surface protein of the immunoglobulin super-family which is associated with invariant proteins of the CD3 complex involved in mediating signal transduction. The TCR is composed of two cognate protein chains: an alpha (a) chain and a beta (p) chain (encoded by TRA (Entrez gene: 6955) and the TRB gene (Entrez gene: 6957), respectively), or a gamma (γ) chain and a delta (6) chain (encoded by TRG (Entrez gene: 6965) and TRD gene (Entrez gene: 6964), respectively). Each chain is composed of two extracellular domains: a variable (V) region and a constant (C) region. The constant region is proximal to the cell membrane, followed by a transmembrane region and a short cytoplasmic tail, while the variable region binds to the antigen:MHC complex. The variable domain of both the TCR α-chain and β-chain each have three hypervariable or complementarity determining regions (CDRs). There is also an additional area of hypervariability on the β-chain (HV4) that does not normally contact antigen and, therefore, is not considered a CDR. CDR3 is the main CDR responsible for recognizing processed antigen, although CDR1 of the alpha chain has also been shown to interact with the N-terminal part of the antigenic peptide, whereas CDR1 of the β-chain interacts with the C-terminal part of the peptide. CDR2 is thought to recognize the MHC. CDR4 of the β-chain is not thought to participate in antigen recognition, but has been shown to interact with superantigens. The constant domain of the TCR consists of short connecting sequences in which a cysteine residue forms disulfide bonds, which form a link between the two chains.

The diverse repertoire of TCRs in a subject is accomplished by V(D)J recombination, a somatic recombination mechanism that rearranges variable (V), joining (J), and diversity (D) gene segments. In humans, the TRA gene locus comprises 54 TRAV (V) segments, 61 TRAJ (J) segments, and a unique constant TRAC (C) segment. The TRB gene locus comprises 64-67 TRBV (V) segments, 2 TRBD (D) segments, 14 TRBJ (J) segments, and 2 TRBC (C) segments. The TRG gene locus comprises 12-15 TRGV (V) segments, 5 TRGJ (J) segments, and 2 TRGC (C) segments. The TRD gene locus is embedded in the TRA gene locus and contains 8 TRDV or TRAV/DV (V) segments, 3 TRDD (D) segments, 4 TRDJ (J) segments, and one TRDC (C) segment. Non-limiting examples of TCR amino acid sequences are known in the art and provided herein.

Human TCR (α) chain TRAC segment:
(SEQ ID NO: 2)
PNIQNPDPAVYQLRDSKSSDKSVCLFTDFDSQTNVSQSKDSDVYITDKTV
LDMRSMDFKSNSAVAWSNKSDFACANAFNNSIIPEDTFFPSPESSCDVKL
VEKSFETDTNLNFQNLSVIGFRILLLKVAGENLLMTLRLWSS
Human TCR (β) chain TRBC1 segment:
(SEQ ID NO: 3)
EDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSVSYQQGVLSATILYE
ILLGKATLYAVLVSALVLMAMVKRKDF
Human TCR (β) chain TRBC2 segment:
(SEQ ID NO: 4)
DLKNVFPPEVAVFEPSEAEISHTQKATLVCLATGFYPDHVELSWWVNGKE
VHSGVSTDPQPLKEQPALNDSRYCLSSRLRVSATFWQNPRNHFRCQVQFY
GLSENDEWTQDRAKPVTQIVSAEAWGRADCGFTSESYQQGVLSATILYEI
LLGKATLYAVLVSALVLMAMVKRKDSRG
Human TCR (γ) chain TRGCI segment:
(SEQ ID NO: 5)
DKQLDADVSPKPTIFLPSIAETKLQKAGTYLCLLEKFFPDVIKIHWQEKK
SNTILGSQEGNTMKTNDTYMKFSWLTVPEKSLDKEHRCIVRHENNKNGVD
QEIIFPPIKTDVITMDPKDNCSKDANDTLLLQLTNTSAYYMYLLLLLKSV
VYFAIITCCLLRRTAFCCNGEKS
Human TCR (γ) chain TRGC2 segment:
(SEQ ID NO: 6)
DKQLDADVSPKPTIFLPSIAETKLQKAGTYLCLLEKFFPDIIKIHWQEKK
SNTILGSQEGNTMKTNDTYMKFSWLTVPEESLDKEHRCIVRHENNKNGID
QEIIFPPIKTDVTTVDPKDSYSKDANDVITMDPKDNWSKDANDTLLLQLT
NTSAYYMYLLLLLKSVVYFAIITCCLLGRTAFCCNGEKS
Human TCR (δ) chain TRDC segment:
(SEQ ID NO: 7)
SQPHTKPSVFVMKNGTNVACLVKEFYPKDIRINLVSSKKITEFDPAIVIS
PSGKYNAVKLGKYEDSNSVTCSVQHDNKTVHSTDFEVKTDSTDHVKPKET
ENTKQPSKSCHKPKAIVHTEKVNMMSLTVLGLRMLFAKTVAVNFLLTAKL
FFL

As used herein, the term “therapeutic agent” is intended to mean a nucleic acid, recombinant TCR, vector, cell, or population of cells that, when present in an effective amount, produces a desired therapeutic effect on a subject in need thereof.

Amino acid sequence modification(s) of the TCRs described herein are contemplated. For example, it may be desirable to improve the binding affinity and/or other biological properties of the TCR. Amino acid sequence variants of an TCR are prepared by introducing appropriate nucleotide changes into the TCR nucleic acid, or by peptide synthesis. Such modifications include, for example, deletions from, and/or insertions into and/or substitutions of, residues within the amino acid sequences of the TCR. Any combination of deletion, insertion, and substitution is made to obtain the TCR of interest, as long as the obtained TCR possesses the desired properties. The modification also includes the change of the pattern of glycosylation of the protein. The sites of greatest interest for substitutional mutagenesis include the hypervariable regions, but FR alterations are also contemplated. “Conservative substitutions” are shown in the Table below.

TABLE 1
Amino Acid Substitutions

| | | | Conservative |
|---|---|---|---|
| Original Residue | | Exemplary Substitutions | Substitutions |
| Ala | (A) | val; leu; ile | val |
| Arg | (R) | lys; gln; asn | lys |
| Asn | (N) | gln; his; asp, lys; arg | gln |
| Asp | (D) | glu; asn | glu |
| Cys | (C) | ser; ala | ser |
| Gln | (Q) | asn; glu | asn |
| Glu | (E) | asp; gln | asp |
| Gly | (G) | ala | ala |
| His | (H) | asn; gln; lys; arg | arg |
| Ile | (I) | leu; val; met; ala; phe; norleucine | leu |
| Leu | (L) | norleucine; ile; val; met; ala; phe | ile |
| Lys | (K) | arg; gln; asn | arg |
| Met | (M) | leu; phe; ile | leu |
| Phe | (F) | leu; val; ile; ala; tyr | tyr |
| Pro | (P) | ala | ala |
| Ser | (S) | thr | thr |
| Thr | (T) | ser | ser |
| Trp | (W) | tyr; phe | tyr |
| Tyr | (Y) | trp; phe; thr; ser | phe |
| Val | (V) | ile; leu; met; phe; ala; norleucine | leu |

As used herein, “specifically binds” refers to a molecule (e.g., a TCR) which recognizes and binds another molecule (e.g., an antigen:MHC complex), but that does not substantially recognize and bind other molecules. The terms “specific binding,” “specifically binds to,” or is “specific for” a particular molecule (e.g., a particular cell, antigen, epitope, or antigen:MHC complex), as used herein, can be exhibited, for example, by a molecule having a K_{d} for the molecule to which it binds to of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, or greater. The term “specifically binds” may also refer to binding where a molecule (e.g., a TCR) binds to a particular cell, antigen, epitope, or antigen:MHC complex without substantially binding to any other cell, antigen, epitope, or antigen:MHC complex. For example, the TCR may have, for example, at least 10- to 100-fold greater affinity (e.g., 10¹-, 10²-, 10³-, 10⁴-, 10⁵-, 10⁶-, 10⁷-, 10⁸-, 10⁹-, or 10¹⁰-fold greater affinity) to one antigen:MHC complex than to another antigen:MHC complex.

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting examples of promoters include p2A, CMV, and E1α.

The term “transduce” or “transduction” as it is applied to the production of recombinant cells refers to the process whereby a foreign nucleotide sequence is introduced into a cell. In some embodiments, this transduction is done via a vector.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. By “treating cancer” it is meant that the cancer or cancer cells are, e.g., alleviated, reduced, cured, or placed in a state of remission. By “treating a viral infection” it is meant that the virus or viral load is, e.g., alleviated, reduced, cured, or placed in a state of remission

It is also to be appreciated that the various modes of treatment of cancer and viral infections as described herein are intended to mean “substantial,” which includes total but also less than total treatment, and wherein some biologically or medically relevant result is achieved such as extended lifespan of the subject. The treatment may be a continuous prolonged treatment to prevent recurrence, or few time administrations for acute treatment.

### I. Compositions of the Present Technology

In one aspect, the present technology provides a recombinant T cell receptor (TCR) library vector comprising: (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA isolated from a single lysed T cell that is present in a compartment. In some embodiments, the mRNA of the single lysed T cell is isolated using an mRNA capture reagent or reverse transcription-PCR (RT-PCR). Additionally or alternatively, in some embodiments, the compartment containing the contents of the single lysed T cell is a microwell (e.g., a microwell within a 96-well plate) or a droplet. In some embodiments, the mRNA of a single lysed T cell is isolated inside a compartment, without the use of an mRNA capture reagent.

The term “vector” intends a recombinant vector that retains the ability to infect and transduce non-dividing and/or slowly-dividing cells and integrate into the target cell's genome. In some embodiments, the vector is derived from or based on a wild-type virus. In further embodiments, the vector is derived from or based on a wild-type lentivirus, retrovirus, adenovirus, or adeno-associated virus. Examples of such, include without limitation, human immunodeficiency virus (HIV), human T-lymphotropic virus type 1 (HTLV-1), human T-lymphotropic virus type 2 (HTLV-2), human adenovirus (HadV-1 to 57), adeno-associated virus (AAV), equine infectious anaemia virus (EIAV), simian immunodeficiency virus (SIV) and feline immunodeficiency virus (FIV), and murine leukemia virus (MLV). It will be evident that a viral vector according to the present disclosure need not be confined to the components of a particular virus. The viral vector may comprise components derived from two or more different viruses, and may also comprise synthetic components.

In certain embodiments of the vector, the vector backbone is selected from a group consisting of a retroviral, a lentiviral, an adenoviral, and an adeno-associated viral vector backbone. The genome of the vector backbone comprises components from the virus from which it was originally derived. For example, in some embodiments, a vector backbone contains essential vector components compatible with the RNA genome, including reverse transcription and integration systems. In some embodiments, these include gag and pol proteins derived from a particular retrovirus. In some embodiments, the structural components of the vector backbone have been altered genetically or otherwise so as to provide desired useful properties. For example, the vector host range and target cell types can be altered by using different env genes in the vector production system to give the vector a different target specificity.

In a particular embodiment, the vector backbone is a lentiviral vector backbone, e.g., pLVX. Lentiviral vectors are based on or derived from oncoretroviruses (the sub-group of retroviruses containing MLV) and lentiviruses (the sub-group of retroviruses containing HIV). Nonlimiting examples include ASLV, SNV and RSV, all of which have been split into packaging and vector components for lentiviral vector particle production systems. The lentiviral vector particle according to the present disclosure may be based on a genetically or otherwise (e.g., by specific choice of packaging cell system) altered version of a particular retrovirus.

Non-limiting, exemplary vector backbones are known in the art, e.g., see U.S. Pat. Nos. 6,924,123; 7,056,699; 7,07,993; 7,419,829 and 7,442,551, incorporated herein by reference, and Invitrogen's pLenti series versions 4, 6, and 6.2 “ViraPower” system, pHIV-7-GFP, lab generated and used by the City of Hope Research Institute; “Lenti-X” lentiviral vector, pLVX, manufactured by Clontech; pLKO.1-puro, manufactured by Sigma-Aldrich; pLemiR, manufactured by Open Biosystems; and pLV, lab generated and used by Charite Medical School, Institute of Virology (CBF), Berlin, Germany.

Certain retroviral sequences facilitate integration into the target cell genome (see, e.g. U.S. Pat. No. 6,924,123). Each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences capable of controlling the expression of the viral genes. Encapsidation of the retroviral genome occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′end of the RNA. The sizes of the three elements can vary considerably among different retroviruses. U3 contains most of the expression control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

Viral particles are produced by expressing the vector RNA genome from a DNA construct encoding it in a host cell. In some embodiments, the components of the viral particles that are not encoded by the vector backbone are provided in trans by additional nucleic acid sequences (the “packaging system”, which usually includes either or both of the gag/pol and env genes) expressed in the host cell. In some embodiments, the set of sequences required for the production of the viral vector particles are introduced into the host cell by transient transfection, or integrated into the host cell genome, or provided through use of a packaging cell line. The techniques involved are known to those skilled in the art.

In some embodiments, the method or process of derivation of the first polynucleotide and second polynucleotide results in structural features in the first polynucleotide and/or the second polynucleotide that are distinct from other vectors and methods known in the art. For example, use of an mRNA capture reagent with the individual compartment for the lysed T cell allows for capture of a cognate pair of TCR polynucleotides. Other non-limiting examples of structural features include restriction enzyme recognition sites, integrated primer sites, and sequences derived from the mRNA capture reagent.

In some embodiments of the vector, the mRNA capture reagent is selected from the group consisting of a poly(dT) coated bead, an oligonucleotide-coated bead, a hydrogel bead, and a printed oligo on the surface of a microarray well. For example, the mRNA capture agent can be a solid support, such as a bead, comprising immobilized oligonucleotides or polymer networks such as dextran and agarose. In some embodiments, the bead is a silica bead or a magnetic bead. In some embodiments, the mRNA capture agent comprises oligonucleotides which hybridize mRNA. For example, the oligonucleotides may comprise at least one poly(T) and/or primers specific to a transcript of interest. In certain embodiments, a bead of the mRNA capture reagent is smaller than the individual cells that being isolated (e.g., T cells). In some embodiments, sequestering single T cells with an mRNA capture agent is performed prior to lysis of the T cell. In other embodiments, sequestering single T cells with the mRNA capture agent is performed concurrently with T cell lysis. Thus, in some embodiments, single T cells and an mRNA capture agents are isolated into individual microvesicles in an emulsion in the presence of a cell lysis solution.

In some embodiments, the individual compartment has a volume of 100 nL or less, 50 nL or less, 40 nL or less, 30 nL or less, 20 nL or less, 10 nL or less, 5 nL or less. In particular embodiments, the individual compartment has a volume of 5 nL or less. In some embodiments, the individual compartment is a droplet or microvesicle, optionally in an emulsion. In some embodiments, the compartment is a well. In certain embodiments, the well is located in a printed polymer slide, a plastic plate, a microtiter plate, or a gel. In some embodiments, the well is sealed with a permeable membrane prior to lysis of the T cell or prior to performing RT-PCR. Compartmentalized preparation as described herein enables characterization of the library and minimizes the likelihood of PCR error variants which may be included in the final drug product. This reduces the risk that the final therapeutic cell composition contains uncharacterizable and potentially very dangerous variants, which may induce side effects and/or off-target TCR binding specificity.

In some embodiments, the mRNA of the single lysed T cell is reverse transcribed into cDNA using any method known in the art. For example, in some embodiments, reverse transcription is performed using overlap extension (OE) reverse transcription PCR (RT-PCR). The reaction mix for OE-RT-PCR includes primers designed to create a single PCR product comprising the cDNA of two or more transcripts of interest covalently linked together. Primer design for OE-RT-PCR determines which transcripts of interest (e.g. TCR gene transcripts) expressed by a given cell are linked together. For example, in certain embodiments, primers are designed that cause the respective cDNAs from cognate pair TCR chain transcripts to be covalently linked together. Non-limiting examples of OE-RT-PCR reaction conditions are provided in Table 3 herein. Non-limiting examples of PCR primers suitable for performing the reaction to obtain linked TCR cDNAs are provided in Table 4 herein. The linked cDNA products of OE RT-PCR are recovered and used as a template for nested PCR, which amplifies the linked transcripts of interest. Exemplary reaction conditions for nested PCR are provided in Table 5 herein. In some embodiments, the purified products of nested PCR are then sequenced and pairing information is analyzed. In some embodiments, restriction and ligation may be used to link cDNA of multiple transcripts of interest. In other embodiments, recombination may be used to link cDNA of multiple transcripts of interest.

In some embodiments, the TCRα polypeptide comprises at least one TRAV segment and a TRAC segment or an equivalent of each thereof. In some embodiments, the TCRα polypeptide comprises at least one TRAV segment, a TRAC segment, and at least one TRAD segment. In some embodiments, the TCRα polypeptide comprises at least one TRAV segment, a TRAC segment, at least one TRAD segment, and at least one TRAJ segment. In some embodiments, the TCRα polypeptide comprises at least one TRAV segment, a TRAC segment, and at least one TRAJ segment.

In some embodiments, the TCRβ polypeptide comprises at least one TRBV segment and a TRBC segment or an equivalent of each thereof. In some embodiments, the TCRβ polypeptide comprises at least one TRBV segment, at least one TRBC segment, and at least one TRBD segment. In some embodiments, the TCRβ polypeptide comprises at least one TRBV segment, at least one TRBC segment, at least one TRBD segment, and at least one TRBJ segment. In some embodiments, the TCRβ polypeptide comprises at least one TRBV segment, at least one TRBC segment, and at least one TRBJ segment.

In some embodiments, the TCRγ polypeptide comprises at least one TRGV segment and a TRGC segment or an equivalent of each thereof. In some embodiments, the TCRγ polypeptide comprises at least one TRGV segment, at least one TRGC segment, and at least one TRGD segment. In some embodiments, the TCRγ polypeptide comprises at least one TRGV segment, at least one TRGC segment, at least one TRGD segment, and at least one TRGJ segment. In some embodiments, the TCRγ polypeptide comprises at least one TRGV segment, at least one TRGC segment, and at least one TRGJ segment.

In some embodiments, the TCRδ polypeptide comprises at least one TRDV segment and a TRDC segment or an equivalent of each thereof. In some embodiments, the TCRδ polypeptide comprises at least one TRDV segment, at least one TRDC segment, and at least one TRDD segment. In some embodiments, the TCRδ polypeptide comprises at least one TRDV segment, at least one TRDC segment, at least one TRDD segment, and at least one TRDJ segment. In some embodiments, the TCRδ polypeptide comprises at least one TRDV segment, at least one TRDC segment, and at least one TRDJ segment.

In some embodiments of the vector, the first polynucleotide and the second polynucleotide are operably linked, optionally via a linker polynucleotide. In some embodiments, the linker polynucleotide encodes a linker polypeptide. As used herein, the term “linker polypeptide” relates to any amino acid sequence comprising from 1 to 10, or alternatively, 8 amino acids, or alternatively 6 amino acids, or alternatively 5 amino acids that may be repeated from 1 to 10, or alternatively to about 8, or alternatively to about 6, or alternatively about 5, or 4 or alternatively 3, or alternatively 2 times. For example, the linker may comprise up to 15 amino acid residues consisting of a pentapeptide repeated three times. In one aspect, the linker sequence is a (Glycine4Serine)3 (SEQ ID NO: 8) flexible polypeptide linker comprising three copies of gly-gly-gly-gly-ser (SEQ ID NO: 9), or equivalents thereof. Non-limiting examples of linker sequences are known in the art, e.g., GGGGSGGGGSGGGG (SEQ ID NO: 10) (and equivalents thereof); the tripeptide EFM; or Glu-Phe-Gly-Ala-Gly-Leu-Val-Leu-Gly-Gly-Gln-Phe-Met (SEQ ID NO: 11), and equivalents of each thereof. In some embodiments of the vector, the first polynucleotide and the second polynucleotide have been operably linked by reverse transcription and PCR amplification of the captured T cell mRNA.

In some embodiments of the vector, the first polynucleotide and the second polynucleotide have been linked and/or cloned into the vector backbone using a restriction enzyme that cleaves at a target restriction endonuclease site that is natively found in TCR genes. In certain embodiments, the target restriction endonuclease site occurs in TCR genes with low frequency. “Low frequency” refers to a site that occurs fewer than 20, fewer than 15, fewer than 10, fewer than 5, or fewer than 2 times in a TCR gene. In certain embodiments, the target restriction endonuclease site comprises a silent mutation that does not alter the expressed TCR polypeptide sequence. Non-limiting examples of restriction endonuclease recognition sites are provided in Tables 6 and 7 herein. In some embodiments, the first polynucleotide and/or the second polynucleotide have been altered to incorporate at least one, at least two, at least 3, at least four, at least 5, at least 5, at least 7, at least 8, at least 9, or at least 10 target restriction endonuclease sites disclosed in Table 6 or Table 7.

In certain embodiments of the vector, the vector further comprises at least one, at least 2, at least 3, or at least 4 polynucleotides encoding an expression control element. As used herein, an “expression control element” intends a polynucleotide that directly or indirectly facilitates, promotes, regulates, or influences the expression of a polynucleotide. In some embodiments, the expression control element activates expression of a polynucleotide. In some embodiments, the expression control element maintains expression of a polynucleotide. In some embodiments, the expression control element enhances expression of a polynucleotide. In some embodiments, the expression control element stabilizes a transcript of a polynucleotide. In some embodiments, the expression control element suppresses expression of a polynucleotide. In some embodiments, the activity of the expression control element is inducible. In some embodiments, the expression control element is operably linked to the first polynucleotide and/or the second polynucleotide. In some embodiments, the expression control element is upstream (5′) to the first polynucleotide and/or the second polynucleotide. In some embodiments, the expression control element is downstream (3′) to the first polynucleotide and/or the second polynucleotide. In certain embodiments, the polynucleotide encoding the expression control element is located between the first polynucleotide and the second polynucleotide. In some embodiments, the expression control element is selected from the group consisting of: a promoter, a p2A sequence, an enhancer, and an internal ribosome entry site (IRES) sequence.

A p2A sequence is a short peptide (about 20 amino acids) that produces equimolar levels of multiple genes from the same mRNA. The peptides are thought to function by causing the ribosome skip the synthesis of a peptide bond at the C-terminus of a 2A element, leading to separation between the end of the 2A sequence and the next peptide downstream. The resulting “cleavage” occurs between the Glycine and Proline residues found on the C-terminus, meaning the upstream cistron will have a few additional residues added to the end, while the downstream cistron will start with the Proline. In particular embodiments, the p2A sequence is selected from the group consisting of:

T2A:
(SEQ ID NO: 12)
(GSG)EGRGSLLTCGDVEENPGP
P2A:
(SEQ ID NO: 13)
(GSG)ATNFSLLKQAGDVEENPGP
E2A:
(SEQ ID NO: 14)
(GSG)QCTNYALLKLAGDVESNPGP
F2A:
(SEQ ID NO: 15)
(GSG)VKQTLNFDLLKLAGDVESNPG

A promoter is a regulatory polynucleotide that provides a control point for regulated transcription of a polynucleotide. In some embodiments, the promoter is selected from the group consisting of: CMV, EFla, SV40, PGK1, UBC, MNDU3, human beta actin, CAG, TRE, UAS, Ac5, Polyhedrin, CaMKIIa, GAL1, GAL10, GDS, ADH1, CaMV35S, Ubi, H1, and U6. In particular embodiments, the promoter is an EF1α promoter or a CMV promoter.

An internal ribosome entry site is an RNA element that allows for translation initiation in a cap-independent manner, as part of the greater process of protein synthesis. In eukaryotic translation, initiation typically occurs at the 5′ end of mRNA molecules, since 5′ cap recognition is required for the assembly of the initiation complex. The location for IRES elements is often in the 5′UTR, but can also occur elsewhere in mRNAs. In certain embodiments, the IRES is an FMDV or an EMCV IRES sequence. A non-limiting example of a polynucleotide encoding an IRES sequence (EMCV) is provided herein:

(SEQ ID NO: 16)
TATGCTAGTACGTCTCTCAAGGATAAGTAAGTAATATTAAGGTACGGGAG
GTATTGGACAGGCCGCAATAAAATATCTTTATTTTCATTACATCTGTGTG
TTGGTTTTTTGTGTGAATCGATAGTACTAACATACGCTCTCCATCAAAAC
AAAACGAAACAAAACAAACTAGCAAAATAGGCTGTCCCCAGTGCAAGTGC
AGGTGCCAGAACATTTCTCTGGCCTAACTGGCCGGTACCTGAGCTCTAGT
TTCACTTTCCCTAGTTTCACTTTCCCTAGTTTCACTTTCCCTAGTTTCAC
TTTCCCTAGTTTCACTTTCCCCTCGAGGATATCAAGATCTGGCCTCGGCG
GCCAG

An enhancer is a region of DNA that can be bound by proteins (e.g. transcription factors) to increase the likelihood that transcription of a particular target polynucleotide will occur. Enhancers are cis-acting. In some embodiments, they are between 50-1500 base pairs in length. In some embodiments, they are located upstream, downstream, within a target polynucleotide. In some embodiments, the enhancer is selected from the group consisting of CENTG2, GADD45G, and WPRE enhancers. In particular embodiments, the enhancer is Woodchuck Hepatitis Virus (WHP) Posttranscriptional Regulatory Element (WPRE), a DNA sequence that, when transcribed, creates a tertiary structure enhancing expression. WPRE is a tripartite regulatory element with gamma, alpha, and beta components. In some embodiments, the enhancer comprises just the alpha component of WPRE. In other embodiments, the enhancer comprises the full tripartite WPRE sequence. When used alone without the gamma and beta WPRE components, the alpha component is only 9% as active as the full tripartite WPRE.

WPRE alpha sequence:
(SEQ ID NO: 17)
GCCACGGCGGAACTCATCGCCGCCTGCCTTGCCCGCTGCTGGACAGGGGC
TCGGCTGTTGGGCACTGACAATTCCGTGGT
Full tripartite WPRE sequence:
(SEQ ID NO: 18)
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGG
GGCTCGGCTGTTGGGCACTGACAATTCCGTGGTGTTGTCGGGGAAGCTGA
CGTCCTTTCCATGGCTGCTCGCCTGTGTTGCCACCTGGATTCTGCGCGGG
ACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCC
CTCAGACGAGTCGGATCTCCCTTTGGGCCGCCTCCCCGCCTG

In some embodiments of the vector, the vector is circularized. In some embodiments, the vector has been circularized prior to incorporation of the expression control element into the vector. In other embodiments, the vector has been circularized after incorporation of the expression control element into the vector.

In certain embodiments of the vector, the expression control element has been incorporated near a protospacer adjacent motif (PAM). In these embodiments, the expression control element is inserted into the vector via a CRISPR/Cas mediated mechanism. In other embodiments, the expression control element has been incorporated into the vector using a DNA-modifying enzyme selected from a restriction enzyme or a TALEN.

In certain embodiments, the vector further comprises one or more polynucleotides encoding a transposon to facilitate integration of the at least one of the first polynucleotide and the second polynucleotide into a target cell or a host cell. Sleeping Beauty transposase inserts a transposon into a TA dinucleotide base pair in a recipient DNA sequence. For example, in some embodiments, transposons flanking the first polynucleotide and the second polynucleotide facilitate integration into the recombinant cell genome or the immune cell genome at a TA dinucleotide. In some embodiments, the vector backbone is derived from or comprises a non-viral vector. Advantages of non-viral vectors include the ease and relatively low cost of producing sufficient amounts required to meet the entire patient population, stability during storage and lack of immunogenicity. A non-limiting example of a transposon system suitable for use in the vectors of the present technology is a Sleeping Beauty transposon system (see, e.g., Kebriaei, P. et al. (2017) Trends in Genetics 33: 852-70, incorporated herein by reference). A Sleeping Beauty transposon system consists of two components: (i) a transposon containing a gene-expression cassette and (ii) a source of transposase enzyme. By transposing the expression cassette from a plasmid into the genome, sustained transcription of a transgene can be achieved. Exemplary Sleeping beauty transposase vectors include but are not limited to: pSBbi (Kowarz, E. et al. Biotechnol J. 10(4):647-53, available from Addgene), pCMV(CAT)T7-SB100 (Mátés, L. et al. Nat Genet. 2009 June; 41(6):753-61, available from Addgene), and pT2/LTR7 (Wang, J. et al. Nature. 2014 Dec. 18; 516(7531):405-9, available from Addgene).

In some embodiments of the vector, the vector further comprises one or more polynucleotides encoding a detectable marker or a purification marker. In particular embodiments, the detectable marker is a fluorescent protein selected from the group consisting of GFP, YFP, RFP, dTomato, cherry, Cy3, Cy 5, Cy 5.5, Cy 7, DNP, AMCA, Biotin, Digoxigenin, Tamra, Texas Red, rhodamine, Alexafluors, FITC, and TRITC.

In some embodiments of the vector, the vector further comprises one or more polynucleotides encoding a selectable marker. In particular embodiments, the selectable marker confers a positive selection trait in a eukaryotic cell, e.g., blasticidin (bsd gene), G418/Geneticin (neo gene), hygromycin B (hygB gene), puromycin (pac gene), or zeocin (Sh bla gene). In some embodiments, the selectable marker confers a positive selection trait in a bacterial cell, e.g., beta-lactamase gene.

In some embodiments of the vector, the vector further comprises one or more polynucleotides encoding a switch mechanism for controlling expression and/or activation of the first polynucleotide and the second polynucleotide. In other embodiments of the vector, the recombinant TCR encoded by the first polynucleotide and the second polynucleotide comprises a switch mechanism. In particular embodiments, the switch mechanism is a suicide switch, e.g. iCaspase 9, a safety mechanism which can be activated to cause the apoptosis or death of cells comprising a TCR library.

For example, in some embodiments a TCR may comprise an extracellular domain with a target-specific binding element that comprises a label, binding domain, or tag that is specific for a molecule other than the target antigen that is expressed on or by a target cell. In such embodiments, the specificity of the TCR is provided by a second construct that comprises, consists, or consists essentially of a target antigen binding domain and a domain that is recognized by or binds to the label, binding domain, or tag on the TCR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the TCR can be administered to a subject, but it cannot bind its target antigen until the second composition comprising the antigen-specific binding domain is administered.

In other embodiments, a TCR is modified to require multimerization in order to be activated (see, e.g., US 2015/0368342, US 2016/0175359, US 2015/0368360) and/or an exogenous signal, such as a small molecule drug (US 2016/0166613, Yung et al., Science, 2015).

In some embodiments, the vector comprises a polynucleotide encoding a “suicide switch” or “safety switch” to induce cell death of the vector-expressing cells following treatment (Buddee et al., PLoS One, 2013) or to downregulate expression of the TCR following binding to the target antigen (WO 2016/011210). For example, vectors can comprise a suicide gene that confers sensitivity to an antibody or prodrug that can be administered to cease TCR activity. In some embodiments, the antibody or prodrug is administered to a subject that has received TCR library therapy upon the occurrence of an adverse event. Exemplary suicide genes include but are not limited to herpes simplex virus-thymidine kinase (HSV-TK) which renders cells susceptible to ganciclovir (Bonini et al. Science 276: 1719-1724 (1997)), inducible Caspase 9 (iCaspase9) which allows for dimerization and activation of apoptosis when activated by a dimerizer drug (Gargett et al., Front Pharmacol, 2014 5:235), and truncated EGFR which renders cells susceptible to cetuximab (Wang et al. Blood 118: 1255-63 (2011)).

In some embodiments of the vector, the vector further comprises one or more polynucleotides encoding a Kozak consensus sequence. In some embodiments, the Kozak consensus sequence is strong, adequate, or weak.

In some embodiments of the vector, the T cell was screened for reactivity with a target cell or disease antigen prior to lysis. In certain embodiments of the vector, the TCR encoded by the vector has binding specificity for or is activated by a target cell or disease antigen. In certain embodiments, the target cell is a cancer cell, a cell infected with a virus, a cell derived from a subject infected with a virus, a tumor cell, or a tissue biopsy cell isolated from a subject suspected of having a viral infection or cancer. In some embodiments, the cell was isolated from a subject. In some embodiments of the vector, the TCR is screened for specific binding to an disease antigen:MHC complex.

In certain embodiments, the disease antigen is a viral antigen derived from a virus selected from the group consisting of adenovirus, CMV, coronavirus, coxsackievirus, Dengue virus, Epstein-Barr virus (EBV), enterovirus 71 (EV71), Ebola virus, hepatitis A (HAV), hepatitis B (HBV), cytomegalovirus (CMV), hepatitis C (HCV), hepatitis D (HDV), hepatitis E (HEV), human immunodeficiency virus (HIV), human papillomavirus (HPV), herpes simplex virus (HSV), human T-lymphotropic virus (HTLV), influenza A virus, influenza B virus, Japanese encephalitis, leukemia virus, measles virus, molluscum contagiosum, orf virus, parvovirus, rabies virus, respiratory syncytial virus, rift valley fever virus, rubella virus, rotavirus, tick-borne encephalitis (TBEV), simian immunodeficiency virus, tobacco etch virus (TEV), varicella zoster virus, variola, West Nile virus, Zika virus, and Chikungunya virus.

In other embodiments, the disease antigen is a tumor antigen selected from the group consisting of CD45, glypican-3, IGF2B3, Kallikrein 4, KIF20A, Lengsin, Meloe, mucin 5AC (MUC5AC), survivin, cyclin-A1, MAGE-A1, MAGE-C1, MAGE-C2, SSX2, XAGElb/GAGED2A, CD19, CD20, CD22, CD52, epidermal growth factor receptor (EGFR), human epidermal growth factor receptor 2 (HER2), tumor necrosis factor receptor superfamily, member 10a (TRAILR1), receptor activator of nuclear factor kappa-B ligand (RANKL), insulin-like growth factor 1 receptor (IGF1R), epithelial cell adhesion molecule (EpCAM), and carcinoembryonic antigen (CEA).

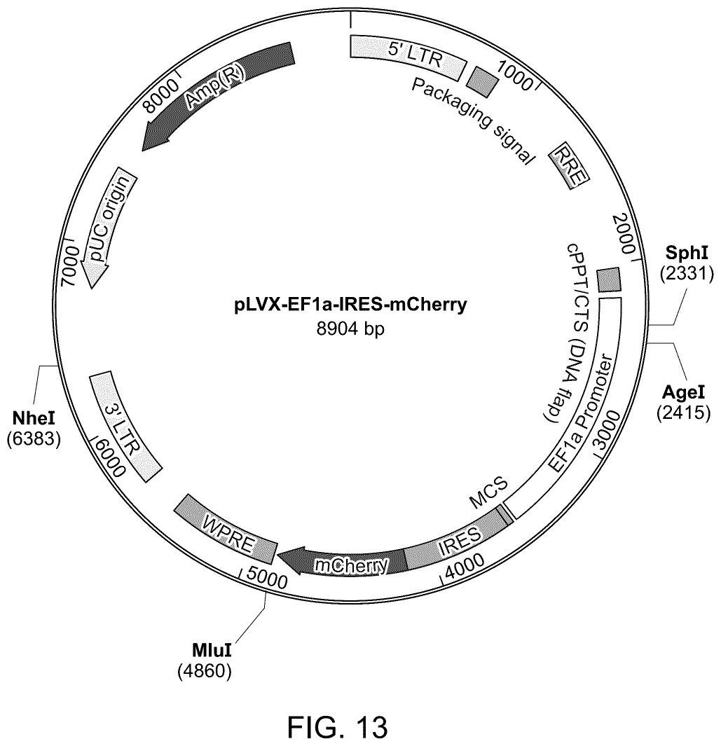

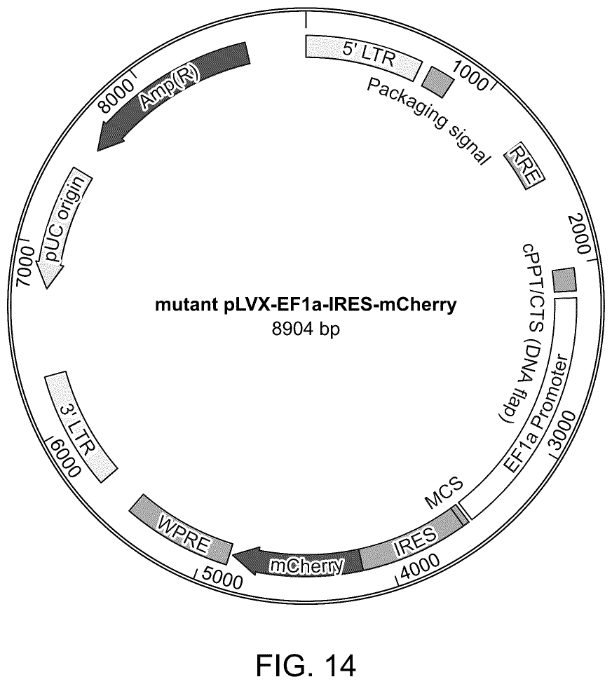

By way of example only, in some embodiments, the original destination cloning vector (for example, pLVX-EF1α-IRES-mCherry) may contain multiple recognition sites for restriction enzymes, such as AgeI (2415), SphI (2331), NheI (8192) and MluI (6669) cutting sites (numbers indicate the location of these cutting sites). See FIG. 13. In some embodiments, the excess restriction enzyme sites for AgeI, SphI, NheI and MluI may be eliminated by performing site-directed mutagenesis (see FIG. 14). In some embodiments, the excess restriction enzyme sites to be removed from the destination cloning vector may be any of those listed in Table 7 or Table 8. In other embodiments, the excess restriction enzyme sites may be substituted with other known restriction enzyme sites that permit T cell receptor paired alpha:beta cloning into linear or circularized vector formats.

Original pLVX-EFla-IRES Mcherry Vector Sequence

(SEQ ID NO: 19)
tggaagggctaattcactcccaaagaagacaagatatccttgatctgtgg
atctaccacacacaaggctacttccctgattagcagaactacacaccagg
gccaggggtcagatatccactgacctttggatggtgctacaagctagtac
cagttgagccagataaggtagaagaggccaataaaggagagaacaccagc
ttgttacaccctgtgagcctgcatgggatggatgacccggagagagaagt
gttagagtggaggtttgacagccgcctagcatttcatcacgtggcccgag
agctgcatccggagtacttcaagaactgctgatatcgagcttgctacaag
ggactttccgctggggactttccagggaggcgtggcctgggcgggactgg
ggagtggcgagccctcagatcctgcatataagcagctgctttttgcctgt
actgggtctctctggttagaccagatctgagcctgggagctctctggcta
actagggaacccactgcttaagcctcaataaagcttgccttgagtgcttc
aagtagtgtgtgcccgtctgttgtgtgactctggtaactagagatccctc
agacccttttagtcagtgtggaaaatctctagcagtggcgcccgaacagg
gacttgaaagcgaaagggaaaccagaggagctctctcgacgcaggactcg
gcttgctgaagcgcgcacggcaagaggcgaggggcggcgactggtgagta
cgccaaaaattttgactagcggaggctagaaggagagagatgggtgcgag
agcgtcagtattaagcgggggagaattagatcgcgatgggaaaaaattcg
gttaaggccagggggaaagaaaaaatataaattaaaacatatagtatggg
caagcagggagctagaacgattcgcagttaatcctggcctgttagaaaca
tcagaaggctgtagacaaatactgggacagctacaaccatcccttcagac
aggatcagaagaacttagatcattatataatacagtagcaaccctctatt
gtgtgcatcaaaggatagagataaaagacaccaaggaagctttagacaag
atagaggaagagcaaaacaaaagtaagaccaccgcacagcaagcggccgg
ccgctgatcttcagacctggaggaggagatatgagggacaattggagaag
tgaattatataaatataaagtagtaaaaattgaaccattaggagtagcac
ccaccaaggcaaagagaagagtggtgcagagagaaaaaagagcagtggga
ataggagctttgttccttgggttcttgggagcagcaggaagcactatggg
cgcagcgtcaatgacgctgacggtacaggccagacaattattgtctggta
tagtgcagcagcagaacaatttgctgagggctattgaggcgcaacagcat
ctgttgcaactcacagtctggggcatcaagcagctccaggcaagaatcct
ggctgtggaaagatacctaaaggatcaacagctcctggggatttggggtt
gctctggaaaactcatttgcaccactgctgtgccttggaatgctagttgg
agtaataaatctctggaacagatttggaatcacacgacctggatggagtg
ggacagagaaattaacaattacacaagcttaatacactccttaattgaag
aatcgcaaaaccagcaagaaaagaatgaacaagaattattggaattagat
aaatgggcaagtttgtggaattggtttaacataacaaattggctgtggta
tataaaattattcataatgatagtaggaggcttggtaggtttaagaatag
tttttgctgtactttctatagtgaatagagttaggcagggatattcacca
ttatcgtttcagacccacctcccaaccccgaggggacccgacaggcccga
aggaatagaagaagaaggtggagagagagacagagacagatccattcgat
tagtgaacggatctcgacggtatcgcctttaaaagaaaaggggggattgg
ggggtacagtgcaggggaaagaatagtagacataatagcaacagacatac
aaactaaagaattacaaaaacaaattacaaaaattcaaaattttcgggtt
tattacagggacagcagagatccagtttatcgatgagtaattcatacaaa
aggactcgcccctgccttggggaatcccagggaccgtcgttaaactccca
ctaacgtagaacccagagatcgctgcgttcccgccccctcacccgcccgc
tctcgtcatcactgaggtggagaagagcatgcgtgaggctccggtgcccg
tcagtgggcagagcgcacatcgcccacagtccccgagaagttggggggag
gggtcggcaattgaaccggtgcctagagaaggtggcgcggggtaaactgg
gaaagtgatgtcgtgtactggctccgcctttttcccgagggtgggggaga
accgtatataagtgcagtagtcgccgtgaacgttctttttcgcaacgggt
ttgccgccagaacacaggtaagtgccgtgtgtggttcccgcgggcctggc
ctctttacgggttatggcccttgcgtgccttgaattacttccacgcccct
ggctgcagtacgtgattcttgatcccgagcttcgggttggaagtgggtgg
gagagttcgaggccttgcgcttaaggagccccttcgcctcgtgcttgagt
tgaggcctggcttgggcgctggggccgccgcgtgcgaatctggtggcacc
ttcgcgcctgtctcgctgctttcgataagtctctagccatttaaaatttt
tgatgacctgctgcgacgctttttttctggcaagatagtcttgtaaatgc
gggccaagatctgcacactggtatttcggtttttggggccgcgggcggcg
acggggcccgtgcgtcccagcgcacatgttcggcgaggcggggcctgcga
gcgcggccaccgagaatcggacgggggtagtctcaagctggccggcctgc
tctggtgcctggcctcgcgccgccgtgtatcgccccgccctgggcggcaa
ggctggcccggtcggcaccagttgcgtgagcggaaagatggccgcttccc
ggccctgctgcagggagctcaaaatggaggacgcggcgctcgggagagcg
ggcgggtgagtcacccacacaaaggaaaagggcctttccgtcctcagccg
tcgcttcatgtgactccacggagtaccgggcgccgtccaggcacctcgat
tagttctcgagcttttggagtacgtcgtctttaggttggggggaggggtt
ttatgcgatggagtttccccacactgagtgggtggagactgaagttaggc
cagcttggcacttgatgtaattctccttggaatttgccctttttgagttt
ggatcttggttcattctcaagcctcagacagtggttcaaagtttttttct
tccatttcaggtgtcgtgaggatctatttccggtgaattcctcgagacta
gttctagagcggccgcggatcccgcccctctccctcccccccccctaacg
ttactggccgaagccgcttggaataaggccggtgtgcgtttgtctatatg
ttattttccaccatattgccgtcttttggcaatgtgagggcccggaaacc
tggccctgtcttcttgacgagcattcctaggggtctttcccctctcgcca
aaggaatgcaaggtctgttgaatgtcgtgaaggaagcagttcctctggaa
gcttcttgaagacaaacaacgtctgtagcgaccctttgcaggcagcggaa
ccccccacctggcgacaggtgcctctgcggccaaaagccacgtgtataag
atacacctgcaaaggcggcacaaccccagtgccacgttgtgagttggata
gttgtggaaagagtcaaatggctcacctcaagcgtattcaacaaggggct
gaaggatgcccagaaggtaccccattgtatgggatctgatctggggcctc
ggtgcacatgctttacatgtgtttagtcgaggttaaaaaacgtctaggcc
ccccgaaccacggggacgtggttttcctttgaaaaacacgatgataatat
ggtgagcaagggcgaggaggataacatggccatcatcaaggagttcatgc
gcttcaaggtgcacatggagggctccgtgaacggccacgagttcgagatc
gagggcgagggcgagggccgcccctacgagggcacccagaccgccaagct
gaaggtgaccaagggtggccccctgcccttcgcctgggacatcctgtccc
ctcagttcatgtacggctccaaggcctacgtgaagcaccccgccgacatc
cccgactacttgaagctgtccttccccgagggcttcaagtgggagcgcgt
gatgaacttcgaggacggcggcgtggtgaccgtgacccaggactcctccc
tgcaggacggcgagttcatctacaaggtgaagctgcgcggcaccaacttc
ccctccgacggccccgtaatgcagaagaagaccatgggctgggaggcctc
ctccgagcggatgtaccccgaggacggcgccctgaagggcgagatcaagc
agaggctgaagctgaaggacggcggccactacgacgctgaggtcaagacc
acctacaaggccaagaagcccgtgcagctgcccggcgcctacaacgtcaa
catcaagttggacatcacctcccacaacgaggactacaccatcgtggaac
agtacgaacgcgccgagggccgccactccaccggcggcatggacgagctg
tacaagtgaacgcgtctggaacaatcaacctctggattacaaaatttgtg
aaagattgactggtattcttaactatgttgctccttttacgctatgtgga
tacgctgctttaatgcctttgtatcatgctattgcttcccgtatggcttt
cattttctcctccttgtataaatcctggttgctgtctctttatgaggagt
tgtggcccgttgtcaggcaacgtggcgtggtgtgcactgtgtttgctgac
gcaacccccactggttggggcattgccaccacctgtcagctcctttccgg
gactttcgctttccccctccctattgccacggcggaactcatcgccgcct
gccttgcccgctgctggacaggggctcggctgttgggcactgacaattcc
gtggtgttgtcggggaagctgacgtcctttccatggctgctcgcctgtgt
tgccacctggattctgcgcgggacgtccttctgctacgtcccttcggccc
tcaatccagcggaccttccttcccgcggcctgctgccggctctgcggcct
cttccgcgtcttcgccttcgccctcagacgagtcggatctccctttgggc
cgcctccccgcctggaattaattctgcagtcgagacctagaaaaacatgg
agcaatcacaagtagcaatacagcagctaccaatgctgattgtgcctggc
tagaagcacaagaggaggaggaggtgggttttccagtcacacctcaggta
cctttaagaccaatgacttacaaggcagctgtagatcttagccacttttt
aaaagaaaagaggggactggaagggctaattcactcccaacgaagacaag
atatccttgatctgtggatctaccacacacaaggctacttccctgattag
cagaactacacaccagggccaggggtcagatatccactgacctttggatg
gtgctacaagctagtaccagttgagccagataaggtagaagaggccaata
aaggagagaacaccagcttgttacaccctgtgagcctgcatgggatggat
gacccggagagagaagtgttagagtggaggtttgacagccgcctagcatt
tcatcacgtggcccgagagctgcatccggagtacttcaagaactgctgat
atcgagcttgctacaagggactttccgctggggactttccagggaggcgt
ggcctgggcgggactggggagtggcgagccctcagatcctgcatataagc
agctgctttttgcctgtactgggtctctctggttagaccagatctgagcc
tgggagctctctggctaactagggaacccactgcttaagcctcaataaag
cttgccttgagtgcttcaagtagtgtgtgcccgtctgttgtgtgactctg
gtaactagagatccctcagacccttttagtcagtgtggaaaatctctagc
agtagtagttcatgtcatcttattattcagtatttataacttgcaaagaa
atgaatatcagagagtgagaggccttgacattgctagcgtttaccgtcga
cctctagctagagcttggcgtaatcatggtcatagctgtttcctgtgtga
aattgttatccgctcacaattccacacaacatacgagccggaagcataaa
gtgtaaagcctggggtgcctaatgagtgagctaactcacattaattgcgt
tgcgctcactgcccgctttccagtcgggaaacctgtcgtgccagctgcat
taatgaatcggccaacgcgcggggagaggcggtttgcgtattgggcgctc
ttccgcttcctcgctcactgactcgctgcgctcggtcgttcggctgcggc
gagcggtatcagctcactcaaaggcggtaatacggttatccacagaatca
ggggataacgcaggaaagaacatgtgagcaaaaggccagcaaaaggccag
gaaccgtaaaaaggccgcgttgctggcgtttttccataggctccgccccc
ctgacgagcatcacaaaaatcgacgctcaagtcagaggtggcgaaacccg
acaggactataaagataccaggcgtttccccctggaagctccctcgtgcg
ctctcctgttccgaccctgccgcttaccggatacctgtccgcctttctcc
cttcgggaagcgtggcgctttctcatagctcacgctgtaggtatctcagt
tcggtgtaggtcgttcgctccaagctgggctgtgtgcacgaaccccccgt
tcagcccgaccgctgcgccttatccggtaactatcgtcttgagtccaacc
cggtaagacacgacttatcgccactggcagcagccactggtaacaggatt
agcagagcgaggtatgtaggcggtgctacagagttcttgaagtggtggcc
taactacggctacactagaagaacagtatttggtatctgcgctctgctga
agccagttaccttcggaaaaagagttggtagctcttgatccggcaaacaa
accaccgctggtagcggtggtttttttgtttgcaagcagcagattacgcg
cagaaaaaaaggatctcaagaagatcctttgatcttttctacggggtctg
acgctcagtggaacgaaaactcacgttaagggattttggtcatgagatta
tcaaaaaggatcttcacctagatccttttaaattaaaaatgaagttttaa
atcaatctaaagtatatatgagtaaacttggtctgacagttaccaatgct
taatcagtgaggcacctatctcagcgatctgtctatttcgttcatccata
gttgcctgactccccgtcgtgtagataactacgatacgggagggcttacc
atctggccccagtgctgcaatgataccgcgagacccacgctcaccggctc
cagatttatcagcaataaaccagccagccggaagggccgagcgcagaagt
ggtcctgcaactttatccgcctccatccagtctattaattgttgccggga
agctagagtaagtagttcgccagttaatagtttgcgcaacgttgttgcca
ttgctacaggcatcgtggtgtcacgctcgtcgtttggtatggcttcattc
agctccggttcccaacgatcaaggcgagttacatgatcccccatgttgtg
caaaaaagcggttagctccttcggtcctccgatcgttgtcagaagtaagt
tggccgcagtgttatcactcatggttatggcagcactgcataattctctt
actgtcatgccatccgtaagatgcttttctgtgactggtgagtactcaac
caagtcattctgagaatagtgtatgcggcgaccgagttgctcttgcccgg
cgtcaatacgggataataccgcgccacatagcagaactttaaaagtgctc
atcattggaaaacgttcttcggggcgaaaactctcaaggatcttaccgct
gttgagatccagttcgatgtaacccactcgtgcacccaactgatcttcag
catcttttactttcaccagcgtttctgggtgagcaaaaacaggaaggcaa
aatgccgcaaaaaagggaataagggcgacacggaaatgttgaatactcat
actcttcctttttcaatattattgaagcatttatcagggttattgtctca
tgagcggatacatatttgaatgtatttagaaaaataaacaaataggggtt
ccgcgcacatttccccgaaaagtgccacctgacgtcgacggatcgggaga
tcaacttgtttattgcagcttataatggttacaaataaagcaatagcatc
acaaatttcacaaataaagcatttttttcactgcattctagttgtggttt
gtccaaactcatcaatgtatcttatcatgtctggatcaactggataactc
aagctaaccaaaatcatcccaaacttcccaccccataccctattaccact
gccaattacctgtggtttcatttactctaaacctgtgattcctctgaatt
attttcattttaaagaaattgtatttgttaaatatgtactacaaacttag
tagt

Modified pLVX-EFla-IRES Mcherry Vector Sequence

(SEQ ID NO: 20)
tggaagggctaattcactcccaaagaagacaagatatccttgatctgtgg
atctaccacacacaaggctacttccctgattagcagaactacacaccagg
gccaggggtcagatatccactgacctttggatggtgctacaagctagtac
cagttgagccagataaggtagaagaggccaataaaggagagaacaccagc
ttgttacaccctgtgagcctgcatgggatggatgacccggagagagaagt
gttagagtggaggtttgacagccgcctagcatttcatcacgtggcccgag
agctgcatccggagtacttcaagaactgctgatatcgagcttgctacaag
ggactttccgctggggactttccagggaggcgtggcctgggcgggactgg
ggagtggcgagccctcagatcctgcatataagcagctgctttttgcctgt
actgggtctctctggttagaccagatctgagcctgggagctctctggcta
actagggaacccactgcttaagcctcaataaagcttgccttgagtgcttc
aagtagtgtgtgcccgtctgttgtgtgactctggtaactagagatccctc
agacccttttagtcagtgtggaaaatctctagcagtggcgcccgaacagg
gacttgaaagcgaaagggaaaccagaggagctctctcgacgcaggactcg
gcttgctgaagcgcgcacggcaagaggcgaggggcggcgactggtgagta
cgccaaaaattttgactagcggaggctagaaggagagagatgggtgcgag
agcgtcagtattaagcgggggagaattagatcgcgatgggaaaaaattcg
gttaaggccagggggaaagaaaaaatataaattaaaacatatagtatggg
caagcagggagctagaacgattcgcagttaatcctggcctgttagaaaca
tcagaaggctgtagacaaatactgggacagctacaaccatcccttcagac
aggatcagaagaacttagatcattatataatacagtagcaaccctctatt
gtgtgcatcaaaggatagagataaaagacaccaaggaagctttagacaag
atagaggaagagcaaaacaaaagtaagaccaccgcacagcaagcggccgg
ccgctgatcttcagacctggaggaggagatatgagggacaattggagaag
tgaattatataaatataaagtagtaaaaattgaaccattaggagtagcac
ccaccaaggcaaagagaagagtggtgcagagagaaaaaagagcagtggga
ataggagctttgttccttgggttcttgggagcagcaggaagcactatggg
cgcagcgtcaatgacgctgacggtacaggccagacaattattgtctggta
tagtgcagcagcagaacaatttgctgagggctattgaggcgcaacagcat
ctgttgcaactcacagtctggggcatcaagcagctccaggcaagaatcct
ggctgtggaaagatacctaaaggatcaacagctcctggggatttggggtt
gctctggaaaactcatttgcaccactgctgtgccttggaatgctagttgg
agtaataaatctctggaacagatttggaatcacacgacctggatggagtg
ggacagagaaattaacaattacacaagcttaatacactccttaattgaag
aatcgcaaaaccagcaagaaaagaatgaacaagaattattggaattagat
aaatgggcaagtttgtggaattggtttaacataacaaattggctgtggta
tataaaattattcataatgatagtaggaggcttggtaggtttaagaatag
tttttgctgtactttctatagtgaatagagttaggcagggatattcacca
ttatcgtttcagacccacctcccaaccccgaggggacccgacaggcccga
aggaatagaagaagaaggtggagagagagacagagacagatccattcgat
tagtgaacggatctcgacggtatcgcctttaaaagaaaaggggggattgg
ggggtacagtgcaggggaaagaatagtagacataatagcaacagacatac
aaactaaagaattacaaaaacaaattacaaaaattcaaaattttcgggtt
tattacagggacagcagagatccagtttatcgatgagtaattcatacaaa
aggactcgcccctgccttggggaatcccagggaccgtcgttaaactccca
ctaacgtagaacccagagatcgctgcgttcccgccccctcacccgcccgc
tctcgtcatcactgaggtggagaagagcaagcgtgaggctccggtgcccg
tcagtgggcagagcgcacatcgcccacagtccccgagaagttggggggag
gggtcggcaattgatccggagcctagagaaggtggcgcggggtaaactgg
gaaagtgatgtcgtgtactggctccgcctttttcccgagggtgggggaga
accgtatataagtgcagtagtcgccgtgaacgttctttttcgcaacgggt
ttgccgccagaacacaggtaagtgccgtgtgtggttcccgcgggcctggc
ctctttacgggttatggcccttgcgtgccttgaattacttccacgcccct
ggctgcagtacgtgattcttgatcccgagcttcgggttggaagtgggtgg
gagagttcgaggccttgcgcttaaggagccccttcgcctcgtgcttgagt
tgaggcctggcttgggcgctggggccgccgcgtgcgaatctggtggcacc
ttcgcgcctgtctcgctgctttcgataagtctctagccatttaaaatttt
tgatgacctgctgcgacgctttttttctggcaagatagtcttgtaaatgc
gggccaagatctgcacactggtatttcggtttttggggccgcgggcggcg
acggggcccgtgcgtcccagcgcacatgttcggcgaggcggggcctgcga
gcgcggccaccgagaatcggacgggggtagtctcaagctggccggcctgc
tctggtgcctggcctcgcgccgccgtgtatcgccccgccctgggcggcaa
ggctggcccggtcggcaccagttgcgtgagcggaaagatggccgcttccc
ggccctgctgcagggagctcaaaatggaggacgcggcgctcgggagagcg
ggcgggtgagtcacccacacaaaggaaaagggcctttccgtcctcagccg
tcgcttcatgtgactccacggagtaccgggcgccgtccaggcacctcgat
tagttctcgagcttttggagtacgtcgtctttaggttggggggaggggtt
ttatgcgatggagtttccccacactgagtgggtggagactgaagttaggc
cagcttggcacttgatgtaattctccttggaatttgccctttttgagttt
ggatcttggttcattctcaagcctcagacagtggttcaaagtttttttct
tccatttcaggtgtcgtgaggatctatttccggtgaattcctcgagacta
gttctagagcggccgcggatcccgcccctctccctcccccccccctaacg
ttactggccgaagccgcttggaataaggccggtgtgcgtttgtctatatg
ttattttccaccatattgccgtcttttggcaatgtgagggcccggaaacc
tggccctgtcttcttgacgagcattcctaggggtctttcccctctcgcca
aaggaatgcaaggtctgttgaatgtcgtgaaggaagcagttcctctggaa
gcttcttgaagacaaacaacgtctgtagcgaccctttgcaggcagcggaa
ccccccacctggcgacaggtgcctctgcggccaaaagccacgtgtataag
atacacctgcaaaggcggcacaaccccagtgccacgttgtgagttggata
gttgtggaaagagtcaaatggctcacctcaagcgtattcaacaaggggct
gaaggatgcccagaaggtaccccattgtatgggatctgatctggggcctc
ggtgcacatgctttacatgtgtttagtcgaggttaaaaaacgtctaggcc
ccccgaaccacggggacgtggttttcctttgaaaaacacgatgataatat
ggtgagcaagggcgaggaggataacatggccatcatcaaggagttcatgc
gcttcaaggtgcacatggagggctccgtgaacggccacgagttcgagatc
gagggcgagggcgagggccgcccctacgagggcacccagaccgccaagct
gaaggtgaccaagggtggccccctgcccttcgcctgggacatcctgtccc
ctcagttcatgtacggctccaaggcctacgtgaagcaccccgccgacatc
cccgactacttgaagctgtccttccccgagggcttcaagtgggagcgcgt
gatgaacttcgaggacggcggcgtggtgaccgtgacccaggactcctccc
tgcaggacggcgagttcatctacaaggtgaagctgcgcggcaccaacttc
ccctccgacggccccgtaatgcagaagaagaccatgggctgggaggcctc
ctccgagcggatgtaccccgaggacggcgccctgaagggcgagatcaagc
agaggctgaagctgaaggacggcggccactacgacgctgaggtcaagacc
acctacaaggccaagaagcccgtgcagctgcccggcgcctacaacgtcaa
catcaagttggacatcacctcccacaacgaggactacaccatcgtggaac
agtacgaacgcgccgagggccgccactccaccggcggcatggacgagctg
tacaagtgaacccgtctggaacaatcaacctctggattacaaaatttgtg
aaagattgactggtattcttaactatgttgctccttttacgctatgtgga
tacgctgctttaatgcctttgtatcatgctattgcttcccgtatggcttt
cattttctcctccttgtataaatcctggttgctgtctctttatgaggagt
tgtggcccgttgtcaggcaacgtggcgtggtgtgcactgtgtttgctgac
gcaacccccactggttggggcattgccaccacctgtcagctcctttccgg
gactttcgctttccccctccctattgccacggcggaactcatcgccgcct
gccttgcccgctgctggacaggggctcggctgttgggcactgacaattcc
gtggtgttgtcggggaagctgacgtcctttccatggctgctcgcctgtgt
tgccacctggattctgcgcgggacgtccttctgctacgtcccttcggccc
tcaatccagcggaccttccttcccgcggcctgctgccggctctgcggcct
cttccgcgtcttcgccttcgccctcagacgagtcggatctccctttgggc
cgcctccccgcctggaattaattctgcagtcgagacctagaaaaacatgg
agcaatcacaagtagcaatacagcagctaccaatgctgattgtgcctggc
tagaagcacaagaggaggaggaggtgggttttccagtcacacctcaggta
cctttaagaccaatgacttacaaggcagctgtagatcttagccacttttt
aaaagaaaagaggggactggaagggctaattcactcccaacgaagacaag
atatccttgatctgtggatctaccacacacaaggctacttccctgattag
cagaactacacaccagggccaggggtcagatatccactgacctttggatg
gtgctacaagctagtaccagttgagccagataaggtagaagaggccaata
aaggagagaacaccagcttgttacaccctgtgagcctgcatgggatggat
gacccggagagagaagtgttagagtggaggtttgacagccgcctagcatt
tcatcacgtggcccgagagctgcatccggagtacttcaagaactgctgat
atcgagcttgctacaagggactttccgctggggactttccagggaggcgt
ggcctgggcgggactggggagtggcgagccctcagatcctgcatataagc
agctgctttttgcctgtactgggtctctctggttagaccagatctgagcc
tgggagctctctggctaactagggaacccactgcttaagcctcaataaag
cttgccttgagtgcttcaagtagtgtgtgcccgtctgttgtgtgactctg
gtaactagagatccctcagacccttttagtcagtgtggaaaatctctagc
agtagtagttcatgtcatcttattattcagtatttataacttgcaaagaa
atgaatatcagagagtgagaggccttgacattgcttgcgtttaccgtcga
cctctagctagagcttggcgtaatcatggtcatagctgtttcctgtgtga
aattgttatccgctcacaattccacacaacatacgagccggaagcataaa
gtgtaaagcctggggtgcctaatgagtgagctaactcacattaattgcgt
tgcgctcactgcccgctttccagtcgggaaacctgtcgtgccagctgcat
taatgaatcggccaacgcgcggggagaggcggtttgcgtattgggcgctc
ttccgcttcctcgctcactgactcgctgcgctcggtcgttcggctgcggc
gagcggtatcagctcactcaaaggcggtaatacggttatccacagaatca
ggggataacgcaggaaagaacatgtgagcaaaaggccagcaaaaggccag
gaaccgtaaaaaggccgcgttgctggcgtttttccataggctccgccccc
ctgacgagcatcacaaaaatcgacgctcaagtcagaggtggcgaaacccg
acaggactataaagataccaggcgtttccccctggaagctccctcgtgcg
ctctcctgttccgaccctgccgcttaccggatacctgtccgcctttctcc
cttcgggaagcgtggcgctttctcatagctcacgctgtaggtatctcagt
tcggtgtaggtcgttcgctccaagctgggctgtgtgcacgaaccccccgt
tcagcccgaccgctgcgccttatccggtaactatcgtcttgagtccaacc
cggtaagacacgacttatcgccactggcagcagccactggtaacaggatt
agcagagcgaggtatgtaggcggtgctacagagttcttgaagtggtggcc
taactacggctacactagaagaacagtatttggtatctgcgctctgctga
agccagttaccttcggaaaaagagttggtagctcttgatccggcaaacaa
accaccgctggtagcggtggtttttttgtttgcaagcagcagattacgcg
cagaaaaaaaggatctcaagaagatcctttgatcttttctacggggtctg
acgctcagtggaacgaaaactcacgttaagggattttggtcatgagatta
tcaaaaaggatcttcacctagatccttttaaattaaaaatgaagttttaa
atcaatctaaagtatatatgagtaaacttggtctgacagttaccaatgct
taatcagtgaggcacctatctcagcgatctgtctatttcgttcatccata
gttgcctgactccccgtcgtgtagataactacgatacgggagggcttacc
atctggccccagtgctgcaatgataccgcgagacccacgctcaccggctc
cagatttatcagcaataaaccagccagccggaagggccgagcgcagaagt
ggtcctgcaactttatccgcctccatccagtctattaattgttgccggga
agctagagtaagtagttcgccagttaatagtttgcgcaacgttgttgcca
ttgctacaggcatcgtggtgtcacgctcgtcgtttggtatggatcattca
gctccggttcccaacgatcaaggcgagttacatgatcccccatgttgtgc
aaaaaagcggttagctccttcggtcctccgatcgttgtcagaagtaagtt
ggccgcagtgttatcactcatggttatggcagcactgcataattctctta
ctgtcatgccatccgtaagatgcttttctgtgactggtgagtactcaacc
aagtcattctgagaatagtgtatgcggcgaccgagttgctcttgcccggc
gtcaatacgggataataccgcgccacatagcagaactttaaaagtgctca
tcattggaaaacgttcttcggggcgaaaactctcaaggatcttaccgctg
ttgagatccagttcgatgtaacccactcgtgcacccaactgatcttcagc
atcttttactttcaccagcgtttctgggtgagcaaaaacaggaaggcaaa
atgccgcaaaaaagggaataagggcgacacggaaatgttgaatactcata
ctatcattttcaatattattgaagcatttatcagggttattgtctcatga
gcggatacatatttgaatgtatttagaaaaataaacaaataggggttccg
cgcacatttccccgaaaagtgccacctgacgtcgacggatcgggagatca
acttgtttattgcagcttataatggttacaaataaagcaatagcatcaca
aatttcacaaataaagcatttttttcactgcattctagttgtggtttgtc
caaactcatcaatgtatcttatcatgtctggatcaactggataactcaag
ctaaccaaaatcatcccaaacttcccaccccataccctattaccactgcc
aattacctgtggtttcatttactctaaacctgtgattcctctgaattatt
ttcattttaaagaaattgtatttgttaaatatgtactacaaacttagtag
t

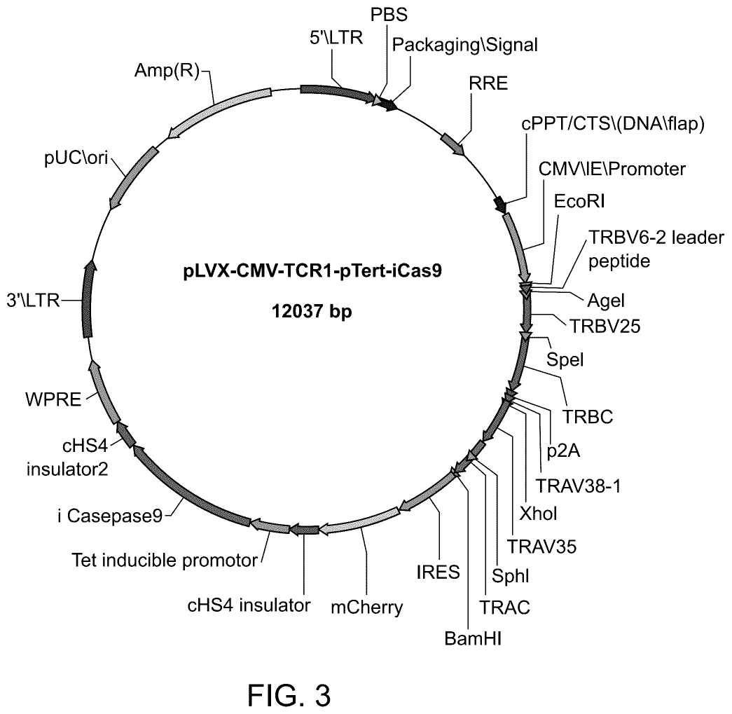

Non-limiting examples of nucleic acid sequences of vectors of the present disclosure are provided herein in Table 2 and illustrated in FIG. 3-12.

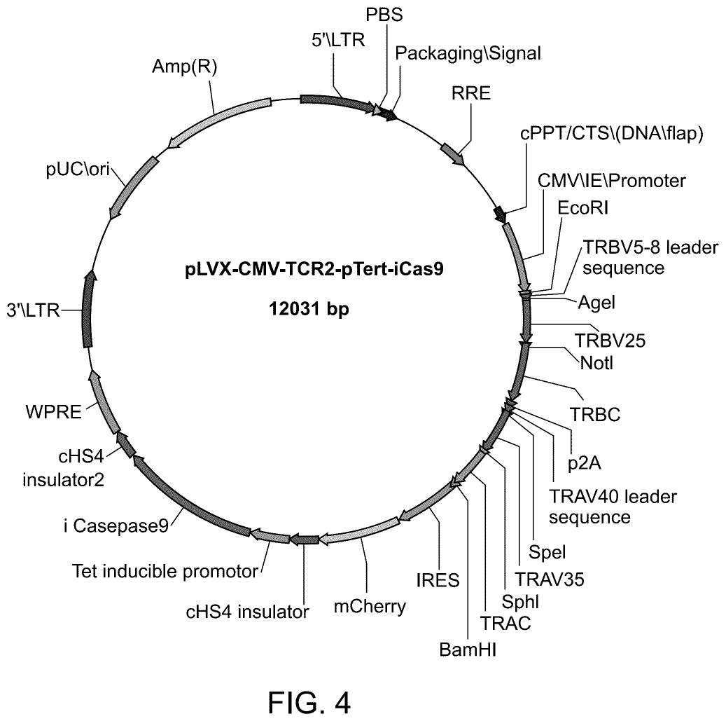

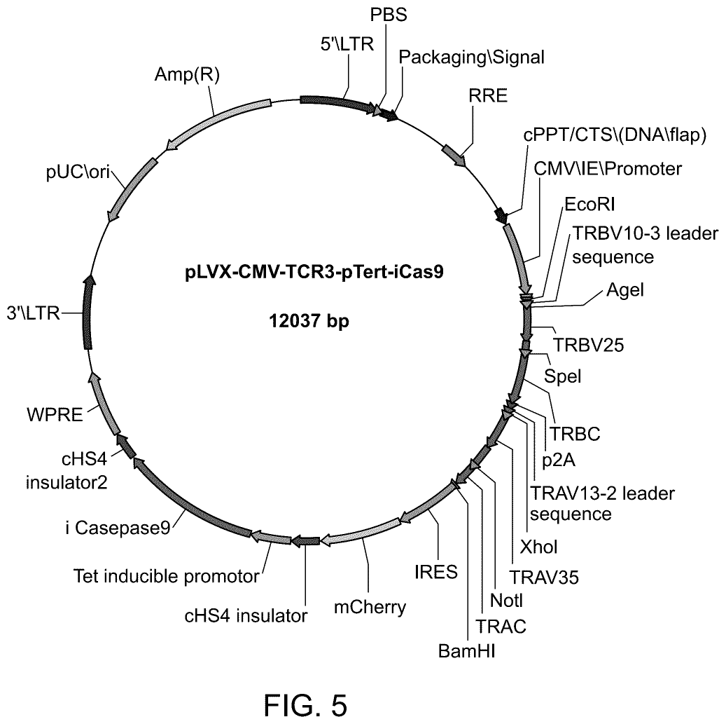

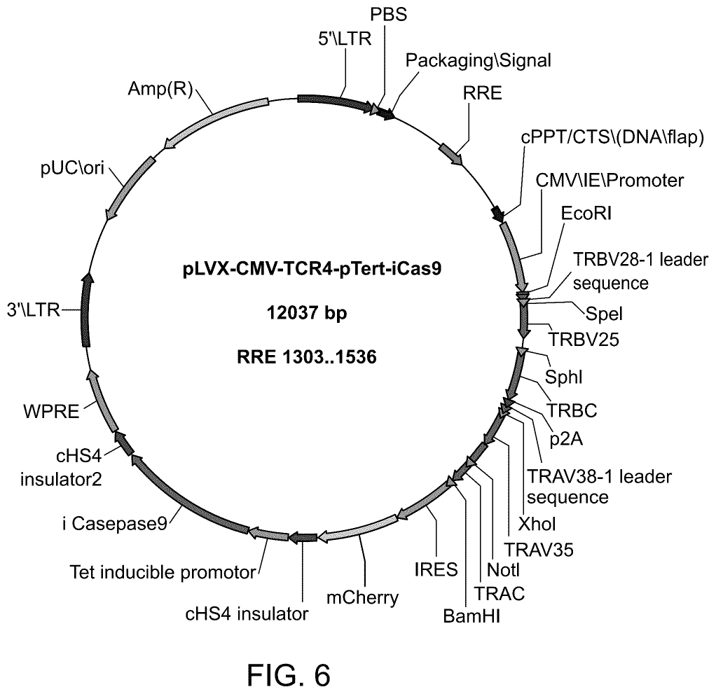

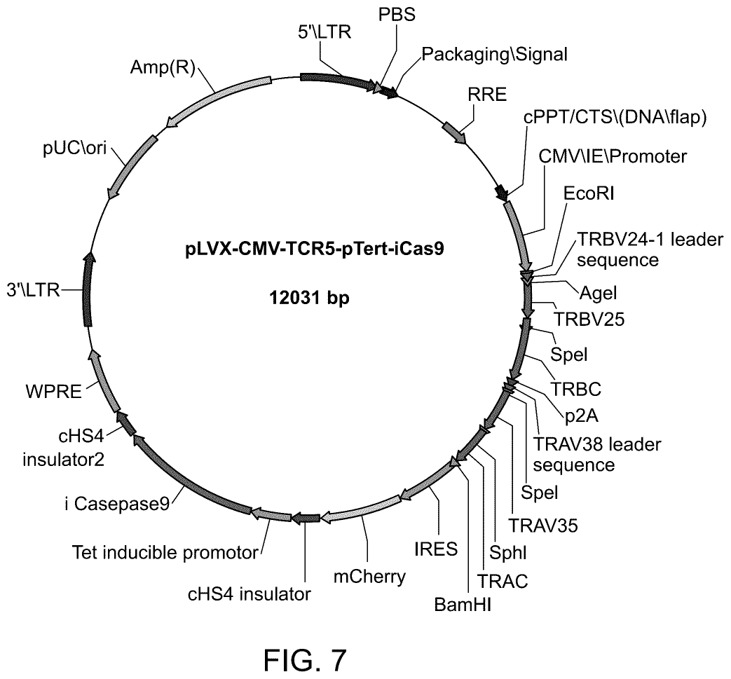

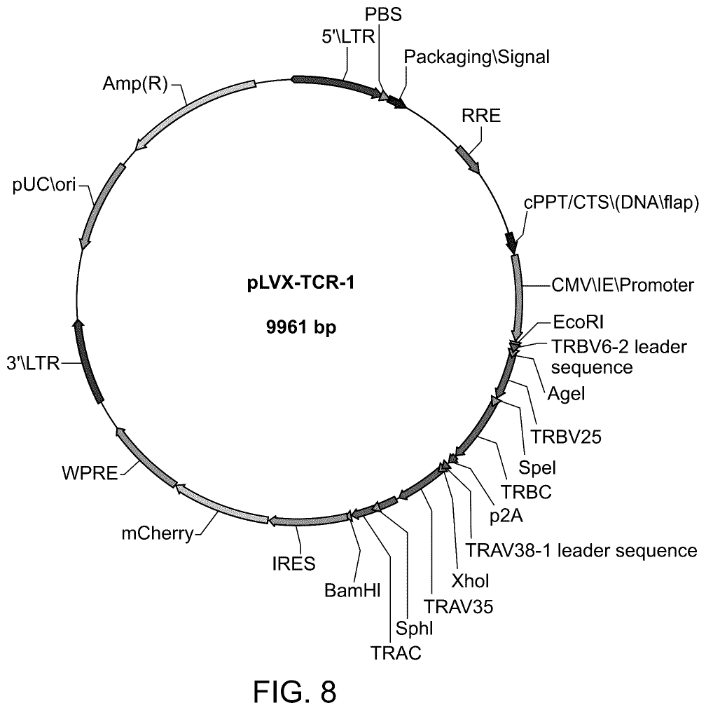

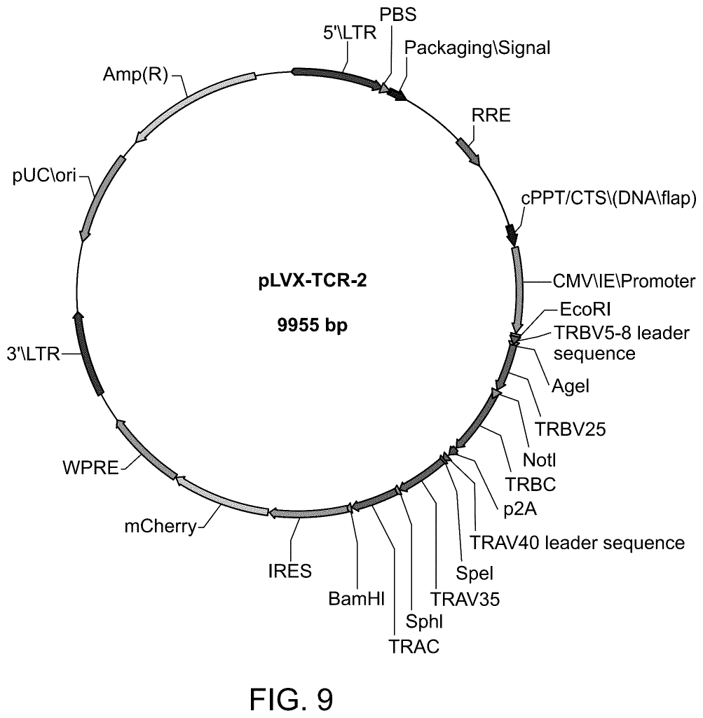

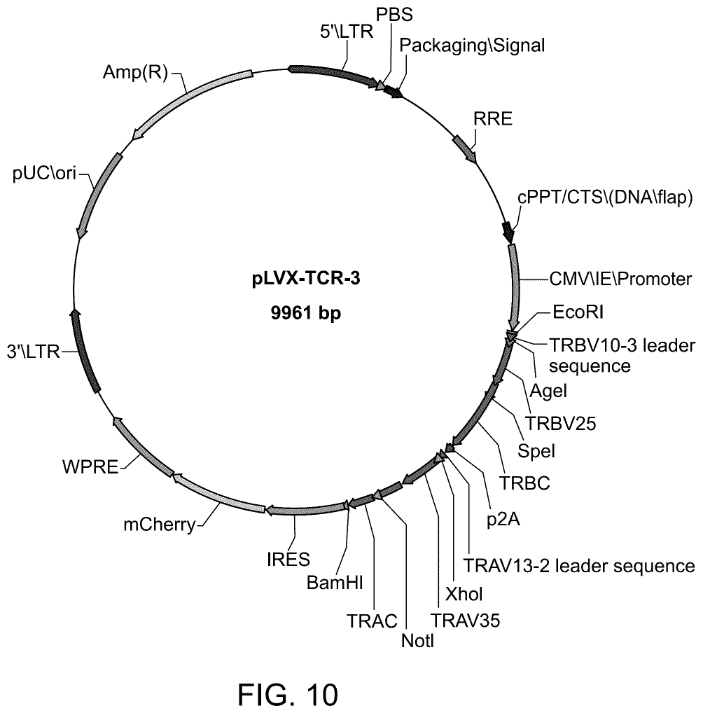

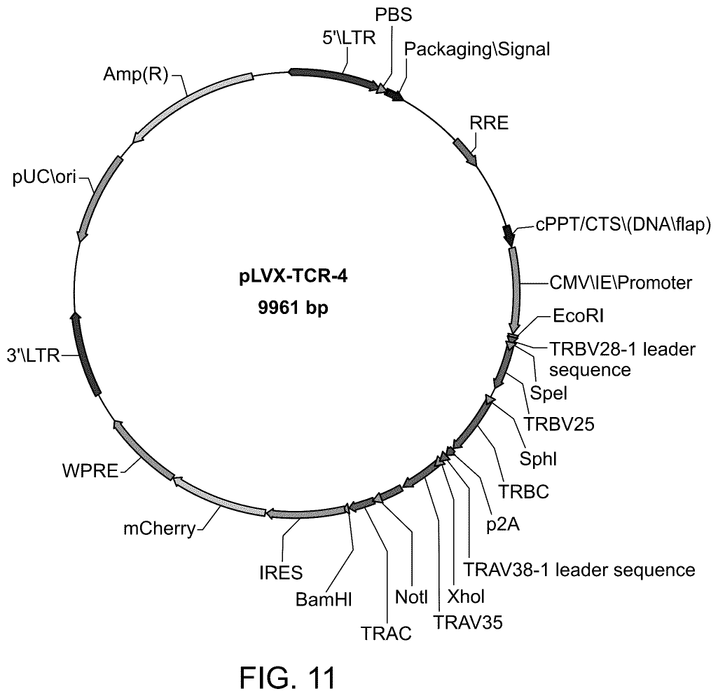

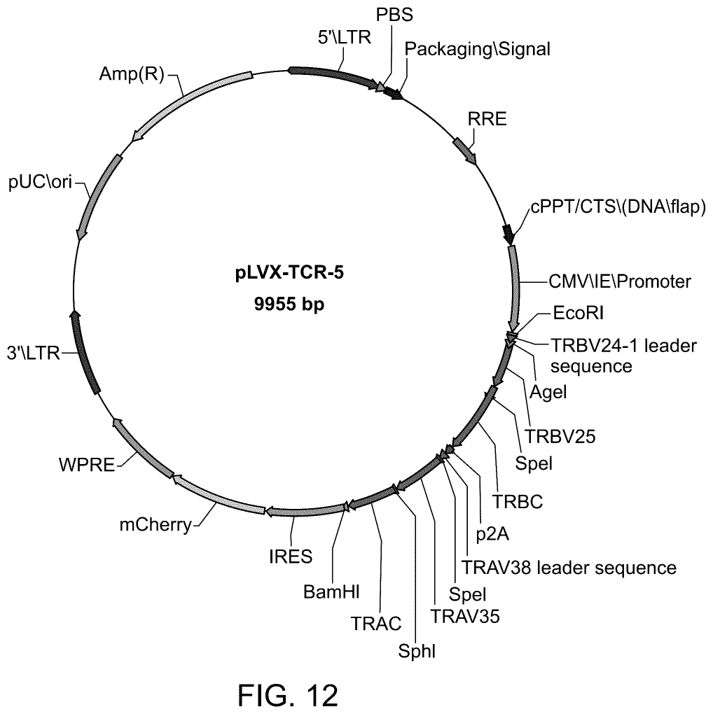

TABLE 2
Exemplary Vector Sequences
pLVX-CMV-TCR1-pTert-iCas9 (FIG. 3):
tggaagggctaattcactcccaaagaagacaagatatccttgatctgtggatctaccacacacaaggctacttccctgattagcagaact
acacaccagggccaggggtcagatatccactgacctttggatggtgctacaagctagtaccagttgagccagataaggtagaagagg
ccaataaaggagagaacaccagcttgttacaccctgtgagcctgcatgggatggatgacccggagagagaagtgttagagtggaggt
ttgacagccgcctagcatttcatcacgtggcccgagagctgcatccggagtacttcaagaactgctgatatcgagcttgctacaaggga
ctttccgctggggactttccagggaggcgtggcctgggcgggactggggagtggcgagccctcagatcctgcatataagcagctgct
ttttgcctgtactgggtctctctggttagaccagatctgagcctgggagctctctggctaactagggaacccactgcttaagcctcaataa
agcttgccttgagtgcttcaagtagtgtgtgcccgtctgttgtgtgactctggtaactagagatccctcagacccttttagtcagtgtggaa
aatctctagcagtggcgcccgaacagggacttgaaagcgaaagggaaaccagaggagctctctcgacgcaggactcggcttgctga
agcgcgcacggcaagaggcgaggggcggcgactggtgagtacgccaaaaattttgactagcggaggctagaaggagagagatgg
gtgcgagagcgtcagtattaagcgggggagaattagatcgcgatgggaaaaaattcggttaaggccagggggaaagaaaaaatata
aattaaaacatatagtatgggcaagcagggagctagaacgattcgcagttaatcctggcctgttagaaacatcagaaggctgtagacaa
atactgggacagctacaaccatcccttcagacaggatcagaagaacttagatcattatataatacagtagcaaccctctattgtgtgcatc
aaaggatagagataaaagacaccaaggaagctttagacaagatagaggaagagcaaaacaaaagtaagaccaccgcacagcaag
cggccggccgctgatcttcagacctggaggaggagatatgagggacaattggagaagtgaattatataaatataaagtagtaaaaattg
aaccattaggagtagcacccaccaaggcaaagagaagagtggtgcagagagaaaaaagagcagtgggaataggagctttgttcctt
gggttcttgggagcagcaggaagcactatgggcgcagcgtcaatgacgctgacggtacaggccagacaattattgtctggtatagtgc
agcagcagaacaatttgctgagggctattgaggcgcaacagcatctgttgcaactcacagtctggggcatcaagcagctccaggcaa
gaatcctggctgtggaaagatacctaaaggatcaacagctcctggggatttggggttgctctggaaaactcatttgcaccactgctgtgc
cttggaatgctagttggagtaataaatctctggaacagatttggaatcacacgacctggatggagtgggacagagaaattaacaattaca
caagataatacactccttaattgaagaatcgcaaaaccagcaagaaaagaatgaacaagaattattggaattagataaatgggcaagtt
tgtggaattggtttaacataacaaattggctgtggtatataaaattattcataatgatagtaggaggcttggtaggtttaagaatagtttttgct
gtactttctatagtgaatagagttaggcagggatattcaccattatcgtttcagacccacctcccaaccccgaggggacccgacaggcc
cgaaggaatagaagaagaaggtggagagagagacagagacagatccattcgattagtgaacggatctcgacggtatcgcctttaaaa
gaaaaggggggattggggggtacagtgcaggggaaagaatagtagacataatagcaacagacatacaaactaaagaattacaaaaa
caaattacaaaaattcaaaattttcgggtttattacagggacagcagagatccagtttatcgataagcttgggagttccgcgttacataactt
acggtaaatggcccgcctggctgaccgcccaacgacccccgcccattgacgtcaataatgacgtatgttcccatagtaacgccaatag
ggactttccattgacgtcaatgggtggagtatttacggtaaactgcccacttggcagtacatcaagtgtatcatatgccaagtacgccccc
tattgacgtcaatgacggtaaatggcccgcctggcattatgcccagtacatgaccttatgggactttcctacttggcagtacatctacgtat
tagtcatcgctattaccatggtgatgcggttttggcagtacatcaatgggcgtggatagcggtttgactcacggggatttccaagtctcca
ccccattgacgtcaatgggagtttgttttggcaccaaaatcaacgggactttccaaaatgtcgtaacaactccgccccattgacgcaaat
gggcggtaggcgtgtacggtgggaggtctatataagcagagctcgtttagtgaaccgtcagatcgcctggagacgccatccacgctgt
tttgacctccatagaagacaccgactctactagaggatctatttccggtgAATTCGCCACCatgagcctcgggctcctgtgctg
tggggccttttctctcctgtgggcaggaccggtgGAAGCTGACATCTACCAGACCCCAAGATACCTTG
TTATAGGGACAGGAAAGAAGATCACTCTGGAATGTTCTCAAACCATGGGCCATG
ACAAAATGTACTGGTATCAACAAGATCCAGGAATGGAACTACACCTCATCCACT
ATTCCTATGGAGTTAATTCCACAGAGAAGGGAGATCTTTCCTCTGAGTCAACAGT
CTCCAGAATAAGGACGGAGCATTTTCCCCTGACCCTGGAGTCTGCCAGGCCCTCA
CATACCTCTCAGTACCTCTGTGCCAGCAGTCGGACGCCCAACATTCAGTACTTCG
GCGCCGGGACCCGGCTCTCAGTGCTGGAGGACCTGAAAAACGTGTTCCCACCCG
AACTAGTCgtgtttgagccatcagaagcagagatctcccacacccaaaaggccacactggtgtgcctggccacaggcttctac
cccgaccacgtggagctgagctggtgggtgaatgggaaggaggtgcacagtggggtcagcacagacccgcagcccctcaaggag
cagcccgccctcaatgactccagatactgcctgagcagccgcctgagggtctcggccaccttctggcagaacccccgcaaccacttc
cgctgtcaagtccagttctacgggctctcggagaatgacgagtggacccaggatagggccaaacctgtcacccagatcgtcagcgcc
gaggcctggggtagagcagactgtggatcacctccgagtataccagcaaggggtcctgtctgccaccatcctctatgagatcttgct
agggaaggccaccttgtatgccgtgctggtcagtgccctcgtgctgatggccatggtcaagagaaaggattccagaggcGGGAG
CGGAGCCACGAACTTCTCTCTGTTAAAGCAAGCAGGAGACGTGGAAGAAAACCC
CGGTCCTatgacacgagttagcttgctgtgggcagtcgtggtctccacctgtctcgagtccggcatgGGTCAACAGCT
GAATCAGAGTCCTCAATCTATGTTTATCCAGGAAGGAGAAGATGTCTCCATGAAC
TGCACTTCTTCAAGCATATTTAACACCTGGCTATGGTACAAGCAGGAACCTGGGG
AAGGTCCTGTCCTCTTGATAGCCTTATATAAGGCTGGTGAATTGACCTCAAATGG
AAGACTGACTGCTCAGTTTGGTATAACCAGAAAGGACAGCTTCCTGAATATCTCA
GCATCCATACCTAGTGATGTAGGCATCTACTTCTGTGCTGGCACATACCAGAAAG
TTACCTTTGGAACTGGAACAAAGCTCCAAGTCATCCCAAATATCCAGAACCCTGA
CCCTGCCGTGTACCAGCTGAGAGACTCTAAATCCAGTGACAAGTCTGTCTGCctattc
accgattttgattctcaaacaaatgtgtcacaaagtaaggattctgatgtgtatatcacagacaaaactgtgctagacatgaggtctatgga
cttcaagagcaacagtgctgtggcctggagcaacaaatctgactttgcatgcgcaaacgccttcaacaacagcattattccagaagaca
ccttcttccccagcccagaaagttcctgtgatgtcaagctggtcgagaaaagctttgaaacagatacgaacctaaactttcaaaacctgtc
agtgattgggttccgaatcctcctcctgaaagtggccgggtttaatctgctcatgacgctgcggctgtggtccagcTGActcgaggga
tcccgcccctctccctcccccccccctaacgttactggccgaagccgcttggaataaggccggtgtgcgtttgtctatatgttattttccac
catattgccgtcttttggcaatgtgagggcccggaaacctggccctgtcttcttgacgagcattcctaggggtctttcccctctcgccaaa
ggaatgcaaggtctgttgaatgtcgtgaaggaagcagttcctctggaagatcttgaagacaaacaacgtctgtagcgaccctttgcag
gcagcggaaccccccacctggcgacaggtgcctctgcggccaaaagccacgtgtataagatacacctgcaaaggcggcacaaccc
cagtgccacgttgtgagttggatagttgtggaaagagtcaaatggctcacctcaagcgtattcaacaaggggctgaaggatgcccaga
aggtaccccattgtatgggatctgatctggggcctcggtgcacatgattacatgtgtttagtcgaggttaaaaaacgtctaggccccccg
aaccacggggacgtggttttcctttgaaaaacacgatgataatatggtgagcaagggcgaggaggataacatggccatcatcaagga
gttcatgcgcttcaaggtgcacatggagggctccgtgaacggccacgagttcgagatcgagggcgagggcgagggccgcccctac
gagggcacccagaccgccaagctgaaggtgaccaagggtggccccctgcccttcgcctgggacatcctgtcccctcagttcatgtac
ggctccaaggcctacgtgaagcaccccgccgacatccccgactacttgaagctgtccttccccgagggcttcaagtgggagcgcgtg
atgaacttcgaggacggcggcgtggtgaccgtgacccaggactcctccctgcaggacggcgagttcatctacaaggtgaagctgcg
cggcaccaacttcccctccgacggccccgtaatgcagaagaagaccatgggctgggaggcctcctccgagcggatgtaccccgag
gacggcgccctgaagggcgagatcaagcagaggctgaagctgaaggacggcggccactacgacgctgaggtcaagaccacctac
aaggccaagaagcccgtgcagctgcccggcgcctacaacgtcaacatcaagttggacatcacctcccacaacgaggactacaccat
cgtggaacagtacgaacgcgccgagggccgccactccaccggcggcatggacgagctgtacaagtgaacgcgtctggaacaagct
ttttccccgtatccccccaggtgtctgcaggctcaaagagcagcgagaagcgttcagaggaaagcgatcccgtgccaccttccccgtg
cccgggctgtccccgcacgctgccggctcggggatgcggggggagcgccggaccggagcggagccccgggcggctcgctgctg
ccccctagcgggggagggacgtaattacatccctgggggctttgggggggggctgtccccgtgagctcttactccctatcagtgatag
agaacgtatgaagagtttactccctatcagtgatagagaacgtatgcagactttactccctatcagtgatagagaacgtataaggagttta
ctccctatcagtgatagagaacgtatgaccagtttactccctatcagtgatagagaacgtatctacagtttactccctatcagtgatagaga
acgtatatccagtttactccctatcagtgatagagaacgtatgtcgaggtaggcgtgtacggtgggcgcctataaaagcagagctcgttt
agtgaaccgtcagatcgcctggagcaattccacaacacttttgtatatacttATGCTCGAGGGAGTGCAGGTGGA
GACTATCTCCCCAGGAGACGGGCGCACCTTCCCCAAGCGCGGCCAGACCTGCGT
GGTGCACTACACCGGGATGCTTGAAGATGGAAAGAAAGTTGATTCCTCCCGGGA
CAGAAACAAGCCCTTTAAGTTTATGCTAGGCAAGCAGGAGGTGATCCGAGGCTG
GGAAGAAGGGGTTGCCCAGATGAGTGTGGGTCAGAGAGCCAAACTGACTATATC
TCCAGATTATGCCTATGGTGCCACTGGGCACCCAGGCATCATCCCACCACATGCC
ACTCTCGTCTTCGATGTGGAGCTTCTAAAACTGGAATCTGGCGGTGGATCCGGAG
TCGACGGATTTGGTGATGTCGGTGCTCTTGAGAGTTTGAGGGGAAATGCAGATTT
GGCTTACATCCTGAGCATGGAGCCCTGTGGCCACTGCCTCATTATCAACAATGTG
AACTTCTGCCGTGAGTCCGGGCTCCGCACCCGCACTGGCTCCAACATCGACTGTG
AGAAGTTGCGGCGTCGCTTCTCCTCGCTGCATTTCATGGTGGAGGTGAAGGGCGA
CCTGACTGCCAAGAAAATGGTGCTGGCTTTGCTGGAGCTGGCGCGGCAGGACCA
CGGTGCTCTGGACTGCTGCGTGGTGGTCATTCTCTCTCACGGCTGTCAGGCCAGC
CACCTGCAGTTCCCAGGGGCTGTCTACGGCACAGATGGATGCCCTGTGTCGGTCG
AGAAGATTGTGAACATCTTCAATGGGACCAGCTGCCCCAGCCTGGGAGGGAAGC
CCAAGCTCTTTTTCATCCAGGCCTGTGGTGGGGAGCAGAAAGACCATGGGTTTGA
GGTGGCCTCCACTTCCCCTGAAGACGAGTCCCCTGGCAGTAACCCCGAGCCAGAT
GCCACCCCGTTCCAGGAAGGTTTGAGGACCTTCGACCAGCTGGACGCCATATCTA
GTTTGCCCACACCCAGTGACATCTTTGTGTCCTACTCTACTTTCCCAGGTTTTGTT
TCCTGGAGGGACCCCAAGAGTGGCTCCTGGTACGTTGAGACCCTGGACGACATCT
TTGAGCAGTGGGCTCACTCTGAAGACCTGCAGTCCCTCCTGCTTAGGGTCGCTAA
TGCTGTTTCGGTGAAAGGGATTTATAAACAGATGCCTGGTTGCTTTAATTTCCTCC
GGAAAAAACTTTTCTTTAAAACATCAGTCGACTATCCGTACGACGTACCAGACTA
CGCACTCGACTAAaagctttttccccgtatccccccaggtgtctgcaggctcaaagagcagcgagaagcgttcagaggaa
agcgatcccgtgccaccttccccgtgcccgggctgtccccgcacgctgccggctcggggatgcggggggagcgccggaccggag
cggagccccgggcggctcgctgctgccccctagcgggggagggacgtaattacatccctgggggctttgggagggggctgtcccc
gtgagctcaatcaacctctggattacaaaatttgtgaaagattgactggtattcttaactatgttgctccttttacgctatgtggatacgctgct
ttaatgcctttgtatcatgctattgcttcccgtatggctttcattttctcctccttgtataaatcctggttgctgtctctttatgaggagttgtggcc
cgttgtcaggcaacgtggcgtggtgtgcactgtgtttgctgacgcaacccccactggttggggcattgccaccacctgtcagctcctttc
cgggactttcgctttccccctccctattgccacggcggaactcatcgccgcctgccttgcccgctgctggacaggggctcggctgttgg
gcactgacaattccgtggtgttgtcggggaagctgacgtcctttccatggctgctcgcctgtgttgccacctggattctgcgcgggacgt
ccttctgctacgtccatcggccctcaatccagcggaccttccttcccgcggcctgctgccggctctgcggcctcttccgcgtcttcgcct
tcgccctcagacgagtcggatctccctttgggccgcctccccgcctggaattaattctgcagtcgagacctagaaaaacatggagcaat
cacaagtagcaatacagcagctaccaatgctgattgtgcctggctagaagcacaagaggaggaggaggtgggttttccagtcacacct
caggtacctttaagaccaatgacttacaaggcagctgtagatcttagccactttttaaaagaaaagaggggactggaagggctaattcac
tcccaacgaagacaagatatccttgatctgtggatctaccacacacaaggctacttccctgattagcagaactacacaccagggccagg
ggtcagatatccactgacctttggatggtgctacaagctagtaccagttgagccagataaggtagaagaggccaataaaggagagaac
accagcttgttacaccctgtgagcctgcatgggatggatgacccggagagagaagtgttagagtggaggtttgacagccgcctagcat
ttcatcacgtggcccgagagctgcatccggagtacttcaagaactgctgatatcgagcttgctacaagggactttccgctggggactttc
cagggaggcgtggcctgggcgggactggggagtggcgagccctcagatcctgcatataagcagctgctttttgcctgtactgggtctc
tctggttagaccagatctgagcctgggagctctctggctaactagggaacccactgcttaagcctcaataaagcttgccttgagtgcttca
agtagtgtgtgcccgtctgttgtgtgactctggtaactagagatccctcagacccttttagtcagtgtggaaaatctctagcagtagtagttc
atgtcatcttattattcagtatttataacttgcaaagaaatgaatatcagagagtgagaggccttgacattgctagcgtttaccgtcgacctct
agctagagcttggcgtaatcatggtcatagctgtttcctgtgtgaaattgttatccgctcacaattccacacaacatacgagccggaagca
taaagtgtaaagcctggggtgcctaatgagtgagctaactcacattaattgcgttgcgctcactgcccgctttccagtcgggaaacctgt
cgtgccagctgcattaatgaatcggccaacgcgcggggagaggcggtttgcgtattgggcgctcttccgcttcctcgctcactgactcg
ctgcgctcggtcgttcggctgcggcgagcggtatcagctcactcaaaggcggtaatacggttatccacagaatcaggggataacgca
ggaaagaacatgtgagcaaaaggccagcaaaaggccaggaaccgtaaaaaggccgcgttgctggcgtttttccataggctccgccc
ccctgacgagcatcacaaaaatcgacgctcaagtcagaggtggcgaaacccgacaggactataaagataccaggcgtttccccctgg
aagctccctcgtgcgctctcctgttccgaccctgccgcttaccggatacctgtccgcctttctcccttcgggaagcgtggcgctttctcata
gctcacgctgtaggtatctcagttcggtgtaggtcgttcgctccaagctgggctgtgtgcacgaaccccccgttcagcccgaccgctgc
gccttatccggtaactatcgtcttgagtccaacccggtaagacacgacttatcgccactggcagcagccactggtaacaggattagcag
agcgaggtatgtaggcggtgctacagagttcttgaagtggtggcctaactacggctacactagaagaacagtatttggtatctgcgctct
gctgaagccagttaccttcggaaaaagagttggtagctcttgatccggcaaacaaaccaccgctggtagcggtggtttttttgtttgcaag
cagcagattacgcgcagaaaaaaaggatctcaagaagatcctttgatcttttctacggggtctgacgctcagtggaacgaaaactcacg
ttaagggattttggtcatgagattatcaaaaaggatcttcacctagatccttttaaattaaaaatgaagttttaaatcaatctaaagtatatatg
agtaaacttggtctgacagttaccaatgcttaatcagtgaggcacctatctcagcgatctgtctatttcgttcatccatagttgcctgactcc
ccgtcgtgtagataactacgatacgggagggcttaccatctggccccagtgctgcaatgataccgcgagacccacgctcaccggctc
cagatttatcagcaataaaccagccagccggaagggccgagcgcagaagtggtcctgcaactttatccgcctccatccagtctattaat
tgttgccgggaagctagagtaagtagttcgccagttaatagtttgcgcaacgttgttgccattgctacaggcatcgtggtgtcacgctcgt
cgtttggtatggcttcattcagctccggttcccaacgatcaaggcgagttacatgatcccccatgttgtgcaaaaaagcggttagctcctt
cggtcctccgatcgttgtcagaagtaagttggccgcagtgttatcactcatggttatggcagcactgcataattctcttactgtcatgccatc
cgtaagatgcttttctgtgactggtgagtactcaaccaagtcattctgagaatagtgtatgcggcgaccgagttgctcttgcccggcgtca
atacgggataataccgcgccacatagcagaactttaaaagtgctcatcattggaaaacgttcttcggggcgaaaactctcaaggatctta
ccgctgttgagatccagttcgatgtaacccactcgtgcacccaactgatcttcagcatcttttactttcaccagcgtttctgggtgagcaaa
aacaggaaggcaaaatgccgcaaaaaagggaataagggcgacacggaaatgttgaatactcatactcttcctttttcaatattattgaag
catttatcagggttattgtctcatgagcggatacatatttgaatgtatttagaaaaataaacaaataggggttccgcgcacatttccccgaaa
agtgccacctgacgtcgacggatcgggagatcaacttgtttattgcagcttataatggttacaaataaagcaatagcatcacaaatttcac
aaataaagcatttttttcactgcattctagttgtggtttgtccaaactcatcaatgtatcttatcatgtctggatcaactggataactcaagctaa
ccaaaatcatcccaaacttcccaccccataccctattaccactgccaattacctgtggtttcatttactctaaacctgtgattcctctgaattat
tttcattttaaagaaattgtatttgttaaatatgtactacaaacttagtagt (SEQ ID NO: 21)
pLVX-CMV-TCR2-pTert-iCas9 (FIG. 4):
tggaagggctaattcactcccaaagaagacaagatatccttgatctgtggatctaccacacacaaggctacttccctgattagcagaact
acacaccagggccaggggtcagatatccactgacctttggatggtgctacaagctagtaccagttgagccagataaggtagaagagg
ccaataaaggagagaacaccagcttgttacaccctgtgagcctgcatgggatggatgacccggagagagaagtgttagagtggaggt
ttgacagccgcctagcatttcatcacgtggcccgagagctgcatccggagtacttcaagaactgctgatatcgagcttgctacaaggga
ctttccgctggggactttccagggaggcgtggcctgggcgggactggggagtggcgagccctcagatcctgcatataagcagctgct
ttttgcctgtactgggtctctctggttagaccagatctgagcctgggagctctctggctaactagggaacccactgcttaagcctcaataa
agcttgccttgagtgcttcaagtagtgtgtgcccgtctgttgtgtgactctggtaactagagatccctcagacccttttagtcagtgtggaa
aatctctagcagtggcgcccgaacagggacttgaaagcgaaagggaaaccagaggagctctctcgacgcaggactcggcttgctga
agcgcgcacggcaagaggcgaggggcggcgactggtgagtacgccaaaaattttgactagcggaggctagaaggagagagatgg
gtgcgagagcgtcagtattaagcgggggagaattagatcgcgatgggaaaaaattcggttaaggccagggggaaagaaaaaatata
aattaaaacatatagtatgggcaagcagggagctagaacgattcgcagttaatcctggcctgttagaaacatcagaaggctgtagacaa
atactgggacagctacaaccatcccttcagacaggatcagaagaacttagatcattatataatacagtagcaaccctctattgtgtgcatc
aaaggatagagataaaagacaccaaggaagctttagacaagatagaggaagagcaaaacaaaagtaagaccaccgcacagcaag
cggccggccgctgatcttcagacctggaggaggagatatgagggacaattggagaagtgaattatataaatataaagtagtaaaaattg
aaccattaggagtagcacccaccaaggcaaagagaagagtggtgcagagagaaaaaagagcagtgggaataggagctttgttcctt
gggttcttgggagcagcaggaagcactatgggcgcagcgtcaatgacgctgacggtacaggccagacaattattgtctggtatagtgc
agcagcagaacaatttgctgagggctattgaggcgcaacagcatctgttgcaactcacagtctggggcatcaagcagctccaggcaa
gaatcctggctgtggaaagatacctaaaggatcaacagctcctggggatttggggttgctctggaaaactcatttgcaccactgctgtgc
cttggaatgctagttggagtaataaatctctggaacagatttggaatcacacgacctggatggagtgggacagagaaattaacaattaca
caagcttaatacactccttaattgaagaatcgcaaaaccagcaagaaaagaatgaacaagaattattggaattagataaatgggcaagtt
tgtggaattggtttaacataacaaattggctgtggtatataaaattattcataatgatagtaggaggcttggtaggtttaagaatagtttttgct
gtactttctatagtgaatagagttaggcagggatattcaccattatcgtttcagacccacctcccaaccccgaggggacccgacaggcc
cgaaggaatagaagaagaaggtggagagagagacagagacagatccattcgattagtgaacggatctcgacggtatcgcctttaaaa
gaaaaggggggattggggggtacagtgcaggggaaagaatagtagacataatagcaacagacatacaaactaaagaattacaaaaa
caaattacaaaaattcaaaattttcgggtttattacagggacagcagagatccagtttatcgataagcttgggagttccgcgttacataactt
acggtaaatggcccgcctggctgaccgcccaacgacccccgcccattgacgtcaataatgacgtatgttcccatagtaacgccaatag
ggactttccattgacgtcaatgggtggagtatttacggtaaactgcccacttggcagtacatcaagtgtatcatatgccaagtacgccccc
tattgacgtcaatgacggtaaatggcccgcctggcattatgcccagtacatgaccttatgggactttcctacttggcagtacatctacgtat
tagtcatcgctattaccatggtgatgcggttttggcagtacatcaatgggcgtggatagcggtttgactcacggggatttccaagtctcca
ccccattgacgtcaatgggagtttgttttggcaccaaaatcaacgggactttccaaaatgtcgtaacaactccgccccattgacgcaaat
gggcggtaggcgtgtacggtgggaggtctatataagcagagctcgtttagtgaaccgtcagatcgcctggagacgccatccacgctgt
tttgacctccatagaagacaccgactctactagaggatctatttccggtgAATTCGCCACCatgggacccaggctcctatctg
ggcactgattgtctcctcggaaCCGGTCCGGTTGAAGCTGACATCTACCAGACCCCAAGATAC
CTTGTTATAGGGACAGGAAAGAAGATCACTCTGGAATGTTCTCAAACCATGGGC
CATGACAAAATGTACTGGTATCAACAAGATCCAGGAATGGAACTACACCTCATC
CACTATTCCTATGGAGTTAATTCCACAGAGAAGGGAGATCTTTCCTCTGAGTCAA
CAGTCTCCAGAATAAGGACGGAGCATTTTCCCCTGACCCTGGAGTCTGCCAGGCC
CTCACATACCTCTCAGTACCTCTGTGCCAGCAGTCGGACGCCCAACATTCAGTAC
TTCGGCGCCGGGACCCGGCTCTCAGTGCTGGAGGACCTGAAAAACGTGTTCCCAC
CCGAgGCGGCCGCgtttgagccatcagaagcagagatctcccacacccaaaaggccacactggtgtgcctggccacagg
cttctaccccgaccacgtggagctgagctggtgggtgaatgggaaggaggtgcacagtggggtcagcacagacccgcagcccctc
aaggagcagcccgccctcaatgactccagatactgcctgagcagccgcctgagggtctcggccaccttctggcagaacccccgcaa
ccacttccgctgtcaagtccagttctacgggctctcggagaatgacgagtggacccaggatagggccaaacctgtcacccagatcgtc
agcgccgaggcctggggtagagcagactgtggcttcacctccgagtcttaccagcaaggggtcctgtctgccaccatcctctatgaga
tcttgctagggaaggccaccttgtatgccgtgctggtcagtgccctcgtgctgatggccatggtcaagagaaaggattccagaggcG
GGAGCGGAGCCACGAACTTCTCTCTGTTAAAGCAAGCAGGAGACGTGGAAGAAA
ACCCCGGTCCTatgaactcctctctggactttctaattctgatcttaatgtttggaggaACTAGTGGTCAACAGC
TGAATCAGAGTCCTCAATCTATGTTTATCCAGGAAGGAGAAGATGTCTCCATGAA
CTGCACTTCTTCAAGCATATTTAACACCTGGCTATGGTACAAGCAGGAACCTGGG
GAAGGTCCTGTCCTCTTGATAGCCTTATATAAGGCTGGTGAATTGACCTCAAATG
GAAGACTGACTGCTCAGTTTGGTATAACCAGAAAGGACAGCTTCCTGAATATCTC
AGCATCCATACCTAGTGATGTAGGCATCTACTTCTGTGCTGGCACATACCAGAAA
GTTACCTTTGGAACTGGAACAAAGCTCCAAGTCATCCCAAATATCCAGAACCCTG
ACCCTGCCGTGTACCAGCTGAGAGACTCTAAATCCAGTGACAAGTCTGCATGCctat
tcaccgattttgattctcaaacaaatgtgtcacaaagtaaggattctgatgtgtatatcacagacaaaactgtgctagacatgaggtctatg
gacttcaagagcaacagtgctgtggcctggagcaacaaatctgactttgcatgtgcaaacgccttcaacaacagcattattccagaaga
caccttcttccccagcccagaaagttcctgtgatgtcaagctggtcgagaaaagctttgaaacagatacgaacctaaactttcaaaacct
gtcagtgattgggttccgaatcctcctcctgaaagtggccgggtttaatctgctcatgacgctgcggctgtggtccagcTGActcgag
ggatcccgcccctctccctcccccccccctaacgttactggccgaagccgcttggaataaggccggtgtgcgtttgtctatatgttattttc
caccatattgccgtcttttggcaatgtgagggcccggaaacctggccctgtcttcttgacgagcattcctaggggtctttcccctctcgcc
aaaggaatgcaaggtctgttgaatgtcgtgaaggaagcagttcctctggaagcttcttgaagacaaacaacgtctgtagcgaccctttgc
aggcagcggaaccccccacctggcgacaggtgcctctgcggccaaaagccacgtgtataagatacacctgcaaaggcggcacaac
cccagtgccacgttgtgagttggatagttgtggaaagagtcaaatggctcacctcaagcgtattcaacaaggggctgaaggatgccca
gaaggtaccccattgtatgggatctgatctggggcctcggtgcacatgctttacatgtgtttagtcgaggttaaaaaacgtctaggccccc
cgaaccacggggacgtggttttcctttgaaaaacacgatgataatatggtgagcaagggcgaggaggataacatggccatcatcaag
gagttcatgcgcttcaaggtgcacatggagggctccgtgaacggccacgagttcgagatcgagggcgagggcgagggccgcccct
acgagggcacccagaccgccaagctgaaggtgaccaagggtggccccctgcccttcgcctgggacatcctgtcccctcagttcatgt
acggctccaaggcctacgtgaagcaccccgccgacatccccgactacttgaagctgtccttccccgagggcttcaagtgggagcgcg
tgatgaacttcgaggacggcggcgtggtgaccgtgacccaggactcctccctgcaggacggcgagttcatctacaaggtgaagctgc
gcggcaccaacttcccctccgacggccccgtaatgcagaagaagaccatgggctgggaggcctcctccgagcggatgtaccccga
ggacggcgccctgaagggcgagatcaagcagaggctgaagctgaaggacggcggccactacgacgctgaggtcaagaccaccta
caaggccaagaagcccgtgcagctgcccggcgcctacaacgtcaacatcaagttggacatcacctcccacaacgaggactacacca
tcgtggaacagtacgaacgcgccgagggccgccactccaccggcggcatggacgagctgtacaagtgaacgcgtctggaacaagc
tttttccccgtatccccccaggtgtctgcaggctcaaagagcagcgagaagcgttcagaggaaagcgatcccgtgccaccttccccgt
gcccgggctgtccccgcacgctgccggctcggggatgcggggggagcgccggaccggagcggagccccgggcggctcgctgct
gccccctagcgggggagggacgtaattacatccctgggggctttgggggggggctgtccccgtgagctcttactccctatcagtgata
gagaacgtatgaagagtttactccctatcagtgatagagaacgtatgcagactttactccctatcagtgatagagaacgtataaggagttt
actccctatcagtgatagagaacgtatgaccagtttactccctatcagtgatagagaacgtatctacagtttactccctatcagtgatagag
aacgtatatccagtttactccctatcagtgatagagaacgtatgtcgaggtaggcgtgtacggtgggcgcctataaaagcagagctcgtt
tagtgaaccgtcagatcgcctggagcaattccacaacacttttgtcttatacttATGCTCGAGGGAGTGCAGGTGGA
GACTATCTCCCCAGGAGACGGGCGCACCTTCCCCAAGCGCGGCCAGACCTGCGT
GGTGCACTACACCGGGATGCTTGAAGATGGAAAGAAAGTTGATTCCTCCCGGGA
CAGAAACAAGCCCTTTAAGTTTATGCTAGGCAAGCAGGAGGTGATCCGAGGCTG
GGAAGAAGGGGTTGCCCAGATGAGTGTGGGTCAGAGAGCCAAACTGACTATATC
TCCAGATTATGCCTATGGTGCCACTGGGCACCCAGGCATCATCCCACCACATGCC
ACTCTCGTCTTCGATGTGGAGCTTCTAAAACTGGAATCTGGCGGTGGATCCGGAG
TCGACGGATTTGGTGATGTCGGTGCTCTTGAGAGTTTGAGGGGAAATGCAGATTT
GGCTTACATCCTGAGCATGGAGCCCTGTGGCCACTGCCTCATTATCAACAATGTG
AACTTCTGCCGTGAGTCCGGGCTCCGCACCCGCACTGGCTCCAACATCGACTGTG
AGAAGTTGCGGCGTCGCTTCTCCTCGCTGCATTTCATGGTGGAGGTGAAGGGCGA
CCTGACTGCCAAGAAAATGGTGCTGGCTTTGCTGGAGCTGGCGCGGCAGGACCA
CGGTGCTCTGGACTGCTGCGTGGTGGTCATTCTCTCTCACGGCTGTCAGGCCAGC
CACCTGCAGTTCCCAGGGGCTGTCTACGGCACAGATGGATGCCCTGTGTCGGTCG
AGAAGATTGTGAACATCTTCAATGGGACCAGCTGCCCCAGCCTGGGAGGGAAGC
CCAAGCTCTTTTTCATCCAGGCCTGTGGTGGGGAGCAGAAAGACCATGGGTTTGA
GGTGGCCTCCACTTCCCCTGAAGACGAGTCCCCTGGCAGTAACCCCGAGCCAGAT
GCCACCCCGTTCCAGGAAGGTTTGAGGACCTTCGACCAGCTGGACGCCATATCTA
GTTTGCCCACACCCAGTGACATCTTTGTGTCCTACTCTACTTTCCCAGGTTTTGTT
TCCTGGAGGGACCCCAAGAGTGGCTCCTGGTACGTTGAGACCCTGGACGACATCT
TTGAGCAGTGGGCTCACTCTGAAGACCTGCAGTCCCTCCTGCTTAGGGTCGCTAA
TGCTGTTTCGGTGAAAGGGATTTATAAACAGATGCCTGGTTGCTTTAATTTCCTCC
GGAAAAAACTTTTCTTTAAAACATCAGTCGACTATCCGTACGACGTACCAGACTA
CGCACTCGACTAAaagctttttccccgtatccccccaggtgtctgcaggctcaaagagcagcgagaagcgttcagaggaa
agcgatcccgtgccaccttccccgtgcccgggctgtccccgcacgctgccggctcggggatgcggggggagcgccggaccggag
cggagccccgggcggctcgctgctgccccctagcgggggagggacgtaattacatccctgggggctttgggagggggctgtcccc
gtgagctcaatcaacctctggattacaaaatttgtgaaagattgactggtattcttaactatgttgctccttttacgctatgtggatacgctgct
ttaatgcattgtatcatgctattgatcccgtatggctttcattttctcctccttgtataaatcctggttgctgtctctttatgaggagttgtggcc
cgttgtcaggcaacgtggcgtggtgtgcactgtgtttgctgacgcaacccccactggttggggcattgccaccacctgtcagctcctttc
cgggactttcgctttccccctccctattgccacggcggaactcatcgccgcctgccttgcccgctgctggacaggggctcggctgttgg
gcactgacaattccgtggtgttgtcggggaagctgacgtcctttccatggctgctcgcctgtgttgccacctggattctgcgcgggacgt
ccttctgctacgtccatcggccctcaatccagcggaccttccttcccgcggcctgctgccggctctgcggcctcttccgcgtcttcgcct
tcgccctcagacgagtcggatctccctttgggccgcctccccgcctggaattaattctgcagtcgagacctagaaaaacatggagcaat
cacaagtagcaatacagcagctaccaatgctgattgtgcctggctagaagcacaagaggaggaggaggtgggttttccagtcacacct
caggtacctttaagaccaatgacttacaaggcagctgtagatcttagccactttttaaaagaaaagaggggactggaagggctaattcac
tcccaacgaagacaagatatccttgatctgtggatctaccacacacaaggctacttccctgattagcagaactacacaccagggccagg
ggtcagatatccactgacctttggatggtgctacaagctagtaccagttgagccagataaggtagaagaggccaataaaggagagaac
accagcttgttacaccctgtgagcctgcatgggatggatgacccggagagagaagtgttagagtggaggtttgacagccgcctagcat
ttcatcacgtggcccgagagctgcatccggagtacttcaagaactgctgatatcgagcttgctacaagggactttccgctggggactttc
cagggaggcgtggcctgggcgggactggggagtggcgagccctcagatcctgcatataagcagctgctttttgcctgtactgggtctc
tctggttagaccagatctgagcctgggagctctctggctaactagggaacccactgcttaagcctcaataaagcttgccttgagtgcttca
agtagtgtgtgcccgtctgttgtgtgactctggtaactagagatccctcagacccttttagtcagtgtggaaaatctctagcagtagtagttc
atgtcatcttattattcagtatttataacttgcaaagaaatgaatatcagagagtgagaggccttgacattgctagcgtttaccgtcgacctct
agctagagcttggcgtaatcatggtcatagctgtttcctgtgtgaaattgttatccgctcacaattccacacaacatacgagccggaagca
taaagtgtaaagcctggggtgcctaatgagtgagctaactcacattaattgcgttgcgctcactgcccgctttccagtcgggaaacctgt
cgtgccagctgcattaatgaatcggccaacgcgcggggagaggcggtttgcgtattgggcgctcttccgcttcctcgctcactgactcg
ctgcgctcggtcgttcggctgcggcgagcggtatcagctcactcaaaggcggtaatacggttatccacagaatcaggggataacgca
ggaaagaacatgtgagcaaaaggccagcaaaaggccaggaaccgtaaaaaggccgcgttgctggcgtttttccataggctccgccc
ccctgacgagcatcacaaaaatcgacgctcaagtcagaggtggcgaaacccgacaggactataaagataccaggcgtttccccctgg
aagctccctcgtgcgctctcctgttccgaccctgccgcttaccggatacctgtccgcctttctcccttcgggaagcgtggcgctttctcata
gctcacgctgtaggtatctcagttcggtgtaggtcgttcgctccaagctgggctgtgtgcacgaaccccccgttcagcccgaccgctgc
gccttatccggtaactatcgtcttgagtccaacccggtaagacacgacttatcgccactggcagcagccactggtaacaggattagcag
agcgaggtatgtaggcggtgctacagagttcttgaagtggtggcctaactacggctacactagaagaacagtatttggtatctgcgctct
gctgaagccagttaccttcggaaaaagagttggtagctcttgatccggcaaacaaaccaccgctggtagcggtggtttttttgtttgcaag
cagcagattacgcgcagaaaaaaaggatctcaagaagatcctttgatcttttctacggggtctgacgctcagtggaacgaaaactcacg
ttaagggattttggtcatgagattatcaaaaaggatcttcacctagatccttttaaattaaaaatgaagttttaaatcaatctaaagtatatatg
agtaaacttggtctgacagttaccaatgcttaatcagtgaggcacctatctcagcgatctgtctatttcgttcatccatagttgcctgactcc
ccgtcgtgtagataactacgatacgggagggcttaccatctggccccagtgctgcaatgataccgcgagacccacgctcaccggctc
cagatttatcagcaataaaccagccagccggaagggccgagcgcagaagtggtcctgcaactttatccgcctccatccagtctattaat
tgttgccgggaagctagagtaagtagttcgccagttaatagtttgcgcaacgttgttgccattgctacaggcatcgtggtgtcacgctcgt
cgtttggtatggcttcattcagctccggttcccaacgatcaaggcgagttacatgatcccccatgttgtgcaaaaaagcggttagctcctt
cggtcctccgatcgttgtcagaagtaagttggccgcagtgttatcactcatggttatggcagcactgcataattctcttactgtcatgccatc
cgtaagatgatttctgtgactggtgagtactcaaccaagtcattctgagaatagtgtatgcggcgaccgagttgctcttgcccggcgtca
atacgggataataccgcgccacatagcagaactttaaaagtgctcatcattggaaaacgttcttcggggcgaaaactctcaaggatctta
ccgctgttgagatccagttcgatgtaacccactcgtgcacccaactgatcttcagcatcttttactttcaccagcgtttctgggtgagcaaa
aacaggaaggcaaaatgccgcaaaaaagggaataagggcgacacggaaatgttgaatactcatactatcattttcaatattattgaag
catttatcagggttattgtctcatgagcggatacatatttgaatgtatttagaaaaataaacaaataggggttccgcgcacatttccccgaaa
agtgccacctgacgtcgacggatcgggagatcaacttgtttattgcagcttataatggttacaaataaagcaatagcatcacaaatttcac
aaataaagcatttttttcactgcattctagttgtggtttgtccaaactcatcaatgtatcttatcatgtctggatcaactggataactcaagctaa
ccaaaatcatcccaaacttcccaccccataccctattaccactgccaattacctgtggtttcatttactctaaacctgtgattcctctgaattat
tttcattttaaagaaattgtatttgttaaatatgtactacaaacttagtagt (SEQ ID NO: 22)
pLVX-CMV-TCR3-pTert-iCas9 (FIG. 5):
tggaagggctaattcactcccaaagaagacaagatatccttgatctgtggatctaccacacacaaggctacttccctgattagcagaact
acacaccagggccaggggtcagatatccactgacctttggatggtgctacaagctagtaccagttgagccagataaggtagaagagg
ccaataaaggagagaacaccagcttgttacaccctgtgagcctgcatgggatggatgacccggagagagaagtgttagagtggaggt
ttgacagccgcctagcatttcatcacgtggcccgagagctgcatccggagtacttcaagaactgctgatatcgagcttgctacaaggga
ctttccgctggggactttccagggaggcgtggcctgggcgggactggggagtggcgagccctcagatcctgcatataagcagctgct
ttttgcctgtactgggtctctctggttagaccagatctgagcctgggagctctctggctaactagggaacccactgcttaagcctcaataa
agcttgccttgagtgatcaagtagtgtgtgcccgtctgttgtgtgactctggtaactagagatccctcagacccttttagtcagtgtggaa
aatctctagcagtggcgcccgaacagggacttgaaagcgaaagggaaaccagaggagctctctcgacgcaggactcggcttgctga
agcgcgcacggcaagaggcgaggggcggcgactggtgagtacgccaaaaattttgactagcggaggctagaaggagagagatgg
gtgcgagagcgtcagtattaagcgggggagaattagatcgcgatgggaaaaaattcggttaaggccagggggaaagaaaaaatata
aattaaaacatatagtatgggcaagcagggagctagaacgattcgcagttaatcctggcctgttagaaacatcagaaggctgtagacaa
atactgggacagctacaaccatcccttcagacaggatcagaagaacttagatcattatataatacagtagcaaccctctattgtgtgcatc
aaaggatagagataaaagacaccaaggaagctttagacaagatagaggaagagcaaaacaaaagtaagaccaccgcacagcaag
cggccggccgctgatcttcagacctggaggaggagatatgagggacaattggagaagtgaattatataaatataaagtagtaaaaattg
aaccattaggagtagcacccaccaaggcaaagagaagagtggtgcagagagaaaaaagagcagtgggaataggagctttgttcctt
gggttcttgggagcagcaggaagcactatgggcgcagcgtcaatgacgctgacggtacaggccagacaattattgtctggtatagtgc
agcagcagaacaatttgctgagggctattgaggcgcaacagcatctgttgcaactcacagtctggggcatcaagcagctccaggcaa
gaatcctggctgtggaaagatacctaaaggatcaacagctcctggggatttggggttgctctggaaaactcatttgcaccactgctgtgc
cttggaatgctagttggagtaataaatctctggaacagatttggaatcacacgacctggatggagtgggacagagaaattaacaattaca
caagataatacactccttaattgaagaatcgcaaaaccagcaagaaaagaatgaacaagaattattggaattagataaatgggcaagtt
tgtggaattggtttaacataacaaattggctgtggtatataaaattattcataatgatagtaggaggcttggtaggtttaagaatagtttttgct
gtactttctatagtgaatagagttaggcagggatattcaccattatcgtttcagacccacctcccaaccccgaggggacccgacaggcc
cgaaggaatagaagaagaaggtggagagagagacagagacagatccattcgattagtgaacggatctcgacggtatcgcctttaaaa
gaaaaggggggattggggggtacagtgcaggggaaagaatagtagacataatagcaacagacatacaaactaaagaattacaaaaa
caaattacaaaaattcaaaattttcgggtttattacagggacagcagagatccagtttatcgataagcttgggagttccgcgttacataactt
acggtaaatggcccgcctggctgaccgcccaacgacccccgcccattgacgtcaataatgacgtatgttcccatagtaacgccaatag
ggactttccattgacgtcaatgggtggagtatttacggtaaactgcccacttggcagtacatcaagtgtatcatatgccaagtacgccccc
tattgacgtcaatgacggtaaatggcccgcctggcattatgcccagtacatgaccttatgggactttcctacttggcagtacatctacgtat
tagtcatcgctattaccatggtgatgcggttttggcagtacatcaatgggcgtggatagcggtttgactcacggggatttccaagtctcca
ccccattgacgtcaatgggagtttgttttggcaccaaaatcaacgggactttccaaaatgtcgtaacaactccgccccattgacgcaaat
gggcggtaggcgtgtacggtgggaggtctatataagcagagctcgtttagtgaaccgtcagatcgcctggagacgccatccacgctgt
tttgacctccatagaagacaccgactctactagaggatctatttccggtgAATTCGCCACCatgggcacaaggttgttcttctat
gtggccctttgtctcctgtggaccggtcacatgGAAGCTGACATCTACCAGACCCCAAGATACCTTGT
TATAGGGACAGGAAAGAAGATCACTCTGGAATGTTCTCAAACCATGGGCCATGA
CAAAATGTACTGGTATCAACAAGATCCAGGAATGGAACTACACCTCATCCACTAT
TCCTATGGAGTTAATTCCACAGAGAAGGGAGATCTTTCCTCTGAGTCAACAGTCT
CCAGAATAAGGACGGAGCATTTTCCCCTGACCCTGGAGTCTGCCAGGCCCTCACA
TACCTCTCAGTACCTCTGTGCCAGCAGTCGGACGCCCAACATTCAGTACTTCGGC
GCCGGGACCCGGCTCTCAGTGCTGgaggacctgaaaaacgtgttcccacccgaggtcgctgtgtttgagccatc
agaagcagagatctcccacacccaaaaggccacACTAGTgtgcctggccacaggcttctaccccgaccacgtggagctgagct
ggtgggtgaatgggaaggaggtgcacagtggggtcagcacagacccgcagcccctcaaggagcagcccgccctcaatgactcca
gatactgcctgagcagccgcctgagggtctcggccaccttctggcagaacccccgcaaccacttccgctgtcaagtccagttctacgg
gctctcggagaatgacgagtggacccaggatagggccaaacctgtcacccagatcgtcagcgccgaggcctggggtagagcagac
tgtggcttcacctccgagtcttaccagcaaggggtcctgtctgccaccatcctctatgagatcttgctagggaaggccaccttgtatgcc
gtgctggtcagtgccctcgtgctgatggccatggtcaagagaaaggattccagaggcGGGAGCGGAGCCACGAAC
TTCTCTCTGTTAAAGCAAGCAGGAGACGTGGAAGAAAACCCCGGTCCTatggcaggca
ttcgagctttatttatgtacttgtggctgcagctggactgggtctcgagaGGTCAACAGCTGAATCAGAGTCCTC
AATCTATGTTTATCCAGGAAGGAGAAGATGTCTCCATGAACTGCACTTCTTCAAG
CATATTTAACACCTGGCTATGGTACAAGCAGGAACCTGGGGAAGGTCCTGTCCTC
TTGATAGCCTTATATAAGGCTGGTGAATTGACCTCAAATGGAAGACTGACTGCTC
AGTTTGGTATAACCAGAAAGGACAGCTTCCTGAATATCTCAGCATCCATACCTAG
TGATGTAGGCATCTACTTCTGTGCTGGCACATACCAGAAAGTTACCTTTGGAACT
GGAACAAAGCTCCAAGTCATCCCAaatatccagaaccctgaccctgccgtgtaccagctgagagactctaaatc
cagtgacaagtctgtctgcctattcaccgattttgattctcaaacaaatgtgtcacaaagtaaggattctgatgtgtatatcacagacaaaa
ctgtgctagacatgaggtctatggacttcaagagcaacagtGCGGCCgcctggagcaacaaatctgactttgcatgtgcaaacgc
cttcaacaacagcattattccagaagacaccttcttccccagcccagaaagttcctgtgatgtcaagctggtcgagaaaagctttgaaac
agatacgaacctaaactttcaaaacctgtcagtgattgggttccgaatcctcctcctgaaagtggccgggtttaatctgctcatgacgctg
cggctgtggtccagcTGActcgagggatcccgcccctctccctcccccccccctaacgttactggccgaagccgcttggaataagg
ccggtgtgcgtttgtctatatgttattttccaccatattgccgtcttttggcaatgtgagggcccggaaacctggccctgtcttcttgacgag
cattcctaggggtattcccctctcgccaaaggaatgcaaggtctgttgaatgtcgtgaaggaagcagttcctctggaagcttcttgaaga
caaacaacgtctgtagcgaccctttgcaggcagcggaaccccccacctggcgacaggtgcctctgcggccaaaagccacgtgtata
agatacacctgcaaaggcggcacaaccccagtgccacgttgtgagttggatagttgtggaaagagtcaaatggctcacctcaagcgta
ttcaacaaggggctgaaggatgcccagaaggtaccccattgtatgggatctgatctggggcctcggtgcacatgctttacatgtgtttag
tcgaggttaaaaaacgtctaggccccccgaaccacggggacgtggttttcctttgaaaaacacgatgataatatggtgagcaagggcg
aggaggataacatggccatcatcaaggagttcatgcgcttcaaggtgcacatggagggctccgtgaacggccacgagttcgagatcg
agggcgagggcgagggccgcccctacgagggcacccagaccgccaagctgaaggtgaccaagggtggccccctgcccttcgcct
gggacatcctgtcccctcagttcatgtacggctccaaggcctacgtgaagcaccccgccgacatccccgactacttgaagctgtccttc
cccgagggcttcaagtgggagcgcgtgatgaacttcgaggacggcggcgtggtgaccgtgacccaggactcctccctgcaggacg
gcgagttcatctacaaggtgaagctgcgcggcaccaacttcccctccgacggccccgtaatgcagaagaagaccatgggctgggag
gcctcctccgagcggatgtaccccgaggacggcgccctgaagggcgagatcaagcagaggctgaagctgaaggacggcggcca
ctacgacgctgaggtcaagaccacctacaaggccaagaagcccgtgcagctgcccggcgcctacaacgtcaacatcaagttggaca
tcacctcccacaacgaggactacaccatcgtggaacagtacgaacgcgccgagggccgccactccaccggcggcatggacgagct
gtacaagtgaacgcgtctggaacaagattttccccgtatccccccaggtgtctgcaggctcaaagagcagcgagaagcgttcagagg
aaagcgatcccgtgccaccttccccgtgcccgggctgtccccgcacgctgccggctcggggatgcggggggagcgccggaccgg
agcggagccccgggcggctcgctgctgccccctagcgggggagggacgtaattacatccctgggggctttgggggggggctgtcc
ccgtgagctcttactccctatcagtgatagagaacgtatgaagagtttactccctatcagtgatagagaacgtatgcagactttactcccta
tcagtgatagagaacgtataaggagtttactccctatcagtgatagagaacgtatgaccagtttactccctatcagtgatagagaacgtat
ctacagtttactccctatcagtgatagagaacgtatatccagtttactccctatcagtgatagagaacgtatgtcgaggtaggcgtgtacg
gtgggcgcctataaaagcagagctcgtttagtgaaccgtcagatcgcctggagcaattccacaacacttttgtcttatacttATGCTC
GAGGGAGTGCAGGTGGAGACTATCTCCCCAGGAGACGGGCGCACCTTCCCCAAG
CGCGGCCAGACCTGCGTGGTGCACTACACCGGGATGCTTGAAGATGGAAAGAAA
GTTGATTCCTCCCGGGACAGAAACAAGCCCTTTAAGTTTATGCTAGGCAAGCAGG
AGGTGATCCGAGGCTGGGAAGAAGGGGTTGCCCAGATGAGTGTGGGTCAGAGAG
CCAAACTGACTATATCTCCAGATTATGCCTATGGTGCCACTGGGCACCCAGGCAT
CATCCCACCACATGCCACTCTCGTCTTCGATGTGGAGCTTCTAAAACTGGAATCT
GGCGGTGGATCCGGAGTCGACGGATTTGGTGATGTCGGTGCTCTTGAGAGTTTGA
GGGGAAATGCAGATTTGGCTTACATCCTGAGCATGGAGCCCTGTGGCCACTGCCT
CATTATCAACAATGTGAACTTCTGCCGTGAGTCCGGGCTCCGCACCCGCACTGGC
TCCAACATCGACTGTGAGAAGTTGCGGCGTCGCTTCTCCTCGCTGCATTTCATGG
TGGAGGTGAAGGGCGACCTGACTGCCAAGAAAATGGTGCTGGCTTTGCTGGAGC
TGGCGCGGCAGGACCACGGTGCTCTGGACTGCTGCGTGGTGGTCATTCTCTCTCA
CGGCTGTCAGGCCAGCCACCTGCAGTTCCCAGGGGCTGTCTACGGCACAGATGG
ATGCCCTGTGTCGGTCGAGAAGATTGTGAACATCTTCAATGGGACCAGCTGCCCC
AGCCTGGGAGGGAAGCCCAAGCTCTTTTTCATCCAGGCCTGTGGTGGGGAGCAG
AAAGACCATGGGTTTGAGGTGGCCTCCACTTCCCCTGAAGACGAGTCCCCTGGCA
GTAACCCCGAGCCAGATGCCACCCCGTTCCAGGAAGGTTTGAGGACCTTCGACC
AGCTGGACGCCATATCTAGTTTGCCCACACCCAGTGACATCTTTGTGTCCTACTCT
ACTTTCCCAGGTTTTGTTTCCTGGAGGGACCCCAAGAGTGGCTCCTGGTACGTTG
AGACCCTGGACGACATCTTTGAGCAGTGGGCTCACTCTGAAGACCTGCAGTCCCT
CCTGCTTAGGGTCGCTAATGCTGTTTCGGTGAAAGGGATTTATAAACAGATGCCT
GGTTGCTTTAATTTCCTCCGGAAAAAACTTTTCTTTAAAACATCAGTCGACTATCC
GTACGACGTACCAGACTACGCACTCGACTAAaagattttccccgtatccccccaggtgtctgcaggctc
aaagagcagcgagaagcgttcagaggaaagcgatcccgtgccaccttccccgtgcccgggctgtccccgcacgctgccggctcgg
ggatgcggggggagcgccggaccggagcggagccccgggcggctcgctgctgccccctagcgggggagggacgtaattacatc
cctgggggattgggagggggctgtccccgtgagctcaatcaacctctggattacaaaatttgtgaaagattgactggtattcttaactat
gttgctccttttacgctatgtggatacgctgctttaatgcctttgtatcatgctattgcttcccgtatggctttcattttctcctccttgtataaatc
ctggttgctgtctctttatgaggagttgtggcccgttgtcaggcaacgtggcgtggtgtgcactgtgtttgctgacgcaacccccactggt
tggggcattgccaccacctgtcagctcctttccgggactttcgctttccccctccctattgccacggcggaactcatcgccgcctgccttg
cccgctgctggacaggggctcggctgttgggcactgacaattccgtggtgttgtcggggaagctgacgtcctttccatggctgctcgcc
tgtgttgccacctggattctgcgcgggacgtccttctgctacgtcccttcggccctcaatccagcggaccttccttcccgcggcctgctg
ccggctctgcggcctcttccgcgtcttcgccttcgccctcagacgagtcggatctccctttgggccgcctccccgcctggaattaattctg
cagtcgagacctagaaaaacatggagcaatcacaagtagcaatacagcagctaccaatgctgattgtgcctggctagaagcacaaga
ggaggaggaggtgggttttccagtcacacctcaggtacctttaagaccaatgacttacaaggcagctgtagatcttagccactttttaaaa
gaaaagaggggactggaagggctaattcactcccaacgaagacaagatatccttgatctgtggatctaccacacacaaggctacttcc
ctgattagcagaactacacaccagggccaggggtcagatatccactgacctttggatggtgctacaagctagtaccagttgagccagat
aaggtagaagaggccaataaaggagagaacaccagcttgttacaccctgtgagcctgcatgggatggatgacccggagagagaagt
gttagagtggaggtttgacagccgcctagcatttcatcacgtggcccgagagctgcatccggagtacttcaagaactgctgatatcgag
cttgctacaagggactttccgctggggactttccagggaggcgtggcctgggcgggactggggagtggcgagccctcagatcctgc
atataagcagctgctttttgcctgtactgggtctctctggttagaccagatctgagcctgggagctctctggctaactagggaacccactg
cttaagcctcaataaagcttgccttgagtgcttcaagtagtgtgtgcccgtctgttgtgtgactctggtaactagagatccctcagacccttt
tagtcagtgtggaaaatctctagcagtagtagttcatgtcatcttattattcagtatttataacttgcaaagaaatgaatatcagagagtgaga
ggccttgacattgctagcgtttaccgtcgacctctagctagagcttggcgtaatcatggtcatagctgtttcctgtgtgaaattgttatccgc
tcacaattccacacaacatacgagccggaagcataaagtgtaaagcctggggtgcctaatgagtgagctaactcacattaattgcgttg
cgctcactgcccgctttccagtcgggaaacctgtcgtgccagctgcattaatgaatcggccaacgcgcggggagaggcggtttgcgta
ttgggcgctcttccgcttcctcgctcactgactcgctgcgctcggtcgttcggctgcggcgagcggtatcagctcactcaaaggcggta
atacggttatccacagaatcaggggataacgcaggaaagaacatgtgagcaaaaggccagcaaaaggccaggaaccgtaaaaagg
ccgcgttgctggcgtttttccataggctccgcccccctgacgagcatcacaaaaatcgacgctcaagtcagaggtggcgaaacccgac
aggactataaagataccaggcgtttccccctggaagctccctcgtgcgctctcctgttccgaccctgccgcttaccggatacctgtccgc
ctttctcccttcgggaagcgtggcgctttctcatagctcacgctgtaggtatctcagttcggtgtaggtcgttcgctccaagctgggctgtg
tgcacgaaccccccgttcagcccgaccgctgcgccttatccggtaactatcgtcttgagtccaacccggtaagacacgacttatcgcca
ctggcagcagccactggtaacaggattagcagagcgaggtatgtaggcggtgctacagagttcttgaagtggtggcctaactacggct
acactagaagaacagtatttggtatctgcgctctgctgaagccagttaccttcggaaaaagagttggtagctcttgatccggcaaacaaa
ccaccgctggtagcggtggtttttttgtttgcaagcagcagattacgcgcagaaaaaaaggatctcaagaagatcctttgatcttttctacg
gggtctgacgctcagtggaacgaaaactcacgttaagggattttggtcatgagattatcaaaaaggatcttcacctagatccttttaaatta
aaaatgaagttttaaatcaatctaaagtatatatgagtaaacttggtctgacagttaccaatgcttaatcagtgaggcacctatctcagcgat
ctgtctatttcgttcatccatagttgcctgactccccgtcgtgtagataactacgatacgggagggcttaccatctggccccagtgctgca
atgataccgcgagacccacgctcaccggctccagatttatcagcaataaaccagccagccggaagggccgagcgcagaagtggtcc
tgcaactttatccgcctccatccagtctattaattgttgccgggaagctagagtaagtagttcgccagttaatagtttgcgcaacgttgttgc
cattgctacaggcatcgtggtgtcacgctcgtcgtttggtatggcttcattcagctccggttcccaacgatcaaggcgagttacatgatcc
cccatgttgtgcaaaaaagcggttagctccttcggtcctccgatcgttgtcagaagtaagttggccgcagtgttatcactcatggttatgg
cagcactgcataattctcttactgtcatgccatccgtaagatgcttttctgtgactggtgagtactcaaccaagtcattctgagaatagtgtat
gcggcgaccgagttgctcttgcccggcgtcaatacgggataataccgcgccacatagcagaactttaaaagtgctcatcattggaaaa
cgttcttcggggcgaaaactctcaaggatcttaccgctgttgagatccagttcgatgtaacccactcgtgcacccaactgatcttcagcat
cttttactttcaccagcgtttctgggtgagcaaaaacaggaaggcaaaatgccgcaaaaaagggaataagggcgacacggaaatgttg
aatactcatactcttcctttttcaatattattgaagcatttatcagggttattgtctcatgagcggatacatatttgaatgtatttagaaaaataaa
caaataggggttccgcgcacatttccccgaaaagtgccacctgacgtcgacggatcgggagatcaacttgtttattgcagcttataatgg
ttacaaataaagcaatagcatcacaaatttcacaaataaagcatttttttcactgcattctagttgtggtttgtccaaactcatcaatgtatctta
tcatgtctggatcaactggataactcaagctaaccaaaatcatcccaaacttcccaccccataccctattaccactgccaattacctgtggt
ttcatttactctaaacctgtgattcctctgaattattttcattttaaagaaattgtatttgttaaatatgtactacaaacttagtagt (SEQ ID
NO: 23)
pLVX-CMV-TCR4-pTert-iCas9 (FIG. 6):
tggaagggctaattcactcccaaagaagacaagatatccttgatctgtggatctaccacacacaaggctacttccctgattagcagaact
acacaccagggccaggggtcagatatccactgacctttggatggtgctacaagctagtaccagttgagccagataaggtagaagagg
ccaataaaggagagaacaccagcttgttacaccctgtgagcctgcatgggatggatgacccggagagagaagtgttagagtggaggt
ttgacagccgcctagcatttcatcacgtggcccgagagctgcatccggagtacttcaagaactgctgatatcgagcttgctacaaggga
ctttccgctggggactttccagggaggcgtggcctgggcgggactggggagtggcgagccctcagatcctgcatataagcagctgct
ttttgcctgtactgggtctctctggttagaccagatctgagcctgggagctctctggctaactagggaacccactgcttaagcctcaataa
agcttgccttgagtgatcaagtagtgtgtgcccgtctgttgtgtgactctggtaactagagatccctcagacccttttagtcagtgtggaa
aatctctagcagtggcgcccgaacagggacttgaaagcgaaagggaaaccagaggagctctctcgacgcaggactcggcttgctga
agcgcgcacggcaagaggcgaggggcggcgactggtgagtacgccaaaaattttgactagcggaggctagaaggagagagatgg
gtgcgagagcgtcagtattaagcgggggagaattagatcgcgatgggaaaaaattcggttaaggccagggggaaagaaaaaatata
aattaaaacatatagtatgggcaagcagggagctagaacgattcgcagttaatcctggcctgttagaaacatcagaaggctgtagacaa
atactgggacagctacaaccatcccttcagacaggatcagaagaacttagatcattatataatacagtagcaaccctctattgtgtgcatc
aaaggatagagataaaagacaccaaggaagctttagacaagatagaggaagagcaaaacaaaagtaagaccaccgcacagcaag
cggccggccgctgatcttcagacctggaggaggagatatgagggacaattggagaagtgaattatataaatataaagtagtaaaaattg
aaccattaggagtagcacccaccaaggcaaagagaagagtggtgcagagagaaaaaagagcagtgggaataggagctttgttcctt
gggttcttgggagcagcaggaagcactatgggcgcagcgtcaatgacgctgacggtacaggccagacaattattgtctggtatagtgc
agcagcagaacaatttgctgagggctattgaggcgcaacagcatctgttgcaactcacagtctggggcatcaagcagctccaggcaa
gaatcctggctgtggaaagatacctaaaggatcaacagctcctggggatttggggttgctctggaaaactcatttgcaccactgctgtgc
cttggaatgctagttggagtaataaatctctggaacagatttggaatcacacgacctggatggagtgggacagagaaattaacaattaca
caagataatacactccttaattgaagaatcgcaaaaccagcaagaaaagaatgaacaagaattattggaattagataaatgggcaagtt
tgtggaattggtttaacataacaaattggctgtggtatataaaattattcataatgatagtaggaggcttggtaggtttaagaatagtttttgct
gtactttctatagtgaatagagttaggcagggatattcaccattatcgtttcagacccacctcccaaccccgaggggacccgacaggcc
cgaaggaatagaagaagaaggtggagagagagacagagacagatccattcgattagtgaacggatctcgacggtatcgcctttaaaa
gaaaaggggggattggggggtacagtgcaggggaaagaatagtagacataatagcaacagacatacaaactaaagaattacaaaaa
caaattacaaaaattcaaaattttcgggtttattacagggacagcagagatccagtttatcgataagcttgggagttccgcgttacataactt
acggtaaatggcccgcctggctgaccgcccaacgacccccgcccattgacgtcaataatgacgtatgttcccatagtaacgccaatag
ggactttccattgacgtcaatgggtggagtatttacggtaaactgcccacttggcagtacatcaagtgtatcatatgccaagtacgccccc
tattgacgtcaatgacggtaaatggcccgcctggcattatgcccagtacatgaccttatgggactttcctacttggcagtacatctacgtat
tagtcatcgctattaccatggtgatgcggttttggcagtacatcaatgggcgtggatagcggtttgactcacggggatttccaagtctcca
ccccattgacgtcaatgggagtttgttttggcaccaaaatcaacgggactttccaaaatgtcgtaacaactccgccccattgacgcaaat
gggcggtaggcgtgtacggtgggaggtctatataagcagagctcgtttagtgaaccgtcagatcgcctggagacgccatccacgctgt
tttgacctccatagaagacaccgactctactagaggatctatttccggtgAATTCGCCACCatgggaatcaggctcctgtgtcg
tgtggccttttgtttcctggctgtaggactagtaGAAGCTGACATCTACCAGACCCCAAGATACCTTGT
TATAGGGACAGGAAAGAAGATCACTCTGGAATGTTCTCAAACCATGGGCCATGA
CAAAATGTACTGGTATCAACAAGATCCAGGAATGGAACTACACCTCATCCACTAT
TCCTATGGAGTTAATTCCACAGAGAAGGGAGATCTTTCCTCTGAGTCAACAGTCT
CCAGAATAAGGACGGAGCATTTTCCCCTGACCCTGGAGTCTGCCAGGCCCTCACA
TACCTCTCAGTACCTCTGTGCCAGCAGTCGGACGCCCAACATTCAGTACTTCGGC
GCCGGGACCCGGCTCTCAGTGCTGgaggacctgaaaaacgtgttcccacccgaggtcgctgtgtttgagccatc
agaagcagagatctcccacacccaaaaggccacACTgGCAtgcctggccacaggcttctaccccgaccacgtggagctgagc
tggtgggtgaatgggaaggaggtgcacagtggggtcagcacagacccgcagcccctcaaggagcagcccgccctcaatgactcca
gatactgcctgagcagccgcctgagggtctcggccaccttctggcagaacccccgcaaccacttccgctgtcaagtccagttctacgg
gctctcggagaatgacgagtggacccaggatagggccaaacctgtcacccagatcgtcagcgccgaggcctggggtagagcagac
tgtggcttcacctccgagtcttaccagcaaggggtcctgtctgccaccatcctctatgagatcttgctagggaaggccaccttgtatgcc
gtgctggtcagtgccctcgtgctgatggccatggtcaagagaaaggattccagaggcGGGAGCGGAGCCACGAAC
TTCTCTCTGTTAAAGCAAGCAGGAGACGTGGAAGAAAACCCCGGTCCTatgacacgagt
tagcttgctgtgggcagtcgtggtctccacctgtctcgagtccggcatgGGTCAACAGCTGAATCAGAGTCCTC
AATCTATGTTTATCCAGGAAGGAGAAGATGTCTCCATGAACTGCACTTCTTCAAG
CATATTTAACACCTGGCTATGGTACAAGCAGGAACCTGGGGAAGGTCCTGTCCTC
TTGATAGCCTTATATAAGGCTGGTGAATTGACCTCAAATGGAAGACTGACTGCTC
AGTTTGGTATAACCAGAAAGGACAGCTTCCTGAATATCTCAGCATCCATACCTAG
TGATGTAGGCATCTACTTCTGTGCTGGCACATACCAGAAAGTTACCTTTGGAACT
GGAACAAAGCTCCAAGTCATCCCAaatatccagaaccctgaccctgccgtgtaccagctgagagactctaaatc
cagtgacaagtctgtctgcctattcaccgattttgattctcaaacaaatgtgtcacaaagtaaggattctgatgtgtatatcacagacaaaa
ctgtgctagacatgaggtctatggacttcaagagcaacagtGCGGCCgcctggagcaacaaatctgactttgcatgtgcaaacgc
cttcaacaacagcattattccagaagacaccttcttccccagcccagaaagttcctgtgatgtcaagctggtcgagaaaagctttgaaac
agatacgaacctaaactttcaaaacctgtcagtgattgggttccgaatcctcctcctgaaagtggccgggtttaatctgctcatgacgctg
cggctgtggtccagcTGActcgagggatcccgcccctctccctcccccccccctaacgttactggccgaagccgcttggaataagg
ccggtgtgcgtttgtctatatgttattttccaccatattgccgtcttttggcaatgtgagggcccggaaacctggccctgtcttcttgacgag
cattcctaggggtattcccctctcgccaaaggaatgcaaggtctgttgaatgtcgtgaaggaagcagttcctctggaagcttcttgaaga
caaacaacgtctgtagcgaccctttgcaggcagcggaaccccccacctggcgacaggtgcctctgcggccaaaagccacgtgtata
agatacacctgcaaaggcggcacaaccccagtgccacgttgtgagttggatagttgtggaaagagtcaaatggctcacctcaagcgta
ttcaacaaggggctgaaggatgcccagaaggtaccccattgtatgggatctgatctggggcctcggtgcacatgctttacatgtgtttag
tcgaggttaaaaaacgtctaggccccccgaaccacggggacgtggttttcctttgaaaaacacgatgataatatggtgagcaagggcg
aggaggataacatggccatcatcaaggagttcatgcgcttcaaggtgcacatggagggctccgtgaacggccacgagttcgagatcg
agggcgagggcgagggccgcccctacgagggcacccagaccgccaagctgaaggtgaccaagggtggccccctgcccttcgcct
gggacatcctgtcccctcagttcatgtacggctccaaggcctacgtgaagcaccccgccgacatccccgactacttgaagctgtccttc
cccgagggcttcaagtgggagcgcgtgatgaacttcgaggacggcggcgtggtgaccgtgacccaggactcctccctgcaggacg
gcgagttcatctacaaggtgaagctgcgcggcaccaacttcccctccgacggccccgtaatgcagaagaagaccatgggctgggag
gcctcctccgagcggatgtaccccgaggacggcgccctgaagggcgagatcaagcagaggctgaagctgaaggacggcggcca
ctacgacgctgaggtcaagaccacctacaaggccaagaagcccgtgcagctgcccggcgcctacaacgtcaacatcaagttggaca
tcacctcccacaacgaggactacaccatcgtggaacagtacgaacgcgccgagggccgccactccaccggcggcatggacgagct
gtacaagtgaacgcgtctggaacaagctttttccccgtatccccccaggtgtctgcaggctcaaagagcagcgagaagcgttcagagg
aaagcgatcccgtgccaccttccccgtgcccgggctgtccccgcacgctgccggctcggggatgcggggggagcgccggaccgg
agcggagccccgggcggctcgctgctgccccctagcgggggagggacgtaattacatccctgggggctttgggggggggctgtcc
ccgtgagctcttactccctatcagtgatagagaacgtatgaagagtttactccctatcagtgatagagaacgtatgcagactttactcccta
tcagtgatagagaacgtataaggagtttactccctatcagtgatagagaacgtatgaccagtttactccctatcagtgatagagaacgtat
ctacagtttactccctatcagtgatagagaacgtatatccagtttactccctatcagtgatagagaacgtatgtcgaggtaggcgtgtacg
gtgggcgcctataaaagcagagctcgtttagtgaaccgtcagatcgcctggagcaattccacaacacttttgtcttatacttATGCTC
GAGGGAGTGCAGGTGGAGACTATCTCCCCAGGAGACGGGCGCACCTTCCCCAAG
CGCGGCCAGACCTGCGTGGTGCACTACACCGGGATGCTTGAAGATGGAAAGAAA
GTTGATTCCTCCCGGGACAGAAACAAGCCCTTTAAGTTTATGCTAGGCAAGCAGG
AGGTGATCCGAGGCTGGGAAGAAGGGGTTGCCCAGATGAGTGTGGGTCAGAGAG
CCAAACTGACTATATCTCCAGATTATGCCTATGGTGCCACTGGGCACCCAGGCAT
CATCCCACCACATGCCACTCTCGTCTTCGATGTGGAGCTTCTAAAACTGGAATCT
GGCGGTGGATCCGGAGTCGACGGATTTGGTGATGTCGGTGCTCTTGAGAGTTTGA
GGGGAAATGCAGATTTGGCTTACATCCTGAGCATGGAGCCCTGTGGCCACTGCCT
CATTATCAACAATGTGAACTTCTGCCGTGAGTCCGGGCTCCGCACCCGCACTGGC
TCCAACATCGACTGTGAGAAGTTGCGGCGTCGCTTCTCCTCGCTGCATTTCATGG
TGGAGGTGAAGGGCGACCTGACTGCCAAGAAAATGGTGCTGGCTTTGCTGGAGC
TGGCGCGGCAGGACCACGGTGCTCTGGACTGCTGCGTGGTGGTCATTCTCTCTCA
CGGCTGTCAGGCCAGCCACCTGCAGTTCCCAGGGGCTGTCTACGGCACAGATGG
ATGCCCTGTGTCGGTCGAGAAGATTGTGAACATCTTCAATGGGACCAGCTGCCCC
AGCCTGGGAGGGAAGCCCAAGCTCTTTTTCATCCAGGCCTGTGGTGGGGAGCAG
AAAGACCATGGGTTTGAGGTGGCCTCCACTTCCCCTGAAGACGAGTCCCCTGGCA
GTAACCCCGAGCCAGATGCCACCCCGTTCCAGGAAGGTTTGAGGACCTTCGACC
AGCTGGACGCCATATCTAGTTTGCCCACACCCAGTGACATCTTTGTGTCCTACTCT
ACTTTCCCAGGTTTTGTTTCCTGGAGGGACCCCAAGAGTGGCTCCTGGTACGTTG
AGACCCTGGACGACATCTTTGAGCAGTGGGCTCACTCTGAAGACCTGCAGTCCCT
CCTGCTTAGGGTCGCTAATGCTGTTTCGGTGAAAGGGATTTATAAACAGATGCCT
GGTTGCTTTAATTTCCTCCGGAAAAAACTTTTCTTTAAAACATCAGTCGACTATCC
GTACGACGTACCAGACTACGCACTCGACTAAaagattttccccgtatccccccaggtgtctgcaggctc
aaagagcagcgagaagcgttcagaggaaagcgatcccgtgccaccttccccgtgcccgggctgtccccgcacgctgccggctcgg
ggatgcggggggagcgccggaccggagcggagccccgggcggctcgctgctgccccctagcgggggagggacgtaattacatc
cctgggggctttgggagggggctgtccccgtgagctcaatcaacctctggattacaaaatttgtgaaagattgactggtattcttaactat
gttgctccttttacgctatgtggatacgctgctttaatgcctttgtatcatgctattgcttcccgtatggctttcattttctcctccttgtataaatc
ctggttgctgtctctttatgaggagttgtggcccgttgtcaggcaacgtggcgtggtgtgcactgtgtttgctgacgcaacccccactggt
tggggcattgccaccacctgtcagctcctttccgggactttcgctttccccctccctattgccacggcggaactcatcgccgcctgccttg
cccgctgctggacaggggctcggctgttgggcactgacaattccgtggtgttgtcggggaagctgacgtcctttccatggctgctcgcc
tgtgttgccacctggattctgcgcgggacgtccttctgctacgtcccttcggccctcaatccagcggaccttccttcccgcggcctgctg
ccggctctgcggcctatccgcgtatcgccttcgccctcagacgagtcggatctccattgggccgcctccccgcctggaattaattctg
cagtcgagacctagaaaaacatggagcaatcacaagtagcaatacagcagctaccaatgctgattgtgcctggctagaagcacaaga
ggaggaggaggtgggttttccagtcacacctcaggtacctttaagaccaatgacttacaaggcagctgtagatcttagccactttttaaaa
gaaaagaggggactggaagggctaattcactcccaacgaagacaagatatccttgatctgtggatctaccacacacaaggctacttcc
ctgattagcagaactacacaccagggccaggggtcagatatccactgacctttggatggtgctacaagctagtaccagttgagccagat
aaggtagaagaggccaataaaggagagaacaccagcttgttacaccctgtgagcctgcatgggatggatgacccggagagagaagt
gttagagtggaggtttgacagccgcctagcatttcatcacgtggcccgagagctgcatccggagtacttcaagaactgctgatatcgag
cttgctacaagggactttccgctggggactttccagggaggcgtggcctgggcgggactggggagtggcgagccctcagatcctgc
atataagcagctgctttttgcctgtactgggtctctctggttagaccagatctgagcctgggagctctctggctaactagggaacccactg
cttaagcctcaataaagcttgccttgagtgatcaagtagtgtgtgcccgtctgttgtgtgactctggtaactagagatccctcagacccttt
tagtcagtgtggaaaatctctagcagtagtagttcatgtcatcttattattcagtatttataacttgcaaagaaatgaatatcagagagtgaga
ggccttgacattgctagcgtttaccgtcgacctctagctagagcttggcgtaatcatggtcatagctgtttcctgtgtgaaattgttatccgc
tcacaattccacacaacatacgagccggaagcataaagtgtaaagcctggggtgcctaatgagtgagctaactcacattaattgcgttg
cgctcactgcccgctttccagtcgggaaacctgtcgtgccagctgcattaatgaatcggccaacgcgcggggagaggcggtttgcgta
ttgggcgctatccgcttcctcgctcactgactcgctgcgctcggtcgttcggctgcggcgagcggtatcagctcactcaaaggcggta
atacggttatccacagaatcaggggataacgcaggaaagaacatgtgagcaaaaggccagcaaaaggccaggaaccgtaaaaagg
ccgcgttgctggcgtttttccataggctccgcccccctgacgagcatcacaaaaatcgacgctcaagtcagaggtggcgaaacccgac
aggactataaagataccaggcgtttccccctggaagctccctcgtgcgctctcctgttccgaccctgccgcttaccggatacctgtccgc
ctttctcccttcgggaagcgtggcgctttctcatagctcacgctgtaggtatctcagttcggtgtaggtcgttcgctccaagctgggctgtg
tgcacgaaccccccgttcagcccgaccgctgcgccttatccggtaactatcgtcttgagtccaacccggtaagacacgacttatcgcca
ctggcagcagccactggtaacaggattagcagagcgaggtatgtaggcggtgctacagagttcttgaagtggtggcctaactacggct
acactagaagaacagtatttggtatctgcgctctgctgaagccagttaccttcggaaaaagagttggtagctcttgatccggcaaacaaa
ccaccgctggtagcggtggtttttttgtttgcaagcagcagattacgcgcagaaaaaaaggatctcaagaagatcctttgatcttttctacg
gggtctgacgctcagtggaacgaaaactcacgttaagggattttggtcatgagattatcaaaaaggatcttcacctagatccttttaaatta
aaaatgaagttttaaatcaatctaaagtatatatgagtaaacttggtctgacagttaccaatgcttaatcagtgaggcacctatctcagcgat
ctgtctatttcgttcatccatagttgcctgactccccgtcgtgtagataactacgatacgggagggcttaccatctggccccagtgctgca
atgataccgcgagacccacgctcaccggctccagatttatcagcaataaaccagccagccggaagggccgagcgcagaagtggtcc
tgcaactttatccgcctccatccagtctattaattgttgccgggaagctagagtaagtagttcgccagttaatagtttgcgcaacgttgttgc
cattgctacaggcatcgtggtgtcacgctcgtcgtttggtatggcttcattcagctccggttcccaacgatcaaggcgagttacatgatcc
cccatgttgtgcaaaaaagcggttagctccttcggtcctccgatcgttgtcagaagtaagttggccgcagtgttatcactcatggttatgg
cagcactgcataattctcttactgtcatgccatccgtaagatgcttttctgtgactggtgagtactcaaccaagtcattctgagaatagtgtat
gcggcgaccgagttgctcttgcccggcgtcaatacgggataataccgcgccacatagcagaactttaaaagtgctcatcattggaaaa
cgttcttcggggcgaaaactctcaaggatcttaccgctgttgagatccagttcgatgtaacccactcgtgcacccaactgatcttcagcat
cttttactttcaccagcgtttctgggtgagcaaaaacaggaaggcaaaatgccgcaaaaaagggaataagggcgacacggaaatgttg
aatactcatactatcattttcaatattattgaagcatttatcagggttattgtctcatgagcggatacatatttgaatgtatttagaaaaataaa
caaataggggttccgcgcacatttccccgaaaagtgccacctgacgtcgacggatcgggagatcaacttgtttattgcagcttataatgg
ttacaaataaagcaatagcatcacaaatttcacaaataaagcatttttttcactgcattctagttgtggtttgtccaaactcatcaatgtatctta
tcatgtctggatcaactggataactcaagctaaccaaaatcatcccaaacttcccaccccataccctattaccactgccaattacctgtggt
ttcatttactctaaacctgtgattcctctgaattattttcattttaaagaaattgtatttgttaaatatgtactacaaacttagtagt (SEQ ID
NO: 24)
pLVX-CMV-TCR5-pTert-iCas9 (FIG. 7):
tggaagggctaattcactcccaaagaagacaagatatccttgatctgtggatctaccacacacaaggctacttccctgattagcagaact
acacaccagggccaggggtcagatatccactgacctttggatggtgctacaagctagtaccagttgagccagataaggtagaagagg
ccaataaaggagagaacaccagcttgttacaccctgtgagcctgcatgggatggatgacccggagagagaagtgttagagtggaggt
ttgacagccgcctagcatttcatcacgtggcccgagagctgcatccggagtacttcaagaactgctgatatcgagcttgctacaaggga
ctttccgctggggactttccagggaggcgtggcctgggcgggactggggagtggcgagccctcagatcctgcatataagcagctgct
ttttgcctgtactgggtctctctggttagaccagatctgagcctgggagctctctggctaactagggaacccactgcttaagcctcaataa
agcttgccttgagtgcttcaagtagtgtgtgcccgtctgttgtgtgactctggtaactagagatccctcagacccttttagtcagtgtggaa
aatctctagcagtggcgcccgaacagggacttgaaagcgaaagggaaaccagaggagctctctcgacgcaggactcggcttgctga
agcgcgcacggcaagaggcgaggggcggcgactggtgagtacgccaaaaattttgactagcggaggctagaaggagagagatgg
gtgcgagagcgtcagtattaagcgggggagaattagatcgcgatgggaaaaaattcggttaaggccagggggaaagaaaaaatata
aattaaaacatatagtatgggcaagcagggagctagaacgattcgcagttaatcctggcctgttagaaacatcagaaggctgtagacaa
atactgggacagctacaaccatcccttcagacaggatcagaagaacttagatcattatataatacagtagcaaccctctattgtgtgcatc
aaaggatagagataaaagacaccaaggaagctttagacaagatagaggaagagcaaaacaaaagtaagaccaccgcacagcaag
cggccggccgctgatcttcagacctggaggaggagatatgagggacaattggagaagtgaattatataaatataaagtagtaaaaattg
aaccattaggagtagcacccaccaaggcaaagagaagagtggtgcagagagaaaaaagagcagtgggaataggagctttgttcctt
gggttcttgggagcagcaggaagcactatgggcgcagcgtcaatgacgctgacggtacaggccagacaattattgtctggtatagtgc
agcagcagaacaatttgctgagggctattgaggcgcaacagcatctgttgcaactcacagtctggggcatcaagcagctccaggcaa
gaatcctggctgtggaaagatacctaaaggatcaacagctcctggggatttggggttgctctggaaaactcatttgcaccactgctgtgc
cttggaatgctagttggagtaataaatctctggaacagatttggaatcacacgacctggatggagtgggacagagaaattaacaattaca
caagataatacactccttaattgaagaatcgcaaaaccagcaagaaaagaatgaacaagaattattggaattagataaatgggcaagtt
tgtggaattggtttaacataacaaattggctgtggtatataaaattattcataatgatagtaggaggcttggtaggtttaagaatagtttttgct
gtactttctatagtgaatagagttaggcagggatattcaccattatcgtttcagacccacctcccaaccccgaggggacccgacaggcc
cgaaggaatagaagaagaaggtggagagagagacagagacagatccattcgattagtgaacggatctcgacggtatcgcctttaaaa
gaaaaggggggattggggggtacagtgcaggggaaagaatagtagacataatagcaacagacatacaaactaaagaattacaaaaa
caaattacaaaaattcaaaattttcgggtttattacagggacagcagagatccagtttatcgataagcttgggagttccgcgttacataactt
acggtaaatggcccgcctggctgaccgcccaacgacccccgcccattgacgtcaataatgacgtatgttcccatagtaacgccaatag
ggactttccattgacgtcaatgggtggagtatttacggtaaactgcccacttggcagtacatcaagtgtatcatatgccaagtacgccccc
tattgacgtcaatgacggtaaatggcccgcctggcattatgcccagtacatgaccttatgggactttcctacttggcagtacatctacgtat
tagtcatcgctattaccatggtgatgcggttttggcagtacatcaatgggcgtggatagcggtttgactcacggggatttccaagtctcca
ccccattgacgtcaatgggagtttgttttggcaccaaaatcaacgggactttccaaaatgtcgtaacaactccgccccattgacgcaaat
gggcggtaggcgtgtacggtgggaggtctatataagcagagctcgtttagtgaaccgtcagatcgcctggagacgccatccacgctgt
tttgacctccatagaagacaccgactctactagaggatctatttccggtgAATTCGCCACCatggcctccctgctcttcttctgtg
gggccttttatctcctgggaaccggttccatgGAAGCTGACATCTACCAGACCCCAAGATACCTTGTT
ATAGGGACAGGAAAGAAGATCACTCTGGAATGTTCTCAAACCATGGGCCATGAC
AAAATGTACTGGTATCAACAAGATCCAGGAATGGAACTACACCTCATCCACTATT
CCTATGGAGTTAATTCCACAGAGAAGGGAGATCTTTCCTCTGAGTCAACAGTCTC
CAGAATAAGGACGGAGCATTTTCCCCTGACCCTGGAGTCTGCCAGGCCCTCACAT
ACCTCTCAGTACCTCTGTGCCAGCAGTCGGACGCCCAACATTCAGTACTTCGGCG
CCGGGACCCGGCTCTCAGTGCTGgaggacctgaaaaacgtgttcccacccgaggtcgctgtgtttgagccatca
gaagcagagatctcccacacccaaaaggccacACTAGTgtgcctggccacaggcttctaccccgaccacgtggagctgagct
ggtgggtgaatgggaaggaggtgcacagtggggtcagcacagacccgcagcccctcaaggagcagcccgccctcaatgactcca
gatactgcctgagcagccgcctgagggtctcggccaccttctggcagaacccccgcaaccacttccgctgtcaagtccagttctacgg
gctctcggagaatgacgagtggacccaggatagggccaaacctgtcacccagatcgtcagcgccgaggcctggggtagagcagac
tgtggcttcacctccgagtcttaccagcaaggggtcctgtctgccaccatcctctatgagatcttgctagggaaggccaccttgtatgcc
gtgctggtcagtgccctcgtgctgatggccatggtcaagagaaaggattccagaggcGGGAGCGGAGCCACGAAC
TTCTCTCTGTTAAAGCAAGCAGGAGACGTGGAAGAAAACCCCGGTCCTatgaactcctct
ctggactttctaattctgatcttaatgtttggaggaACTAGTGGTCAACAGCTGAATCAGAGTCCTCAAT
CTATGTTTATCCAGGAAGGAGAAGATGTCTCCATGAACTGCACTTCTTCAAGCAT
ATTTAACACCTGGCTATGGTACAAGCAGGAACCTGGGGAAGGTCCTGTCCTCTTG
ATAGCCTTATATAAGGCTGGTGAATTGACCTCAAATGGAAGACTGACTGCTCAGT
TTGGTATAACCAGAAAGGACAGCTTCCTGAATATCTCAGCATCCATACCTAGTGA
TGTAGGCATCTACTTCTGTGCTGGCACATACCAGAAAGTTACCTTTGGAACTGGA
ACAAAGCTCCAAGTCATCCCAAATATCCAGAACCCTGACCCTGCCGTGTACCAGC
TGAGAGACTCTAAATCCAGTGACAAGTCTGCATGCctattcaccgattttgattctcaaacaaatgtgtc
acaaagtaaggattctgatgtgtatatcacagacaaaactgtgctagacatgaggtctatggacttcaagagcaacagtgctgtggcctg
gagcaacaaatctgactttgcatgtgcaaacgccttcaacaacagcattattccagaagacaccttcttccccagcccagaaagttcctg
tgatgtcaagctggtcgagaaaagctttgaaacagatacgaacctaaactttcaaaacctgtcagtgattgggttccgaatcctcctcctg
aaagtggccgggtttaatctgctcatgacgctgcggctgtggtccagcTGActcgagggatcccgcccctctccctcccccccccct
aacgttactggccgaagccgcttggaataaggccggtgtgcgtttgtctatatgttattttccaccatattgccgtcttttggcaatgtgagg
gcccggaaacctggccctgtatcttgacgagcattcctaggggtctttcccctctcgccaaaggaatgcaaggtctgttgaatgtcgtg
aaggaagcagttcctctggaagatcttgaagacaaacaacgtctgtagcgaccctttgcaggcagcggaaccccccacctggcgac
aggtgcctctgcggccaaaagccacgtgtataagatacacctgcaaaggcggcacaaccccagtgccacgttgtgagttggatagttg
tggaaagagtcaaatggctcacctcaagcgtattcaacaaggggctgaaggatgcccagaaggtaccccattgtatgggatctgatct
ggggcctcggtgcacatgctttacatgtgtttagtcgaggttaaaaaacgtctaggccccccgaaccacggggacgtggttttcctttga
aaaacacgatgataatatggtgagcaagggcgaggaggataacatggccatcatcaaggagttcatgcgcttcaaggtgcacatgga
gggctccgtgaacggccacgagttcgagatcgagggcgagggcgagggccgcccctacgagggcacccagaccgccaagctga
aggtgaccaagggtggccccctgcccttcgcctgggacatcctgtcccctcagttcatgtacggctccaaggcctacgtgaagcaccc
cgccgacatccccgactacttgaagctgtccttccccgagggcttcaagtgggagcgcgtgatgaacttcgaggacggcggcgtggt
gaccgtgacccaggactcctccctgcaggacggcgagttcatctacaaggtgaagctgcgcggcaccaacttcccctccgacggcc
ccgtaatgcagaagaagaccatgggctgggaggcctcctccgagcggatgtaccccgaggacggcgccctgaagggcgagatca
agcagaggctgaagctgaaggacggcggccactacgacgctgaggtcaagaccacctacaaggccaagaagcccgtgcagctgc
ccggcgcctacaacgtcaacatcaagttggacatcacctcccacaacgaggactacaccatcgtggaacagtacgaacgcgccgag
ggccgccactccaccggcggcatggacgagctgtacaagtgaacgcgtctggaacaagctttttccccgtatccccccaggtgtctgc
aggctcaaagagcagcgagaagcgttcagaggaaagcgatcccgtgccaccttccccgtgcccgggctgtccccgcacgctgccg
gctcggggatgcggggggagcgccggaccggagcggagccccgggcggctcgctgctgccccctagcgggggagggacgtaa
ttacatccctgggggctttgggggggggctgtccccgtgagctcttactccctatcagtgatagagaacgtatgaagagtttactccctat
cagtgatagagaacgtatgcagactttactccctatcagtgatagagaacgtataaggagtttactccctatcagtgatagagaacgtatg
accagtttactccctatcagtgatagagaacgtatctacagtttactccctatcagtgatagagaacgtatatccagtttactccctatcagt
gatagagaacgtatgtcgaggtaggcgtgtacggtgggcgcctataaaagcagagctcgtttagtgaaccgtcagatcgcctggagc
aattccacaacacttttgtcttatacttATGCTCGAGGGAGTGCAGGTGGAGACTATCTCCCCAGGA
GACGGGCGCACCTTCCCCAAGCGCGGCCAGACCTGCGTGGTGCACTACACCGGG
ATGCTTGAAGATGGAAAGAAAGTTGATTCCTCCCGGGACAGAAACAAGCCCTTT
AAGTTTATGCTAGGCAAGCAGGAGGTGATCCGAGGCTGGGAAGAAGGGGTTGCC
CAGATGAGTGTGGGTCAGAGAGCCAAACTGACTATATCTCCAGATTATGCCTATG
GTGCCACTGGGCACCCAGGCATCATCCCACCACATGCCACTCTCGTCTTCGATGT
GGAGCTTCTAAAACTGGAATCTGGCGGTGGATCCGGAGTCGACGGATTTGGTGA
TGTCGGTGCTCTTGAGAGTTTGAGGGGAAATGCAGATTTGGCTTACATCCTGAGC
ATGGAGCCCTGTGGCCACTGCCTCATTATCAACAATGTGAACTTCTGCCGTGAGT
CCGGGCTCCGCACCCGCACTGGCTCCAACATCGACTGTGAGAAGTTGCGGCGTCG
CTTCTCCTCGCTGCATTTCATGGTGGAGGTGAAGGGCGACCTGACTGCCAAGAAA
ATGGTGCTGGCTTTGCTGGAGCTGGCGCGGCAGGACCACGGTGCTCTGGACTGCT
GCGTGGTGGTCATTCTCTCTCACGGCTGTCAGGCCAGCCACCTGCAGTTCCCAGG
GGCTGTCTACGGCACAGATGGATGCCCTGTGTCGGTCGAGAAGATTGTGAACATC
TTCAATGGGACCAGCTGCCCCAGCCTGGGAGGGAAGCCCAAGCTCTTTTTCATCC
AGGCCTGTGGTGGGGAGCAGAAAGACCATGGGTTTGAGGTGGCCTCCACTTCCC
CTGAAGACGAGTCCCCTGGCAGTAACCCCGAGCCAGATGCCACCCCGTTCCAGG
AAGGTTTGAGGACCTTCGACCAGCTGGACGCCATATCTAGTTTGCCCACACCCAG
TGACATCTTTGTGTCCTACTCTACTTTCCCAGGTTTTGTTTCCTGGAGGGACCCCA
AGAGTGGCTCCTGGTACGTTGAGACCCTGGACGACATCTTTGAGCAGTGGGCTCA
CTCTGAAGACCTGCAGTCCCTCCTGCTTAGGGTCGCTAATGCTGTTTCGGTGAAA
GGGATTTATAAACAGATGCCTGGTTGCTTTAATTTCCTCCGGAAAAAACTTTTCTT
TAAAACATCAGTCGACTATCCGTACGACGTACCAGACTACGCACTCGACTAAaagc
tttttccccgtatccccccaggtgtctgcaggctcaaagagcagcgagaagcgttcagaggaaagcgatcccgtgccaccttccccgt
gcccgggctgtccccgcacgctgccggctcggggatgcggggggagcgccggaccggagcggagccccgggcggctcgctgct
gccccctagcgggggagggacgtaattacatccctgggggctttgggagggggctgtccccgtgagctcaatcaacctctggattac
aaaatttgtgaaagattgactggtattcttaactatgttgctccttttacgctatgtggatacgctgctttaatgcctttgtatcatgctattgctt
cccgtatggattcattttctcctccttgtataaatcctggttgctgtctctttatgaggagttgtggcccgttgtcaggcaacgtggcgtggt
gtgcactgtgtttgctgacgcaacccccactggttggggcattgccaccacctgtcagctcctttccgggactttcgctttccccctcccta
ttgccacggcggaactcatcgccgcctgccttgcccgctgctggacaggggctcggctgttgggcactgacaattccgtggtgttgtc
ggggaagctgacgtcctttccatggctgctcgcctgtgttgccacctggattctgcgcgggacgtccttctgctacgtcccttcggccct
caatccagcggaccttccttcccgcggcctgctgccggctctgcggcctcttccgcgtcttcgccttcgccctcagacgagtcggatct
ccctttgggccgcctccccgcctggaattaattctgcagtcgagacctagaaaaacatggagcaatcacaagtagcaatacagcagct
accaatgctgattgtgcctggctagaagcacaagaggaggaggaggtgggttttccagtcacacctcaggtacctttaagaccaatga
cttacaaggcagctgtagatcttagccactttttaaaagaaaagaggggactggaagggctaattcactcccaacgaagacaagatatc
cttgatctgtggatctaccacacacaaggctacttccctgattagcagaactacacaccagggccaggggtcagatatccactgaccttt
ggatggtgctacaagctagtaccagttgagccagataaggtagaagaggccaataaaggagagaacaccagcttgttacaccctgtg
agcctgcatgggatggatgacccggagagagaagtgttagagtggaggtttgacagccgcctagcatttcatcacgtggcccgagag
ctgcatccggagtacttcaagaactgctgatatcgagcttgctacaagggactttccgctggggactttccagggaggcgtggcctggg
cgggactggggagtggcgagccctcagatcctgcatataagcagctgctttttgcctgtactgggtctctctggttagaccagatctgag
cctgggagctctctggctaactagggaacccactgcttaagcctcaataaagcttgccttgagtgcttcaagtagtgtgtgcccgtctgtt
gtgtgactctggtaactagagatccctcagacccttttagtcagtgtggaaaatctctagcagtagtagttcatgtcatcttattattcagtatt
tataacttgcaaagaaatgaatatcagagagtgagaggccttgacattgctagcgtttaccgtcgacctctagctagagcttggcgtaatc
atggtcatagctgtttcctgtgtgaaattgttatccgctcacaattccacacaacatacgagccggaagcataaagtgtaaagcctggggt
gcctaatgagtgagctaactcacattaattgcgttgcgctcactgcccgctttccagtcgggaaacctgtcgtgccagctgcattaatgaa
tcggccaacgcgcggggagaggcggtttgcgtattgggcgctcttccgcttcctcgctcactgactcgctgcgctcggtcgttcggctg
cggcgagcggtatcagctcactcaaaggcggtaatacggttatccacagaatcaggggataacgcaggaaagaacatgtgagcaaa
aggccagcaaaaggccaggaaccgtaaaaaggccgcgttgctggcgtttttccataggctccgcccccctgacgagcatcacaaaa
atcgacgctcaagtcagaggtggcgaaacccgacaggactataaagataccaggcgtttccccctggaagctccctcgtgcgctctcc
tgttccgaccctgccgcttaccggatacctgtccgcattctccatcgggaagcgtggcgctttctcatagctcacgctgtaggtatctca
gttcggtgtaggtcgttcgctccaagctgggctgtgtgcacgaaccccccgttcagcccgaccgctgcgccttatccggtaactatcgt
cttgagtccaacccggtaagacacgacttatcgccactggcagcagccactggtaacaggattagcagagcgaggtatgtaggcggt
gctacagagttcttgaagtggtggcctaactacggctacactagaagaacagtatttggtatctgcgctctgctgaagccagttaccttcg
gaaaaagagttggtagctcttgatccggcaaacaaaccaccgctggtagcggtggtttttttgtttgcaagcagcagattacgcgcaga
aaaaaaggatctcaagaagatcctttgatcttttctacggggtctgacgctcagtggaacgaaaactcacgttaagggattttggtcatga
gattatcaaaaaggatcttcacctagatccttttaaattaaaaatgaagttttaaatcaatctaaagtatatatgagtaaacttggtctgacagt
taccaatgcttaatcagtgaggcacctatctcagcgatctgtctatttcgttcatccatagttgcctgactccccgtcgtgtagataactacg
atacgggagggcttaccatctggccccagtgctgcaatgataccgcgagacccacgctcaccggctccagatttatcagcaataaacc
agccagccggaagggccgagcgcagaagtggtcctgcaactttatccgcctccatccagtctattaattgttgccgggaagctagagt
aagtagttcgccagttaatagtttgcgcaacgttgttgccattgctacaggcatcgtggtgtcacgctcgtcgtttggtatggcttcattcag
ctccggttcccaacgatcaaggcgagttacatgatcccccatgttgtgcaaaaaagcggttagctccttcggtcctccgatcgttgtcag
aagtaagttggccgcagtgttatcactcatggttatggcagcactgcataattctatactgtcatgccatccgtaagatgatttctgtgact
ggtgagtactcaaccaagtcattctgagaatagtgtatgcggcgaccgagttgctcttgcccggcgtcaatacgggataataccgcgcc
acatagcagaactttaaaagtgctcatcattggaaaacgttcttcggggcgaaaactctcaaggatcttaccgctgttgagatccagttcg
atgtaacccactcgtgcacccaactgatcttcagcatcttttactttcaccagcgtttctgggtgagcaaaaacaggaaggcaaaatgcc
gcaaaaaagggaataagggcgacacggaaatgttgaatactcatactatcattttcaatattattgaagcatttatcagggttattgtctc
atgagcggatacatatttgaatgtatttagaaaaataaacaaataggggttccgcgcacatttccccgaaaagtgccacctgacgtcgac
ggatcgggagatcaacttgtttattgcagcttataatggttacaaataaagcaatagcatcacaaatttcacaaataaagcatttttttcactg
cattctagttgtggtttgtccaaactcatcaatgtatcttatcatgtctggatcaactggataactcaagctaaccaaaatcatcccaaacttc
ccaccccataccctattaccactgccaattacctgtggtttcatttactctaaacctgtgattcctctgaattattttcattttaaagaaattgtat
ttgttaaatatgtactacaaacttagtagt (SEQ ID NO: 25)
pLVX-CMV-TCR1 (FIG. 8):
tggaagggctaattcactcccaaagaagacaagatatccttgatctgtggatctaccacacacaaggctacttccctgattagcagaact
acacaccagggccaggggtcagatatccactgacctttggatggtgctacaagctagtaccagttgagccagataaggtagaagagg
ccaataaaggagagaacaccagcttgttacaccctgtgagcctgcatgggatggatgacccggagagagaagtgttagagtggaggt
ttgacagccgcctagcatttcatcacgtggcccgagagctgcatccggagtacttcaagaactgctgatatcgagcttgctacaaggga
ctttccgctggggactttccagggaggcgtggcctgggcgggactggggagtggcgagccctcagatcctgcatataagcagctgct
ttttgcctgtactgggtctctctggttagaccagatctgagcctgggagctctctggctaactagggaacccactgcttaagcctcaataa
agcttgccttgagtgcttcaagtagtgtgtgcccgtctgttgtgtgactctggtaactagagatccctcagacccttttagtcagtgtggaa
aatctctagcagtggcgcccgaacagggacttgaaagcgaaagggaaaccagaggagctctctcgacgcaggactcggcttgctga
agcgcgcacggcaagaggcgaggggcggcgactggtgagtacgccaaaaattttgactagcggaggctagaaggagagagatgg
gtgcgagagcgtcagtattaagcgggggagaattagatcgcgatgggaaaaaattcggttaaggccagggggaaagaaaaaatata
aattaaaacatatagtatgggcaagcagggagctagaacgattcgcagttaatcctggcctgttagaaacatcagaaggctgtagacaa
atactgggacagctacaaccatcccttcagacaggatcagaagaacttagatcattatataatacagtagcaaccctctattgtgtgcatc
aaaggatagagataaaagacaccaaggaagctttagacaagatagaggaagagcaaaacaaaagtaagaccaccgcacagcaag
cggccggccgctgatcttcagacctggaggaggagatatgagggacaattggagaagtgaattatataaatataaagtagtaaaaattg
aaccattaggagtagcacccaccaaggcaaagagaagagtggtgcagagagaaaaaagagcagtgggaataggagctttgttcctt
gggttcttgggagcagcaggaagcactatgggcgcagcgtcaatgacgctgacggtacaggccagacaattattgtctggtatagtgc
agcagcagaacaatttgctgagggctattgaggcgcaacagcatctgttgcaactcacagtctggggcatcaagcagctccaggcaa
gaatcctggctgtggaaagatacctaaaggatcaacagctcctggggatttggggttgctctggaaaactcatttgcaccactgctgtgc
cttggaatgctagttggagtaataaatctctggaacagatttggaatcacacgacctggatggagtgggacagagaaattaacaattaca
caagataatacactccttaattgaagaatcgcaaaaccagcaagaaaagaatgaacaagaattattggaattagataaatgggcaagtt
tgtggaattggtttaacataacaaattggctgtggtatataaaattattcataatgatagtaggaggcttggtaggtttaagaatagtttttgct
gtactttctatagtgaatagagttaggcagggatattcaccattatcgtttcagacccacctcccaaccccgaggggacccgacaggcc
cgaaggaatagaagaagaaggtggagagagagacagagacagatccattcgattagtgaacggatctcgacggtatcgcctttaaaa
gaaaaggggggattggggggtacagtgcaggggaaagaatagtagacataatagcaacagacatacaaactaaagaattacaaaaa
caaattacaaaaattcaaaattttcgggtttattacagggacagcagagatccagtttatcgataagcttgggagttccgcgttacataactt
acggtaaatggcccgcctggctgaccgcccaacgacccccgcccattgacgtcaataatgacgtatgttcccatagtaacgccaatag
ggactttccattgacgtcaatgggtggagtatttacggtaaactgcccacttggcagtacatcaagtgtatcatatgccaagtacgccccc
tattgacgtcaatgacggtaaatggcccgcctggcattatgcccagtacatgaccttatgggactttcctacttggcagtacatctacgtat
tagtcatcgctattaccatggtgatgcggttttggcagtacatcaatgggcgtggatagcggtttgactcacggggatttccaagtctcca
ccccattgacgtcaatgggagtttgttttggcaccaaaatcaacgggactttccaaaatgtcgtaacaactccgccccattgacgcaaat
gggcggtaggcgtgtacggtgggaggtctatataagcagagctcgtttagtgaaccgtcagatcgcctggagacgccatccacgctgt
tttgacctccatagaagacaccgactctactagaggatctatttccggtgAATTCGCCACCatgagcctcgggctcctgtgctg
tggggccttttctctcctgtgggcaggaccggtgGAAGCTGACATCTACCAGACCCCAAGATACCTTG
TTATAGGGACAGGAAAGAAGATCACTCTGGAATGTTCTCAAACCATGGGCCATG
ACAAAATGTACTGGTATCAACAAGATCCAGGAATGGAACTACACCTCATCCACT
ATTCCTATGGAGTTAATTCCACAGAGAAGGGAGATCTTTCCTCTGAGTCAACAGT
CTCCAGAATAAGGACGGAGCATTTTCCCCTGACCCTGGAGTCTGCCAGGCCCTCA
CATACCTCTCAGTACCTCTGTGCCAGCAGTCGGACGCCCAACATTCAGTACTTCG
GCGCCGGGACCCGGCTCTCAGTGCTGGAGGACCTGAAAAACGTGTTCCCACCCG
AACTAGTCgtgtttgagccatcagaagcagagatctcccacacccaaaaggccacactggtgtgcctggccacaggcttctac
cccgaccacgtggagctgagctggtgggtgaatgggaaggaggtgcacagtggggtcagcacagacccgcagcccctcaaggag
cagcccgccctcaatgactccagatactgcctgagcagccgcctgagggtctcggccaccttctggcagaacccccgcaaccacttc
cgctgtcaagtccagttctacgggctctcggagaatgacgagtggacccaggatagggccaaacctgtcacccagatcgtcagcgcc
gaggcctggggtagagcagactgtggatcacctccgagtataccagcaaggggtcctgtctgccaccatcctctatgagatcttgct
agggaaggccaccttgtatgccgtgctggtcagtgccctcgtgctgatggccatggtcaagagaaaggattccagaggcGGGAG
CGGAGCCACGAACTTCTCTCTGTTAAAGCAAGCAGGAGACGTGGAAGAAAACCC
CGGTCCTatgacacgagttagcttgctgtgggcagtcgtggtctccacctgtctcgagtccggcatgGGTCAACAGCT
GAATCAGAGTCCTCAATCTATGTTTATCCAGGAAGGAGAAGATGTCTCCATGAAC
TGCACTTCTTCAAGCATATTTAACACCTGGCTATGGTACAAGCAGGAACCTGGGG
AAGGTCCTGTCCTCTTGATAGCCTTATATAAGGCTGGTGAATTGACCTCAAATGG
AAGACTGACTGCTCAGTTTGGTATAACCAGAAAGGACAGCTTCCTGAATATCTCA
GCATCCATACCTAGTGATGTAGGCATCTACTTCTGTGCTGGCACATACCAGAAAG
TTACCTTTGGAACTGGAACAAAGCTCCAAGTCATCCCAAATATCCAGAACCCTGA
CCCTGCCGTGTACCAGCTGAGAGACTCTAAATCCAGTGACAAGTCTGTCTGCctattc
accgattttgattctcaaacaaatgtgtcacaaagtaaggattctgatgtgtatatcacagacaaaactgtgctagacatgaggtctatgga
cttcaagagcaacagtgctgtggcctggagcaacaaatctgactttgcatgcgcaaacgccttcaacaacagcattattccagaagaca
ccttcttccccagcccagaaagttcctgtgatgtcaagctggtcgagaaaagctttgaaacagatacgaacctaaactttcaaaacctgtc
agtgattgggttccgaatcctcctcctgaaagtggccgggtttaatctgctcatgacgctgcggctgtggtccagcTGActcgaggga
tcccgcccctctccctcccccccccctaacgttactggccgaagccgcttggaataaggccggtgtgcgtttgtctatatgttattttccac
catattgccgtcttttggcaatgtgagggcccggaaacctggccctgtcttcttgacgagcattcctaggggtctttcccctctcgccaaa
ggaatgcaaggtctgttgaatgtcgtgaaggaagcagttcctctggaagatcttgaagacaaacaacgtctgtagcgaccctttgcag
gcagcggaaccccccacctggcgacaggtgcctctgcggccaaaagccacgtgtataagatacacctgcaaaggcggcacaaccc
cagtgccacgttgtgagttggatagttgtggaaagagtcaaatggctcacctcaagcgtattcaacaaggggctgaaggatgcccaga
aggtaccccattgtatgggatctgatctggggcctcggtgcacatgattacatgtgtttagtcgaggttaaaaaacgtctaggccccccg
aaccacggggacgtggttttcctttgaaaaacacgatgataatatggtgagcaagggcgaggaggataacatggccatcatcaagga
gttcatgcgcttcaaggtgcacatggagggctccgtgaacggccacgagttcgagatcgagggcgagggcgagggccgcccctac
gagggcacccagaccgccaagctgaaggtgaccaagggtggccccctgcccttcgcctgggacatcctgtcccctcagttcatgtac
ggctccaaggcctacgtgaagcaccccgccgacatccccgactacttgaagctgtccttccccgagggcttcaagtgggagcgcgtg
atgaacttcgaggacggcggcgtggtgaccgtgacccaggactcctccctgcaggacggcgagttcatctacaaggtgaagctgcg
cggcaccaacttcccctccgacggccccgtaatgcagaagaagaccatgggctgggaggcctcctccgagcggatgtaccccgag
gacggcgccctgaagggcgagatcaagcagaggctgaagctgaaggacggcggccactacgacgctgaggtcaagaccacctac
aaggccaagaagcccgtgcagctgcccggcgcctacaacgtcaacatcaagttggacatcacctcccacaacgaggactacaccat
cgtggaacagtacgaacgcgccgagggccgccactccaccggcggcatggacgagctgtacaagtgaacgcgtctggaacaatca
acctctggattacaaaatttgtgaaagattgactggtattcttaactatgttgctccttttacgctatgtggatacgctgctttaatgcctttgtat
catgctattgatcccgtatggctttcattttctcctccttgtataaatcctggttgctgtctctttatgaggagttgtggcccgttgtcaggcaa
cgtggcgtggtgtgcactgtgtttgctgacgcaacccccactggttggggcattgccaccacctgtcagctcctttccgggactttcgctt
tccccctccctattgccacggcggaactcatcgccgcctgccttgcccgctgctggacaggggctcggctgttgggcactgacaattc
cgtggtgttgtcggggaagctgacgtcctttccatggctgctcgcctgtgttgccacctggattctgcgcgggacgtccttctgctacgtc
ccttcggccctcaatccagcggaccttccttcccgcggcctgctgccggctctgcggcctatccgcgtatcgccttcgccctcagacg
agtcggatctccctttgggccgcctccccgcctggaattaattctgcagtcgagacctagaaaaacatggagcaatcacaagtagcaat
acagcagctaccaatgctgattgtgcctggctagaagcacaagaggaggaggaggtgggttttccagtcacacctcaggtacctttaa
gaccaatgacttacaaggcagctgtagatcttagccactttttaaaagaaaagaggggactggaagggctaattcactcccaacgaag
acaagatatccttgatctgtggatctaccacacacaaggctacttccctgattagcagaactacacaccagggccaggggtcagatatc
cactgacctttggatggtgctacaagctagtaccagttgagccagataaggtagaagaggccaataaaggagagaacaccagcttgtt
acaccctgtgagcctgcatgggatggatgacccggagagagaagtgttagagtggaggtttgacagccgcctagcatttcatcacgtg
gcccgagagctgcatccggagtacttcaagaactgctgatatcgagcttgctacaagggactttccgctggggactttccagggaggc
gtggcctgggcgggactggggagtggcgagccctcagatcctgcatataagcagctgctttttgcctgtactgggtctctctggttaga
ccagatctgagcctgggagctctctggctaactagggaacccactgcttaagcctcaataaagcttgccttgagtgcttcaagtagtgtg
tgcccgtctgttgtgtgactctggtaactagagatccctcagacccttttagtcagtgtggaaaatctctagcagtagtagttcatgtcatctt
attattcagtatttataacttgcaaagaaatgaatatcagagagtgagaggccttgacattgctagcgtttaccgtcgacctctagctagag
cttggcgtaatcatggtcatagctgtttcctgtgtgaaattgttatccgctcacaattccacacaacatacgagccggaagcataaagtgta
aagcctggggtgcctaatgagtgagctaactcacattaattgcgttgcgctcactgcccgctttccagtcgggaaacctgtcgtgccagc
tgcattaatgaatcggccaacgcgcggggagaggcggtttgcgtattgggcgctcttccgcttcctcgctcactgactcgctgcgctcg
gtcgttcggctgcggcgagcggtatcagctcactcaaaggcggtaatacggttatccacagaatcaggggataacgcaggaaagaac
atgtgagcaaaaggccagcaaaaggccaggaaccgtaaaaaggccgcgttgctggcgtttttccataggctccgcccccctgacga
gcatcacaaaaatcgacgctcaagtcagaggtggcgaaacccgacaggactataaagataccaggcgtttccccctggaagctccct
cgtgcgctctcctgttccgaccctgccgcttaccggatacctgtccgcctttctcccttcgggaagcgtggcgctttctcatagctcacgc
tgtaggtatctcagttcggtgtaggtcgttcgctccaagctgggctgtgtgcacgaaccccccgttcagcccgaccgctgcgccttatcc
ggtaactatcgtcttgagtccaacccggtaagacacgacttatcgccactggcagcagccactggtaacaggattagcagagcgaggt
atgtaggcggtgctacagagttcttgaagtggtggcctaactacggctacactagaagaacagtatttggtatctgcgctctgctgaagc
cagttaccttcggaaaaagagttggtagctcttgatccggcaaacaaaccaccgctggtagcggtggtttttttgtttgcaagcagcagat
tacgcgcagaaaaaaaggatctcaagaagatcctttgatcttttctacggggtctgacgctcagtggaacgaaaactcacgttaaggga
ttttggtcatgagattatcaaaaaggatcttcacctagatccttttaaattaaaaatgaagttttaaatcaatctaaagtatatatgagtaaactt
ggtctgacagttaccaatgcttaatcagtgaggcacctatctcagcgatctgtctatttcgttcatccatagttgcctgactccccgtcgtgt
agataactacgatacgggagggcttaccatctggccccagtgctgcaatgataccgcgagacccacgctcaccggctccagatttatc
agcaataaaccagccagccggaagggccgagcgcagaagtggtcctgcaactttatccgcctccatccagtctattaattgttgccgg
gaagctagagtaagtagttcgccagttaatagtttgcgcaacgttgttgccattgctacaggcatcgtggtgtcacgctcgtcgtttggtat
ggcttcattcagctccggttcccaacgatcaaggcgagttacatgatcccccatgttgtgcaaaaaagcggttagctccttcggtcctcc
gatcgttgtcagaagtaagttggccgcagtgttatcactcatggttatggcagcactgcataattctcttactgtcatgccatccgtaagat
gcttttctgtgactggtgagtactcaaccaagtcattctgagaatagtgtatgcggcgaccgagttgctcttgcccggcgtcaatacggg
ataataccgcgccacatagcagaactttaaaagtgctcatcattggaaaacgttcttcggggcgaaaactctcaaggatcttaccgctgtt
gagatccagttcgatgtaacccactcgtgcacccaactgatcttcagcatcttttactttcaccagcgtttctgggtgagcaaaaacagga
aggcaaaatgccgcaaaaaagggaataagggcgacacggaaatgttgaatactcatactcttcctttttcaatattattgaagcatttatca
gggttattgtctcatgagcggatacatatttgaatgtatttagaaaaataaacaaataggggttccgcgcacatttccccgaaaagtgcca
cctgacgtcgacggatcgggagatcaacttgtttattgcagcttataatggttacaaataaagcaatagcatcacaaatttcacaaataaa
gcatttttttcactgcattctagttgtggtttgtccaaactcatcaatgtatcttatcatgtctggatcaactggataactcaagctaaccaaaat
catcccaaacttcccaccccataccctattaccactgccaattacctgtggtttcatttactctaaacctgtgattcctctgaattattttcatttt
aaagaaattgtatttgttaaatatgtactacaaacttagtagt (SEQ ID NO: 26)
pLVX-CMV-TCR2 (FIG. 9):
tggaagggctaattcactcccaaagaagacaagatatccttgatctgtggatctaccacacacaaggctacttccctgattagcagaact
acacaccagggccaggggtcagatatccactgacctttggatggtgctacaagctagtaccagttgagccagataaggtagaagagg
ccaataaaggagagaacaccagcttgttacaccctgtgagcctgcatgggatggatgacccggagagagaagtgttagagtggaggt
ttgacagccgcctagcatttcatcacgtggcccgagagctgcatccggagtacttcaagaactgctgatatcgagcttgctacaaggga
ctttccgctggggactttccagggaggcgtggcctgggcgggactggggagtggcgagccctcagatcctgcatataagcagctgct
ttttgcctgtactgggtctctctggttagaccagatctgagcctgggagctctctggctaactagggaacccactgcttaagcctcaataa
agcttgccttgagtgcttcaagtagtgtgtgcccgtctgttgtgtgactctggtaactagagatccctcagacccttttagtcagtgtggaa
aatctctagcagtggcgcccgaacagggacttgaaagcgaaagggaaaccagaggagctctctcgacgcaggactcggcttgctga
agcgcgcacggcaagaggcgaggggcggcgactggtgagtacgccaaaaattttgactagcggaggctagaaggagagagatgg
gtgcgagagcgtcagtattaagcgggggagaattagatcgcgatgggaaaaaattcggttaaggccagggggaaagaaaaaatata
aattaaaacatatagtatgggcaagcagggagctagaacgattcgcagttaatcctggcctgttagaaacatcagaaggctgtagacaa
atactgggacagctacaaccatcccttcagacaggatcagaagaacttagatcattatataatacagtagcaaccctctattgtgtgcatc
aaaggatagagataaaagacaccaaggaagctttagacaagatagaggaagagcaaaacaaaagtaagaccaccgcacagcaag
cggccggccgctgatcttcagacctggaggaggagatatgagggacaattggagaagtgaattatataaatataaagtagtaaaaattg
aaccattaggagtagcacccaccaaggcaaagagaagagtggtgcagagagaaaaaagagcagtgggaataggagctttgttcctt
gggttcttgggagcagcaggaagcactatgggcgcagcgtcaatgacgctgacggtacaggccagacaattattgtctggtatagtgc
agcagcagaacaatttgctgagggctattgaggcgcaacagcatctgttgcaactcacagtctggggcatcaagcagctccaggcaa
gaatcctggctgtggaaagatacctaaaggatcaacagctcctggggatttggggttgctctggaaaactcatttgcaccactgctgtgc
cttggaatgctagttggagtaataaatctctggaacagatttggaatcacacgacctggatggagtgggacagagaaattaacaattaca
caagataatacactccttaattgaagaatcgcaaaaccagcaagaaaagaatgaacaagaattattggaattagataaatgggcaagtt
tgtggaattggtttaacataacaaattggctgtggtatataaaattattcataatgatagtaggaggcttggtaggtttaagaatagtttttgct
gtactttctatagtgaatagagttaggcagggatattcaccattatcgtttcagacccacctcccaaccccgaggggacccgacaggcc
cgaaggaatagaagaagaaggtggagagagagacagagacagatccattcgattagtgaacggatctcgacggtatcgcctttaaaa
gaaaaggggggattggggggtacagtgcaggggaaagaatagtagacataatagcaacagacatacaaactaaagaattacaaaaa
caaattacaaaaattcaaaattttcgggtttattacagggacagcagagatccagtttatcgataagcttgggagttccgcgttacataactt
acggtaaatggcccgcctggctgaccgcccaacgacccccgcccattgacgtcaataatgacgtatgttcccatagtaacgccaatag
ggactttccattgacgtcaatgggtggagtatttacggtaaactgcccacttggcagtacatcaagtgtatcatatgccaagtacgccccc
tattgacgtcaatgacggtaaatggcccgcctggcattatgcccagtacatgaccttatgggactttcctacttggcagtacatctacgtat
tagtcatcgctattaccatggtgatgcggttttggcagtacatcaatgggcgtggatagcggtttgactcacggggatttccaagtctcca
ccccattgacgtcaatgggagtttgttttggcaccaaaatcaacgggactttccaaaatgtcgtaacaactccgccccattgacgcaaat
gggcggtaggcgtgtacggtgggaggtctatataagcagagctcgtttagtgaaccgtcagatcgcctggagacgccatccacgctgt
tttgacctccatagaagacaccgactctactagaggatctatttccggtgAATTCGCCACCatgggacccaggctcctatctg
ggcactgattgtctcctcggaaCCGGTCCGGTTGAAGCTGACATCTACCAGACCCCAAGATAC
CTTGTTATAGGGACAGGAAAGAAGATCACTCTGGAATGTTCTCAAACCATGGGC
CATGACAAAATGTACTGGTATCAACAAGATCCAGGAATGGAACTACACCTCATC
CACTATTCCTATGGAGTTAATTCCACAGAGAAGGGAGATCTTTCCTCTGAGTCAA
CAGTCTCCAGAATAAGGACGGAGCATTTTCCCCTGACCCTGGAGTCTGCCAGGCC
CTCACATACCTCTCAGTACCTCTGTGCCAGCAGTCGGACGCCCAACATTCAGTAC
TTCGGCGCCGGGACCCGGCTCTCAGTGCTGGAGGACCTGAAAAACGTGTTCCCAC
CCGAgGCGGCCGCgtttgagccatcagaagcagagatctcccacacccaaaaggccacactggtgtgcctggccacagg
cttctaccccgaccacgtggagctgagctggtgggtgaatgggaaggaggtgcacagtggggtcagcacagacccgcagcccctc
aaggagcagcccgccctcaatgactccagatactgcctgagcagccgcctgagggtctcggccaccttctggcagaacccccgcaa
ccacttccgctgtcaagtccagttctacgggctctcggagaatgacgagtggacccaggatagggccaaacctgtcacccagatcgtc
agcgccgaggcctggggtagagcagactgtggcttcacctccgagtcttaccagcaaggggtcctgtctgccaccatcctctatgaga
tcttgctagggaaggccaccttgtatgccgtgctggtcagtgccctcgtgctgatggccatggtcaagagaaaggattccagaggcG
GGAGCGGAGCCACGAACTTCTCTCTGTTAAAGCAAGCAGGAGACGTGGAAGAAA
ACCCCGGTCCTatgaactcctctctggactttctaattctgatcttaatgtttggaggaACTAGTGGTCAACAGC
TGAATCAGAGTCCTCAATCTATGTTTATCCAGGAAGGAGAAGATGTCTCCATGAA
CTGCACTTCTTCAAGCATATTTAACACCTGGCTATGGTACAAGCAGGAACCTGGG
GAAGGTCCTGTCCTCTTGATAGCCTTATATAAGGCTGGTGAATTGACCTCAAATG
GAAGACTGACTGCTCAGTTTGGTATAACCAGAAAGGACAGCTTCCTGAATATCTC
AGCATCCATACCTAGTGATGTAGGCATCTACTTCTGTGCTGGCACATACCAGAAA
GTTACCTTTGGAACTGGAACAAAGCTCCAAGTCATCCCAAATATCCAGAACCCTG
ACCCTGCCGTGTACCAGCTGAGAGACTCTAAATCCAGTGACAAGTCTGCATGCctat
tcaccgattttgattctcaaacaaatgtgtcacaaagtaaggattctgatgtgtatatcacagacaaaactgtgctagacatgaggtctatg
gacttcaagagcaacagtgctgtggcctggagcaacaaatctgactttgcatgtgcaaacgccttcaacaacagcattattccagaaga
caccttcttccccagcccagaaagttcctgtgatgtcaagctggtcgagaaaagctttgaaacagatacgaacctaaactttcaaaacct
gtcagtgattgggttccgaatcctcctcctgaaagtggccgggtttaatctgctcatgacgctgcggctgtggtccagcTGActcgag
ggatcccgcccctctccctcccccccccctaacgttactggccgaagccgcttggaataaggccggtgtgcgtttgtctatatgttattttc
caccatattgccgtatttggcaatgtgagggcccggaaacctggccctgtatcttgacgagcattcctaggggtattcccctctcgcc
aaaggaatgcaaggtctgttgaatgtcgtgaaggaagcagttcctctggaagcttcttgaagacaaacaacgtctgtagcgaccctttgc
aggcagcggaaccccccacctggcgacaggtgcctctgcggccaaaagccacgtgtataagatacacctgcaaaggcggcacaac
cccagtgccacgttgtgagttggatagttgtggaaagagtcaaatggctcacctcaagcgtattcaacaaggggctgaaggatgccca
gaaggtaccccattgtatgggatctgatctggggcctcggtgcacatgctttacatgtgtttagtcgaggttaaaaaacgtctaggccccc
cgaaccacggggacgtggttttcctttgaaaaacacgatgataatatggtgagcaagggcgaggaggataacatggccatcatcaag
gagttcatgcgcttcaaggtgcacatggagggctccgtgaacggccacgagttcgagatcgagggcgagggcgagggccgcccct
acgagggcacccagaccgccaagctgaaggtgaccaagggtggccccctgcccttcgcctgggacatcctgtcccctcagttcatgt
acggctccaaggcctacgtgaagcaccccgccgacatccccgactacttgaagctgtccttccccgagggcttcaagtgggagcgcg
tgatgaacttcgaggacggcggcgtggtgaccgtgacccaggactcctccctgcaggacggcgagttcatctacaaggtgaagctgc
gcggcaccaacttcccctccgacggccccgtaatgcagaagaagaccatgggctgggaggcctcctccgagcggatgtaccccga
ggacggcgccctgaagggcgagatcaagcagaggctgaagctgaaggacggcggccactacgacgctgaggtcaagaccaccta
caaggccaagaagcccgtgcagctgcccggcgcctacaacgtcaacatcaagttggacatcacctcccacaacgaggactacacca
tcgtggaacagtacgaacgcgccgagggccgccactccaccggcggcatggacgagctgtacaagtgaacgcgtctggaacaatc
aacctctggattacaaaatttgtgaaagattgactggtattataactatgttgctccttttacgctatgtggatacgctgattaatgcattgt
atcatgctattgatcccgtatggctttcattttctcctccttgtataaatcctggttgctgtctctttatgaggagttgtggcccgttgtcaggc
aacgtggcgtggtgtgcactgtgtttgctgacgcaacccccactggttggggcattgccaccacctgtcagctcctttccgggactttcg
ctttccccctccctattgccacggcggaactcatcgccgcctgccttgcccgctgctggacaggggctcggctgttgggcactgacaat
tccgtggtgttgtcggggaagctgacgtcctttccatggctgctcgcctgtgttgccacctggattctgcgcgggacgtccttctgctacg
tccatcggccctcaatccagcggaccttccttcccgcggcctgctgccggctctgcggcctatccgcgtatcgccttcgccctcaga
cgagtcggatctccctttgggccgcctccccgcctggaattaattctgcagtcgagacctagaaaaacatggagcaatcacaagtagc
aatacagcagctaccaatgctgattgtgcctggctagaagcacaagaggaggaggaggtgggttttccagtcacacctcaggtaccttt
aagaccaatgacttacaaggcagctgtagatcttagccactttttaaaagaaaagaggggactggaagggctaattcactcccaacgaa
gacaagatatccttgatctgtggatctaccacacacaaggctacttccctgattagcagaactacacaccagggccaggggtcagatat
ccactgacctttggatggtgctacaagctagtaccagttgagccagataaggtagaagaggccaataaaggagagaacaccagcttgt
tacaccctgtgagcctgcatgggatggatgacccggagagagaagtgttagagtggaggtttgacagccgcctagcatttcatcacgt
ggcccgagagctgcatccggagtacttcaagaactgctgatatcgagcttgctacaagggactttccgctggggactttccagggagg
cgtggcctgggcgggactggggagtggcgagccctcagatcctgcatataagcagctgctttttgcctgtactgggtctctctggttag
accagatctgagcctgggagctctctggctaactagggaacccactgcttaagcctcaataaagcttgccttgagtgcttcaagtagtgt
gtgcccgtctgttgtgtgactctggtaactagagatccctcagacccttttagtcagtgtggaaaatctctagcagtagtagttcatgtcatc
ttattattcagtatttataacttgcaaagaaatgaatatcagagagtgagaggccttgacattgctagcgtttaccgtcgacctctagctaga
gcttggcgtaatcatggtcatagctgtttcctgtgtgaaattgttatccgctcacaattccacacaacatacgagccggaagcataaagtgt
aaagcctggggtgcctaatgagtgagctaactcacattaattgcgttgcgctcactgcccgctttccagtcgggaaacctgtcgtgccag
ctgcattaatgaatcggccaacgcgcggggagaggcggtttgcgtattgggcgctcttccgcttcctcgctcactgactcgctgcgctc
ggtcgttcggctgcggcgagcggtatcagctcactcaaaggcggtaatacggttatccacagaatcaggggataacgcaggaaaga
acatgtgagcaaaaggccagcaaaaggccaggaaccgtaaaaaggccgcgttgctggcgtttttccataggctccgcccccctgacg
agcatcacaaaaatcgacgctcaagtcagaggtggcgaaacccgacaggactataaagataccaggcgtttccccctggaagctccc
tcgtgcgctctcctgttccgaccctgccgcttaccggatacctgtccgcctttctcccttcgggaagcgtggcgctttctcatagctcacg
ctgtaggtatctcagttcggtgtaggtcgttcgctccaagctgggctgtgtgcacgaaccccccgttcagcccgaccgctgcgccttatc
cggtaactatcgtcttgagtccaacccggtaagacacgacttatcgccactggcagcagccactggtaacaggattagcagagcgag
gtatgtaggcggtgctacagagttcttgaagtggtggcctaactacggctacactagaagaacagtatttggtatctgcgctctgctgaa
gccagttaccttcggaaaaagagttggtagctcttgatccggcaaacaaaccaccgctggtagcggtggtttttttgtttgcaagcagca
gattacgcgcagaaaaaaaggatctcaagaagatcctttgatcttttctacggggtctgacgctcagtggaacgaaaactcacgttaag
ggattttggtcatgagattatcaaaaaggatcttcacctagatccttttaaattaaaaatgaagttttaaatcaatctaaagtatatatgagtaa
acttggtctgacagttaccaatgcttaatcagtgaggcacctatctcagcgatctgtctatttcgttcatccatagttgcctgactccccgtc
gtgtagataactacgatacgggagggcttaccatctggccccagtgctgcaatgataccgcgagacccacgctcaccggctccagatt
tatcagcaataaaccagccagccggaagggccgagcgcagaagtggtcctgcaactttatccgcctccatccagtctattaattgttgc
cgggaagctagagtaagtagttcgccagttaatagtttgcgcaacgttgttgccattgctacaggcatcgtggtgtcacgctcgtcgtttg
gtatggcttcattcagctccggttcccaacgatcaaggcgagttacatgatcccccatgttgtgcaaaaaagcggttagctccttcggtcc
tccgatcgttgtcagaagtaagttggccgcagtgttatcactcatggttatggcagcactgcataattctcttactgtcatgccatccgtaag
atgcttttctgtgactggtgagtactcaaccaagtcattctgagaatagtgtatgcggcgaccgagttgctcttgcccggcgtcaatacgg
gataataccgcgccacatagcagaactttaaaagtgctcatcattggaaaacgttcttcggggcgaaaactctcaaggatcttaccgctg
ttgagatccagttcgatgtaacccactcgtgcacccaactgatcttcagcatcttttactttcaccagcgtttctgggtgagcaaaaacagg
aaggcaaaatgccgcaaaaaagggaataagggcgacacggaaatgttgaatactcatactcttcctttttcaatattattgaagcatttatc
agggttattgtctcatgagcggatacatatttgaatgtatttagaaaaataaacaaataggggttccgcgcacatttccccgaaaagtgcc
acctgacgtcgacggatcgggagatcaacttgtttattgcagcttataatggttacaaataaagcaatagcatcacaaatttcacaaataa
agcatttttttcactgcattctagttgtggtttgtccaaactcatcaatgtatcttatcatgtctggatcaactggataactcaagctaaccaaa
atcatcccaaacttcccaccccataccctattaccactgccaattacctgtggtttcatttactctaaacctgtgattcctctgaattattttcatt
ttaaagaaattgtatttgttaaatatgtactacaaacttagtagt (SEQ ID NO: 27)
pLVX-CMV-TCR3 (FIG. 10):
tggaagggctaattcactcccaaagaagacaagatatccttgatctgtggatctaccacacacaaggctacttccctgattagcagaact
acacaccagggccaggggtcagatatccactgacctttggatggtgctacaagctagtaccagttgagccagataaggtagaagagg
ccaataaaggagagaacaccagcttgttacaccctgtgagcctgcatgggatggatgacccggagagagaagtgttagagtggaggt
ttgacagccgcctagcatttcatcacgtggcccgagagctgcatccggagtacttcaagaactgctgatatcgagcttgctacaaggga
ctttccgctggggactttccagggaggcgtggcctgggcgggactggggagtggcgagccctcagatcctgcatataagcagctgct
ttttgcctgtactgggtctctctggttagaccagatctgagcctgggagctctctggctaactagggaacccactgcttaagcctcaataa
agcttgccttgagtgcttcaagtagtgtgtgcccgtctgttgtgtgactctggtaactagagatccctcagacccttttagtcagtgtggaa
aatctctagcagtggcgcccgaacagggacttgaaagcgaaagggaaaccagaggagctctctcgacgcaggactcggcttgctga
agcgcgcacggcaagaggcgaggggcggcgactggtgagtacgccaaaaattttgactagcggaggctagaaggagagagatgg
gtgcgagagcgtcagtattaagcgggggagaattagatcgcgatgggaaaaaattcggttaaggccagggggaaagaaaaaatata
aattaaaacatatagtatgggcaagcagggagctagaacgattcgcagttaatcctggcctgttagaaacatcagaaggctgtagacaa
atactgggacagctacaaccatcccttcagacaggatcagaagaacttagatcattatataatacagtagcaaccctctattgtgtgcatc
aaaggatagagataaaagacaccaaggaagctttagacaagatagaggaagagcaaaacaaaagtaagaccaccgcacagcaag
cggccggccgctgatcttcagacctggaggaggagatatgagggacaattggagaagtgaattatataaatataaagtagtaaaaattg
aaccattaggagtagcacccaccaaggcaaagagaagagtggtgcagagagaaaaaagagcagtgggaataggagctttgttcctt
gggttcttgggagcagcaggaagcactatgggcgcagcgtcaatgacgctgacggtacaggccagacaattattgtctggtatagtgc
agcagcagaacaatttgctgagggctattgaggcgcaacagcatctgttgcaactcacagtctggggcatcaagcagctccaggcaa
gaatcctggctgtggaaagatacctaaaggatcaacagctcctggggatttggggttgctctggaaaactcatttgcaccactgctgtgc
cttggaatgctagttggagtaataaatctctggaacagatttggaatcacacgacctggatggagtgggacagagaaattaacaattaca
caagataatacactccttaattgaagaatcgcaaaaccagcaagaaaagaatgaacaagaattattggaattagataaatgggcaagtt
tgtggaattggtttaacataacaaattggctgtggtatataaaattattcataatgatagtaggaggcttggtaggtttaagaatagtttttgct
gtactttctatagtgaatagagttaggcagggatattcaccattatcgtttcagacccacctcccaaccccgaggggacccgacaggcc
cgaaggaatagaagaagaaggtggagagagagacagagacagatccattcgattagtgaacggatctcgacggtatcgcctttaaaa
gaaaaggggggattggggggtacagtgcaggggaaagaatagtagacataatagcaacagacatacaaactaaagaattacaaaaa
caaattacaaaaattcaaaattttcgggtttattacagggacagcagagatccagtttatcgataagcttgggagttccgcgttacataactt
acggtaaatggcccgcctggctgaccgcccaacgacccccgcccattgacgtcaataatgacgtatgttcccatagtaacgccaatag
ggactttccattgacgtcaatgggtggagtatttacggtaaactgcccacttggcagtacatcaagtgtatcatatgccaagtacgccccc
tattgacgtcaatgacggtaaatggcccgcctggcattatgcccagtacatgaccttatgggactttcctacttggcagtacatctacgtat
tagtcatcgctattaccatggtgatgcggttttggcagtacatcaatgggcgtggatagcggtttgactcacggggatttccaagtctcca
ccccattgacgtcaatgggagtttgttttggcaccaaaatcaacgggactttccaaaatgtcgtaacaactccgccccattgacgcaaat
gggcggtaggcgtgtacggtgggaggtctatataagcagagctcgtttagtgaaccgtcagatcgcctggagacgccatccacgctgt
tttgacctccatagaagacaccgactctactagaggatctatttccggtgAATTCGCCACCatgggcacaaggttgttcttctat
gtggccctttgtctcctgtggaccggtcacatgGAAGCTGACATCTACCAGACCCCAAGATACCTTGT
TATAGGGACAGGAAAGAAGATCACTCTGGAATGTTCTCAAACCATGGGCCATGA
CAAAATGTACTGGTATCAACAAGATCCAGGAATGGAACTACACCTCATCCACTAT
TCCTATGGAGTTAATTCCACAGAGAAGGGAGATCTTTCCTCTGAGTCAACAGTCT
CCAGAATAAGGACGGAGCATTTTCCCCTGACCCTGGAGTCTGCCAGGCCCTCACA
TACCTCTCAGTACCTCTGTGCCAGCAGTCGGACGCCCAACATTCAGTACTTCGGC
GCCGGGACCCGGCTCTCAGTGCTGgaggacctgaaaaacgtgttcccacccgaggtcgctgtgtttgagccatc
agaagcagagatctcccacacccaaaaggccacACTAGTgtgcctggccacaggcttctaccccgaccacgtggagctgagct
ggtgggtgaatgggaaggaggtgcacagtggggtcagcacagacccgcagcccctcaaggagcagcccgccctcaatgactcca
gatactgcctgagcagccgcctgagggtctcggccaccttctggcagaacccccgcaaccacttccgctgtcaagtccagttctacgg
gctctcggagaatgacgagtggacccaggatagggccaaacctgtcacccagatcgtcagcgccgaggcctggggtagagcagac
tgtggcttcacctccgagtcttaccagcaaggggtcctgtctgccaccatcctctatgagatcttgctagggaaggccaccttgtatgcc
gtgctggtcagtgccctcgtgctgatggccatggtcaagagaaaggattccagaggcGGGAGCGGAGCCACGAAC
TTCTCTCTGTTAAAGCAAGCAGGAGACGTGGAAGAAAACCCCGGTCCTatggcaggca
ttcgagctttatttatgtacttgtggctgcagctggactgggtctcgagaGGTCAACAGCTGAATCAGAGTCCTC
AATCTATGTTTATCCAGGAAGGAGAAGATGTCTCCATGAACTGCACTTCTTCAAG
CATATTTAACACCTGGCTATGGTACAAGCAGGAACCTGGGGAAGGTCCTGTCCTC
TTGATAGCCTTATATAAGGCTGGTGAATTGACCTCAAATGGAAGACTGACTGCTC
AGTTTGGTATAACCAGAAAGGACAGCTTCCTGAATATCTCAGCATCCATACCTAG
TGATGTAGGCATCTACTTCTGTGCTGGCACATACCAGAAAGTTACCTTTGGAACT
GGAACAAAGCTCCAAGTCATCCCAaatatccagaaccctgaccctgccgtgtaccagctgagagactctaaatc
cagtgacaagtctgtctgcctattcaccgattttgattctcaaacaaatgtgtcacaaagtaaggattctgatgtgtatatcacagacaaaa
ctgtgctagacatgaggtctatggacttcaagagcaacagtGCGGCCgcctggagcaacaaatctgactttgcatgtgcaaacgc
cttcaacaacagcattattccagaagacaccttcttccccagcccagaaagttcctgtgatgtcaagctggtcgagaaaagctttgaaac
agatacgaacctaaactttcaaaacctgtcagtgattgggttccgaatcctcctcctgaaagtggccgggtttaatctgctcatgacgctg
cggctgtggtccagcTGActcgagggatcccgcccctctccctcccccccccctaacgttactggccgaagccgcttggaataagg
ccggtgtgcgtttgtctatatgttattttccaccatattgccgtcttttggcaatgtgagggcccggaaacctggccctgtcttcttgacgag
cattcctaggggtattcccctctcgccaaaggaatgcaaggtctgttgaatgtcgtgaaggaagcagttcctctggaagcttcttgaaga
caaacaacgtctgtagcgaccctttgcaggcagcggaaccccccacctggcgacaggtgcctctgcggccaaaagccacgtgtata
agatacacctgcaaaggcggcacaaccccagtgccacgttgtgagttggatagttgtggaaagagtcaaatggctcacctcaagcgta
ttcaacaaggggctgaaggatgcccagaaggtaccccattgtatgggatctgatctggggcctcggtgcacatgctttacatgtgtttag
tcgaggttaaaaaacgtctaggccccccgaaccacggggacgtggttttcctttgaaaaacacgatgataatatggtgagcaagggcg
aggaggataacatggccatcatcaaggagttcatgcgcttcaaggtgcacatggagggctccgtgaacggccacgagttcgagatcg
agggcgagggcgagggccgcccctacgagggcacccagaccgccaagctgaaggtgaccaagggtggccccctgcccttcgcct
gggacatcctgtcccctcagttcatgtacggctccaaggcctacgtgaagcaccccgccgacatccccgactacttgaagctgtccttc
cccgagggcttcaagtgggagcgcgtgatgaacttcgaggacggcggcgtggtgaccgtgacccaggactcctccctgcaggacg
gcgagttcatctacaaggtgaagctgcgcggcaccaacttcccctccgacggccccgtaatgcagaagaagaccatgggctgggag
gcctcctccgagcggatgtaccccgaggacggcgccctgaagggcgagatcaagcagaggctgaagctgaaggacggcggcca
ctacgacgctgaggtcaagaccacctacaaggccaagaagcccgtgcagctgcccggcgcctacaacgtcaacatcaagttggaca
tcacctcccacaacgaggactacaccatcgtggaacagtacgaacgcgccgagggccgccactccaccggcggcatggacgagct
gtacaagtgaacgcgtctggaacaatcaacctctggattacaaaatttgtgaaagattgactggtattcttaactatgttgctccttttacgct
atgtggatacgctgattaatgcattgtatcatgctattgatcccgtatggctttcattttctcctccttgtataaatcctggttgctgtctatta
tgaggagttgtggcccgttgtcaggcaacgtggcgtggtgtgcactgtgtttgctgacgcaacccccactggttggggcattgccacca
cctgtcagctcctttccgggactttcgctttccccctccctattgccacggcggaactcatcgccgcctgccttgcccgctgctggacag
gggctcggctgttgggcactgacaattccgtggtgttgtcggggaagctgacgtcctttccatggctgctcgcctgtgttgccacctgga
ttctgcgcgggacgtccttctgctacgtcccttcggccctcaatccagcggaccttccttcccgcggcctgctgccggctctgcggcctc
ttccgcgtcttcgccttcgccctcagacgagtcggatctccctttgggccgcctccccgcctggaattaattctgcagtcgagacctaga
aaaacatggagcaatcacaagtagcaatacagcagctaccaatgctgattgtgcctggctagaagcacaagaggaggaggaggtgg
gttttccagtcacacctcaggtacctttaagaccaatgacttacaaggcagctgtagatcttagccactttttaaaagaaaagaggggact
ggaagggctaattcactcccaacgaagacaagatatccttgatctgtggatctaccacacacaaggctacttccctgattagcagaacta
cacaccagggccaggggtcagatatccactgacctttggatggtgctacaagctagtaccagttgagccagataaggtagaagaggc
caataaaggagagaacaccagcttgttacaccctgtgagcctgcatgggatggatgacccggagagagaagtgttagagtggaggtt
tgacagccgcctagcatttcatcacgtggcccgagagctgcatccggagtacttcaagaactgctgatatcgagcttgctacaagggac
tttccgctggggactttccagggaggcgtggcctgggcgggactggggagtggcgagccctcagatcctgcatataagcagctgcttt
ttgcctgtactgggtctctctggttagaccagatctgagcctgggagctctctggctaactagggaacccactgcttaagcctcaataaa
gcttgccttgagtgcttcaagtagtgtgtgcccgtctgttgtgtgactctggtaactagagatccctcagacccttttagtcagtgtggaaa
atctctagcagtagtagttcatgtcatcttattattcagtatttataacttgcaaagaaatgaatatcagagagtgagaggccttgacattgct
agcgtttaccgtcgacctctagctagagcttggcgtaatcatggtcatagctgtttcctgtgtgaaattgttatccgctcacaattccacaca
acatacgagccggaagcataaagtgtaaagcctggggtgcctaatgagtgagctaactcacattaattgcgttgcgctcactgcccgct
ttccagtcgggaaacctgtcgtgccagctgcattaatgaatcggccaacgcgcggggagaggcggtttgcgtattgggcgctcttccg
cttcctcgctcactgactcgctgcgctcggtcgttcggctgcggcgagcggtatcagctcactcaaaggcggtaatacggttatccaca
gaatcaggggataacgcaggaaagaacatgtgagcaaaaggccagcaaaaggccaggaaccgtaaaaaggccgcgttgctggcg
tttttccataggctccgcccccctgacgagcatcacaaaaatcgacgctcaagtcagaggtggcgaaacccgacaggactataaagat
accaggcgtttccccctggaagctccctcgtgcgctctcctgttccgaccctgccgcttaccggatacctgtccgcctttctcccttcggg
aagcgtggcgctttctcatagctcacgctgtaggtatctcagttcggtgtaggtcgttcgctccaagctgggctgtgtgcacgaaccccc
cgttcagcccgaccgctgcgccttatccggtaactatcgtcttgagtccaacccggtaagacacgacttatcgccactggcagcagcca
ctggtaacaggattagcagagcgaggtatgtaggcggtgctacagagttcttgaagtggtggcctaactacggctacactagaagaac
agtatttggtatctgcgctctgctgaagccagttaccttcggaaaaagagttggtagctcttgatccggcaaacaaaccaccgctggtag
cggtggtttttttgtttgcaagcagcagattacgcgcagaaaaaaaggatctcaagaagatcctttgatcttttctacggggtctgacgctc
agtggaacgaaaactcacgttaagggattttggtcatgagattatcaaaaaggatcttcacctagatccttttaaattaaaaatgaagtttta
aatcaatctaaagtatatatgagtaaacttggtctgacagttaccaatgcttaatcagtgaggcacctatctcagcgatctgtctatttcgttc
atccatagttgcctgactccccgtcgtgtagataactacgatacgggagggcttaccatctggccccagtgctgcaatgataccgcgag
acccacgctcaccggctccagatttatcagcaataaaccagccagccggaagggccgagcgcagaagtggtcctgcaactttatccg
cctccatccagtctattaattgttgccgggaagctagagtaagtagttcgccagttaatagtttgcgcaacgttgttgccattgctacaggc
atcgtggtgtcacgctcgtcgtttggtatggcttcattcagctccggttcccaacgatcaaggcgagttacatgatcccccatgttgtgcaa
aaaagcggttagctccttcggtcctccgatcgttgtcagaagtaagttggccgcagtgttatcactcatggttatggcagcactgcataatt
ctcttactgtcatgccatccgtaagatgcttttctgtgactggtgagtactcaaccaagtcattctgagaatagtgtatgcggcgaccgagt
tgctcttgcccggcgtcaatacgggataataccgcgccacatagcagaactttaaaagtgctcatcattggaaaacgttcttcggggcg
aaaactctcaaggatcttaccgctgttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaatgttgaatactcatactctt
cctttttcaatattattgaagcatttatcagggttattgtctcatgagcggatacatatttgaatgtatttagaaaaataaacaaataggggttc
cgcgcacatttccccgaaaagtgccacctgacgtcgacggatcgggagatcaacttgtttattgcagcttataatggttacaaataaagc
aatagcatcacaaatttcacaaataaagcatttttttcactgcattctagttgtggtttgtccaaactcatcaatgtatcttatcatgtctggatc
aactggataactcaagctaaccaaaatcatcccaaacttcccaccccataccctattaccactgccaattacctgtggtttcatttactctaa
acctgtgattcctctgaattattttcattttaaagaaattgtatttgttaaatatgtactacaaacttagtagt (SEQ ID NO: 28)
pLVX-CMV-TCR4 (FIG. 11):
tggaagggctaattcactcccaaagaagacaagatatccttgatctgtggatctaccacacacaaggctacttccctgattagcagaact
acacaccagggccaggggtcagatatccactgacctttggatggtgctacaagctagtaccagttgagccagataaggtagaagagg
ccaataaaggagagaacaccagcttgttacaccctgtgagcctgcatgggatggatgacccggagagagaagtgttagagtggaggt
ttgacagccgcctagcatttcatcacgtggcccgagagctgcatccggagtacttcaagaactgctgatatcgagcttgctacaaggga
ctttccgctggggactttccagggaggcgtggcctgggcgggactggggagtggcgagccctcagatcctgcatataagcagctgct
ttttgcctgtactgggtctctctggttagaccagatctgagcctgggagctctctggctaactagggaacccactgcttaagcctcaataa
agcttgccttgagtgcttcaagtagtgtgtgcccgtctgttgtgtgactctggtaactagagatccctcagacccttttagtcagtgtggaa
aatctctagcagtggcgcccgaacagggacttgaaagcgaaagggaaaccagaggagctctctcgacgcaggactcggcttgctga
agcgcgcacggcaagaggcgaggggcggcgactggtgagtacgccaaaaattttgactagcggaggctagaaggagagagatgg
gtgcgagagcgtcagtattaagcgggggagaattagatcgcgatgggaaaaaattcggttaaggccagggggaaagaaaaaatata
aattaaaacatatagtatgggcaagcagggagctagaacgattcgcagttaatcctggcctgttagaaacatcagaaggctgtagacaa
atactgggacagctacaaccatcccttcagacaggatcagaagaacttagatcattatataatacagtagcaaccctctattgtgtgcatc
aaaggatagagataaaagacaccaaggaagctttagacaagatagaggaagagcaaaacaaaagtaagaccaccgcacagcaag
cggccggccgctgatcttcagacctggaggaggagatatgagggacaattggagaagtgaattatataaatataaagtagtaaaaattg
aaccattaggagtagcacccaccaaggcaaagagaagagtggtgcagagagaaaaaagagcagtgggaataggagctttgttcctt
gggttcttgggagcagcaggaagcactatgggcgcagcgtcaatgacgctgacggtacaggccagacaattattgtctggtatagtgc
agcagcagaacaatttgctgagggctattgaggcgcaacagcatctgttgcaactcacagtctggggcatcaagcagctccaggcaa
gaatcctggctgtggaaagatacctaaaggatcaacagctcctggggatttggggttgctctggaaaactcatttgcaccactgctgtgc
cttggaatgctagttggagtaataaatctctggaacagatttggaatcacacgacctggatggagtgggacagagaaattaacaattaca
caagataatacactccttaattgaagaatcgcaaaaccagcaagaaaagaatgaacaagaattattggaattagataaatgggcaagtt
tgtggaattggtttaacataacaaattggctgtggtatataaaattattcataatgatagtaggaggcttggtaggtttaagaatagtttttgct
gtactttctatagtgaatagagttaggcagggatattcaccattatcgtttcagacccacctcccaaccccgaggggacccgacaggcc
cgaaggaatagaagaagaaggtggagagagagacagagacagatccattcgattagtgaacggatctcgacggtatcgcctttaaaa
gaaaaggggggattggggggtacagtgcaggggaaagaatagtagacataatagcaacagacatacaaactaaagaattacaaaaa
caaattacaaaaattcaaaattttcgggtttattacagggacagcagagatccagtttatcgataagcttgggagttccgcgttacataactt
acggtaaatggcccgcctggctgaccgcccaacgacccccgcccattgacgtcaataatgacgtatgttcccatagtaacgccaatag
ggactttccattgacgtcaatgggtggagtatttacggtaaactgcccacttggcagtacatcaagtgtatcatatgccaagtacgccccc
tattgacgtcaatgacggtaaatggcccgcctggcattatgcccagtacatgaccttatgggactttcctacttggcagtacatctacgtat
tagtcatcgctattaccatggtgatgcggttttggcagtacatcaatgggcgtggatagcggtttgactcacggggatttccaagtctcca
ccccattgacgtcaatgggagtttgttttggcaccaaaatcaacgggactttccaaaatgtcgtaacaactccgccccattgacgcaaat
gggcggtaggcgtgtacggtgggaggtctatataagcagagctcgtttagtgaaccgtcagatcgcctggagacgccatccacgctgt
tttgacctccatagaagacaccgactctactagaggatctatttccggtgAATTCGCCACCatgggaatcaggctcctgtgtcg
tgtggccttttgtttcctggctgtaggactagtaGAAGCTGACATCTACCAGACCCCAAGATACCTTGT
TATAGGGACAGGAAAGAAGATCACTCTGGAATGTTCTCAAACCATGGGCCATGA
CAAAATGTACTGGTATCAACAAGATCCAGGAATGGAACTACACCTCATCCACTAT
TCCTATGGAGTTAATTCCACAGAGAAGGGAGATCTTTCCTCTGAGTCAACAGTCT
CCAGAATAAGGACGGAGCATTTTCCCCTGACCCTGGAGTCTGCCAGGCCCTCACA
TACCTCTCAGTACCTCTGTGCCAGCAGTCGGACGCCCAACATTCAGTACTTCGGC
GCCGGGACCCGGCTCTCAGTGCTGgaggacctgaaaaacgtgttcccacccgaggtcgctgtgtttgagccatc
agaagcagagatctcccacacccaaaaggccacACTgGCAtgcctggccacaggcttctaccccgaccacgtggagctgagc
tggtgggtgaatgggaaggaggtgcacagtggggtcagcacagacccgcagcccctcaaggagcagcccgccctcaatgactcca
gatactgcctgagcagccgcctgagggtctcggccaccttctggcagaacccccgcaaccacttccgctgtcaagtccagttctacgg
gctctcggagaatgacgagtggacccaggatagggccaaacctgtcacccagatcgtcagcgccgaggcctggggtagagcagac
tgtggcttcacctccgagtcttaccagcaaggggtcctgtctgccaccatcctctatgagatcttgctagggaaggccaccttgtatgcc
gtgctggtcagtgccctcgtgctgatggccatggtcaagagaaaggattccagaggcGGGAGCGGAGCCACGAAC
TTCTCTCTGTTAAAGCAAGCAGGAGACGTGGAAGAAAACCCCGGTCCTatgacacgagt
tagcttgctgtgggcagtcgtggtctccacctgtctcgagtccggcatgGGTCAACAGCTGAATCAGAGTCCTC
AATCTATGTTTATCCAGGAAGGAGAAGATGTCTCCATGAACTGCACTTCTTCAAG
CATATTTAACACCTGGCTATGGTACAAGCAGGAACCTGGGGAAGGTCCTGTCCTC
TTGATAGCCTTATATAAGGCTGGTGAATTGACCTCAAATGGAAGACTGACTGCTC
AGTTTGGTATAACCAGAAAGGACAGCTTCCTGAATATCTCAGCATCCATACCTAG
TGATGTAGGCATCTACTTCTGTGCTGGCACATACCAGAAAGTTACCTTTGGAACT
GGAACAAAGCTCCAAGTCATCCCAaatatccagaaccctgaccctgccgtgtaccagctgagagactctaaatc
cagtgacaagtctgtctgcctattcaccgattttgattctcaaacaaatgtgtcacaaagtaaggattctgatgtgtatatcacagacaaaa
ctgtgctagacatgaggtctatggacttcaagagcaacagtGCGGCCgcctggagcaacaaatctgactttgcatgtgcaaacgc
cttcaacaacagcattattccagaagacaccttcttccccagcccagaaagttcctgtgatgtcaagctggtcgagaaaagctttgaaac
agatacgaacctaaactttcaaaacctgtcagtgattgggttccgaatcctcctcctgaaagtggccgggtttaatctgctcatgacgctg
cggctgtggtccagcTGActcgagggatcccgcccctctccctcccccccccctaacgttactggccgaagccgcttggaataagg
ccggtgtgcgtttgtctatatgttattttccaccatattgccgtcttttggcaatgtgagggcccggaaacctggccctgtcttcttgacgag
cattcctaggggtattcccctctcgccaaaggaatgcaaggtctgttgaatgtcgtgaaggaagcagttcctctggaagcttcttgaaga
caaacaacgtctgtagcgaccctttgcaggcagcggaaccccccacctggcgacaggtgcctctgcggccaaaagccacgtgtata
agatacacctgcaaaggcggcacaaccccagtgccacgttgtgagttggatagttgtggaaagagtcaaatggctcacctcaagcgta
ttcaacaaggggctgaaggatgcccagaaggtaccccattgtatgggatctgatctggggcctcggtgcacatgctttacatgtgtttag
tcgaggttaaaaaacgtctaggccccccgaaccacggggacgtggttttcctttgaaaaacacgatgataatatggtgagcaagggcg
aggaggataacatggccatcatcaaggagttcatgcgcttcaaggtgcacatggagggctccgtgaacggccacgagttcgagatcg
agggcgagggcgagggccgcccctacgagggcacccagaccgccaagctgaaggtgaccaagggtggccccctgcccttcgcct
gggacatcctgtcccctcagttcatgtacggctccaaggcctacgtgaagcaccccgccgacatccccgactacttgaagctgtccttc
cccgagggcttcaagtgggagcgcgtgatgaacttcgaggacggcggcgtggtgaccgtgacccaggactcctccctgcaggacg
gcgagttcatctacaaggtgaagctgcgcggcaccaacttcccctccgacggccccgtaatgcagaagaagaccatgggctgggag
gcctcctccgagcggatgtaccccgaggacggcgccctgaagggcgagatcaagcagaggctgaagctgaaggacggcggcca
ctacgacgctgaggtcaagaccacctacaaggccaagaagcccgtgcagctgcccggcgcctacaacgtcaacatcaagttggaca
tcacctcccacaacgaggactacaccatcgtggaacagtacgaacgcgccgagggccgccactccaccggcggcatggacgagct
gtacaagtgaacgcgtctggaacaatcaacctctggattacaaaatttgtgaaagattgactggtattcttaactatgttgctccttttacgct
atgtggatacgctgattaatgcattgtatcatgctattgatcccgtatggctttcattttctcctccttgtataaatcctggttgctgtctatta
tgaggagttgtggcccgttgtcaggcaacgtggcgtggtgtgcactgtgtttgctgacgcaacccccactggttggggcattgccacca
cctgtcagctcctttccgggactttcgctttccccctccctattgccacggcggaactcatcgccgcctgccttgcccgctgctggacag
gggctcggctgttgggcactgacaattccgtggtgttgtcggggaagctgacgtcctttccatggctgctcgcctgtgttgccacctgga
ttctgcgcgggacgtccttctgctacgtcccttcggccctcaatccagcggaccttccttcccgcggcctgctgccggctctgcggcctc
ttccgcgtcttcgccttcgccctcagacgagtcggatctccctttgggccgcctccccgcctggaattaattctgcagtcgagacctaga
aaaacatggagcaatcacaagtagcaatacagcagctaccaatgctgattgtgcctggctagaagcacaagaggaggaggaggtgg
gttttccagtcacacctcaggtacctttaagaccaatgacttacaaggcagctgtagatcttagccactttttaaaagaaaagaggggact
ggaagggctaattcactcccaacgaagacaagatatccttgatctgtggatctaccacacacaaggctacttccctgattagcagaacta
cacaccagggccaggggtcagatatccactgacctttggatggtgctacaagctagtaccagttgagccagataaggtagaagaggc
caataaaggagagaacaccagcttgttacaccctgtgagcctgcatgggatggatgacccggagagagaagtgttagagtggaggtt
tgacagccgcctagcatttcatcacgtggcccgagagctgcatccggagtacttcaagaactgctgatatcgagcttgctacaagggac
tttccgctggggactttccagggaggcgtggcctgggcgggactggggagtggcgagccctcagatcctgcatataagcagctgcttt
ttgcctgtactgggtctctctggttagaccagatctgagcctgggagctctctggctaactagggaacccactgcttaagcctcaataaa
gcttgccttgagtgcttcaagtagtgtgtgcccgtctgttgtgtgactctggtaactagagatccctcagacccttttagtcagtgtggaaa
atctctagcagtagtagttcatgtcatcttattattcagtatttataacttgcaaagaaatgaatatcagagagtgagaggccttgacattgct
agcgtttaccgtcgacctctagctagagcttggcgtaatcatggtcatagctgtttcctgtgtgaaattgttatccgctcacaattccacaca
acatacgagccggaagcataaagtgtaaagcctggggtgcctaatgagtgagctaactcacattaattgcgttgcgctcactgcccgct
ttccagtcgggaaacctgtcgtgccagctgcattaatgaatcggccaacgcgcggggagaggcggtttgcgtattgggcgctcttccg
cttcctcgctcactgactcgctgcgctcggtcgttcggctgcggcgagcggtatcagctcactcaaaggcggtaatacggttatccaca
gaatcaggggataacgcaggaaagaacatgtgagcaaaaggccagcaaaaggccaggaaccgtaaaaaggccgcgttgctggcg
tttttccataggctccgcccccctgacgagcatcacaaaaatcgacgctcaagtcagaggtggcgaaacccgacaggactataaagat
accaggcgtttccccctggaagctccctcgtgcgctctcctgttccgaccctgccgcttaccggatacctgtccgcctttctcccttcggg
aagcgtggcgctttctcatagctcacgctgtaggtatctcagttcggtgtaggtcgttcgctccaagctgggctgtgtgcacgaaccccc
cgttcagcccgaccgctgcgccttatccggtaactatcgtcttgagtccaacccggtaagacacgacttatcgccactggcagcagcca
ctggtaacaggattagcagagcgaggtatgtaggcggtgctacagagttcttgaagtggtggcctaactacggctacactagaagaac
agtatttggtatctgcgctctgctgaagccagttaccttcggaaaaagagttggtagctcttgatccggcaaacaaaccaccgctggtag
cggtggtttttttgtttgcaagcagcagattacgcgcagaaaaaaaggatctcaagaagatcctttgatcttttctacggggtctgacgctc
agtggaacgaaaactcacgttaagggattttggtcatgagattatcaaaaaggatcttcacctagatccttttaaattaaaaatgaagtttta
aatcaatctaaagtatatatgagtaaacttggtctgacagttaccaatgcttaatcagtgaggcacctatctcagcgatctgtctatttcgttc
atccatagttgcctgactccccgtcgtgtagataactacgatacgggagggcttaccatctggccccagtgctgcaatgataccgcgag
acccacgctcaccggctccagatttatcagcaataaaccagccagccggaagggccgagcgcagaagtggtcctgcaactttatccg
cctccatccagtctattaattgttgccgggaagctagagtaagtagttcgccagttaatagtttgcgcaacgttgttgccattgctacaggc
atcgtggtgtcacgctcgtcgtttggtatggcttcattcagctccggttcccaacgatcaaggcgagttacatgatcccccatgttgtgcaa
aaaagcggttagctccttcggtcctccgatcgttgtcagaagtaagttggccgcagtgttatcactcatggttatggcagcactgcataatt
ctcttactgtcatgccatccgtaagatgcttttctgtgactggtgagtactcaaccaagtcattctgagaatagtgtatgcggcgaccgagt
tgctcttgcccggcgtcaatacgggataataccgcgccacatagcagaactttaaaagtgctcatcattggaaaacgttcttcggggcg
aaaactctcaaggatcttaccgctgttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaatgttgaatactcatactctt
cctttttcaatattattgaagcatttatcagggttattgtctcatgagcggatacatatttgaatgtatttagaaaaataaacaaataggggttc
cgcgcacatttccccgaaaagtgccacctgacgtcgacggatcgggagatcaacttgtttattgcagcttataatggttacaaataaagc
aatagcatcacaaatttcacaaataaagcatttttttcactgcattctagttgtggtttgtccaaactcatcaatgtatcttatcatgtctggatc
aactggataactcaagctaaccaaaatcatcccaaacttcccaccccataccctattaccactgccaattacctgtggtttcatttactctaa
acctgtgattectctgaattattttcattttaaagaaattgtatttgttaaatatgtactacaaacttagtagt (SEQ ID NO: 29)
pLVX-CMV-TCR5 (FIG. 12):
tggaagggctaattcactcccaaagaagacaagatatccttgatctgtggatctaccacacacaaggctacttccctgattagcagaact
acacaccagggccaggggtcagatatccactgacctttggatggtgctacaagctagtaccagttgagccagataaggtagaagagg
ccaataaaggagagaacaccagcttgttacaccctgtgagcctgcatgggatggatgacccggagagagaagtgttagagtggaggt
ttgacagccgcctagcatttcatcacgtggcccgagagctgcatccggagtacttcaagaactgctgatatcgagcttgctacaaggga
ctttccgctggggactttccagggaggcgtggcctgggcgggactggggagtggcgagccctcagatcctgcatataagcagctgct
ttttgcctgtactgggtctctctggttagaccagatctgagcctgggagctctctggctaactagggaacccactgcttaagcctcaataa
agcttgccttgagtgcttcaagtagtgtgtgcccgtctgttgtgtgactctggtaactagagatccctcagacccttttagtcagtgtggaa
aatctctagcagtggcgcccgaacagggacttgaaagcgaaagggaaaccagaggagctctctcgacgcaggactcggcttgctga
agcgcgcacggcaagaggcgaggggcggcgactggtgagtacgccaaaaattttgactagcggaggctagaaggagagagatgg
gtgcgagagcgtcagtattaagcgggggagaattagatcgcgatgggaaaaaattcggttaaggccagggggaaagaaaaaatata
aattaaaacatatagtatgggcaagcagggagctagaacgattcgcagttaatcctggcctgttagaaacatcagaaggctgtagacaa
atactgggacagctacaaccatcccttcagacaggatcagaagaacttagatcattatataatacagtagcaaccctctattgtgtgcatc
aaaggatagagataaaagacaccaaggaagctttagacaagatagaggaagagcaaaacaaaagtaagaccaccgcacagcaag
cggccggccgctgatcttcagacctggaggaggagatatgagggacaattggagaagtgaattatataaatataaagtagtaaaaattg
aaccattaggagtagcacccaccaaggcaaagagaagagtggtgcagagagaaaaaagagcagtgggaataggagctttgttcctt
gggttcttgggagcagcaggaagcactatgggcgcagcgtcaatgacgctgacggtacaggccagacaattattgtctggtatagtgc
agcagcagaacaatttgctgagggctattgaggcgcaacagcatctgttgcaactcacagtctggggcatcaagcagctccaggcaa
gaatcctggctgtggaaagatacctaaaggatcaacagctcctggggatttggggttgctctggaaaactcatttgcaccactgctgtgc
cttggaatgctagttggagtaataaatctctggaacagatttggaatcacacgacctggatggagtgggacagagaaattaacaattaca
caagataatacactccttaattgaagaatcgcaaaaccagcaagaaaagaatgaacaagaattattggaattagataaatgggcaagtt
tgtggaattggtttaacataacaaattggctgtggtatataaaattattcataatgatagtaggaggcttggtaggtttaagaatagtttttgct
gtactttctatagtgaatagagttaggcagggatattcaccattatcgtttcagacccacctcccaaccccgaggggacccgacaggcc
cgaaggaatagaagaagaaggtggagagagagacagagacagatccattcgattagtgaacggatctcgacggtatcgcctttaaaa
gaaaaggggggattggggggtacagtgcaggggaaagaatagtagacataatagcaacagacatacaaactaaagaattacaaaaa
caaattacaaaaattcaaaattttcgggtttattacagggacagcagagatccagtttatcgataagcttgggagttccgcgttacataactt
acggtaaatggcccgcctggctgaccgcccaacgacccccgcccattgacgtcaataatgacgtatgttcccatagtaacgccaatag
ggactttccattgacgtcaatgggtggagtatttacggtaaactgcccacttggcagtacatcaagtgtatcatatgccaagtacgccccc
tattgacgtcaatgacggtaaatggcccgcctggcattatgcccagtacatgaccttatgggactttcctacttggcagtacatctacgtat
tagtcatcgctattaccatggtgatgcggttttggcagtacatcaatgggcgtggatagcggtttgactcacggggatttccaagtctcca
ccccattgacgtcaatgggagtttgttttggcaccaaaatcaacgggactttccaaaatgtcgtaacaactccgccccattgacgcaaat
gggcggtaggcgtgtacggtgggaggtctatataagcagagctcgtttagtgaaccgtcagatcgcctggagacgccatccacgctgt
tttgacctccatagaagacaccgactctactagaggatctatttccggtgAATTCGCCACCatggcctccctgctcttcttctgtg
gggccttttatctcctgggaaccggttccatgGAAGCTGACATCTACCAGACCCCAAGATACCTTGTT
ATAGGGACAGGAAAGAAGATCACTCTGGAATGTTCTCAAACCATGGGCCATGAC
AAAATGTACTGGTATCAACAAGATCCAGGAATGGAACTACACCTCATCCACTATT
CCTATGGAGTTAATTCCACAGAGAAGGGAGATCTTTCCTCTGAGTCAACAGTCTC
CAGAATAAGGACGGAGCATTTTCCCCTGACCCTGGAGTCTGCCAGGCCCTCACAT
ACCTCTCAGTACCTCTGTGCCAGCAGTCGGACGCCCAACATTCAGTACTTCGGCG
CCGGGACCCGGCTCTCAGTGCTGgaggacctgaaaaacgtgttcccacccgaggtcgctgtgtttgagccatca
gaagcagagatctcccacacccaaaaggccacACTAGTgtgcctggccacaggcttctaccccgaccacgtggagctgagct
ggtgggtgaatgggaaggaggtgcacagtggggtcagcacagacccgcagcccctcaaggagcagcccgccctcaatgactcca
gatactgcctgagcagccgcctgagggtctcggccaccttctggcagaacccccgcaaccacttccgctgtcaagtccagttctacgg
gctctcggagaatgacgagtggacccaggatagggccaaacctgtcacccagatcgtcagcgccgaggcctggggtagagcagac
tgtggcttcacctccgagtcttaccagcaaggggtcctgtctgccaccatcctctatgagatcttgctagggaaggccaccttgtatgcc
gtgctggtcagtgccctcgtgctgatggccatggtcaagagaaaggattccagaggcGGGAGCGGAGCCACGAAC
TTCTCTCTGTTAAAGCAAGCAGGAGACGTGGAAGAAAACCCCGGTCCTatgaactcctct
ctggactttctaattctgatcttaatgtttggaggaACTAGTGGTCAACAGCTGAATCAGAGTCCTCAAT
CTATGTTTATCCAGGAAGGAGAAGATGTCTCCATGAACTGCACTTCTTCAAGCAT
ATTTAACACCTGGCTATGGTACAAGCAGGAACCTGGGGAAGGTCCTGTCCTCTTG
ATAGCCTTATATAAGGCTGGTGAATTGACCTCAAATGGAAGACTGACTGCTCAGT
TTGGTATAACCAGAAAGGACAGCTTCCTGAATATCTCAGCATCCATACCTAGTGA
TGTAGGCATCTACTTCTGTGCTGGCACATACCAGAAAGTTACCTTTGGAACTGGA
ACAAAGCTCCAAGTCATCCCAAATATCCAGAACCCTGACCCTGCCGTGTACCAGC
TGAGAGACTCTAAATCCAGTGACAAGTCTGCATGCctattcaccgattttgattctcaaacaaatgtgtc
acaaagtaaggattctgatgtgtatatcacagacaaaactgtgctagacatgaggtctatggacttcaagagcaacagtgctgtggcctg
gagcaacaaatctgactttgcatgtgcaaacgccttcaacaacagcattattccagaagacaccttcttccccagcccagaaagttcctg
tgatgtcaagctggtcgagaaaagctttgaaacagatacgaacctaaactttcaaaacctgtcagtgattgggttccgaatcctcctcctg
aaagtggccgggtttaatctgctcatgacgctgcggctgtggtccagcTGActcgagggatcccgcccctctccctcccccccccct
aacgttactggccgaagccgcttggaataaggccggtgtgcgtttgtctatatgttattttccaccatattgccgtcttttggcaatgtgagg
gcccggaaacctggccctgtatcttgacgagcattcctaggggtctttcccctctcgccaaaggaatgcaaggtctgttgaatgtcgtg
aaggaagcagttcctctggaagatcttgaagacaaacaacgtctgtagcgaccctttgcaggcagcggaaccccccacctggcgac
aggtgcctctgcggccaaaagccacgtgtataagatacacctgcaaaggcggcacaaccccagtgccacgttgtgagttggatagttg
tggaaagagtcaaatggctcacctcaagcgtattcaacaaggggctgaaggatgcccagaaggtaccccattgtatgggatctgatct
ggggcctcggtgcacatgctttacatgtgtttagtcgaggttaaaaaacgtctaggccccccgaaccacggggacgtggttttcctttga
aaaacacgatgataatatggtgagcaagggcgaggaggataacatggccatcatcaaggagttcatgcgcttcaaggtgcacatgga
gggctccgtgaacggccacgagttcgagatcgagggcgagggcgagggccgcccctacgagggcacccagaccgccaagctga
aggtgaccaagggtggccccctgcccttcgcctgggacatcctgtcccctcagttcatgtacggctccaaggcctacgtgaagcaccc
cgccgacatccccgactacttgaagctgtccttccccgagggcttcaagtgggagcgcgtgatgaacttcgaggacggcggcgtggt
gaccgtgacccaggactcctccctgcaggacggcgagttcatctacaaggtgaagctgcgcggcaccaacttcccctccgacggcc
ccgtaatgcagaagaagaccatgggctgggaggcctcctccgagcggatgtaccccgaggacggcgccctgaagggcgagatca
agcagaggctgaagctgaaggacggcggccactacgacgctgaggtcaagaccacctacaaggccaagaagcccgtgcagctgc
ccggcgcctacaacgtcaacatcaagttggacatcacctcccacaacgaggactacaccatcgtggaacagtacgaacgcgccgag
ggccgccactccaccggcggcatggacgagctgtacaagtgaacgcgtctggaacaatcaacctctggattacaaaatttgtgaaag
attgactggtattcttaactatgttgctccttttacgctatgtggatacgctgctttaatgcctttgtatcatgctattgcttcccgtatggctttca
ttttctcctccttgtataaatcctggttgctgtctctttatgaggagttgtggcccgttgtcaggcaacgtggcgtggtgtgcactgtgtttgct
gacgcaacccccactggttggggcattgccaccacctgtcagctcctttccgggactttcgctttccccctccctattgccacggcggaa
ctcatcgccgcctgccttgcccgctgctggacaggggctcggctgttgggcactgacaattccgtggtgttgtcggggaagctgacgt
cctttccatggctgctcgcctgtgttgccacctggattctgcgcgggacgtccttctgctacgtcccttcggccctcaatccagcggacct
tccttcccgcggcctgctgccggctctgcggcctcttccgcgtcttcgccttcgccctcagacgagtcggatctccctttgggccgcctc
cccgcctggaattaattctgcagtcgagacctagaaaaacatggagcaatcacaagtagcaatacagcagctaccaatgctgattgtgc
ctggctagaagcacaagaggaggaggaggtgggttttccagtcacacctcaggtacctttaagaccaatgacttacaaggcagctgta
gatcttagccactttttaaaagaaaagaggggactggaagggctaattcactcccaacgaagacaagatatccttgatctgtggatctac
cacacacaaggctacttccctgattagcagaactacacaccagggccaggggtcagatatccactgacattggatggtgctacaagct
agtaccagttgagccagataaggtagaagaggccaataaaggagagaacaccagcttgttacaccctgtgagcctgcatgggatgga
tgacccggagagagaagtgttagagtggaggtttgacagccgcctagcatttcatcacgtggcccgagagctgcatccggagtacttc
aagaactgctgatatcgagcttgctacaagggactttccgctggggactttccagggaggcgtggcctgggcgggactggggagtgg
cgagccctcagatcctgcatataagcagctgctttttgcctgtactgggtctctctggttagaccagatctgagcctgggagctctctggc
taactagggaacccactgataagcctcaataaagcttgccttgagtgatcaagtagtgtgtgcccgtctgttgtgtgactctggtaacta
gagatccctcagacccttttagtcagtgtggaaaatctctagcagtagtagttcatgtcatcttattattcagtatttataacttgcaaagaaat
gaatatcagagagtgagaggccttgacattgctagcgtttaccgtcgacctctagctagagcttggcgtaatcatggtcatagctgtttcc
tgtgtgaaattgttatccgctcacaattccacacaacatacgagccggaagcataaagtgtaaagcctggggtgcctaatgagtgagct
aactcacattaattgcgttgcgctcactgcccgctttccagtcgggaaacctgtcgtgccagctgcattaatgaatcggccaacgcgcg
gggagaggcggtttgcgtattgggcgctatccgcttcctcgctcactgactcgctgcgctcggtcgttcggctgcggcgagcggtatc
agctcactcaaaggcggtaatacggttatccacagaatcaggggataacgcaggaaagaacatgtgagcaaaaggccagcaaaag
gccaggaaccgtaaaaaggccgcgttgctggcgtttttccataggctccgcccccctgacgagcatcacaaaaatcgacgctcaagtc
agaggtggcgaaacccgacaggactataaagataccaggcgtttccccctggaagctccctcgtgcgctctcctgttccgaccctgcc
gcttaccggatacctgtccgcctttctcccttcgggaagcgtggcgctttctcatagctcacgctgtaggtatctcagttcggtgtaggtcg
ttcgctccaagctgggctgtgtgcacgaaccccccgttcagcccgaccgctgcgccttatccggtaactatcgtcttgagtccaacccg
gtaagacacgacttatcgccactggcagcagccactggtaacaggattagcagagcgaggtatgtaggcggtgctacagagttcttga
agtggtggcctaactacggctacactagaagaacagtatttggtatctgcgctctgctgaagccagttaccttcggaaaaagagttggta
gctcttgatccggcaaacaaaccaccgctggtagcggtggtttttttgtttgcaagcagcagattacgcgcagaaaaaaaggatctcaa
gaagatcctttgatcttttctacggggtctgacgctcagtggaacgaaaactcacgttaagggattttggtcatgagattatcaaaaaggat
cttcacctagatccttttaaattaaaaatgaagttttaaatcaatctaaagtatatatgagtaaacttggtctgacagttaccaatgcttaatca
gtgaggcacctatctcagcgatctgtctatttcgttcatccatagttgcctgactccccgtcgtgtagataactacgatacgggagggctta
ccatctggccccagtgctgcaatgataccgcgagacccacgctcaccggctccagatttatcagcaataaaccagccagccggaagg
gccgagcgcagaagtggtcctgcaactttatccgcctccatccagtctattaattgttgccgggaagctagagtaagtagttcgccagtt
aatagtttgcgcaacgttgttgccattgctacaggcatcgtggtgtcacgctcgtcgtttggtatggcttcattcagctccggttcccaacg
atcaaggcgagttacatgatcccccatgttgtgcaaaaaagcggttagctccttcggtcctccgatcgttgtcagaagtaagttggccgc
agtgttatcactcatggttatggcagcactgcataattctcttactgtcatgccatccgtaagatgcttttctgtgactggtgagtactcaacc
aagtcattctgagaatagtgtatgcggcgaccgagttgctcttgcccggcgtcaatacgggataataccgcgccacatagcagaacttt
aaaagtgctcatcattggaaaacgttcttcggggcgaaaactctcaaggatcttaccgctgttgagatccagttcgatgtaacccactcgt
gcacccaactgatcttcagcatcttttactttcaccagcgtttctgggtgagcaaaaacaggaaggcaaaatgccgcaaaaaagggaat
aagggcgacacggaaatgttgaatactcatactatcctttttcaatattattgaagcatttatcagggttattgtctcatgagcggatacata
tttgaatgtatttagaaaaataaacaaataggggttccgcgcacatttccccgaaaagtgccacctgacgtcgacggatcgggagatca
acttgtttattgcagatataatggttacaaataaagcaatagcatcacaaatttcacaaataaagcatttttttcactgcattctagttgtggttt
gtccaaactcatcaatgtatcttatcatgtctggatcaactggataactcaagctaaccaaaatcatcccaaacttcccaccccatacccta
ttaccactgccaattacctgtggtttcatttactctaaacctgtgattcctctgaattattttcattttaaagaaattgtatttgttaaatatgtacta
caaacttagtagt (SEQ ID NO: 30)

In another aspect, the present technology provides a recombinant cell comprising a vector, wherein the vector comprises (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent in a compartment, optionally wherein the recombinant cell is a bacterial cell, mammalian cell, or a yeast cell. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet.

In one aspect, the present technology provides a recombinant TCR vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent in a compartment. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet.

In some embodiments, the TCR vector library comprises a TCR repertoire. Thus, in some embodiments, a TCR vector library is a full or partial collection of the different TCRs produced in a single donor or subject as a result of V(D)J rearrangement and/or T cell selection. In other embodiments, a TCR vector library of the present disclosure comprises a defined collection of TCRs specifically selected for their binding specificities or other desired characteristics. For example, the TCR vector library can comprise a subset of TCRs that specifically bind to a particular target cell, antigen, antigen:MHC complex, or combination thereof. In these embodiments, the TCRs can be derived from a single donor or subject, or more than one donor or subject.

In certain embodiments, each TCR in the TCR vector library is genetically distinct and comprises a distinct binding specificity. In some embodiments, the TCR vector comprises about 2 to about 5 different TCRs, about 2 to about 10 different TCRs, about 5 to about 10 different TCRs, about 5 to about 15 different TCRs, about 5 to about 20 different TCRs, about 10 to about 30 different TCRs, about 10 to about 40 different TCRs, about 10 to about 50 TCRs, about 20 to about 60 different TCRs, about 25 to about 75 different TCRs, about 50 to about 100 different TCRs, or greater than 100 different TCRs.

Precise molecular compositions can be advantageous for stability, efficacy, and/or safety of biotherapeutics. Thus, in certain embodiments, each vector and/or TCR in the TCR vector library is characterized to determine the nucleic acid and/or amino acid sequence of the TCR and/or its binding specificity. In some embodiments, molecular characterization is performed by nucleic acid sequencing the vector or a part of the vector (e.g., the first polynucleotide and the second polynucleotide). In certain embodiments, molecular characterization is performed by analysis of the TCR protein using mass spectrometry. In some embodiments, molecular characterization of the TCR vector library is performed by nucleic acid sequencing of 2 or more, or 3 or more, or 4 or more, or 5 or more, or 6 or more, or 7 or more, or 8 or more, or 9 or more, or 10 or more, or 15 or more, or 20 or more, or 30 or more, or 40 or more, or 50 or more isolated colonies, each comprising a recombinant cell transformed with the TCR vector library (e.g., a bacterial cell). In some embodiments, the TCR vector library is refined or selected by single-colony nucleic acid amplification and sequencing of transformed recombinant cells, followed by mixing of different vector colonies at a defined ratio to generate a defined molecular library composition. In some embodiments, the selection of individual vectors for inclusion in the library is informed by aspects of characterization and/or binding specificity of the starting library. Non-limiting examples of such aspects include TCR clonal prevalence, TCR enrichment characteristics from in vitro assays, TCR V segment sequence, TCR D segment sequence, TCR J segment sequence, TCR gene motifs, and/or CDR3 gene motifs.

In some embodiments, the ratio of vectors in the TCR vector library is adjusted or selected to optimize therapeutic activity of the library. For example, a TCR vector library comprising two TCRs may have a vector ratio of about 1:1, about 1:2, about 1:5, about 1:10, etc of each vector. In another non-limiting example, a library comprising three TCRs may have a vector ratio of about 1:1:1, about 2:1:1, about 2:2:1, etc of each vector.

In another aspect, the present technology provides an isolated immune cell comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent in a compartment. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet. In some embodiments the immune cell is a hematopoietic stem cell, a hematopoietic progenitor cell, a T cell, or an natural killer (NK) cell.

In one aspect, the present technology provides a cell population comprising a recombinant TCR vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent in a compartment. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet. In some embodiments, the population comprises hematopoietic stem cells, hematopoietic progenitor cells, T cells, or NK cells. In some embodiments, the cell population comprises a full or partial TCR repertoire of a subject.

Additional aspects of the present technology relate to compositions comprising a carrier and one or more vectors of the present technology. Alternatively, the compositions comprise a carrier and one or more recombinant TCR vector libraries of the present technology. In another embodiment, the compositions comprise a carrier and one or more immune cells comprising one or more vectors of the present technology. In yet another embodiment, the compositions comprise a carrier and a cell population comprising a recombinant TCR vector library of the present technology. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers for binding vectors, cells, cell populations, or vector libraries or will be able to ascertain such, using routine experimentation.

In some embodiments, the carrier is a pharmaceutically acceptable carrier. Pharmaceutical compositions of the present disclosure including but not limited to any one of the claimed compositions may comprise a vector, library, cell, or cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure can be formulated for oral, intravenous, topical, enteral, and/or parenteral administration. In certain embodiments, the compositions of the present disclosure are formulated for intravenous administration.

### II. Methods of Preparing the Compositions of the Present Technology

Provided herein is a method for preparing a recombinant TCR library, the method comprising transforming a population of cells with a vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent in a compartment. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet. In some embodiments, the population comprises hematopoietic stem cells, hematopoietic progenitor cells, T cells, or NK cells.

In some embodiments of the method, the library is screened for specific binding to a target cell. In certain embodiments, the target cell is a cancer cell, a cell infected with a virus, a cell derived from a subject infected with a virus, a tumor cell, or a tissue biopsy cell isolated from a subject suspected of having a viral infection or cancer.

In some embodiments of the method, the library is screened for specific binding to an antigen:MHC complex. In some embodiments the antigen of the antigen:MHC complex is a viral antigen derived from a virus selected from the group consisting of adenovirus, CMV, coronavirus, coxsackievirus, Dengue virus, Epstein-Barr virus (EBV), enterovirus 71 (EV71), Ebola virus, hepatitis A (HAV), hepatitis B (HBV), cytomegalovirus (CMV), hepatitis C (HCV), hepatitis D (HDV), hepatitis E (HEV), human immunodeficiency virus (HIV), human papillomavirus (HPV), herpes simplex virus (HSV), human T-lymphotropic virus (HTLV), influenza A virus, influenza B virus, Japanese encephalitis, leukemia virus, measles virus, molluscum contagiosum, orf virus, parvovirus, rabies virus, respiratory syncytial virus, rift valley fever virus, rubella virus, rotavirus, tick-borne encephalitis (TBEV), simian immunodeficiency virus, tobacco etch virus (TEV), varicella zoster virus, variola, West Nile virus, Zika virus, and Chikungunya virus. In other embodiments, the antigen of the antigen:MHC complex is a tumor antigen selected from the group consisting of CD45, glypican-3, IGF2B3, Kallikrein 4, KIF20A, Lengsin, Meloe, mucin 5AC (MUC5AC), survivin, cyclin-A1, MAGE-A1, MAGE-C1, MAGE-C2, SSX2, XAGElb/GAGED2A, CD19, CD20, CD22, CD52, EGFR, HER2, TRAILR1, RANKL, IGF1R, EpCAM, and CEA.

In some embodiments of the method, the library is screened for T cell phenotypic markers. In certain embodiments, the T cell phenotypic markers identify expression of one or more TCR complex components, e.g., TCRalpha, TCRbeta, TCRgamma, TCRdelta, CD3δ/ε, CD3γ/ε, and CD247 ζ/ζ or ζ/η. In some embodiments, the T cell phenotypic markers aid in the identification of specific T cell subsets such as naïve CD8+ T cells, naïve CD4+ T cells, CD4+ T cells, CD8+ Cytotoxic T cells, gamma/delta T cells, NKT cells, Th1 cells, Th2 cells, Th9 cells, Th22 cells, T follicular helper cells, Th17 cells, and regulatory T cells. Antibodies specific to cell surface markers suitable for identifying these T cell subsets are known in the art and are available, for example, from R&D Systems. Screening can be performed by any method known in the art including but not limited to ELISA, Western blot, Northern blot, PCR, qPCR, and flow cytometry.

In certain embodiments of the method, the library is screened for activity in a co-culture system, wherein the co-culture system comprises at least one of the following: (a) a cancer cell line; (b) a plurality of cells infected with a known virus; (c) a plurality of tumor cells isolated from a cancer patient; (d) an immortalized cell line; or (e) a plurality of cells derived from a patient tissue biopsy. In some embodiments, activity is measured by assaying co-engagement of the recombinant TCR/CD3 complex and a co-stimulatory receptor, e.g., CD28. Co-engagement of these receptors on the cell surface leads to intracellular signaling events and the activation of nuclear transcription factors such as Nuclear Factor of Activated T cells (NFAT), NF-kB and AP-1. Specifically, engagement of the TCR/CD3 complex leads to the phosphorylation and activation of PLC-g, intracellular calcium flux and transcriptional activation of NFAT pathway. In some embodiments, co-engagement of TCR/CD3 with the co-stimulatory receptor CD28 leads to activation of ERK/JNK and IkB kinase (IKK), which in turn regulates transcriptional activation of AP-1 and NF-kB pathways, respectively. The IL-2 promoter contains DNA binding sites for NFAT, NF-kB and AP-1. Therefore, co-engagement of TCR/CD3 and CD28 results in IL-2 production, which is commonly used as a functional readout for T cell activation. In some embodiments, other endpoints used to measure T cell activation include but are not limited to cell proliferation, cytotoxicity (death of the target cell), and production of additional cytokines such as IFNγ. Kits suitable for measuring or detecting T cell activity kits are available from, for example, Promega Corp. (T Cell Activation Bioassay (IL-2)(a,b) (Cat. #J1651 and J1655)).

In some embodiments of the method, the transformed cells are activated in vitro. In particular embodiments, activation is performed using one or more of the following stimulants: anti-CD3 antibody, anti-CD8 antibody, anti-CD27 antibody, IL-2, IL-4, IL-21, anti-PD1 antibody, anti-CTLA4 antibody, anti CD3/CD28 tetrameric antibody, tumor cell lysate, cellular co-culture with virus-infected cells, and tumor cell lines. T cell activation kits are available from, for example, Miltenyi Biotec (T cell Activation/Expansion Kit, human, cat #130-091-441).

In certain embodiments of the method, the population of cells is transformed with a transcription factor. In some embodiments, the transcription factor is selected from the group consisting of forkhead box P3 (FOXP3, Entrez gene: 50943, RefSeq mRNA: NM_001114377; NM_014009), PR domain zinc finger protein 1 (BLIMP-1, Entrez gene: 639, RefSeq mRNA: NM_001198; NM_182907), Helios (IKZF2, Entrez gene: 22807, RefSeq mRNA: NM_001079526.1; NM_016260.2, Uniprot: Q9UKS7), Ikaros (IKZF1, Entrez:gene: 10320, RefSeq mRNA: NM_001220765.2; NM_006060.6 etc.; Uniprot: Q13422) and transforming growth factor beta 1 (TGF-beta 1, Entrez gene: 7040, RefSeq mRNA: NM_000660). FOXP3 is a protein involved in immune system responses and is thought to function as a master regulator of the development and function of regulatory T cells. Regulatory T cells generally suppress the immune response. BLIMP-1 acts as a repressor of beta-interferon (β-IFN) gene expression. Regulatory T cells release TGF-β1 to inhibit the actions of other T cells. Interleukin 1- and interleukin 2-dependent proliferation of activated T cells, and the activation of quiescent helper T cells and cytotoxic T cells is prevented by the activity of TGF-β1. Similarly, TGF-β1 can inhibit the secretion and activity of many other cytokines including interferon-γ, tumor necrosis factor-alpha (TNF-α) and various interleukins. It can also decrease the expression levels of cytokine receptors, such as the IL-2 receptor to down-regulate the activity of immune cells. Non-limiting examples of transcription factor amino acid sequences are provided herein:

FOXP3 (UniProt Q9BZS1):
(SEQ ID NO: 31)
MPNPRPGKPSAPSLALGPSPGASPSWRAAPKASDLLGARGPGGTFQGRDL
RGGAHASSSSLNPMPPSQLQLPTLPLVMVAPSGARLGPLPHLQALLQDRP
HFMHQLSTVDAHARTPVLQVHPLESPAMISLTPPTTATGVFSLKARPGLP
PGINVASLEWVSREPALLCTFPNPSAPRKDSTLSAVPQSSYPLLANGVCK
WPGCEKVFEEPEDFLKHCQADHLLDEKGRAQCLLQREMVQSLEQQLVLEK
EKLSAMQAHLAGKMALTKASSVASSDKGSCCIVAAGSQGPVVPAWSGPRE
APDSLFAVRRHLWGSHGNSTFPEFLHNMDYFKFHNMRPPFTYATLIRWAI
LEAPEKQRTLNEIYHWFTRMFAFFRNHPATWKNAIRHNLSLHKCFVRVES
EKGAVWTVDELEFRKKRSQRPSRCSNPTPGP
BLIMP-1 (UniProt O75626):
(SEQ ID NO: 32)
MLDICLEKRVGTTLAAPKCNSSTVRFQGLAEGTKGTMKMDMEDADMTLWT
EAEFEEKCTYIVNDHPWDSGADGGTSVQAEASLPRNLLFKYATNSEEVIG
VMSKEYIPKGTRFGPLIGEIYTNDTVPKNANRKYFWRIYSRGELHHFIDG
FNEEKSNWMRYVNPAHSPREQNLAACQNGMNIYFYTIKPIPANQELLVWY
CRDFAERLHYPYPGELTMMNLTQTQSSLKQPSTEKNELCPKNVPKREYSV
KEILKLDSNPSKGKDLYRSNISPLTSEKDLDDFRRRGSPEMPFYPRVVYP
IRAPLPEDFLKASLAYGIERPTYITRSPIPSSTTPSPSARSSPDQSLKSS
SPHSSPGNTVSPVGPGSQEHRDSYAYLNASYGTEGLGSYPGYAPLPHLPP
AFIPSYNAHYPKFLLPPYGMNCNGLSAVSSMNGINNFGLFPRLCPVYSNL
LGGGSLPHPMLNPTSLPSSLPSDGARRLLQPEHPREVLVPAPHSAFSFTG
AAASMKDKACSPTSGSPTAGTAATAEHVVQPKATSAAMAAPSSDEAMNLI
KNKRNMTGYKTLPYPLKKQNGKIKYECNVCAKTFGQLSNLKVHLRVHSGE
RPFKCQTCNKGFTQLAHLQKHYLVHTGEKPHECQVCHKRFSSTSNLKTHL
RLHSGEKPYQCKVCPAKFTQFVHLKLHKRLHTRERPHKCSQCHKNYIHLC
SLKVHLKGNCAAAPAPGLPLEDLTRINEEIEKFDISDNADRLEDVEDDIS
VISVVEKEILAVVRKEKEETGLKVSLQRNMGNGLLSSGCSLYESSDLPLM
KLPPSNPLPLVPVKVKQETVEPMDP
TGF-beta 1 (UniProt P01137):
(SEQ ID NO: 33)
MPPSGLRLLLLLLPLLWLLVLTPGRPAAGLSTCKTIDMELVKRKRIEAIR
GQILSKLRLASPPSQGEVPPGPLPEAVLALYNSTRDRVAGESAEPEPEPE
ADYYAKEVTRVLMVETHNEIYDKFKQSTHSIYMFFNTSELREAVPEPVLL
SRAELRLLRLKLKVEQHVELYQKYSNNSWRYLSNRLLAPSDSPEWLSFDV
TGVVRQWLSRGGEIEGFRLSAHCSCDSRDNTLQVDINGFTTGRRGDLATI
HGMNRPFLLLMATPLERAQHLQSSRHRRALDTNYCFSSTEKNCCVRQLYI
DFRKDLGWKWIHEPKGYHANFCLGPCPYIWSLDTQYSKVLALYNQHNPGA
SAAPCCVPQALEPLPIVYYVGRKPKVEQLSNMIVRSCKCS

In some embodiments, the method further comprises administering an effective amount of the recombinant TCR library prepared according to the methods described herein to a subject in need thereof. In some embodiments, the subject in need thereof is suffering from cancer or a viral infection.

In another aspect, provided herein is a recombinant TCR library prepared by a method comprising transforming a population of cells with a vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent in a compartment. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet. In some embodiments, the population comprises hematopoietic stem cells, hematopoietic progenitor cells, T cells, or NK cells.

In one aspect, the present technology provides a composition comprising a carrier and the recombinant TCR library prepared by a method as described herein. In some embodiments, the carrier is a pharmaceutically acceptable carrier.

### III. Methods of Use

The methods of treatment described herein provide a format for isolation and use of specific TCRs that can be rapidly discovered, amplified, and returned to the subject on a timescale that is relevant for bedside therapies (e.g., weeks, rather than months).

Accordingly, in one aspect, provided herein is a method of treating a subject in need thereof, the method comprising administering to the subject an effective amount of a recombinant TCR library or a composition comprising a recombinant TCR library, wherein the recombinant TCR library was prepared by a method comprising transforming a population of cells with a vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent in a compartment. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet.

In another aspect, provided herein is a method of treating cancer in a subject in need thereof, the method comprising administering to the subject an effective amount of a recombinant TCR library or a composition comprising a recombinant TCR library, wherein the recombinant TCR library was prepared by a method comprising transforming a population of cells with a vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent in a compartment. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet.

In some embodiments, the cancer is acute lymphoblastic leukemia (ALL); acute myeloid leukemia (AML); adrenocortical carcinoma; AIDS-related cancers; anal cancer; appendix cancer; astrocytomas; atypical teratoid/rhabdoid tumor, brain cancer; basal cell carcinoma of the skin; bile duct cancer; bladder cancer; bone cancer; breast cancer; bronchial tumors; Burkitt lymphoma; carcinoid tumor (gastrointestinal); germ cell tumor; primary CNS lymphoma; cervical cancer; cholangiocarcinoma; chordoma; chronic lymphocytic leukemia (CLL); chronic myelogenous leukemia (CML); chronic myeloproliferative neoplasms; colorectal cancer; craniopharyngioma; cutaneous T-cell lymphoma; ductal carcinoma in situ (DCIS); endometrial cancer; ependymoma; esophageal cancer; esthesioneuroblastoma; extracranial germ cell tumor; extragonadal germ cell tumor; eye cancer; intraocular melanoma; retinoblastoma; fallopian tube cancer; fibrous histiocytoma of bone, malignant, and osteosarcoma; gallbladder cancer; gastric cancer; gastrointestinal carcinoid tumor; gastrointestinal stromal tumors (GIST); germ cell tumors; gestational trophoblastic disease; hairy cell leukemia; head and neck cancer; heart tumors; hepatocellular cancer; histiocytosis, Langerhans cell; Hodgkin lymphoma; hypopharyngeal cancer; intraocular melanoma; islet cell tumors, pancreatic neuroendocrine tumors; kidney cancer; laryngeal cancer; leukemia; lip and oral cavity cancer; liver cancer; lung cancer; lymphoma; male breast cancer; malignant fibrous histiocytoma of bone and osteosarcoma; melanoma; Merkel cell carcinoma; mesothelioma; metastatic cancer; mouth cancer; multiple endocrine neoplasia syndrome; multiple myeloma/plasma cell neoplasms; mycosis fungoides; myelodysplastic syndrome, myeloproliferative neoplasm, chronic; nasopharyngeal cancer; neuroblastoma; Non-Hodgkin lymphoma; non-small cell lung cancer; oral cancer, oropharyngeal cancer; osteosarcoma; ovarian cancer; pancreatic cancer; pancreatic neuroendocrine tumors; papillomatosis; paraganglioma; paranasal sinus cancer; parathyroid cancer; pharyngeal cancer; pheochromocytoma; pituitary tumor; pleuropulmonary blastoma; prostate cancer; rectal cancer; recurrent cancer; renal cell cancer; retinoblastoma; rhabdomyosarcoma; salivary gland cancer; sarcoma; Ewing sarcoma; Kaposi sarcoma; osteosarcoma; uterine sarcoma; Sézary syndrome; skin cancer; small cell lung cancer; small intestine cancer; soft tissue sarcoma; squamous cell carcinoma of the skin; squamous neck cancer; stomach cancer; T cell lymphoma; testicular cancer; throat cancer; nasopharyngeal cancer; hypopharyngeal cancer; thymic carcinoma; thyroid cancer; urethral cancer; uterine cancer; vaginal cancer; vascular tumors; vulvar cancer; or Wilms tumor.

In one aspect, provided herein is a method of inhibiting tumor growth in a subject in need thereof, the method comprising administering to the subject an effective amount of a recombinant TCR library or a composition comprising a recombinant TCR library, wherein the recombinant TCR library was prepared by a method comprising transforming a population of cells with a vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell in a compartment. In some embodiments, the mRNA of the single lysed T cell is isolated using an mRNA capture reagent in a compartment. In other embodiments, the polynucleotides encoding the paired T cell receptor polypeptides are derived from a single cell, without the use of an mRNA capture reagent. Additionally or alternatively, in some embodiments, the compartment containing the contents of the single lysed T cell is a microwell (e.g., a microwell within a 96-well plate) or a droplet. In some embodiments, the tumor is a solid tumor.

In another aspect, provided herein is a method of treating a viral infection in a subject in need thereof, the method comprising administering to the subject an effective amount of a recombinant TCR library or a composition comprising a recombinant TCR library, wherein the recombinant TCR library was prepared by a method comprising transforming a population of cells with a vector library comprising a plurality of vectors each comprising (a) a vector backbone; and (b) a first polynucleotide encoding a TCRα polypeptide and a second polynucleotide encoding a TCRβ polypeptide; or (b) a first polynucleotide encoding a TCRγ polypeptide and a second polynucleotide encoding a TCRδ polypeptide; wherein the first and second polynucleotides are a cognate pair, and wherein the first polynucleotide and the second polynucleotide are derived from mRNA of a single lysed T cell that was captured by an mRNA capture reagent in a compartment. In some embodiments, the viral infection is caused by a virus selected from the group consisting of adenovirus, CMV, coronavirus, coxsackievirus, Dengue virus, Epstein-Barr virus (EBV), enterovirus 71 (EV71), Ebola virus, hepatitis A (HAV), hepatitis B (HBV), cytomegalovirus (CMV), hepatitis C (HCV), hepatitis D (HDV), hepatitis E (HEV), human immunodeficiency virus (HIV), human papillomavirus (HPV), herpes simplex virus (HSV), human T-lymphotropic virus (HTLV), influenza A virus, influenza B virus, Japanese encephalitis, leukemia virus, measles virus, molluscum contagiosum, orf virus, parvovirus, rabies virus, respiratory syncytial virus, rift valley fever virus, rubella virus, rotavirus, tick-borne encephalitis (TBEV), simian immunodeficiency virus, tobacco etch virus (TEV), varicella zoster virus, variola, West Nile virus, Zika virus, and Chikungunya virus.

In some embodiments, the methods further comprise activating a suicide switch to kill the cells or cell population comprising a vector with a suicide switch (e.g., i-caspase9), thereby reducing the risk of harm to the patient. In some embodiments, the suicide switch is triggered following significant improvement or an apparent cure of the subject's cancer or infection, in order to reduce the risk of long-term side effects.

In certain embodiments, the methods further comprise pre-stimulating the cells in vitro prior to administration to achieve a desired TCR function or T cell identity in vivo, as described in the methods of preparation above.

In some embodiments, the methods further comprise co-expressing one or more transcription factors in the recombinant cells to influence T cell development into a potent anti-cancer or anti-viral phenotype, or to prevent the development of immunosuppressing Tregs, as described in the methods of preparation above.

Administration of the cells, libraries, cell populations, or compositions can be effected in one dose, continuously, or intermittently throughout the course of treatment. Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. Suitable dosage formulations and methods of administering the agents are known in the art. In a further aspect, the cells and composition of the present disclosure can be administered in combination with other treatments. The cells and populations of cell are administered to the host using methods known in the art and described, for example, in PCT/US2011/064191. This administration of the cells or compositions of the present disclosure can be done to generate an animal model of the desired disease, disorder, or condition for experimental and screening assays.

Compositions of the present disclosure may be administered in a manner appropriate to the disease to be treated or prevented. The quantity and frequency of administration will be determined by such factors as the condition of the patient, and the type and severity of the patient's disease, although appropriate dosages may be determined by clinical trials.

In some embodiments, an effective dose of the recombinant TCR library comprises about 50 to about 10² cells, about 10² cells to about 10³ cells, about 10² cells to about 10⁴ cells, about 10³ cells to about 10⁵ cells, about 10⁴ cells to about 10⁶ cells, about 10⁵ cells to about 10⁷ cells, about 10⁶ cells to about 10⁸ cells, about 10⁷ cells to about 10⁹ cells, or about 10⁸ cells to about 10¹⁰ cells. In particular embodiments, the effective dose comprises about 5×10⁵ cells to about 1.5×10⁶ cells or about 1×10⁴ cells to about 5×10⁴ cells, about 5×10⁴ cells to about 5×10⁵ cells, or about 2.5×10⁵ cells to about 7.5×10⁵ cells.

In certain embodiments, the administration is repeated and/or modified as needed in response to the subject's specific response to therapy administration. A repeat administration may be needed, for example, upon the re-appearance of a cancer cells or virus in the subject in need thereof (e.g., tumor immune “escape”). In some embodiments, repeated administration comprises cells, libraries, cell populations, or compositions prepared by a method that is distinct from the initial administration (e.g., the repeat dose comprises an additional step of pre-screening or pre-activating the T cells). In a particular embodiment, the methods of treatment provided herein further comprise administering a second, third, fourth, fifth, sixth, seventh, eighth, ninth, tenth dose of the recombinant TCR library or the composition.

In some embodiments, the recombinant TCR library comprises cells that are autologous or allogenic to the subject being treated.

Immunoassay and Imaging. In some aspects, the recombinant TCR library disclosed herein can be used to assay for the presence of target cells in a biological sample isolated from a subject (e.g. human plasma). The target cells (e.g., cancer cells) can be detected by exposing the recombinant TCR library to the biological sample and assaying for TCR activation and/or binding.

In addition to assaying for the presence of target cells, the recombinant TCR library disclosed herein can be used for in vivo imaging. Detectable labels that can be incorporated with the recombinant TCR library include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the TCR library. The TCR library which has been labeled with an appropriate detectable imaging moiety, such as a radioisotope (e.g., ¹³¹I, ¹¹²In, ^{99m}Tc), a radio-opaque substance, or a material detectable by nuclear magnetic resonance, is introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The recombinant TCR library will then preferentially accumulate at the location of target cells. For example, in vivo tumor imaging is described in S. W. Burchiel et al., Tumor Imaging: The Radiochemical Detection of Cancer 13 (1982).

Diagnostic Uses. The recombinant TCR library disclosed herein can be used for diagnostic methods. As such, the present disclosure provides methods for using the recombinant TCR library disclosed herein in the diagnosis of cancer or viral infections in a subject. The diagnostic methods comprise contacting a biological sample isolated from a subject with a recombinant TCR library of the present disclosure. Biological samples can be obtained from any tissue (including biopsies), cell or body fluid of a subject. The activity or binding of the recombinant TCR library is assayed. If the recombinant TCR library is activated upon exposure to the biological sample, the subject's biological sample contains cells that are recognized by the TCR library. Accordingly, the subject is diagnosed with cancer or a viral infection.

Prognostic Uses. The recombinant TCR library disclosed herein can be used for prognostic methods. As such, the present disclosure provides methods for using the recombinant TCR library disclosed herein in predicting the prognosis of a subject with cancer or a viral infection. The prognostic methods comprise contacting a biological sample isolated from a subject with a recombinant TCR library of the present disclosure. Biological samples can be obtained from any tissue (including biopsies), cell or body fluid of a subject. The activity or binding of the recombinant TCR library is assayed. If the recombinant TCR library is activated upon exposure to the biological sample, the subject's biological sample contains cells that are recognized by the TCR library. Accordingly, the subject is identified as having or at risk for developing cancer and/or solid tumors, or a viral infection.

In some embodiments, the subject is a human, an animal, a non-human primate, a dog, cat, a sheep, a mouse, a horse, or a cow. In a particular embodiment, the subject is a human.

### IV. Kits

As set forth herein, the present disclosure provides methods of TCR library preparation, methods of treatment, diagnostic methods, and prognostic methods. In one particular aspect, the present disclosure provides kits for performing these methods as well as instructions for carrying out the methods of the present disclosure such as collecting donor cells and/or performing a screen, and/or analyzing the results.

In some embodiments, the kit comprises, or alternatively consists essentially of, or yet further consists of the recombinant TCR library disclosed herein, and instructions for use. In some embodiments, the kit comprises, or alternatively consists essentially of, or yet further consists of one or more vectors disclosed herein, and instructions for use. In some embodiments, the kit comprises, or alternatively consists essentially of, or yet further consists of a pharmaceutical composition as disclosed herein, and instructions for use.

In some aspects, the kit can comprise: one or more vectors, cells, populations, or recombinant TCR libraries as disclosed herein; means for determining the amount of a reactive antigen or cell in a biological sample or means of assaying the activity of the TCR library; and means for comparison with a standard.

The kit components, (e.g., reagents) can be packaged in a suitable container. For example, these suggested kit components may be provided in solution or as a liquid dispersion or the like. The kit can also comprise, e.g., a buffering agent, a preservative or a protein-stabilizing agent. The kit can further comprise components necessary for detecting a detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

### EXAMPLES

The present technology is further illustrated by the following Examples, which should not be construed as limiting in any way.

### Example 1: Paired Native TCRα:β Amplicon Generation and Cloning into Expression Vectors

First, a population of human T cells were isolated for the collection of paired TCRα:β amplicons as a source of natively paired T cell receptor genes. Peripheral blood mononuclear cells (PBMC) were isolated from anticoagulated whole blood from healthy human patients using Histopaque®-1077 by centrifugation at 400×g for 30 minutes. After removing the upper plasma layer, the mononuclear cells between the plasma and Histopaque®-1077 were collected and resuspended in phosphate buffered saline. Cells were mixed gently and centrifuge at 250×g for 10 min for the removal of the platelets. Removed the supernatant and resuspended cell pellet with PBMCs culture media (RPMI with 10% fetal bovine serum) or cell freezing media (RPMI with 10% fetal bovine serum and 10% DMSO) for cryopreservation.

PBMCs were then cultured with RPMI-media supplied with 10% fetal bovine serum for 4 hours before the emulsion PCR for amplifying the full-length T cell receptor alpha and beta chain, Phorbol 12-myristate 13-acetate (PMA) and ionomycin were added to the PBMC culture media to a final concentration of 100 ng/mL for stimulation of T cells. A custom flow-focusing nozzle was used to isolate single PBMC cells into emulsion droplets with cell lysis buffer and poly(dT) beads which capture mRNA (Ref #1-4). The nozzle design ensured that cells are not exposed to lysis buffer until after they are isolated into single droplets. Within the droplet, cells were co-encapsulated with lysis reagents to release mRNA and poly(dT) magnetic beads for mRNA capture and purification. Emulsions were broken using diethyl ether, whereas poly(dT) beads were recovered and washed using first high-salt hybridization buffers and then PCR buffers. The beads were then re-emulsified in an overlap extension reverse transcription PCR OE-RTRT-PCR mix with primers adapted from Boria et al. (Ref #5) specific for the TCR alpha and beta chain sequences with the incorporation of restriction enzyme sequences and T cell receptor leader sequences for downstream cloning. The linker sequence was also included to enable the physical linkage of the TCR α and β chain during the TCR OE-RT-PCR. SuperScript™ III One-Step RT-PCR System with Platinum™ Taq DNA Polymerase was used for the emulsion PCR reagent. OE-PCR thermocycling conditions are provided in Table 3 and primer sequences are provided in Table 4.

TABLE 3
OE-PCR thermocycling conditions

| Cycle | Temperature | Time | |
|---|---|---|---|
| 1 | 55° C. | 30 | min |
| 1 | 94° C. | 2 | min |
| 5 | 94° C. | 30 | s |
| | 50° C. | 30 | s |
| | 72° C. | 2 | min |
| 5 | 94° C. | 30 | s |
| | 55° C. | 30 | s |
| | 72° C. | 2 | min |
| 30 | 94° C. | 30 | s |
| | 60° C. | 30 | s |
| | 72° C. | 2 | min |
| 1 | 72° C. | 7 | min |

TABLE 4
Primers used for OE-PCR.

| | | SEQ |
|---|---|---|
| Primer | | ID |
| Name | Sequence | NO: |
| TRBV2for | CGCAGTAGCGGTAAACGGC | 34 |
| | GATGCTGAAGTCRCMCAGACTCC | |
| TRBV3for | CGCAGTAGCGGTAAACGGC GATGCWGMTGTTWCCCAGAC | 35 |
| TRBV4for | CGCAGTAGCGGTAAACGGC | 36 |
| | GACACTGRAGTYACSCAGACACC | |
| TRBV5for | CGCAGTAGCGGTAAACGGC GAGGCTGGAGTCACHCAAAS | 37 |
| TRBV6for | CGCAGTAGCGGTAAACGGC GAGCCTGGWGTCASYCAGAC | 38 |
| TRBV7for | CGCAGTAGCGGTAAACGGC GGTGCTGGAGTYKCCCAGW | 39 |
| TRBV10for | CGCAGTAGCGGTAAACGGC GATGCTGRRATCACCCAGR | 40 |
| TRBV11for | CGCAGTAGCGGTAAACGGC GAAGCTGAAGTTGCCCAGTC | 41 |
| TRBV13for | CGCAGTAGCGGTAAACGGC GATGCTGGAGTYATCCAGTC | 42 |
| TRBV14for | CGCAGTAGCGGTAAACGGC GAAGCTGGAGTKRYTCAGT | 43 |
| TRBV15for | CGCAGTAGCGGTAAACGGC GATGCCATGGTCATCCAGAA | 44 |
| TRBV18for | CGCAGTAGCGGTAAACGGC AATGCCGGCGTCATGCAGAA | 45 |
| TRBV19for | CGCAGTAGCGGTAAACGGC GATGGTGGAATCACTCAGTC | 46 |
| TRBV20for | CGCAGTAGCGGTAAACGGC AGTGCTGTCRTCTCTCAAMA | 47 |
| TRBV25for | CGCAGTAGCGGTAAACGGC GAAGCTGACATCTACCAGAC | 48 |
| TRBV27for | CGCAGTAGCGGTAAACGGC GATGTGAAAGTRACCCAGARCYC | 49 |
| TRBV30for | CGCAGTAGCGGTAAACGGC ACACTCCAGGCACAGAGATA | 50 |

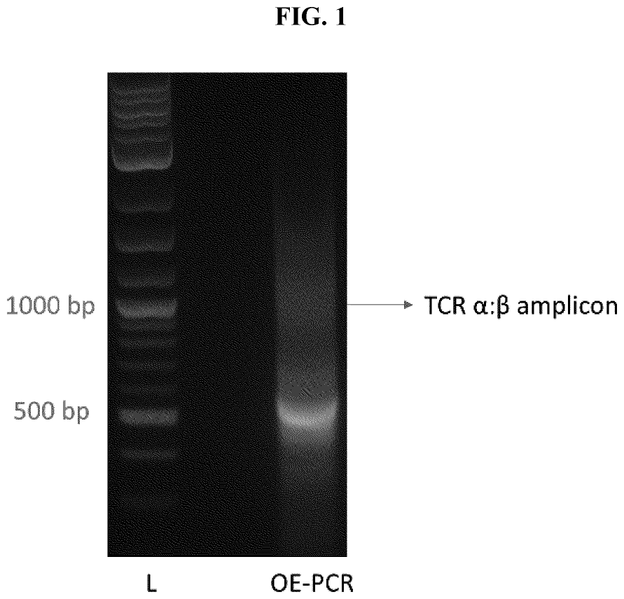

After the OE-RT-PCR reaction, the PCR samples were purified by DNA-cleanup kit and subjected to DNA gel electrophoresis. As shown in FIG. 1, linked TCR alpha and beta amplicons were identified around 1000 b.p., indicating the successful OE-RT-PCR reaction.

After DNA gel electrophoresis, gel extraction was performed to obtain the TCR α:β amplicon. 5 ng purified PCR product was used as a template for performing semi-nested PCR. The nested PCR thermocycling conditions are provided in Table 5.

TABLE 5
Nested PCR thermocycling conditions

| Cycle | Temperature | Time | |
|---|---|---|---|
| 1 | 95° C. | 2 | min |
| 30 | 98° C. | 20 | s |
| | 63° C. | 30 | s |
| | 72° C. | 2 | min |
| 1 | 72° C. | 7 | min |

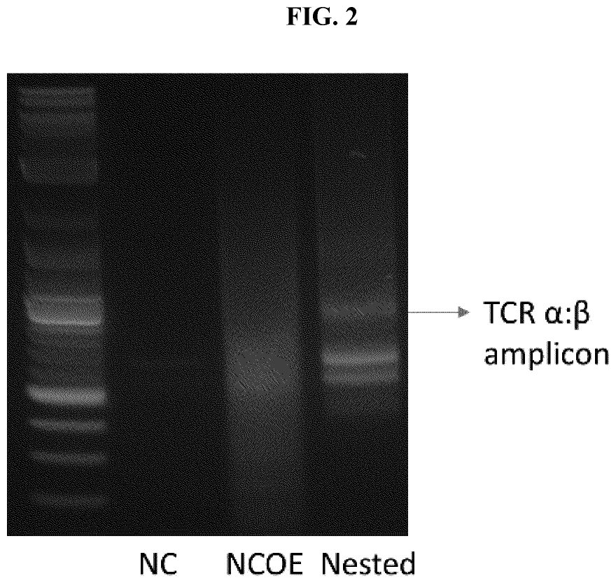

DNA clean-up was performed using Zymo DNA Clean & Concentrator kit (Zymo Research) to purify the PCR products. The purified PCR products were analyzed by DNA electrophoresis. PCR results were showed in FIG. 2, and the band around 1000 b.p. indicated the successful amplification of the TCR α:β amplicon.

After the DNA electrophoresis, the TCR α:β amplicon was excised and gel purification was performed using Zymoclean™ Gel DNA Recovery Kits (Zymo Research). The purified amplicon was subjected to zero-blunt cloning (Thermo Fisher Scientific) to analyze TCR α:β amplicon sequences. The sequences were identified by the NCBI IGBLAST T cell receptor gene database (available at www.ncbi.nlm.nih.gov/igblast/). The sequencing results of the 8 TCR α: amplicons and their respective TCR alpha and beta chain genes are listed in Table 6.

TABLE 6
Gene sequencing results TCR α:β amplicons cloned
into plasmid vectors for single-colony sequencing.

| | TRAV | TRBV |
|---|---|---|
| Amplicon | gene | gene |
| 1 | 26 | 28 |
| 2 | 35 | 4 |
| 3 | 2 | 2 |
| 4 | 21 | 19 |
| 5 | 12 | 11 |
| 6 | 13 | 19 |
| 7 | 12 | 25 |

The sequenced TCR α:β amplicon represented a diverse TCR α:β repertoire from the human PBMC which supported the conclusion that the TCR primer design described in this example can successfully amplify the TCR repertoire. The primer set forth in Table 4 with restriction endonucleases target cleavage sites was incorporated into the amplicon and OE-PCR and nested PCR was successfully performed. The restriction enzymes were chosen to select target cleavage sites which are rarely found (e.g., found in fewer than 5 segments) in T cell receptor genes (including the variable, diversity, joining and constant regions), allowing the expression of original TCR amino acid sequences without losing the TCR library diversity and reducing the TCR library bias due to cleavage during the restriction enzyme digestion and the afterward circulation of expression plasmid process. Several restriction sites were incorporated with silent and non-silent mutations to add additional restriction enzyme cut sites that are rarely found in native TCR genes (Tables 7 and 8).

TABLE 7
Silent mutations used to clone T cell receptors into lentiviral
expression vectors without changing the amino acid sequence of the genes.

| Restriction | Amino acid | Amino acid | TCR gene | Original | Mutated |
|---|---|---|---|---|---|
| enzyme | sequence | sites | name | sequence | sequence |
| Age I | TG | 16-17 | TRBV 10-3 | acagga | accggt |
| Age I | TG | 16-17 | TRBV 15-1 | acaggt | accggt |
| Age I | TG | 16-17 | TRBV 24-1 | acaggg | accggt |
| Age I | TG | 10-11 | TRBV 24-2 | acaggg | accggt |
| Age I | TG | 16-17 | TRBV 24-3 | acaggg | accggt |
| Age I | TG | 16-17 | TRBV 5-8 | acaggc | accggt |
| Age I | GPV | 17-19 | TRBV5-1 | ggcccagta | ggaccggta |
| Age I | GPV | 17-19 | TRBV5-2 | ggcccagtg | ggaccggtg |
| Age I | GPV | 17-19 | TRBV5-3 | ggcccagtg | ggaccggtg |
| Age I | GPV | 17-19 | TRBV5-5 | ggcccagtg | ggaccggtg |
| Age I | GPV | 17-19 | TRBV5-7 | ggcccagtg | ggaccggtg |
| Age I | GPV | 17-19 | TRBV5-8 | ggcccagtg | ggaccggtg |
| Age I | GPV | 17-19 | TRBV6-2 | ggtccagtg | ggaccggtg |
| Age I | GPV | 17-19 | TRBV6-3 | ggtccagtg | ggaccggtg |
| Age I | GPV | 17-19 | TRBV6-4 | ggtccagtg | ggaccggtg |
| Age I | GPV | 17-19 | TRBV6-5 | ggtccagtg | ggaccggtg |
| Age I | GPV | 17-19 | TRBV6-6 | ggtccagtg | ggaccggtg |
| Age I | GPV | 17-19 | TRBV6-8 | ggtcccgtg | ggaccggtg |
| Age I | GPV | 17-19 | TRBV6-9 | ggtcccgtg | ggaccggtg |
| Age I | GPV | 17-19 | TRBV9-1 | ggcccagtg | ggaccggtg |
| Age I | GPV | 17-19 | TRBV9-2 | ggcccagtg | ggaccggtg |
| SpeI | GLV | 17-19 | TRBV 28-1 | ggcctcgta | ggactagta |
| SpeI | GLV | 17-19 | TRBV 5-6 | ggcttagtg | ggactagtg |
| SpeI | TS | 18-19 | TRAV40 | accagc | actagt |
| SpeI | TS | 18-19 | TRAV18 | accagt | actagt |
| XhoI | LE | 16-17 | TRAV38-1 | cttgaa | ctcgag |
| XhoI | LE | 16-17 | TRAV38-2 | cttgaa | ctcgag |
| XhoI | VSR | 18-20 | TRAV13-2 | gtgagcaga | gtctcgaga |
| NheI | QLA | 15-17 | TRAV17-1 | caactggct | caGctAgct |
| NheI | QLA | 13-15 | TRAV27-1 | cagttggca | cagCTAgca |
| BsiWI | RT | 16-17 | TRAV8-5 | agaact | CGTACG |
| BsiWI | RT | 17-18 | TRAV18-5 | aggacc | CGTACG |
| BsiWI | GVR | 15-17 | TRBV30-1 | ggggtcaga | GGCGTACGA |
| BsiWI | GVR | 15-17 | TRBV30-2 | ggggtcaga | GGCGTACGA |
| BsiWI | GVR | 15-17 | TRBV30-3 | ggggtcaga | GGCGTACGA |
| MluI | TR | 18-19 | TRAV8-2 | accaga | ACGCGT |
| MluI | TR | 18-19 | TRAV8-4 | accaga | ACGCGT |
| MluI | TR | 18-19 | TRAV8-6 | accaga | ACGCGT |
| MluI | TR | 17-18 | TRAV8-7 | accaga | ACGCGT |
| MluI | TR | 18-19 | TRAV9-2 | acccgt | ACGCGT |
| MluI | TR | 18-19 | TRAV16-1 | acaaga | ACGCGT |
| SphI | AC | 72-73 | TRAC | gcatgt | gcatgC |
| BstZI7I | VY | 9-10 | TRAC | gtgtac | GTATAC |
| BstBI | DSK | 14-16 | TRAC | GACTCTAA | GATTCGAA |
| SpeI | TLV | 28-29 | TRBC1 | acactggtg | acACTAGTg |
| SpeI | TLV | 28-29 | TRBC2 | acactggtg | acACTAGTg |

TABLE 8
Non-silent mutations used to clone T cell receptors into lentiviral
expression vectors with minimal modifications to native amino acid sequences.

| | Original | | | | | |
|---|---|---|---|---|---|---|
| | amino | Mutated | Amino | TCR | Original | |
| Restriction | acid | amino acid | acid | gene | DNA | Mutated DNA |
| enzyme | sequence | sequence | site | name | sequence | seqence |
| NotI | VAV | AAA | 11-13 | TRBC1 | gtcgctgtg | CGGCCGCGT |
| NotI | VAV | AAA | 11-13 | TRBC2 | gtcgctgtg | CGGCCGCGT |
| SpeI | VAV | LVV | 11-13 | TRBC1 | gtcgctgtg | GAACTAGTC |
| SpeI | VAV | LVV | 11-13 | TRBC2 | gtcgctgtg | GAACTAGTC |
| sphI | VC | AC | 30-31 | TRBC1 | gtgtgc | GcaTGC |
| sphI | VC | AC | 30-31 | TRBC2 | gtgtgc | GcaTGC |
| XhoI | PE | LE | 9-10 | TRBC1 | CCCGAG | CTCGAG |
| XhoI | PE | LE | 9-10 | TRBC2 | CCCGAG | CTCGAG |
| XhoI | FE | LE | 14-15 | TRBC1 | tttgag | CTCGAG |
| XhoI | FE | LE | 14-15 | TRBC2 | tttgag | CTCGAG |
| XhoI | AE | LE | 19-20 | TRBC1 | GCAGAG | CTCGAG |
| XhoI | AE | LE | 19-20 | TRBC2 | GCAGAG | CTCGAG |
| NheI | TLV | TLA | 30 | TRBC1 | acactggtg | acGCTAGCC |
| NheI | TLV | TLA | 30 | TRBC2 | acactggtg | acGCTAGCC |
| NheI | AT | AS | 28 | TRBC1 | GCCACa | GCTAGC |
| NheI | AT | AS | 28 | TRBC2 | GCCACa | GCTAGC |
| NotI | AVA | AAA | 62-64 | TRAC | gctgtggcc | GCGGCCgcc |
| SphI | VC | AC | 22-23 | TRAC | gtctgc | GcaTGC |
| NheI | VS | AS | 34-35 | TRAC | gtgtca | gctagc |

The TCR α:β amplicon was subcloned into a Lentiviral expression vector, pLVX-EF1alpha-IRLS-mCherry (Clontech). Co-transfection of the TCR containing lentiviral expression vector into HLK293T cells along with the lentivirus packaging vector and envelope vector was performed to generate lentiviral particles for TCR expression. HLK293T cells were transfected at the following ratios: 10 μg pLVX, 8 μg psPAX2 packaging plasmid, 3 μg pMID2.g envelope plasmid, 100 μl Fugene transfection reagent. The recombinant lentivirus encoding TCR were transduced into an in vitro display J.RT3 T cell line for the expression of TCR. J.RT3's endogenous TCR alpha and beta genes been knocked out allowing transgenic expression of foreign TCR alpha and beta chain. The transduction protocol was optimized to achieve an MOI ≤0.2 so that the vast majority of cells are only expressing a single TCR paired alpha:beta amplicon. Finally, transduced cells were stained with anti-a chain, anti-P3 chain, anti-CD8, and anti-CD3 antibodies for flow cytometry analyses to determine the fraction of mCherry-expressing cells that display a fully assembled TCR complex ready for functional peptide-MHC binding analysis.

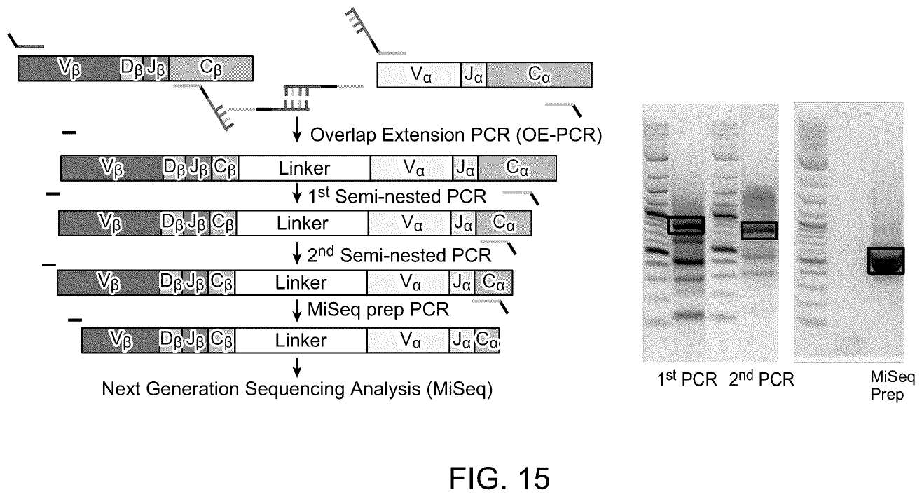

FIG. 15 shows DNA electrophoresis analysis of the T cell receptor (TCR) amplicon (alpha:beta chain) from single-cell emulsification overlap-extension RT-PCR. Briefly, T cells purified from PBMC using CD8 isolation kit were isolated into oil emulsion droplet with lysis buffer and poly(dT) beads to capture mRNA for cell lysis and RNA capture. The poly(dT) beads with T cell RNA were re-emulsified for cDNA synthesis and overlap-extension PCR (OE-PCR). Two semi-nested PCR (1^{st} semi-nested and 2^{nd} semi-nested) were performed to increase the TCR pair α:β chain amplicon concentration. Mi-seq PCR was then performed to add the next-generation sequence barcode for high-throughput sequencing analysis.

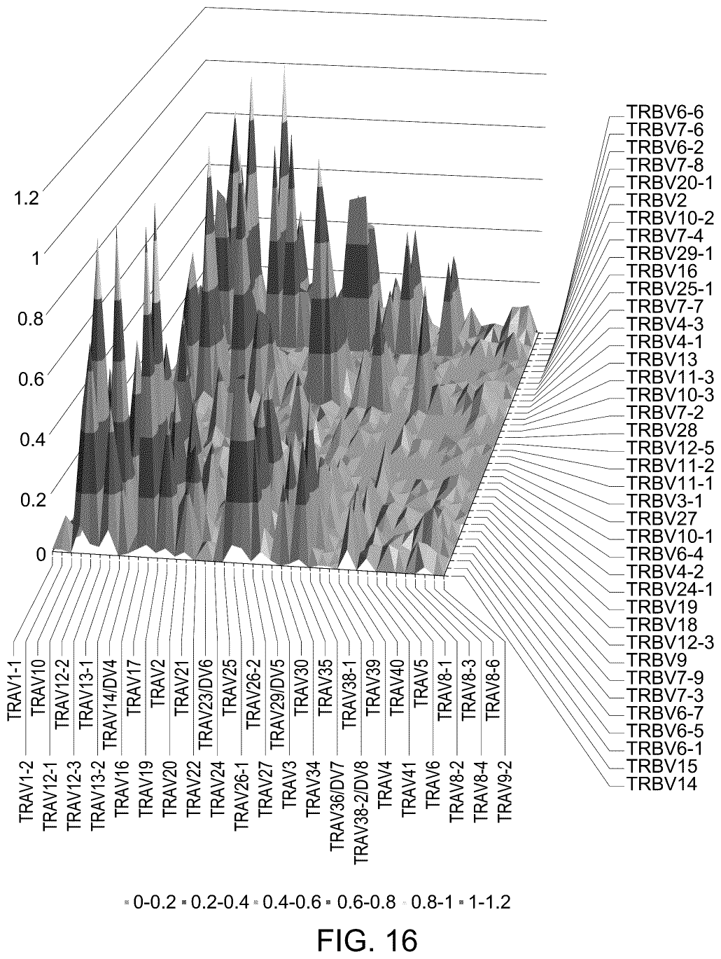

After high-throughput sequencing, raw DNA sequences were quality-filtered and annotated for TCR gene usage via NCBI IgBLAST and a CDR3-motif algorithm, paired by a and R chains, and compiled into a TCR repertoire. FIG. 16 shows a summary of linked alpha:beta T cell receptor gene distribution.

### Example 2: Evaluation of TCR Clones Containing Silent or Non-Silent Mutations

To successfully clone in the TCR genes using restriction enzymes listed herein, the destination cloning vector pLVX-EF1α-IRES-mCherry was modified to remove the following four cutting sites via site-directed mutagenesis: AgeI (2415), SphI (2331), NheI (8192) and MluI (6669) cutting sites (numbers indicate the location of these cutting sites).

A previously identified TCR, anti-HIV-Nef-Rm9, was used as a model TCR for evaluation of the TCR genes with different mutations listed in Table 7 and Table 8. Wild-type anti-HIV-Nef-RM9 TCR gene fragment was subcloned into the Lentiviral vector, pLVX-EF 1α-IRES-mCherry, for expression of active TCR. All anti-HIV-Nef-RM9 mutant clones (Table 7 and Table 8) were introduced by gene synthesis together with restriction enzymes digestion and ligation. Lentiviral expression vectors with Wild-type and mutant anti-HIV-Nef-Rm9 TCRs were transfected separately with envelope and packing plasmid (psPAX2 and pMD2.G) into HEK293FT cell for expression of lentiviruses. An engineered Jurkat cell line that was modified to have no active T cell receptor expression, JRT3/CD8, was used as a TCR expression platform. Lentiviruses encoding wild-type and mutant anti-HIV-Nef-RM9 TCR were transduced separately into JRT3/CD8 cells for TCR expression. The wild-type and mutant anti-HIV-Nef-Rm9 TCRs binding affinity to a HIV peptide, RPQVPLRPM (SEQ ID NO: 1), coupled onto the major histocompatibility complex (MHC) was evaluated. The RPQVPLRPM-MHC (“RPQVPLRPM” is disclosed as SEQ ID NO: 1) complexes were conjugated with Streptavidin-Allophycocyanin (SA-APC) for detection of the binding of TCR to peptide-MHC. In parallel, the transduced JRT3/CD8 cells were stained with anti-TCR antibody conjugated with a BV421 fluorescent marker for evaluation of T cell receptor expression of anti-HIV-Nef-Rm9 with different mutations.

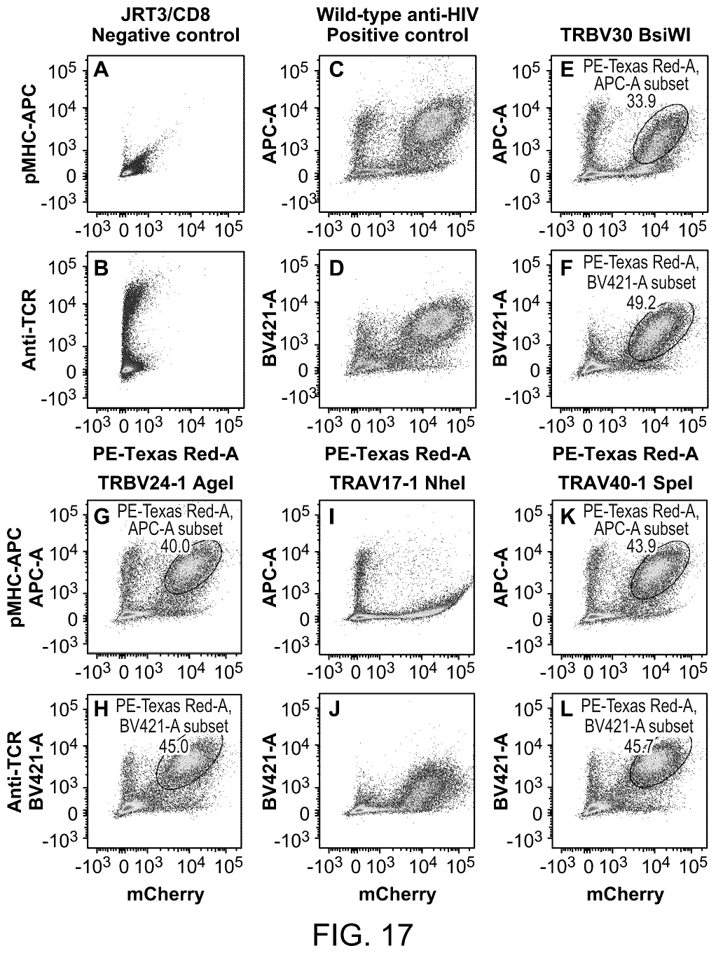

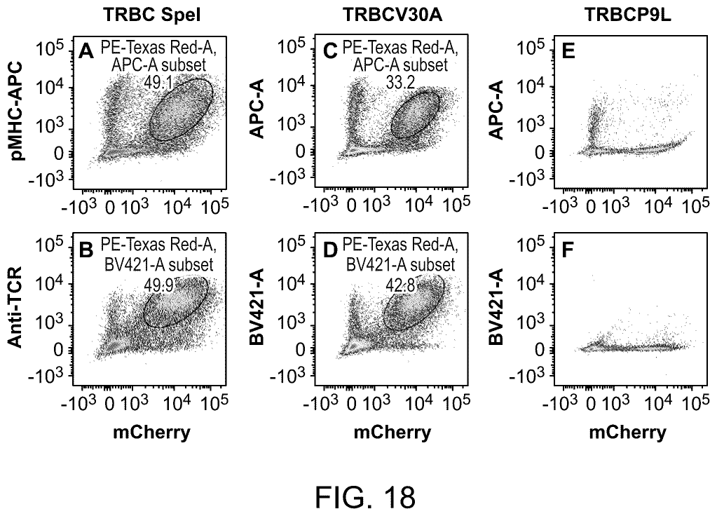

FIG. 17 and FIG. 18 demonstrates that mutant anti-HIV-Nef-Rm9 TCRs with different non-native leader peptides can lead to different levels of TCR expression. Compared with the wild-type anti-HIV-TCR expression level and its binding affinity to the pMHC (FIG. 17 Panel C and D), mutant TCRs including a TRBV24-1 or a TRAV40-1 leader peptide showed comparable levels of TCR expression and pMHC binding affinities (FIG. 17 Panel G and H for TRBV24-1 and Panel K and L for TRAV40-1), whereas the mutant TCR with a TRBV30 leader peptide exhibited reduced TCR expression and pMHC binding capacity (FIG. 17 Panel E and F). Mutant anti-HIV TCR with TRAV17-1 leader peptide demonstrated no pMHC binding tendency and a low level of TCR expression (FIG. 17 Panel I and J). Compared with the wild-type anti-HIV-TCR expression level and its binding affinity to the pMHC (FIG. 17 Panel C and D), TCRs with a silent mutation (for incorporation of a SpeI restriction site) had a comparable level of TCR expression and pMHC binding affinities (FIG. 18 Panel A and B). The mutant TCR with a single amino acid mutation, TRBCV30A, exhibited a similar TCR expression and pMHC binding capacity to those of the wild-type anti-HIV TCR (FIG. 18 Panel C and D). Mutant anti-HIV TCR with a single amino acid mutation, TRBCP9L, demonstrated no pMHC binding tendency and no TCR expression (FIG. 18 Panel E and F).

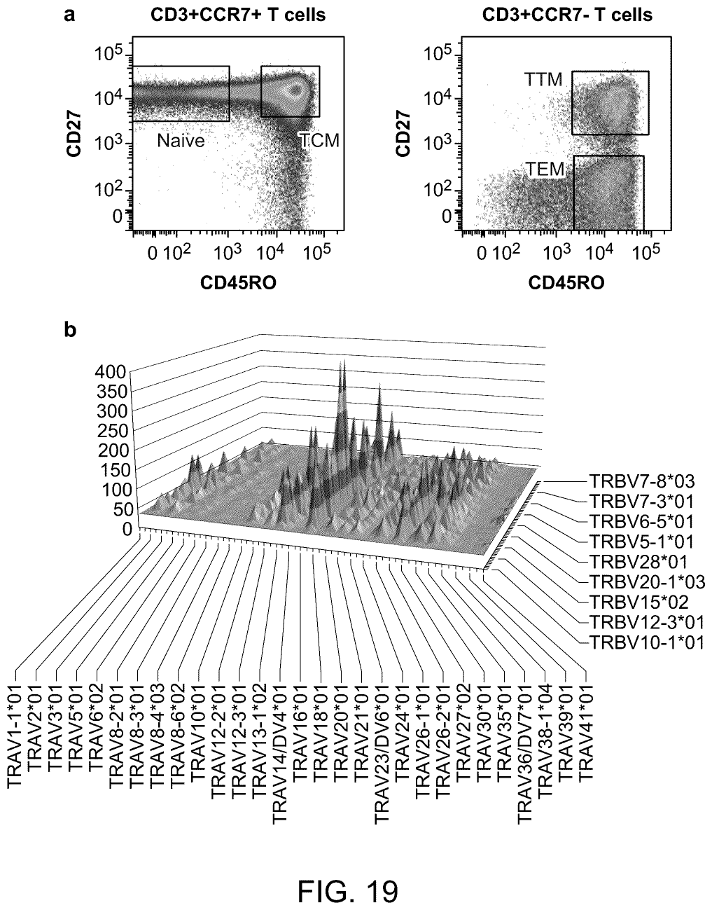

FIG. 19(a) shows isolation of human effector T cells for tumor-specific T cell analysis of humanized mouse models and cancer patients. FIG. 19(b) shows Vα:Vβ gene usage in 31,718 human CD8+ TCR clusters that were isolated, RT-PCR amplified, analyzed via NGS.

### Example 3: Screening Native TCRα:β for Binding to Known Peptide:MHC Combinations

In this example, tetramer staining is performed using recombinant human leukocyte antigen (HLA) tetramer complexes loaded with known Epstein-Barr virus (EBV) peptide. The EBV reactive T cells with TCR on the cell surface will recognize the EBV peptide-HLA tetramer complexes. The HLA complexes were biotinylated and conjugated with streptavidin linked to a fluorophore permitting the sorting of the EBV-reactive T cells. The PBMC are isolated from an Epstein-Barr virus positive donor with known HLA alleles, and emulsion overlap reverse transcription extension PCR (OE-RT-PCR) is performed to isolate the donor's T cell receptor repertoire, as described in Example 1. The TCR amplicons were inserted into the developed TCR expression lentiviral vector constructed in Example 1. After generation of vectors for expressing natively paired T cell receptor libraries, J.RT3 cell lines are transduced with the lentiviral vectors at an MOI of ≤0.2, allowing the expression of TCR α and β chain on in vitro cell libraries.

Several previously characterized EBV TCR-peptide-MHC interactions are assessed (Ref #6-8). In particular, HLA B8 loaded with known immunodominant EBV peptide, HLA-B8-restricted epitope FLRGRAYGL (SEQ ID NO: 79) derived from the latent EBV antigen protein EBNA3A (Ref #9). The transgenic J.RT3 cells' response to an HLA-A*02.01-restricted epitopes from LMP2, a known EBV oncogenic protein, which is thought to have important, potentially protective effects for EBV protection but comprises a more measured EBV response is also tested (Ref #10). The biotinylated HLA tetramer is then conjugated with streptavidin allophycocyanin conjugate from Thermo Fisher Scientific (Catalog #S32362) with a 5:1 molar ratio of MHC monomer to streptavidin as cell sorting marker. The streptavidin-labeled MHC complex is incubated with the lentivirus-transduced J.RT3 cells with recombinant TCR on their cell surface and subjected to fluorescence-activated cell sorting as described in Altman et al (Ref #11).

The sorted J.RT3 cells that express EBV-reactive T cell receptor sequences (α:β chains) are sorted by flow cytometry and recovered in RPMI with 10% FBS overnight. TCR amplicon sequences re then identified by next generation sequencing analysis of the sorted T cell receptor libraries, as previously described (Refs #12-16). Alternatively, the EBV-reactive containing J.RT3 cells are seeded onto 96 well plate with a density of 1 cell/well to isolate single cell clones. Once the single cell is propagated into a colony, the RNA is extracted and the cDNA is synthesized by reverse transcriptase. PCR is performed to amply the TCR alpha:beta pair sequences, as previously described (Refs #17-19). Plasmids are extracted and the sequences are identified by Sanger sequencing.

### Example 4: Screening Native TCRα:β for Activation and EBV-Infected Cell Killing by Cell Lines In Vitro

Naïve T cells are isolated using EasySep™ Human Naïve CD8+ T Cell Isolation Kit (Stemcell Technology) from the PBMC of a healthy human donor. Sleeping Beauty transposon/transposase system is used to deliver the TCRα:β gene sequences as recovered in Examples 1 and 2 into the isolated naïve T cells. The TCR sequence libraries are subcloned in the pLVX vector from Example, 2 along with an EF1alpha promoter for gene expression, into the sleeping beauty transposon cloning vector pT2/BH, which contains the SB transposase (Ref #20). Naïve T cells are transfected with both pT2/BH vector and pCMV(CAT)T7-SB100 (Addgene Plasmid #34879). The pCMAT7-SB100 expressed hyperactive Sleeping Beauty transposase, allowing expression of full-length TCR. Gene delivery transposon plasmids containing the EBV TCR and SB100X transposase plasmids are transfected into T cell populations using a 4DNucleofector according to the manufacturer's instructions (Lonza, Cologne, Germany). In parallel, naïve T cells are transfected with a non-EBV responsive TCR identified in Example 2 as a negative control. Transduced T cell libraries are identified by staining for TCR surface markers that indicated stable TCR complex assembly (anti-α, anti-β, anti-CD8, anti-CD3) and analyzed via flow cytometry. The efficiency of productive TCR display on transduced cells is analyzed.

Next the transfected TCR libraries are analyzed for in vitro function by co-culturing with EBV infected B cells (Raji cells, a cancer cell line which has been tested positive for the presence of EBV), and also for activity against autologous donor B cells infected with EBV. T cells are seeded with EBV positive B cells at a ratio of 10:1 and co-cultured for 24 hours. After 24 hours of in vitro co-culture, the T cells are recovered, stained, and sorted for T cell markers and the expression of CD107/membrane TNF-α upregulation (Ref #21-23). The TCRs they encode are analyzed by NextGen sequence analysis to reveal EBV-targeting tumor infiltrating lymphocytes (TIL) sequences in the repertoire.

Monoclonal paired TCRα:β cDNAs are then recovered, transduced as monoclonal TCRs into naïve human T cells, and assayed by in vitro co-culture with the same populations of EBV-infected B cells to validate the ability of those TCRs to target EBV peptides. The co-culture assay is repeated and pro-inflammatory cytokines are measured using an ELISA kit for measuring IFN gamma, TNF-α, perforin and Granzyme. Compared to a negative control TCR transfected naïve T cells, the naïve T cells expressing EBV-specific TCRs demonstrate increased levels of all pro-inflammatory cytokines.

An in vitro cytotoxicity assay is also performed to evaluate the transformed naïve T cell's activity toward the EBV infected B cells or Raji cells. The Raji cells and the autologous B cells were diluted separately to a concentration of 5×10⁶/mL and incubated with 0.25 μM carboxyfluorescein diacetate succinimidyl ester (CFSE) for 30 minutes at 37° C. CO₂ incubator, allowing the CFSE dye binds covalently to all free amines from cells. The CFSE dye enabled the evaluation of the viability and proliferation of the B cells. Stained B cells are washed three times with the RPMI media. The naïve T cells expressed EBV-reactive TCR alone with control TCR repertoire were then co-cultured with CFSE-stained B cells or Raji cells with a range of effector cell to target cells ratio. Cell cytotoxicity is evaluated by flow cytometry with excitation and emission wavelengths at 492 and 517 respectively. Compared to the naïve T cells in a control TCR experiment (TCR with no EBV peptide affinity), the EBV-reactive TCRs transduced into healthy naïve T cell populations exhibit superior cytotoxicity toward EBV-infected autologous B cells or Raji cells.

### Example 5: Gene Transfer of Native TCRα:β Libraries for Treatment of Cancer

An animal model is used to demonstrate cancer-specific TCR isolation, recovery, and follow-up application of recovered TCR libraries as cell-based therapeutics. Human PBMC-engrafted CD34-NSG humanized mice are implanted with a human RKO colon carcinoma xenograft (Charles River Labs). CD34-NSG were first engrafted with human PBMC by intravenous injection (i.v.) and then engrafted with 1×10⁷ RKO tumors in 50% Martigel® Matrix (Corning) by subcutaneous injection. The mice were then given two checkpoint inhibitors pembrolizumab (anti-PD-1) and ipilimumab (anti-CTLA-4) to enhance the anti-tumor TCR responses. Mice were given 100 μg of Pembrolizumab and 100 μg of Ipilimumab bi-weekly. On day 15, the spleen and tumor tissue was removed from the mice to isolate spleen and tumor-infiltrating T cells. The central memory (TCM) and transitional memory (TTM) T cells were obtained by FACS analysis for CD3+CD8+CD45RA CCR7 expression. TCM and TTM populations were then expanded and subjected to emulsion OE-PCR and nested PCR to obtain the TCR repertoire, as in Examples 1 and 2, and cloned into the Sleeping Beauty transposon/transposase transduction plasmids as described in Example 3.

The Illumina MiSeq 2×300 paired-end read platform was used to precisely define and determine the molecular features of the TCR libraries. Raw Illumina sequences were quality-filtered, mapped to V-, D-, and J-genes and CDR3's extracted using the International Immunogenetics Information System (IMGT, Ref #24). Sequence data is filtered for in-frame V(D)J junctions and productive TCRα and TCRβ sequences are paired by Illumina read ID and compiled by exact CDR3 nucleotide and V(D)J gene usage match. CDR-03 nucleotide sequences were extracted and clustered to 96% nt identity with terminal gaps ignored (USEARCH v5.2.32, Ref #25) and resulting Vα:Vβ pairs with ≥2 reads comprised the list of Vα:Vβ clusters.

Alternatively, after performing the emulsion RT-PCR and nested PCR, the TCR α:β amplicon library can be ligated into sleeping beauty transposons vector(pT2/BH) and transformed into high efficacy competent E. coli cells such as XL-Gold (Agilent #200314) to clone the highly diverse TCR library. Paired alpha beta TCRs are then sequenced individually by bacterial colony Sanger sequencing. Approximately 100 colonies are combined with known TCR sequences to generate a precisely defined paired alpha beta TCR library for delivery into the naïve T cells. A TCR repertoire acquired from the TCM and TTM T cells derived from humanized mice without the engraft of RKO cancer cell line is used as a negative control group.

Next both TCR repertoires (derived from RKO xenograft TCR, or from mice without RKO xenograft TCR) are expressed in naïve T cells using the Sleeping Beauty transposon/transposase system as reported in Example 3. The transformed TCR libraries are separately administered to different RKO xenograft mice via i.v. injection. Tumor volume and body weight change of individual mouse is measured on a daily basis to track the tumor size and progression of disease. The RKO-xenograft mice treated with T cell populations transduced with TILs from other RKO-xenograft mice exhibit a delay in tumor growth as compared to mice treated with T cells transduced with TCR libraries from the control group (i.e., derived from human PBMC CD34-engrafted mice with no RKO xenograft). This demonstrates the applicability of precisely defined TCR libraries as cell-based therapeutics that could effectively reduce tumor progression.

### Example 6: Gene Transfer of Native TCRα:β Libraries after Pre-Selection for Anti-Cancer Activity for Treatment of Cancer

Similar procedures as Example 4 and the same mouse model (human PBMC-engrafted CD34-NSG humanized mice with RKO colon cancer xenograft model) is used in this example, but with an additional T cell pre-selection step to isolate anti-cancer TCRs by in vitro selection prior to use as a cell-based therapeutic. The splenocyte and tumor-infiltrating lymphocyte populations are isolated after the RKO colon cancer engraft. TCM and TTM are isolated by FACS with the markers CD3+CD8+CD45RA and CCR7. The TCR repertoire is isolated using emulsion OE-PCR, and nested PCR, and then subcloned into the Sleeping Beauty transposon vector and redelivered into the naïve T cells isolated from PBMC as described in Example 4. The transgenic naïve T cells are stimulated by co-culture with irradiated RKO colon cancer cell lines and isolated using flow cytometry for CD107 and membrane TNF-α, as described in Example 3.

Alternatively, dendritic cells (DCs) are isolated from the humanized mice using the blood dendritic cell isolation kit II (Miltenyi Biotec) and DCs are cultured in RPMI with 10% FBS supplemented with 50 ng/mL of granulocyte-macrophage colony-stimulating factor (GM-CSF). The dendritic cells are pulsed with RKO cancer cell lysates or human colon cancer cell lysates for two hours. Naïve T cells transduced with the TCR libraries that had been cloned into Sleeping Beauty transposon/transposase vectors are then co-cultured with the DC cells with a ratio of 10:1 for 4 days. The stimulated anti-cancer cytotoxic T cells are gated with membrane-bound CD107, membrane-bound TNF-α expression or CD137 (4-1BB) expression. CD107a is a marker for degranulation of activated CD8+ T cells (Refs #26-33). CD137 belongs to the TNFR family and is associated with T cells proliferation and survival (Ref #34-35). The sorted T cells (anti-cancer T cells) are delivered into the RKO colon cancer mouse model as described in Example 4. Compared to mice treated with naïve T cells transduced with TCR from the non-RKO xenograft mice, the mice treated with the naïve T cells transduced with RKO-targeting TCRs isolated from in vitro functional screening exhibited a higher survival rate and reduced tumor growth. The T cell are selected for activation in the anti-cancer co-culture assays and enriched for cancer-specific TCRs to increase the anti-cancer efficacy of the final cell-based therapeutics.

### Example 7: Gene Transfer of Native TCRα:β Libraries after Pre-Selection for Anti-Cancer Activity and In Vitro Pre-Activation to Enhance Anti-Tumor Cell Killing

The experiment procedures in Example 6 are performed as described in Example 5, with an additional T cell activation step after transduction of natively paired TCR libraries into naïve T cells. This activation step pre-primes the T cells and activates them for enhanced cancer cell killing. Briefly, after the OE-PCR and first nested PCR, the TCR libraries are cloned into the sleeping beauty transposon vector for transgenic expression of TCR. Next generation sequencing or Sanger sequencing of individual plasmids is performed to define TCR library size and diversity. The pooled TCR transposon plasmids are co-transfected with sleeping beauty transposase expression vector SB100 permitting the expression of TCR in naïve T cells. The transgenic T cells are first stimulated with RKO cell line or cancer antigen pulsed T cells and CD137/CD107/TFN-α expressing anti-cancer T cells are sorted by FACS. These cells are then activated in vitro prior to delivery as cell-based therapeutics into the RKO tumor mouse model. Gibco™ Dynabeads™ Human T-Activator CD3/CD28 (Thermo Fisher Scientific) are added to the TCR-transduced naïve T cell cultures at a final ratio of 1:1 of beads to cells and incubated in 37° C. CO₂ incubator for 3 days. The Dynabeads are conjugated with antiCD3 and antiCD28 antibody mimicking the in vivo interaction of T cell with antigen presenting cells (APC), allowing T cell clonal expansion and differentiation (Ref #36). Cell growth and viability are monitored daily after activation.

The expression of IFN-gamma and TNF-alpha is examined by ELISA, which indicates the differentiation of T cells. At day 3 post-bead addition, 20 U/mL of recombinant human interlukin2 (IL2) is added into the culture media to induce further T cell expansion. At day 7, the expanded T cells are counted and delivered into the RKO xenotransplant mice via tail vein i.v. injection. Tumor growth and individual mouse body weight was evaluated and compared to those obtained in Examples 4 and 5. RKO-engrafted mice treated with transgenic, RKO tumor-specific, stimulated, and activated T cells in this example exhibit superior anti-cancer efficiency as compared to the those from Example 4 (transgenic T cells without tumor selection or activation) and Example 5 (T cells screened for reactivity to RKO without in vitro pre-activation). The additional activation step described in this Example permits the differentiation of effector cytotoxic T cells prior to therapeutic delivery to enhance the speed and intensity of tumor immunosuppression in the mouse model.

### Example 8: Gene Transfer of Native TCRα:β Libraries for the Induction of Antigen-Specific Immune Tolerance

The CD34+ human PBMC-engrafted mouse model is known to induce Graft-versus-host disease (GvHD) in the mice. To alleviate this issue, regulatory T cells with TCRs isolated from mice suffering from GvHD were induced and used as therapeutics to induce immune tolerance.

T cells are isolated from the spleen and PBMC of mice that have been prepared as described in Example 4. T cells are isolated at 30 days post-engraftment, when GvHD onset begins. The TCR cloning primer set from the Example 1 is employed to perform overlap extension reverse transcriptase PCR to obtain TCR libraries from both the Treg cells (CD4+CD25+) as well as conventional T cells (CD4+CD25−). Next, the two TCR libraries are subcloned into thepT2/BH Sleeping Beauty transposon vector. Next generation sequencing is performed to analyze these two sets of T cells receptor libraries and to characterize the T cell receptor gene usage.

Naïve T cells are isolated from the PBMC with the following sorting marker setting: CD25-CD44^{low}CD62L^{hi}. The cells are transfected with TCR donor transposon plasmid containing two libraries described above along with Sleeping Beauty transposase plasmid (SB100) for TCR expression, using the TCR sequence libraries that had been isolated from GvHD mice. The transgenic naïve T cells are then induced to T reg cells by Dynabeads™ Human T-Activator CD3/CD28 along with 5 ng/mL of TGF-β1. TGF-β, a master regulator which has been shown to induce Foxp3 expression, allowing differentiation of naïve T cell into regulatory T cells (Ref #37-38). After 5 days of induction, the induced T regulatory cells (iTreg) are supplied with rIL-2 enabling T cell expansion for treatment of GvHD onset mice.

Alternatively, T cells are transduced directly with Foxp3 sequences as described previously (Ref #39-40). After 3 days post-expansion, the iTreg cells with transgenic TCR and without transgenic TCR are injected separately into the GvHD onset mice intravenously via tail vein injection (n=5); these mice had been engrafted with CD34+ human PBMCs from the same donor and time point of sampling as the earlier GvHD mice. The mice treated with iTregs with CD4+CD25+ transgenic TCR exhibit delayed GvHD disease onset as compared with those from the iTreg cells with transgenic CD4+CD25-TCR and iTreg without transgenic TCR.

Body weight changes and GvHD scores between the three groups (no transgenic TCR, CD4+CD25+ TCR and CD4+CD25-TCR) are analyzed. The mice injected with T cell expressing CD4+CD25+ transgenic TCR showed the lowest change in body weight and lowest GvHD score as compared with the mice infused with T cells only and T cells with CD4+CD25− transgenic TCR. Tolerogenic activities and T cell phenotype are monitored. 20 days post-T cell infusion, the splenocytes are isolated by sacrificing the humanized mice, harvesting spleen, and red blood cells lysis buffer digestion. The resultant leukocytes are stained with anti-CD25, anti-CD4+, anti-CD3 and Foxp3, and followed by flow cytometry analysis to evaluate the tolerogenic activities. The leukocytes from the mice with transgenic CD4+CD25+ TCR exhibit the most intense Foxp3 and CD25 signals from the flow cytometric staining analyses as compared with the leukocytes from the other two groups of mice (with no transgenic TCR and CD4+CD25-TCR).

### Example 9: Application of Native TCRα:β Library Gene Transfer for Cancer Therapy in Mammalian or Human Patients

In this example, a mammalian patient such as a human cancer patient is treated. In some embodiments, the cancer type is lung cancer, melanoma, renal cell carcinoma, breast cancer (including triple negative breast cancer), colon cancer, or prostate cancer. The therapeutic procedures may be performed similarly as described in Example 4. Briefly, T cells from a mammalian or human patient are recovered, which may derive from PBMC, tumor infiltrating lymphocytes, spleen tissue, affected organs, or other human tissue from the patient. Then, the TCR genes are recovered and cloned into expression vectors. In some embodiments, the T cell libraries are first pre-screened for anti-tumor activity. The screening can identify TCRs with reactivity against tumor peptide neoantigens, or alternatively reactivity against whole tumor cells in vitro. In some embodiments, the tumor cells may be derived from the cancer patient.

Next, the selected T cell libraries can be used as cell-based therapeutics. In some embodiments, the libraries are analyzed using high-throughput sequencing to precisely define the molecular composition of the cell-based TCR therapeutics. In other embodiments, the libraries are selected by sub-sampling a number of individual plasmid colonies in bacteria (ranging from 3 to 100,000 colonies derived from the library) and sequencing each plasmid colony individually after mini-prep. Then, those individual plasmids are mixed at a defined ratio to regenerate a precisely defined molecular library. These precisely defined molecular libraries can reduce the presence of PCR error variants that can occur when TCR libraries are originally generated by RT-PCR.

In some embodiments, mouse TCR constant region genes will be used to prevent TCR transgenes from associating with native human T cell receptor genes in gene recipient T cells. The libraries will be used to transfer the TCRs and any other plasmid genes to human T cells. In some embodiments, these T cells are derived from the patient. In some embodiments, these T cells will be patient-derived naïve T cells. In other embodiments, these T cells may be derived from other humans or from cell lines. In some embodiments, there may be an activation step to pre-prime the T cells and activate them for enhanced cancer cell killing as described in Example 6.

In some embodiments, transcription factors may be used to affect cell fate, as described in Example 7. The transformed T cells expressing the TCR transgene may be expanded in vitro to generate a cell bank, or they may be directly administered to the patient. In some embodiments, the T cells are injected directly into the tumor. In other embodiments, the T cells are administered intravenously or intrathecally.

The plasmid gene libraries can used again at any time to re-create a population of T cell transgenes for subsequent repeat administration; in other embodiments, the expanded cell banks may be used for repeat therapeutic administration. In the event of continued cancer progression and/or tumor growth, or an inadequate treatment of the tumor in any way, the entire therapeutic process can be repeated. In some embodiments, this may comprise capture the patient's T cell genes again, and repeating the screening and library generation process. In other embodiments, a repeat therapy could comprise re-screening the originally captured TCR gene libraries against a resistant tumor cell population to identify TCRs that target the evolved cancer cells. In other embodiments, a repeat therapy may comprise additional activation of the TCR gene libraries. In some embodiments, the in vitro cell activation process may change over the course of multiple treatment administrations, for example, as cancer progresses then more potently activating steps may be used for the cell-based therapeutics in vitro prior to therapeutic administration.

In some embodiments, an i-Caspase gene or other inducible suicide switch gene may be included in the transgene vectors to control the fate of the cell-based therapy after therapeutic administration.

### Example 10: Application of Native TCRα:β Library Gene Transfer for Treatment of Viral Infections

Posttransplant lymphoproliferative disorder (PTLD) is a severe complication of solid organ transplantation. Primary Epstein-Barr virus (EBV) infection is a major risk factor, and around 60-80% of PTLD cases are EBV seropositive. T cell responses to EBV peptides are crucial to suppress malignancy (Llaurador, G. et al., Curr. Opin. Pediatr. 29, 34-40 (2017)).

The TCRα:β library disclosed herein will be used to treat or prevent PTLD in mouse models as a model for antiviral TCR therapeutics (Ahmed, E. H. & Baiocchi, R. A, ILAR J. 57, 55-62 (2016); Ricciardelli, I. et al., Blood 124, 2514-2522 (2014)). Briefly, anti-EBV TCRs will be identified by single-cell isolation of paired alpha and beta genes, and will be cloned into TCR display vectors, and cell-based screening by FACS for anti-EBV peptide binding. In other embodiments, the anti-EBV TCRs may be discovered by identifying recognition, expansion, and/or in vitro killing activity of T cells transformed with transgenic TCRs. In other embodiments, T cell receptors are discovered that target other viruses to different antiviral therapies, including but not limited to human cytomegalovirus (HCMV), herpes simplex virus 1 or 2 (HSV-1/HSV-2), and yellow fever virus (YFV).

After the antiviral T cell receptor polynucleotides have been isolated, they will be cloned into DNA vectors and utilized for autologous gene therapy to treat PTLD. The transformed T cells might be used to treat other viral infections or for heterologous gene therapy. In other embodiments, they may be used as prophylactic cell therapy to prevent PTLD or other viral infections. In other embodiments, the vectors may be used for gene therapy as virally-associated preventive cancer vaccines.

Lentiviral gene transduction will be used to transform human pan-T cells with antiviral TCRs to evaluate in vitro killing efficacy of virally infected cells. Transformed T cells may be co-cultured with virally infected cells with any of the following stimulation conditions: none, IL-2, anti-CD3/anti-CD28 magnetic beads, or IL-2/anti-CD3/anti-CD28. In vitro virally infected cell killing will be assessed by the IncuCyte Live Cell assay, which measures the loss of fluorescently-labeled tumor cells due to cell killing (Single, A. et al., J. Biomol. Screen. 20, 1286-1293 (2015)).

Next, human pan-T cells will be transduced with antiviral TCRs under the optimized conditions and used to treat viral infections in mouse models. A mouse model of EBV infection and PTLD will be used (Johannessen, I. et al. J. Med. Virol. 83, 1585-1596 (2011)). In other embodiments, mice may be infected with a virus and treated with T cells specific to viral proteins. Transduced T cells will be isolated by flow cytometry for transgenic TCR expression to obtain pure transduced TCR libraries. Next, transformed cells will be stimulated using a T cell stimulation protocol, which could include the conditions described above, and stimulated T cells will be delivered to mice via intravenous tail vein injection (Kurtz, A. Mesenchymal Stem Cell Delivery Routes and Fate. Int. J. Stem Cells 1, 1-7 (2008)).

Mice will be followed for 30 days to record weight loss and PTLD tumor volume. Treated mice will be compared to non-treated mouse controls, as well as other controls treated with antiviral antibodies and antiviral small molecule inhibitors. Any delays in PTLD tumor growth will be quantified as the major study endpoint. It is expected that mice treated with transformed cells including anti-EBV TCRs of the present technology will show a delay in PTLD growth, delayed viral growth kinetics and/or faster disease recovery after viral infections compared to untreated controls. These results will demonstrate that the compositions of the present technology are useful in methods for treating or preventing viral infections and virally-associated cancers in a subject in need thereof.

### Example 11: Evaluation of Antigen-Specific TCRαβ in Treating Cancer in PDX Mouse Models

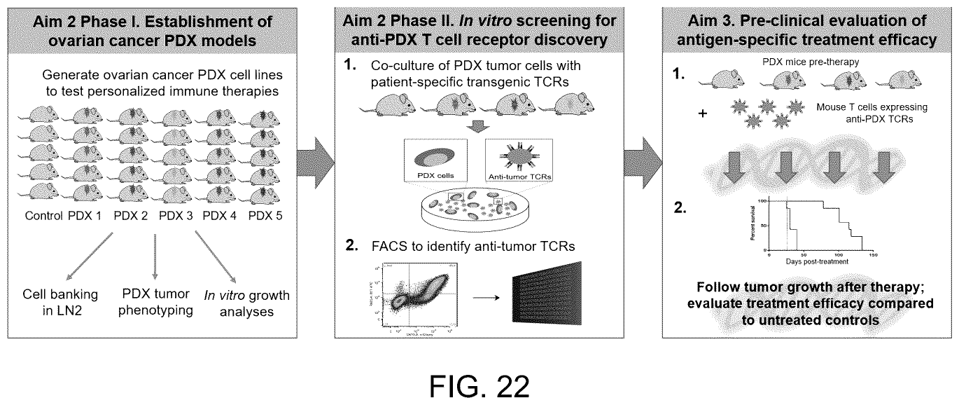

Patient-derived xenograft (PDX) mouse models from ovarian cancer patients will be established as a renewable cell source for anti-tumor T cell discovery (FIG. 22).

Human pan-T cells are transduced at a multiplicity of infection of less than 0.2 (i.e., one TCR transgene per cell) and transduced libraries are screened in vitro for TCR activation against co-cultured PDX tumor cells. Expansion and activator gene expression (CD69/CD107/membrane TNF-α) are quantified as hallmarks of anti-tumor TCR recognition. Illumina MiSeq 2×300 paired-end sequencing is utilized to analyze TCR library diversity and characterize V3 and Va genes at each step in the cloning and selection process (i.e., input cDNA, post-lentiviral particle generation, and mCherry+J.RT3 cells with surface-displayed TCR). 2×300 sequencing permits full coverage of CDRα3 and CDRβ3 regions. High-throughput sequencing will verify that TCR libraries maintain high diversity, and ≥10⁵ native TCRα:βs displayed on mammalian T cells will be generated. NGS will also be used to identify PDX neoantigens and TCR molecular modeling will be performed to determine the neoantigen targets of anti-PDX TCRs.

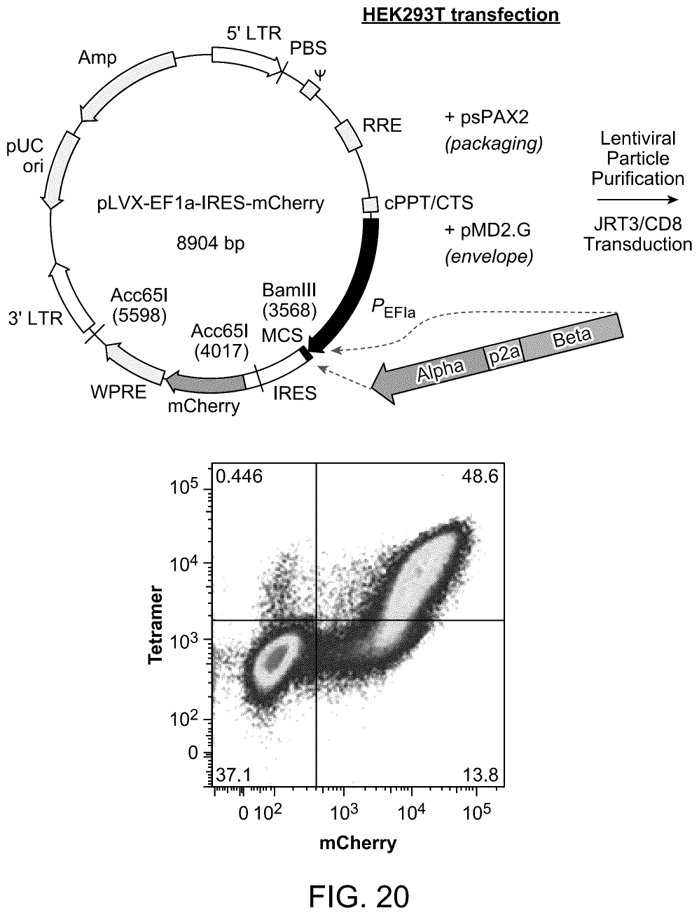

Once anti-PDX TCRs are identified by screening, autologous patient T cells will be transduced with those anti-tumor TCRs and their ability to kill PDX tumor cells in vitro will be assayed. Human T cells will be transfected with anti-tumor TCRα:β genes via lentiviral gene transduction (FIG. 20). Patient T cells are transduced with native human TCR constant region genes, including a cysteine modification to promote preferential expression of the transduced TCR. Transduced T cells are pre-activated with T cell stimulatory agents (as one example, anti-CD3/anti-CD28 magnetic beads/IL-2) and are incubated with IncuCyte-labeled tumor cells to assess TCR-based tumor cell killing in vitro. Autologous T cells that are transduced with linked TCRα:β genes that do not target tumor cells will serve as negative controls. ANOVA will be used for statistical analyses of tumor cell killing compared to negative control TCR genes.

Next, transgenic anti-PDX T cells will be delivered in PDX mice for antigen-specific tumor immunotherapy. Transduced T cells are isolated by flow cytometry to obtain pure transduced TCR libraries. Mouse pan-T cells are transduced with anti-PDX TCRs and pre-stimulated using the conditions determined in cell-killing assays; stimulated T cells are delivered to mice via intravenous tail vein injection. Mice are followed for 30 days and treated mice are compared to control groups and animals treated with checkpoint inhibitors. Delayed tumor growth is quantified as the major study endpoint, applying Kaplan-Meier survival analysis of survival to verify statistical significance. It is anticipated that animals treated with T cells comprising anti-PDX TCRs will match or exceed the therapeutic responses observed in animals receiving checkpoint immune therapy treatments. These results will demonstrate that the TCR libraries of the present technology are useful for personalized antigen-specific therapies in human patients.

### Example 12: Evaluation of Antigen-Specific TCRα:β in Treating Cancer in PDX Mouse Models

Renal cell carcinoma (RCC) is susceptible to immunotherapy and is typically clinically silent until the tumor is locally advanced or metastatic. Thus, late stage RCC diagnosis is common and surgical resection yields large tumors with substantial T cell infiltrates for laboratory study. Advanced RCC is also an FDA-approved indication for checkpoint inhibition and recombinant IL-2 therapies, and RCC can be highly immunogenic with a large T-cell infiltrate that can be reinvigorated with immunotherapy. High-throughput screening of TIL TCRs responding to RCC will be used to identify neoantigen-specific TCRs that can be leveraged for precision therapies.

First, TILs and cancer cells are isolated from tumors using flow cytometry; tumor cells are cryopreserved while TILs are the input for TCR repertoire isolation and cloning. Next, TCR repertoires are transformed into naïve T cells and the resulting display library is seeded in co-culture with autologous patient tumor cells. After 24 hours of co-culture, activated T cells are sorted by FACS for CD69/CD107/membrane TNF-α upregulation, and the encoded TCRs are analyzed by NextGen sequencing to reveal the tumor-targeting TCRs in the repertoire. Finally, monoclonal paired TCRα:β cDNAs are recovered, transduced into human pan-T cells, and assayed by tumor cell co-culture in order to identify anti-RCC TCRs. These results will demonstrate that the TCR libraries of the present technology are useful for personalized antigen-specific therapies in human patients.

### Example 13: Application of Paired TCRα: Gene Therapy to Treat Colon Carcinoma, Ovarian Cancer, and B Cell Cancers

Colon cancer. Paired TCRα:β gene libraries against RKO cells (a human colon carcinoma cell line) are cloned into mouse T cells. Briefly, pan-T cells will be isolated from mouse spleens using magnetic beads and subjected to either lentiviral or transposase-based TCR gene delivery.

According to the lentiviral strategy illustrated in FIG. 20, human TCR constant region genes are used to prevent cross-dimerization with the native mouse TCRs. Next, T cells are stained with antibodies to evaluate surface expression of transgenic human a and p constant region proteins, and TCR expression is quantified by flow cytometry. Lentiviral gene transduction is used to transform mouse pan-T cells with anti-RKO TCRs to evaluate RKO killing efficacy. Transduced T cells are isolated by flow cytometry for human TCR expression to obtain pure transduced TCR libraries. Transformed cells may be co-cultured with RKO tumor cells with any of the following stimulation conditions: none, IL-2, anti-CD3/anti-CD28 magnetic beads, or IL-2/anti-CD3/anti-CD28. In vitro RKO cell killing was assessed by the IncuCyte Live Cell assay, which measures the loss of fluorescently-labeled tumor cells due to cell killing.

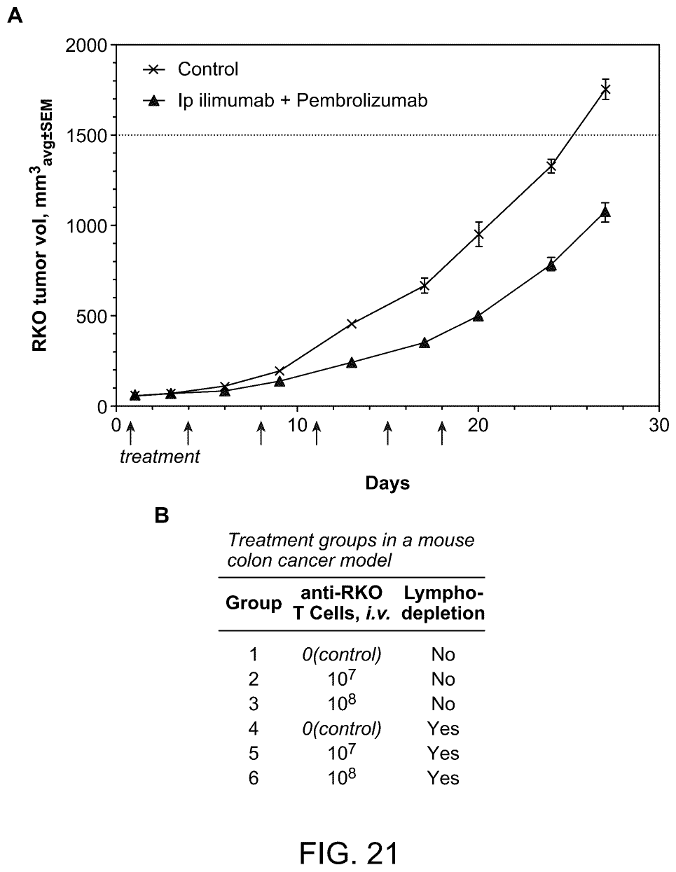

For in vivo studies, stimulated T cells are delivered to mice via intravenous tail vein injection. Mice are followed for 30 days to record weight loss and tumor volume. Treated mice are compared to non-treated mouse controls (Groups 1 and 4, FIG. 21(b)), as well as historical controls treated with currently approved antibody-based immunotherapies (FIG. 21(a)). Delays in tumor growth are quantified as a major study endpoint. Animals treated with T cells comprising anti-RKO TCRs are expected to match or exceed the delay in tumor growth observed in animals treated with antibody-based immunotherapies.

Ovarian Cancer. Human clinical PBMC and TIL samples are collected from excised tumors and/or patient blood. TCRα:β libraries from ovarian cancer patients are isolated. Clinical PBMC and/or TIL samples are obtained following surgical resection and/or blood draws. TCRα:β cloning is performed using a green fluorescent protein (GFP) reporter system for retroviral TCR transduction into J.RT3 T cells. J.RT3 cells do not express an endogenous TCR and provide stabilizing proteins and co-expression factors (including CD3 and CD8) for TCR expression, making J.RT3 an ideal mammalian host for TCR display. Natively paired α:β variable region genes are cloned into the pLVX lentiviral expression vector using restriction sites at the ends of the linked amplicon. Vector libraries are amplified in E. coli. and TCRα:β expression libraries are co-transfected into HEK293T cells along with packaging and envelope vectors (psPax2 and pMD2.g) to generate lentiviral TCRα:β transduction particles (FIG. 20).

B cell cancers. B cell lineage cancers include lymphoma and multiple myeloma that express antibody genes as tumor-specific neoantigens that are targeted by TCRs. Paired TCRα:β gene libraries are isolated from B cell lineage cancer patients. Paired TCRα:β cloning is performed using a green fluorescent protein (GFP) reporter system for retroviral transduction of TCR expression plasmids into J.RT3 T cells. J.RT3 cells do not express an endogenous TCR and also provide the stabilizing proteins and co-expression factors (including CD3 and CD8) needed for stable TCR expression, making J.RT3 a suitable mammalian host for TCR library display. Natively paired α:β variable region genes are cloned into the pLVX lentiviral expression vector using restriction sites at the ends of the linked amplicon (as shown in FIG. 20).

Vector libraries are amplified in E. coli. using strains designed for stable lentiviral plasmid replication (e.g., Stbl3¹²), and then TCRα:β expression libraries are co-transfected into HEK293T cells along with packaging and envelope vectors (psPax2 and pMD2.g) to generate lentiviral TCRα:β transduction particles. In parallel, tumor-specific BCRs are sequenced from PBMC clinical samples. J.RT3 T cells are transduced at a multiplicity of infection (MOI) of less than 0.2 (i.e., one TCR transgene per cell) to assay TCR binding to BCR neoantigens. Transduced cells will be screened against overlapping peptide-MHC from CDR-H3−, CDR-L3−, and somatic mutation-derived neoantigens from the patient-sequenced tumor-encoded antibody genes.

The Illumina MiSeq 2×300 paired-end NGS system is used to analyze library diversity and characterize V3 and Va libraries at each step in the cloning process (i.e., input cDNA, post-lentiviral particle generation, and mCherry+J.RT3/CD8 cells with surface-displayed TCR). 2×300 sequencing permits full coverage of CDRα3 and CDRβ3 regions. High-throughput sequencing will verify that TCR libraries maintain high diversity throughout the library cloning process and that ≥10⁶ native TCRα: s displayed on mammalian cells will be generated. TCRα:βs discovered by peptide:MHC neoantigen screening are tested for affinity using surface plasmon resonance (SPR). These results will demonstrate that the TCR libraries of the present technology are useful for personalized antigen-specific therapies in human patients.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. DeKosky, B. Decoding the Antibody Repertoire: High Throughput Sequencing of Multiple Transcripts from Single B Cells. (Springer, 2017).
2. Hunicke-Smith, S., Dekosky, B., Ellington, A. & Georgiou, G. High throughput sequencing of multiple transcripts of a single cell. (2015), US20150141261A1.
3. McDaniel, J. R., DeKosky, B. J., Tanno, H., Ellington, A. D. & Georgiou, G. Ultra-high-throughput sequencing of the immune receptor repertoire from millions of lymphocytes. Nat Protoc 11, 429-42 (2016).
4. DeKosky, B. J., Kojima, T., Rodin, A., Charab, W., Ippolito, G. C., Ellington, A. D. & Georgiou, G. In-depth determination and analysis of the human paired heavy- and light-chain antibody repertoire. Nat Med 21, 86-91 (2015).
5. Primer sets for cloning the human repertoire of T cell Receptor Variable regions Ilenia Boria doi: 10.1186/1471-2172-9-50
6. Catalina, M. D., Sullivan, J. L., Brody, R. M. & Luzuriaga, K. Phenotypic and Functional Heterogeneity of EBV Epitope-Specific CD8+ T Cells. J. Immunol. 168, 4184-4191 (2002).
7. Hislop, A. D. & Taylor, G. S. in Epstein Barr Virus Volume 2 (ed. Münz, C.) 391, 325-353 (Springer International Publishing, 2015).
8. Ibisch, C., Saulquin, X., Gallot, G., Vivien, R., Ferrand, C., Tiberghien, P., Houssaint, E. & Vid, H. The TCR repertoire Selected In vitro Against EBV: Diversity, Specificity, and Improved Purification Through Early IL-2 Receptor α-Chain (CD25)-Positive Selection. J. Immunol. 164, 4924-4932 (2000).
9. J Immunol Jan. 1, 2012, 188 (1) 311-321; DOI: doi.org/10.4049/jimmunol.1102686
10. Jones, K., Wockner, L., Brennan, R. M., Keane, C., Chattopadhyay, P. K., Roederer, M., Price, D. A., Cole, D. K., Hassan, B., Beck, K., Gottlieb, D., Ritchie, D. S., Seymour, J. F., Vari, F., Crooks, P., Burrows, S. R. & Gandhi, M. K. The impact of HLA class I and EBV latency-II antigen-specific CD8+ T cells on the pathogenesis of EBV+ Hodgkin lymphoma. Clin. Exp. Immunol. 183, 206-220 (2016).
11. Science. 1996 Oct. 4; 274(5284):94-6. Phenotypic analysis of antigen-specific T lymphocytes. Altman JD1, Moss P A, Goulder P J, Barouch D H, McHeyzer-Williams M G, Bell J I, McMichael A J, Davis M M.
12. Golding, A., Darko, S., Wylie, W. H., Douek, D. C. & Shevach, E. M. Deep sequencing of the TCRB repertoire of human Foxp3+ and Foxp3− T cells suggests that they are completely distinct and non-overlapping. Clin Exp Immunol n/a-n/a (2016). doi:10.1111/cei.12904
13. Lee, E. S., Thomas, P. G., Mold, J. E. & Yates, A. J. Identifying T Cell Receptors from High-Throughput Sequencing: Dealing with Promiscuity in TCRα and TCRβ Pairing. PLOS Computational Biology 13, e1005313 (2017).
14. Poschke, I., Flossdorf, M. & Offringa, R. Next-generation TCR sequencing—a tool to understand T-cell infiltration in human cancers. J. Pathol. n/a-n/a (2016). doi:10.1002/path.4800
15. Marrero, I., Aguilera, C., Hamm, D. E., Quinn, A. & Kumar, V. High-throughput sequencing reveals restricted TCR V3 usage and public TCRβ clonotypes among pancreatic lymph node memory CD4+ T cells and their involvement in autoimmune diabetes. Molecular Immunology 74, 82-95 (2016).
16. Lossius, A. et al. High-throughput sequencing of TCR repertoires in multiple sclerosis reveals intrathecal enrichment of EBV-reactive CD8+ T cells. Eur. J. Immunol. 44, 3439-3452 (2014)
17. Sprouse, M. L. et al. Streamlined Single Cell TCR Isolation and Generation of Retroviral Vectors for In vitro and In vivo Expression of Human TCRs. JoVE (Journal of Visualized Experiments) e55379-e55379 (2017). doi:10.3791/55379
18. Dash, P., Wang, G. C. & Thomas, P. G. Single-Cell Analysis of T-Cell Receptor αβ Repertoire. in Immunosenecence (ed. Shaw, A. C.) 1343, 181-197 (Springer New York, 2015).
19. Guo, X. J. et al. Rapid cloning, expression, and functional characterization of paired αβ and γδ T-cell receptor chains from single-cell analysis. Molecular Therapy—Methods & Clinical Development 3, 15054 (2016).
20. JUNG, S. et al. Sleeping Beauty transposon system harboring HRAS, c-Myc and shp53 induces sarcomatoid carcinomas in mouse skin. Oncol Rep 29, 1293-1298 (2013).
21. Aktas, E., Kucuksezer, U. C., Bilgic, S., Erten, G. & Deniz, G. Relationship between CD107a expression and cytotoxic activity. Cell. Immunol. 254, 149-154 (2009).
22. Betts, M. R., Brenchley, J. M., Price, D. A., De Rosa, S. C., Douek, D. C., Roederer, M. & Koup, R. A. Sensitive and viable identification of antigen-specific CD8+ T cells by a flow cytometric assay for degranulation. J. Immunol. Methods 281, 65-78 (2003).
23. Haney, D., Quigley, M. F., Asher, T. E., Ambrozak, D. R., Gostick, E., Price, D. A., Douek, D. C. & Betts, M. R. Isolation of viable antigen-specific CD8+ T cells based on membrane-bound tumor necrosis factor (TNF)-α expression. J. Immunol. Methods 369, 33-41(2011).
24. Brochet, X., Lefranc, M.-P. & Giudicelli, V. IMGT/V-QUEST: the highly customized and integrated system for IG and TR standardized V-J and V-D-J sequence analysis. Nucleic Acids Res. 36, W503-W508 (2008)
25. Edgar, R. C. Search and clustering orders of magnitude faster than BLAST. Bioinformatics 26, 2460-2461 (2010).
26. Betts, M. R., Brenchley, J. M., Price, D. A., De Rosa, S. C., Douek, D. C., Roederer, M. & Koup, R. A. Sensitive and viable identification of antigen-specific CD8+ T cells by a flow cytometric assay for degranulation. J. Immunol. Methods 281, 65-78 (2003).
27. Haney, D., Quigley, M. F., Asher, T. E., Ambrozak, D. R., Gostick, E., Price, D. A., Douek, D. C. & Betts, M. R. Isolation of viable antigen-specific CD8+ T cells based on membrane-bound tumor necrosis factor (TNF)-a expression. J. Immunol. Methods 369, 33-41(2011).
28. Chattopadhyay, P. K., Yu, J. & Roederer, M. A live-cell assay to detect antigen-specific CD4+ T cells with diverse cytokine profiles. Nat. Med. 11, 1113-1117 (2005).
29. Boswell, K. L., Paris, R., Boritz, E., Ambrozak, D., Yamamoto, T., Darko, S., Wloka, K., Wheatley, A., Narpala, S., McDermott, A., Roederer, M., Haubrich, R., Connors, M., Ake, J., Douek, D. C., Kim, J., Petrovas, C. & Koup, R. A. Loss of Circulating CD4 T Cells with B Cell Helper Function during Chronic HIV Infection. PLoS Pathog 10, e1003853 (2014)
30. Isolation of T-Cell Receptors Specifically Reactive with Mutated Tumor-Associated Antigens from Tumor-Infiltrating Lymphocytes Based on CD137 Expression Maria Parkhurst, Alena Gros, Anna Pasetto, Todd Prickett, Jessica S. Crystal, Paul Robbins and Steven A. Rosenberg
31. Activation-induced expression of CD137 permits detection, isolation, and expansion of the full repertoire of CD8+ T cells responding to antigen without requiring knowledge of epitope specificities Matthias Wolfl,1 JUrgen Kuball,1 William Y. Ho,1 Hieu Nguyen,1 Thomas J. Manley,1 Marie Bleakley,1 and Philip D. Greenberg1,2
32. CD137 Accurately Identifies and Enriches for Naturally Occurring Tumor-Reactive T Cells in Tumor Qunrui Ye, De-Gang Song, Mathilde Poussin, Tori Yamamoto, Andrew Best, Chunsheng Li, George Coukos and Daniel J. Powell Jr. DOI: 10.1158/1078-0432.CCR-13-0945 Published January 2014
33. Cell Immunol. 2009; 254(2):149-54. doi: 10.1016/j.cellimm.2008.08.007. Epub 2008 Oct 5. Relationship between CD107a expression and cytotoxic activity. Aktas E1, Kucuksezer U C, Bilgic S, Erten G, Deniz G.
34. Cannons J L, Lau P, Ghumman B, et al. 4-1BB ligand induces cell division, sustains survival, and enhances effector function of CD4 and CD8 T cells with similar efficacy. J Immunol. 2001; 167:1313-1324.]
35. Lee H W, Park S J, Choi B K, Kim H H, Nam K O, Kwon B S. 4-1BB promotes the survival of CD8+ T lymphocytes by increasing expression of Bcl-xL and Bfl-1. J Immunol. 2002; 169:4882-4888.
36. Ex vivo expansion protocol for human tumor specific T cells for adoptive T cell therapy. Journal of Immunological Methods Volume 355, Issues 1-2, 15 Apr. 2010, Pages 52-60
37. Am J Transplant. 2004 October; 4(10):1614-27 TGF-beta induces Foxp3+ T-regulatory cells from CD4+CD25− precursors. Fu S¹, Zhang N, Yopp A C, Chen D, Mao M, Chen D, Zhang H, Ding Y, Bromberg J S.
38. Science. 2003 Feb. 14; 299(5609):1057-61. Epub 2003 Jan. 9. Control of regulatory T cell development by the transcription factor Foxp3. Hori S1, Nomura T, Sakaguchi S.
39. Wang, Y. et al. J Am Soc Nephrol. 2006 March; 17(3):697-706.
40. Dons, E. et al. Hum Immunol. 2012 April; 73(4):328-34.
41. Nat Immunol. 2014 November; 15(11):1070-8. doi: 10.1038/ni.3004. Epub 2014 Sep. 28. Continuous requirement for the TCR in regulatory T cell function. Levine AG1, Arvey A1, Jin W1, Rudensky AY2.
42. Nat Immunol. 2014 November; 15(11):1070-8. doi: 10.1038/ni.3004. Epub 2014 Sep. 28. Continuous requirement for the TCR in regulatory T cell function. Levine AG1, Arvey A1, Jin W1, Rudensky AY2.
43. Boyd, D., Florent, G., Kim, P. & Brattain, M. Determination of the Levels of Urokinase and Its Receptor in Human Colon Carcinoma Cell Lines. Cancer Res. 48, 3112-3116 (1988).
44. Lorz, A., Botesteanu, D.-A. & Levy, D. Universal response in the RKO colon cancer cell line to distinct antimitotic therapies. Sci. Rep. 8, 8979 (2018).
45. June, C. H., O'Connor, R. S., Kawalekar, O. U., Ghassemi, S. & Milone, M. C. CAR T cell immunotherapy for human cancer. Science 359, 1361-1365 (2018).
46. Kebriaei, P. et al. Phase I trials using Sleeping Beauty to generate CD19-specific CAR T cells. J. Clin. Invest. 126, 3363-3376 (2016).
47. Levine, B. L., Miskin, J., Wonnacott, K. & Keir, C. Global Manufacturing of CAR T Cell Therapy. Mol. Ther.—Methods Clin. Dev. 4, 92-101 (2017).
48. Wang, X. & Riviere, I. Clinical manufacturing of CAR T cells: foundation of a promising therapy. Mol. Ther. —Oncolytics 3, 16015 (2016).
49. Single, A., Beetham, H., Telford, B. J., Guilford, P. & Chen, A. A Comparison of Real-Time and Endpoint Cell Viability Assays for Improved Synthetic Lethal Drug Validation. J. Biomol. Screen. 20, 1286-1293 (2015).
50. Kurtz, A. Mesenchymal Stem Cell Delivery Routes and Fate. Int. J. Stem Cells 1, 1-7 (2008).
51. Khodadoust, M. S. et al. Antigen presentation profiling reveals recognition of lymphoma immunoglobulin neoantigens. Nature 543, 723-727 (2017).
52. Gonzilez, S., Volkova, N., Beer, P. & Gerstung, M. Immuno-oncology from the perspective of somatic evolution. Semin. Cancer Biol. (2017). doi:10.1016/j.semcancer.2017.12.001
53. J.RT3-T3.5 ATCC® TIB-153™ Homo sapiens peripheral blood ac. Available at: https://www.atcc.org/Products/All/TIB-153.aspx. (Accessed: 3 Apr. 2017)
54. Al-Allaf, F. A., Tolmachov, O. E., Zambetti, L. P., Tchetchelnitski, V. & Mehmet, H. Remarkable stability of an instability-prone lentiviral vector plasmid in Escherichia coli Stbl3. 3 Biotech 3, 61-70 (2013).
55. Descours, B. et al. SAMHD1 restricts HIV-1 reverse transcription in quiescent CD4+ T-cells. Retrovirology 9, 87 (2012).
56. DeKosky, B. J. et al. High-throughput sequencing of the paired human immunoglobulin heavy and light chain repertoire. Nat Biotech 31, 166-169 (2013).
57. DeKosky, B. J. et al. In-depth determination and analysis of the human paired heavy- and light-chain antibody repertoire. Nat. Med. 21, 86-91 (2015).
58. McDaniel, J. R., DeKosky, B. J., Tanno, H., Ellington, A. D. & Georgiou, G. Ultra-high-throughput sequencing of the immune receptor repertoire from millions of lymphocytes. Nat. Protoc. 11, 429-42 (2016).
59. DeKosky, B. J. et al. Large-scale sequence and structural comparisons of human naïve and antigen-experienced antibody repertoires. Proc. Natl. Acad. Sci. 113, E2636-E2645 (2016).
60. Tian, M. et al. Induction of HIV Neutralizing Antibody Lineages in Mice with Diverse Precursor Repertoires. Cell 166, 1471-1484.e18 (2016).
61. Wang, B. et al. Functional interrogation and mining of natively paired human V_{H}:V_{L} antibody repertoires. Nat. Biotechnol. 36, 152-155 (2018).
62. Wu, L. C., Tuot, D. S., Lyons, D. S., Garcia, K. C. & Davis, M. M. Two-step binding mechanism for T-cell receptor recognition of peptide-MHC. Nature 418, 552-556 (2002).
63. Zhang, S.-Q. et al. Direct measurement of T cell receptor affinity and sequence from naïve antiviral T cells. Sci. Transl. Med. 8, 341ra77-341ra77 (2016).
64. Vargas, J. E. et al. Retroviral vectors and transposons for stable gene therapy: advances, current challenges and perspectives. J. Transl. Med. 14, 288 (2016).
65. Cohen, C. J. et al. Enhanced Antitumor Activity of T Cells Engineered to Express T-Cell Receptors with a Second Disulfide Bond. Cancer Res. 67, 3898-3903 (2007).
66. Dudley,1, W. N., Wickham,2, R. & COOMBS,3, N. An Introduction to Survival Statistics: Kaplan-Meier Analysis. J. Adv. Pract. Oncol. 7, 91-100 (2016).
67. Birnbaum, M. E. et al. Deconstructing the Peptide-MHC Specificity of T Cell Recognition. Cell 157, 1073-1087 (2014).
68. Glanville, J. et al. Identifying specificity groups in the T cell receptor repertoire. Nature advance online publication, (2017).
69. Han, A., Glanville, J., Hansmann, L. & Davis, M. M. Linking T-cell receptor sequence to functional phenotype at the single-cell level. Nat. Biotechnol. 32, 684-692 (2014).
70. Atkins, M. Clinical manifestations, evaluation, and staging of renal cell carcinoma. UpToDate 18, (2017).
71. Betts, M. R. et al. Sensitive and viable identification of antigen-specific CD8+ T cells by a flow cytometric assay for degranulation. J. Immunol. Methods 281, 65-78 (2003).
72. Aktas, E., Kucuksezer, U. C., Bilgic, S., Erten, G. & Deniz, G. Relationship between CD107a expression and cytotoxic activity. Cell. Immunol. 254, 149-154 (2009).
73. Haney, D. et al. Isolation of viable antigen-specific CD8+ T cells based on membrane-bound tumor necrosis factor (TNF)-a expression. J. Immunol. Methods 369, 33-41(2011).
74. Roybal, K. T. et al. Precision Tumor Recognition by T Cells With Combinatorial Antigen-Sensing Circuits. Cell 164, 770-779 (2016).

### EQUIVALENTS

The present technology is not to be limited in terms of the particular embodiments described in this application, which are intended as single illustrations of individual aspects of the present technology. Many modifications and variations of this technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such

A. A recombinant T cell receptor (TCR) library vector comprising:
(a) a vector backbone; and
(b) (i) a first polynucleotide encoding a TCRα polypeptide and a second polynucleotide encoding a TCRβ polypeptide; or
   (ii) a first polynucleotide encoding a TCRγ polypeptide and a second polynucleotide encoding a TCRδ polypeptide;
wherein the first and second polynucleotides are a cognate pair, and wherein the first polynucleotide and the second polynucleotide are derived from mRNA isolated from a single lysed T cell that is present in a compartment.

B. The vector of Paragraph A, wherein the mRNA of the single lysed T cell is isolated using an mRNA capture reagent or reverse transcription-PCR (RT-PCR).

C. The vector of Paragraph A or Paragraph B, wherein the first polynucleotide and the second polynucleotide are operably linked, optionally via a linker polynucleotide, and optionally wherein the first polynucleotide and the second polynucleotide are operably linked by reverse transcription and PCR amplification of the T cell mRNA.

D. The vector of any one of the previous Paragraphs, wherein the first polynucleotide and the second polynucleotide have been cloned into the vector backbone by cleavage at a target restriction endonuclease site that is natively found in TCR genes.

E. The vector of Paragraph D, wherein the target restriction endonuclease site occurs in TCR genes with low frequency.

F. The vector of Paragraph D or Paragraph E, wherein the first polynucleotide and the second polynucleotide have been altered to incorporate at least one target restriction endonuclease site disclosed in Table 7 or 8.

G. The vector of any one of Paragraphs D-F, wherein the target restriction endonuclease site comprises a silent mutation.

H. The vector of any one of Paragraphs D-G, wherein the mRNA capture reagent is selected from the group consisting of a poly(dT) coated bead, an oligonucleotide-coated bead, a hydrogel bead, and a printed oligo on the surface of a microarray well.

I. The vector of any one of the previous Paragraphs, wherein the compartment is an emulsion droplet or a well.

J. The vector of Paragraph I, wherein the well is located in a printed polymer slide, a plastic plate, a microtiter plate, or a gel.

K. The vector of any one of the previous Paragraphs, wherein the compartment has a volume of 5 nL or less.

L. The vector of any one of the previous Paragraphs, further comprising at least one polynucleotide encoding an expression control element operably linked to the first polynucleotide and/or the second polynucleotide.

M. The vector of Paragraph L, wherein the expression control element is selected from the group consisting of: a promoter, a p2A sequence, and an IRES sequence.

N. The vector of Paragraph M, wherein the promoter is an EF1α promoter or a CMV promoter.

O. The vector of any one of Paragraphs L-N, wherein the polynucleotide encoding the expression control element is located between the first polynucleotide and the second polynucleotide.

P. The vector of any one of the previous Paragraphs, wherein the vector is circularized.

Q. The vector of any one of Paragraphs L-P, wherein the vector has been circularized prior to incorporation of the expression control element into the vector.

R. The vector of any one of Paragraphs L-P, wherein the vector has been circularized after incorporation of the expression control element into the vector.

S. The vector of any one of Paragraphs L-R, wherein the expression control element has been incorporated near a protospacer adjacent motif (PAM).

T. The vector of any one of Paragraphs L-R, wherein the expression control element has been incorporated into the vector using a DNA-modifying enzyme selected from a restriction enzyme or a TALEN.

U. The vector of any one of the previous Paragraphs, further comprising one or more polynucleotides encoding a transposon linked to at least one of the first polynucleotide and the second polynucleotide.

V. The vector of any one of the previous Paragraphs, further comprising a polynucleotide encoding a detectable marker.

W. The vector of any one of the previous Paragraphs, further comprising a polynucleotide encoding a selectable marker.

X. The vector of any one of the previous Paragraphs, further comprising a polynucleotide encoding a switch mechanism for controlling expression and/or activation of the first polynucleotide and the second polynucleotide.

Y. The vector of any one of the previous Paragraphs, further comprising a polynucleotide encoding a Kozak consensus sequence or an enhancer.

Z. The vector of any one of the previous Paragraphs, wherein the vector backbone is selected from a group consisting of a retroviral, a lentiviral, an adenoviral, and an adeno-associated viral vector backbone.

AA. The vector of any one of the previous Paragraphs, wherein the vector encodes a TCR that has binding specificity for a target cell or a disease antigen.

BB. The vector of Paragraph AA, wherein the target cell is a cancer cell or a cell infected with a virus, optionally wherein the target cell was isolated from a subject.

CC. The vector of Paragraph AA, wherein the disease antigen is a viral antigen or a tumor antigen, optionally wherein the antigen is loaded into an antigen:MHC complex.

DD. The vector of Paragraph CC, wherein the disease antigen is a viral antigen derived from a virus selected from the group consisting of adenovirus, CMV, coronavirus, coxsackievirus, Dengue virus, Epstein-Barr virus (EBV), enterovirus 71 (EV71), Ebola virus, hepatitis A (HAV), hepatitis B (HBV), cytomegalovirus (CMV), hepatitis C (HCV), hepatitis D (HDV), hepatitis E (HEV), human immunodeficiency virus (HIV), human papillomavirus (HPV), herpes simplex virus (HSV), human T-lymphotropic virus (HTLV), influenza A virus, influenza B virus, Japanese encephalitis, leukemia virus, measles virus, molluscum contagiosum, orf virus, parvovirus, rabies virus, respiratory syncytial virus, rift valley fever virus, rubella virus, rotavirus, tick-borne encephalitis (TBEV), simian immunodeficiency virus, tobacco etch virus (TEV), varicella zoster virus, variola, West Nile virus, Zika virus, and Chikungunya virus.

EE. The vector of Paragraph CC, wherein the disease antigen is a tumor antigen selected from the group consisting of CD45, glypican-3, IGF2B3, Kallikrein 4, KIF20A, Lengsin, Meloe, mucin 5AC (MUC5AC), survivin, cyclin-A1, MAGE-A1, MAGE-C1, MAGE-C2, SSX2, XAGElb/GAGED2A, CD19, CD20, CD22, CD52, epidermal growth factor receptor (EGFR), human epidermal growth factor receptor 2 (HER2), tumor necrosis factor receptor superfamily, member 10a (TRAILR1), receptor activator of nuclear factor kappa-B ligand (RANKL), insulin-like growth factor 1 receptor (IGF1R), epithelial cell adhesion molecule (EpCAM), and carcinoembryonic antigen (CEA).

FF. A recombinant cell comprising the vector of any one of the previous Paragraphs, optionally wherein the recombinant cell is a bacterial cell, mammalian cell, or a yeast cell.

GG. A recombinant TCR vector library comprising a plurality of vectors according to any one of Paragraphs A-EE.

HH. The recombinant TCR vector library of Paragraph GG, wherein the plurality of vectors comprises a TCR repertoire.

II. The recombinant TCR vector library of Paragraph GG or Paragraph HH, wherein each vector in the plurality of vectors has been selected on the basis of one or more of the following characteristics: TCR clonal prevalence, TCR enrichment characteristics from in vitro assays, TCR binding specificity, TCR V segment sequence, TCR D segment sequence, TCR J segment sequence, TCR gene motifs, and/or CDR3 gene motifs.

JJ. The recombinant TCR vector library of any one of Paragraphs GG-II, wherein the TCR vector library has been characterized by nucleic acid sequencing of the first polynucleotide and the second polynucleotide.

KK. An isolated immune cell comprising the vector of any one of Paragraphs A-EE.

LL. The isolated immune cell of Paragraph KK, wherein the immune cell is a hematopoietic stem cell, a hematopoietic progenitor cell, a T cell, or a natural killer (NK) cell.

MM. A cell population comprising the vector of any one of Paragraphs A-EE, or the vector library of any one of Paragraphs GG-JJ.

NN. The cell population of Paragraph MM, wherein the population comprises hematopoietic stem cells, hematopoietic progenitor cells, T cells, and/or NK cells.

OO. A method for preparing a recombinant TCR library, the method comprising transforming a population of cells with the vector library of any one of Paragraphs GG-JJ.

PP. The method of Paragraph OO, wherein the cells are hematopoietic stem cells, hematopoietic progenitor cells, T cells, or NK cells.

QQ. The method of Paragraph OO or Paragraph PP, further comprising screening the library for specific binding to a target cell.

RR. The method of Paragraph QQ, wherein the target cell is a cancer cell or a cell infected with a virus, optionally wherein the cell was isolated from a subject.

SS. The method of Paragraph OO or Paragraph PP, further comprising screening the library for specific binding to an antigen:MHC complex.

TT. The method of Paragraph SS, wherein the antigen of the antigen:MHC complex is a viral antigen derived from a virus selected from the group consisting of adenovirus, CMV, coronavirus, coxsackievirus, Dengue virus, Epstein-Barr virus (EBV), enterovirus 71 (EV71), Ebola virus, hepatitis A (HAV), hepatitis B (HBV), cytomegalovirus (CMV), hepatitis C (HCV), hepatitis D (HDV), hepatitis E (HEV), human immunodeficiency virus (HIV), human papillomavirus (HPV), herpes simplex virus (HSV), human T-lymphotropic virus (HTLV), influenza A virus, influenza B virus, Japanese encephalitis, leukemia virus, measles virus, molluscum contagiosum, orf virus, parvovirus, rabies virus, respiratory syncytial virus, rift valley fever virus, rubella virus, rotavirus, tick-borne encephalitis (TBEV), simian immunodeficiency virus, tobacco etch virus (TEV), varicella zoster virus, variola, West Nile virus, Zika virus, and Chikungunya virus.

UU. The method of Paragraph SS, wherein the antigen of the antigen:MHC complex is a tumor antigen selected from the group consisting of CD45, glypican-3, IGF2B3, Kallikrein 4, KIF20A, Lengsin, Meloe, mucin 5AC (MUC5AC), survivin, cyclin-A1, MAGE-A1, MAGE-C1, MAGE-C2, SSX2, XAGElb/GAGED2A, CD19, CD20, CD22, CD52, EGFR, HER2, TRAILR1, RANKL, IGF1R, EpCAM, and CEA.

VV. The method of any one of Paragraphs OO-UU, further comprising screening the library for T cell phenotypic markers.

WW. The method of any one of Paragraphs OO-VV, further comprising screening the library for activity in a co-culture system, wherein the co-culture system comprises at least one of the following:
(a) a cancer cell line;
(b) a plurality of cells infected with a known virus;
(c) a plurality of tumor cells isolated from a cancer patient;
(d) an immortalized cell line; or
(e) a plurality of cells derived from a patient tissue biopsy.

XX. The method of any one of Paragraphs OO-WW, further comprising in vitro activation of the transformed population of cells.

YY. The method of Paragraph XX, wherein in vitro activation is performed using one or more of the following stimulants: anti-CD3 antibody, anti-CD8 antibody, anti-CD27 antibody, IL-2, IL-4, IL-21, anti-PD1 antibody, anti-CTLA4 antibody, tumor cell lysate, cellular co-culture with virus-infected cells, and tumor cell lines.

ZZ. The method of any one of Paragraphs OO-YY, further comprising transforming the population of cells with a polynucleotide encoding a transcription factor.

AAA. The method of Paragraph ZZ, wherein the transcription factor is selected from the group consisting of FOXP3, BLIMP-1, Ikaros, Helios and TGF-beta.

BBB. The method of any one of Paragraphs OO-AAA, further comprising selecting individual vectors for inclusion in the recombinant TCR library on the basis of one or more of the following characteristics: TCR clonal prevalence, TCR enrichment characteristics from in vitro assays, TCR binding specificity, TCR V segment sequence, TCR D segment sequence, TCR J segment sequence, TCR gene motifs, and/or CDR3 gene motifs.

CCC. The method of Paragraph BBB, wherein selection comprises mixing individual vectors at a defined ratio to generate a synthetically-derived TCR library.

DDD. A recombinant TCR library prepared by a method according to any one of Paragraphs OO-CCC.

EEE. A composition comprising the recombinant TCR library of Paragraph DDD and a carrier.

FFF. The composition of Paragraph EEE, wherein the carrier is a pharmaceutically acceptable carrier.

GGG. A method of treating a subject in need thereof, the method comprising administering to the subject an effective amount of the recombinant TCR library of Paragraph DDD or the composition of Paragraph EEE or Paragraph FFF to the subject.

HHH. A method of treating cancer in a subject in need thereof, the method comprising administering to the subject an effective amount of the recombinant TCR library Paragraph DDD or the composition of Paragraph EEE or Paragraph FFF to the subject.

III. The method of Paragraph HHH, wherein the cancer is acute lymphoblastic leukemia (ALL); acute myeloid leukemia (AML); adrenocortical carcinoma; AIDS-related cancers; anal cancer; appendix cancer; astrocytomas; atypical teratoid/rhabdoid tumor, brain cancer; basal cell carcinoma of the skin; bile duct cancer; bladder cancer; bone cancer; breast cancer; bronchial tumors; Burkitt lymphoma; carcinoid tumor (gastrointestinal); germ cell tumor; primary CNS lymphoma; cervical cancer; cholangiocarcinoma; chordoma; chronic lymphocytic leukemia (CLL); chronic myelogenous leukemia (CML); chronic myeloproliferative neoplasms; colorectal cancer; craniopharyngioma; cutaneous T-cell lymphoma; ductal carcinoma in situ (DCIS); endometrial cancer; ependymoma; esophageal cancer; esthesioneuroblastoma; extracranial germ cell tumor; extragonadal germ cell tumor; eye cancer; intraocular melanoma; retinoblastoma; fallopian tube cancer; fibrous histiocytoma of bone, malignant, and osteosarcoma; gallbladder cancer; gastric cancer; gastrointestinal carcinoid tumor; gastrointestinal stromal tumors (GIST); germ cell tumors; gestational trophoblastic disease; hairy cell leukemia; head and neck cancer; heart tumors; hepatocellular cancer; histiocytosis, Langerhans cell; Hodgkin lymphoma; hypopharyngeal cancer; intraocular melanoma; islet cell tumors, pancreatic neuroendocrine tumors; kidney cancer; laryngeal cancer; leukemia; lip and oral cavity cancer; liver cancer; lung cancer; lymphoma; male breast cancer; malignant fibrous histiocytoma of bone and osteosarcoma; melanoma; Merkel cell carcinoma; mesothelioma; metastatic cancer; mouth cancer; multiple endocrine neoplasia syndrome; multiple myeloma/plasma cell neoplasms; mycosis fungoides; myelodysplastic syndrome, myeloproliferative neoplasm, chronic; nasopharyngeal cancer; neuroblastoma; Non-Hodgkin lymphoma; non-small cell lung cancer; oral cancer, oropharyngeal cancer; osteosarcoma; ovarian cancer; pancreatic cancer; pancreatic neuroendocrine tumors; papillomatosis; paraganglioma; paranasal sinus cancer; parathyroid cancer; pharyngeal cancer; pheochromocytoma; pituitary tumor; pleuropulmonary blastoma; prostate cancer; rectal cancer; recurrent cancer; renal cell cancer; retinoblastoma; rhabdomyosarcoma; salivary gland cancer; sarcoma; Ewing sarcoma; Kaposi sarcoma; osteosarcoma; uterine sarcoma; Sézary syndrome; skin cancer; small cell lung cancer; small intestine cancer; soft tissue sarcoma; squamous cell carcinoma of the skin; squamous neck cancer; stomach cancer; T cell lymphoma; testicular cancer; throat cancer; nasopharyngeal cancer; hypopharyngeal cancer; thymic carcinoma; thyroid cancer; urethral cancer; uterine cancer; vaginal cancer; vascular tumors; vulvar cancer; or Wilms tumor.

JJJ. A method of inhibiting tumor growth in a subject in need thereof, the method comprising administering to the subject an effective amount of the recombinant TCR library of Paragraph DDD or the composition of Paragraph EEE or Paragraph FFF to the subject.

KKK. The method of Paragraph JJJ, wherein the tumor is a solid tumor.

LLL. A method of treating a viral infection in a subject in need thereof, the method comprising administering to the subject an effective amount of the recombinant TCR library of Paragraph DDD or the composition of Paragraph EEE or Paragraph FFF to the subject.

MMM. The method of Paragraph LLL, wherein the viral infection is caused by a virus selected from the group consisting of adenovirus, CMV, coronavirus, coxsackievirus, Dengue virus, Epstein-Barr virus (EBV), enterovirus 71 (EV71), Ebola virus, hepatitis A (HAV), hepatitis B (HBV), cytomegalovirus (CMV), hepatitis C (HCV), hepatitis D (HDV), hepatitis E (HEV), human immunodeficiency virus (HIV), human papillomavirus (HPV), herpes simplex virus (HSV), human T-lymphotropic virus (HTLV), influenza A virus, influenza B virus, Japanese encephalitis, leukemia virus, measles virus, molluscum contagiosum, orf virus, parvovirus, rabies virus, respiratory syncytial virus, rift valley fever virus, rubella virus, rotavirus, tick-borne encephalitis (TBEV), simian immunodeficiency virus, tobacco etch virus (TEV), varicella zoster virus, variola, West Nile virus, Zika virus, and Chikungunya virus.

NNN. The method of any one of Paragraphs GGG-MMM, further comprising administering a second dose of the recombinant TCR library of Paragraph DDD or the composition of Paragraph EEE or Paragraph FFF to the subject.

OOO. The method of any one of Paragraphs GGG-NNN, wherein the recombinant TCR library comprises cells that are autologous or allogenic to the subject being treated.

PPP. The method of any one of Paragraphs GGG-OOO, wherein the subject is a human, an animal, a non-human primate, a dog, cat, a sheep, a mouse, a horse, or a cow.

QQQ. The method of Paragraph PPP, wherein the subject is a human.

Other embodiments are set forth within the following claims.

What is claimed is:

1. A recombinant T cell receptor (TCR) library vector comprising:
(a) a vector backbone; and
(b) (i) a first polynucleotide encoding a TCRα polypeptide and a second polynucleotide encoding a TCRβ polypeptide; or
(ii) a first polynucleotide encoding a TCRγ polypeptide and a second polynucleotide encoding a TCRδ polypeptide;
wherein the first and second polynucleotides are a cognate pair, and wherein the first polynucleotide and the second polynucleotide are derived from mRNA isolated from a single lysed T cell that is present in a compartment.

2. The vector of claim 1, wherein the mRNA of the single lysed T cell is isolated using an mRNA capture reagent or reverse transcription-PCR (RT-PCR).

3. The vector of claim 1, wherein the first polynucleotide and the second polynucleotide have been cloned into the vector backbone by cleavage at a target restriction endonuclease site that is natively found in TCR genes.

4. The vector of claim 1, further comprising at least one polynucleotide encoding an expression control element operably linked to the first polynucleotide and/or the second polynucleotide.

5. The vector of claim 4, wherein the polynucleotide encoding the expression control element is located between the first polynucleotide and the second polynucleotide.

6. The vector of claim 1, further comprising one or more polynucleotides encoding a transposon linked to at least one of the first polynucleotide and the second polynucleotide.

7. The vector of claim 1, wherein the vector encodes a TCR that has binding specificity for a target cell or a disease antigen.

8. The vector of claim 7, wherein the disease antigen is a viral antigen or a tumor antigen, optionally wherein the antigen is loaded into an antigen:MHC complex.

9. A recombinant cell comprising the vector of claim 1, optionally wherein the recombinant cell is a bacterial cell, mammalian cell, or a yeast cell.

10. A recombinant TCR vector library comprising a plurality of vectors according to claim 1.

11. An isolated immune cell comprising the vector of claim 1.

12. A cell population comprising the vector of claim 1.

13. A cell population comprising the vector library of claim 10.

14. A composition comprising the recombinant TCR vector library of claim 10 and a carrier.

15. A method of treating cancer in a subject in need thereof, the method comprising administering to the subject an effective amount of the recombinant TCR vector library of claim 10.

16. A method of treating cancer in a subject in need thereof, the method comprising administering to the subject an effective amount of the composition of claim 14 to the subject.

17. A method of inhibiting tumor growth in a subject in need thereof, the method comprising administering to the subject an effective amount of the recombinant TCR library of claim 10.

18. A method of inhibiting tumor growth in a subject in need thereof, the method comprising administering to the subject an effective amount of the composition of claim 14 to the subject.

19. A method of treating a viral infection in a subject in need thereof, the method comprising administering to the subject an effective amount of the recombinant TCR library of claim 10.

20. A method of treating a viral infection in a subject in need thereof, the method comprising administering to the subject an effective amount of the composition of claim 14 to the subject.
